(19) United States
(12) Patent Application Publication
(10) Pub. No.: US 2025/0206773 A1
(43) Pub. Date: Jun. 26, 2025

(54) FORMS AND COMPOSITIONS OF SODIUM CHENODEOXYCHOLATE

(71) Applicant: Syntis Bio, Inc., Boston, MA (US)

(72) Inventors: Caterina Napoletano, Laveno Mombello (VA) (IT); Marco Marino, Torino (TO) (IT); Chiara Vladiskovic, Milano (MI) (IT); Alessandro Restelli, Bareggio (MI) (IT); Corina-Mihaela Manta, Bucharest (RO); Andrei-Alunel Patrascu, Bucharest (RO)

(21) Appl. No.: 18/851,936

(22) PCT Filed: Mar. 28, 2023
(86) PCT No.: PCT/US2023/016486

§ 371 Date: Sep. 27, 2024

(30) Foreign Application Priority Data
Mar. 29, 2022 (IT) 102022000006161
Sep. 26, 2022 (IT) 102022000019725

(51) Int. Cl. C07J 9/00; A61K 31/575

(52) U.S. Cl. CPC C07J 9/005; A61K 31/575; C07B 2200/13

(57) ABSTRACT

The present disclosure provides solid forms of sodium chenodeoxycholate (NaCDC), as well as compositions thereof and methods of using and preparing the same.

### RELATED APPLICATIONS

This application claims priority to and benefit of Italian Application No. 102022000006161, filed Mar. 29, 2022, and Italian Application No. 102022000019725, filed Sep. 26, 2022, the entire contents of each of which are hereby incorporated by reference.

### BACKGROUND

Chenodeoxycholic acid (CDCA), a bile acid, has been used as therapy, including, for example, for patients suffering from gallstones, albeit with diarrhea as a side effect. Additionally, use of sodium chenodeoxycholate (NaCDC) as a treatment for constipation has been investigated. See, e.g., Rao, A. S., et al., Gastroenterology, 2010 November; 139(5):1549-1558.e.1.

### SUMMARY

The present disclosure recognizes a need for new formulations of chenodeoxycholic acid (CDCA) or salts thereof, and provides such formulations and related technologies (e.g., methods, compositions, etc.).

In particular, although a formulation comprising a CDCA salt (sodium chenodeoxycholate, NaCDC) in a Eudragit S100-coated capsule was shown to be effective in treating symptoms of irritable bowel syndrome with constipation (IBS-C), nearly half of patients suffered abdominal pain and cramping. See Rao, A. S., et al., Gastroenterology, 2010 November; 139(5):1549-1558.e.1. Steiger et al. hypothesized that such side effects may be driven by high local concentrations of chenodeoxycholate (CDC) when released in the intestinal tract, but further recognized that effective release of CDC from a controlled release system may be challenging due to decreasing water content along the colon. Therefore, they preliminarily evaluated a coated bilayer tablet formulation comprising an immediate release layer and an extended release layer, and demonstrated promising in vitro release patterns, as well as reduced incidence of massive contractions when rectally administering the tablet to swine. See Steiger, C., et al., Clin. Transl. Gastroenterology 2020; 11:e00229. The present disclosure provides an insight that development of formulations comprising CDCA or a salt thereof continues to be an important aspect of establishing new therapies for gastrointestinal disorders, such as IBS-C.

The present disclosure provides solid forms of sodium chenodeoxycholate (NaCDC), as well as compositions thereof and methods of using and preparing the same, and technologies related thereto. In some embodiments, provided solid forms are useful for treating gastrointestinal disorders, such as constipation (e.g., IBS-C). In some embodiments, provided solid forms are useful in the preparation of new formulations of NaCDC, e.g., formulations uniquely designed to address certain problems with prior formulations of CDCA or a salt thereof.

### BRIEF DESCRIPTION OF THE DRAWINGS

FIG. 1 is an X-ray powder diffraction (XRPD) pattern of NaCDC Form A.

FIG. 2 is a differential scanning calorimetry (DSC) curve of NaCDC Form A.

FIG. 3 is a thermogravimetric analysis (TGA) curve of NaCDC Form A.

FIG. 4 is a series of XRPD patterns, showing NaCDC Form A before (thick line) and after (thin line) 15 hours of exposure to RH 95% at room temperature.

FIG. 5 is an X-ray powder diffraction (XRPD) pattern of NaCDC Form B.

FIG. 6 is a differential scanning calorimetry (DSC) curve of NaCDC Form B.

FIG. 7 is a thermogravimetric analysis (TGA) curve of NaCDC Form B.

FIG. 8 is a series of XRPD patterns, showing NaCDC Form B before (thick line) and after (thin line) 15 hours of exposure to RH 95% at room temperature.

FIG. 9 is a dynamic vapor sorption (DVS) plot of NaCDC Form A.

FIG. 10 is a DVS plot of NaCDC Form B.

FIG. 11 shows intrinsic dissolution profiles of NaCDC Form A and NaCDC Form B.

FIG. 12 shows linear regression analysis of the intrinsic dissolution profiles of NaCDC Form A and NaCDC Form B.

FIG. 13 shows solubility curves for NaCDC Form A and NaCDC Form B.

FIG. 14 is a DSC curve of NaCDC Form B.

FIG. 15 is a plot of TG (top)/DSC (bottom) analysis of NaCDC Form B.

FIG. 16 is a series of XRPD patterns, showing material obtained after heating NaCDC Form B at 315° C. (bottom) and 360° C. (top).

FIG. 17 is a series of XRPD patterns, showing material obtained after exposing NaCDC Form B to UV 254 nm light under various conditions. From bottom to top: starting material; clear vial; amber vial; control.

FIG. 18 is a series of XRPD patterns, showing NaCDC Form B (bottom) and material obtained from experiment ST19 (middle) and ST20 (top).

FIG. 19A is an XRPD pattern of Form S1, obtained from experiment ST19.

FIG. 19B is a plot of TG (top)/DSC (bottom) analysis of Form S1.

FIG. 20 is a series of XRPD patterns, obtained from certain solubility experiments. From bottom to top: NaCDC Form B; NaCDC Form B after heating at 315° C.; ST19; SASO3; SAS07; SAS08; SAS24; SAS33; SAS39; SAS46; SAS47; and SAS48.

FIG. 21A is a plot of TG (top)/DSC (bottom) analysis of Form S2.

FIG. 21B is an XRPD pattern of Form S3, obtained from experiment SAS07.

FIG. 22 is a plot of TG (top)/DSC (bottom) analysis of Form S4.

FIG. 23A is a series of XRPD patterns, showing material obtained from experiment SAS47 (bottom) and FASO8 (top).

FIG. 23B is a plot of TG (top)/DSC (bottom) analysis of Form S5.

FIG. 24A is a series of XRPD patterns, showing material obtained from experiments ST19, SL13, SL39, and RAS11 (from bottom to top).

FIG. 24B is a plot of TG (top)/DSC (bottom) analysis of Form S1 (or a form similar to and/or isomorphic with Form S1).

FIG. 25 is a plot of TG (top)/DSC (bottom) analysis of Form S2.

FIG. 26A is a series of XRPD patterns, showing material obtained from experiments SL27 (bottom) and SL56 (top).

FIG. 26B is a plot of TG (top)/DSC (bottom) analysis of Form S7.

FIG. 27 is a plot of TG (top)/DSC (bottom) analysis of Form S1 (or a form similar to and/or isomorphic with Form S1).

FIG. 28 is a plot of TG (top)/DSC (bottom) analysis of Form S5.

FIG. 29 is a plot of TG (top)/DSC (bottom) analysis of Form S6.

FIG. 30 is a plot of TG (top)/DSC (bottom) analysis of Form S2.

FIG. 31A is an XRPD pattern of Form S4.

FIG. 31B is a plot of TG (top)/DSC (bottom) analysis of Form S4.

FIG. 32A is a plot of TG (top)/DSC (bottom) analysis of Form S5.

FIG. 32B is a plot of TG (top)/DSC (bottom) analysis of Form S5.

FIG. 33 is an XRPD pattern of Form S2.

FIG. 34A is a series of XRPD patterns, showing material obtained from experiment VDS012 (top) and NaCDC Form B (bottom).

FIG. 34B is a plot of TG (top)/DSC (bottom) analysis of Form S8.

FIG. 35 is a series of XRPD patterns, showing material obtained from experiments ASDS17, ASDS21, ST19, ASDS03, ASDS03 stored for about three weeks, and SDGR03 (bottom to top).

FIG. 36 is a plot of TG (top)/DSC (bottom) analysis of Form S14.

FIG. 37 is a series of XRPD patterns, showing material obtained from experiments ASDS04, ASDS04 stored for 22 days, ASDS08 stored for 23 days, NaCDC monohydrate, ASDS08, and NaCDC hemihydrate (bottom to top).

FIG. 38 is a plot of TG (top)/DSC (bottom) analysis of Form S9-a obtained from a sample from experiment ASDS04 that had been stored for 22 days.

FIG. 39 is a plot of TG (top)/DSC (bottom) analysis of Form S9-a obtained from a sample from experiment ASDS08 that had been stored for 23 days.

FIG. 40A is an XRPD spectrum of Form S10.

FIG. 40B is a plot of TG (top)/DSC (bottom) analysis of Form S10.

FIG. 41 is a plot of TG (top)/DSC (bottom) analysis of Form S11.

FIG. 42 is a series of XRPD patterns, showing material obtained from experiment ASDS19 (bottom), a sample from experiment ASDS19 that had been stored for 22 days (middle), and experiment RAS36.

FIG. 43 is a plot of TG (top)/DSC (bottom) analysis of Form S12.

FIG. 44 is a series of XRPD patterns, showing material obtained from experiment ASDS16 before (bottom) and after (top) storage for 22 days.

FIG. 45 is a plot of TG (top)/DSC (bottom) analysis of Form S13.

FIG. 46A is an XRPD spectrum of Form S14.

FIG. 46B is a plot of TG (top)/DSC (bottom) analysis of Form 514.

FIG. 47 is a plot of TG (top)/DSC (bottom) analysis of Form S5.

FIG. 48 is a series of XRPD patterns, showing material obtained from experiment RAS11 before (bottom) and after (top) storage at RT for two weeks.

FIG. 49 is a plot of TG (top)/DSC (bottom) analysis of Form S1 (or a form similar to and/or isomorphic with Form S1).

FIG. 50A is a series of XRPD patterns, showing material obtained from experiments SAS07, RAS34, RAS33, and ASDS18 (bottom to top).

FIG. 50B is a plot of TG (top)/DSC (bottom) analysis of Form S3 (or a form similar to and/or isomorphic with Form S3).

FIG. 51 is series of XRPD patterns, showing material obtained from experiment RAS33 before (bottom) and after (middle) storage at RT for two weeks and Form S1 (top—obtained from experiment RAS11).

FIG. 52 is a plot of TG (top)/DSC (bottom) analysis of Form S6 (or a form similar to and/or isomorphic with Form S6).

FIG. 53 is an XRPD pattern of material obtained from experiment RAS037.

FIG. 54A is a series of XRPD patterns, showing material obtained from experiment RAS36 before (bottom) and after (middle) storage at RT in solution for two weeks and Form S14 (top—obtained from experiment ASDS17).

FIG. 54B is a plot of TG (top)/DSC (bottom) analysis of material obtained from experiment RAS36 after storage at RT for two weeks.

### DETAILED DESCRIPTION

### Chenodeoxycholic Acid

Chenodeoxycholic acid (CDCA) is a bile acid having the following structure:

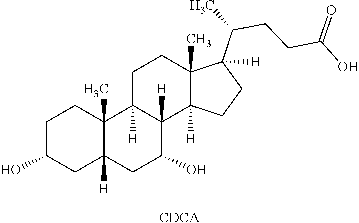

There remains a need for identifying salt and/or solid forms of CDCA useful for various therapeutic applications, such as those described herein. It would be desirable to provide a form (e.g., a salt and/or solid form) of CDCA that, as compared to another form of CDCA (e.g., an amorphous form), imparts characteristics such as improved stability, solubility, hygroscopicity (e.g., provided forms may be less hygroscopic than another form), bioavailability, pharmacokinetics, and/or ease of formulation.

### Sodium Chenodeoxycholate

In some embodiments, the present disclosure provides salt forms of CDCA (e.g., sodium salt forms of CDCA, also referred to as sodium chenodeoxycholate or NaCDC). In some embodiments, the present disclosure provides a crystalline solid form of sodium chenodeoxycholate (NaCDC). In some embodiments, the present disclosure provides one or more polymorphic solid forms of NaCDC. As used herein, the term “polymorph” refers to the ability of a compound to exist in one or more different crystal structures. For example, one or more polymorphs may vary in pharmaceutically relevant physical properties between one form and another, e.g., solubility, stability, and/or hygroscopicity.

It will be appreciated that a crystalline solid form of NaCDC may exist in a neat or unsolvated form, a hydrated form, a solvated form, and/or a heterosolvated form. In some embodiments, a crystalline solid form of NaCDC does not have any water or solvent incorporated into the crystalline structure (i.e., is “unsolvated” or an “anhydrate”). In some embodiments, a crystalline solid form of NaCDC comprises water and/or solvent in the crystalline structure (i.e., are hydrates and/or solvates, respectively). As used herein, the term “solvate” refers to a solid form with a stoichiometric or non-stoichiometric amount of one or more solvents incorporated into the crystal structure. For example, a solvated or heterosolvated polymorph can comprise 0.05, 0.1, 0.2, 0.5, 1.0, 1.5, 2.0, etc. equivalents independently of one or more solvents incorporated into the crystal lattice. As used herein, the term “hydrate” refers to a solvate, wherein the solvent incorporated into the crystal structure is water. It will be appreciated that solvates comprising only certain solvents (most notably, water) are suitable for development as a drug. See Zhang, C., et al., J. Pharm. Sci., 2018, 107(10): 2731-34. Solvates comprising other solvents may be useful for manufacturing and/or testing, inter alia, even if they may not be acceptable for use in an approved pharmaceutical product.

In some embodiments, the present disclosure provides NaCDC as a hydrate (e.g., a sesquihydrate). In some embodiments, the present disclosure provides NaCDC as an anhydrate.

In some embodiments, the present disclosure provides NaCDC as a solvate (e.g., a solvate of ethanol, ethyl acetate, methanol, methyl tert-butyl ether, trifluoroethanol, or water, or any combination thereof).

It will further be appreciated that the term “salt” or “salt form” encompasses complexes of CDCA and an acid or base, including those resulting from an ionic interaction between CDCA and an acid or base, as well as non-ionic associations between CDCA and a neutral species. In some embodiments, provided salt forms result from an ionic interaction between CDCA and a base (e.g., a sodium base, such that the resulting form is NaCDC).

As used herein, the term “about” when used in reference to a degree 2-theta value refers to the stated value ±0.2 degrees 2-theta. In other embodiments, provided degree 2-theta values refer to the stated value ±0.1 degrees 2-theta.

### Form A

In some embodiments, the present disclosure provides sodium chenodeoxycholate as Form A. In some embodiments, Form A is a hydrate (e.g., a sesquihydrate).

In some embodiments, Form A is characterized by one or more peaks in its XRPD pattern selected from those at about 6.07, about 6.55, about 10.72, about 14.64, about 15.06, about 17.58, and about 18.34 degrees 2-theta. In some embodiments, Form A is characterized by two or more peaks in its XRPD pattern selected from those at about 6.07, about 6.55, about 10.72, about 14.64, about 15.06, about 17.58, and about 18.34 degrees 2-theta. In some embodiments, Form A is characterized by three or more peaks in its XRPD pattern selected from those at about 6.07, about 6.55, about 10.72, about 14.64, about 15.06, about 17.58, and about 18.34 degrees 2-theta. In some embodiments, Form A is characterized by four or more peaks in its XRPD pattern selected from those at about 6.07, about 6.55, about 10.72, about 14.64, about 15.06, about 17.58, and about 18.34 degrees 2-theta. In some embodiments, Form A is characterized by five or more peaks in its XRPD pattern selected from those at about 6.07, about 6.55, about 10.72, about 14.64, about 15.06, about 17.58, and about 18.34 degrees 2-theta. In some embodiments, Form A is characterized by six or more peaks in its XRPD pattern selected from those at about 6.07, about 6.55, about 10.72, about 14.64, about 15.06, about 17.58, and about 18.34 degrees 2-theta.

In some embodiments, Form A is characterized by peaks in its XRPD pattern selected from those at about 6.07, about 6.55, about 10.72, about 14.64, about 15.06, about 17.58, and about 18.34 degrees 2-theta. In some embodiments, Form A is characterized by peaks in its XRPD pattern at substantially all of:

2θ ± 0.2 [°]

6.07
6.55
8.36
9.92
10.72
11.55
11.93
12.99
13.36
14.64
15.06
15.54
15.93
16.68
16.99
17.31
17.58
18.07
18.34

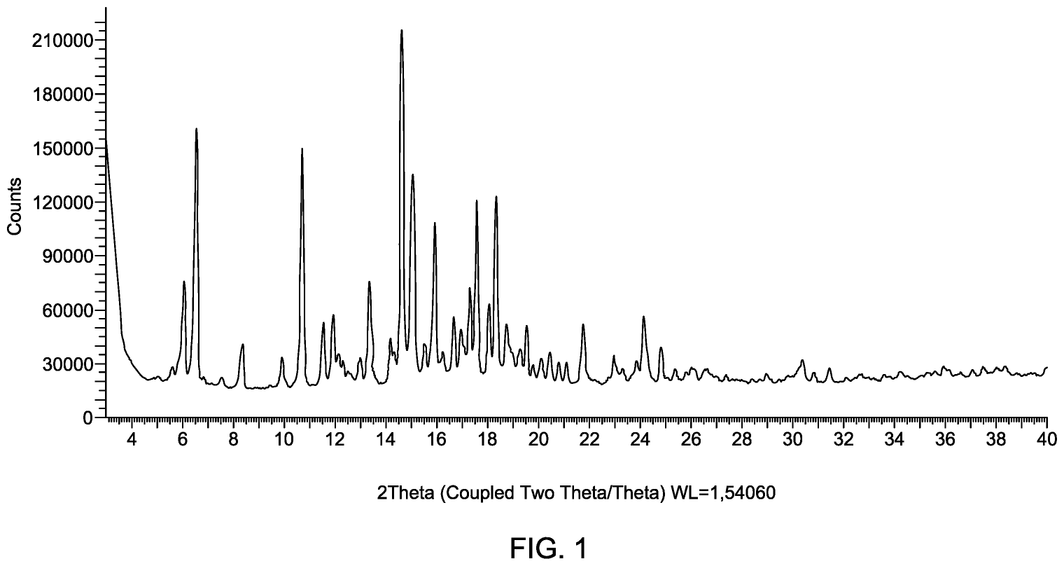

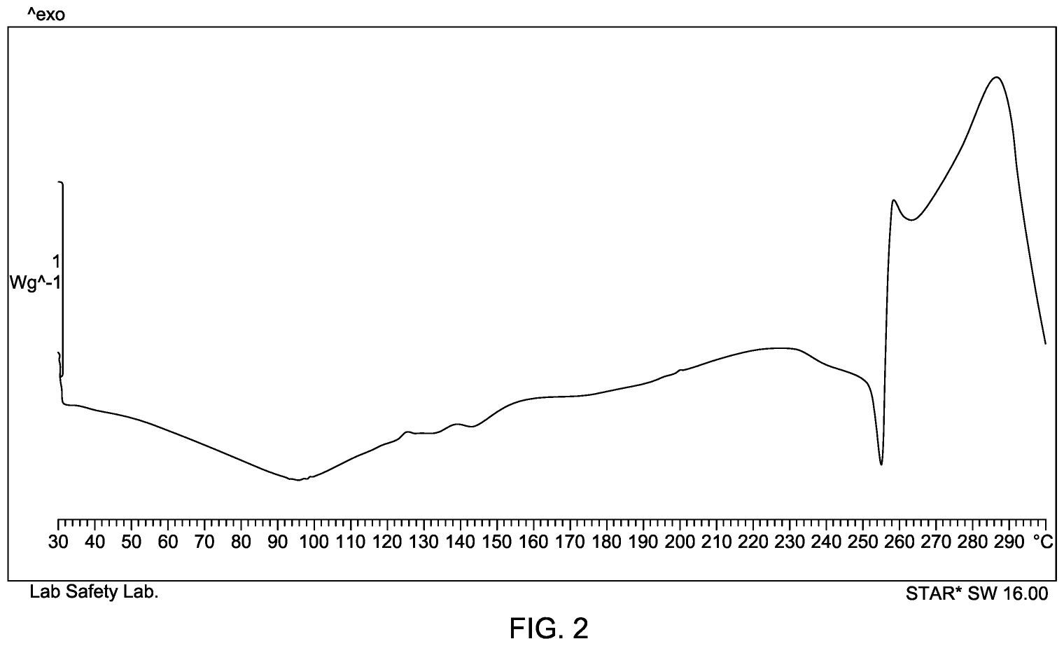

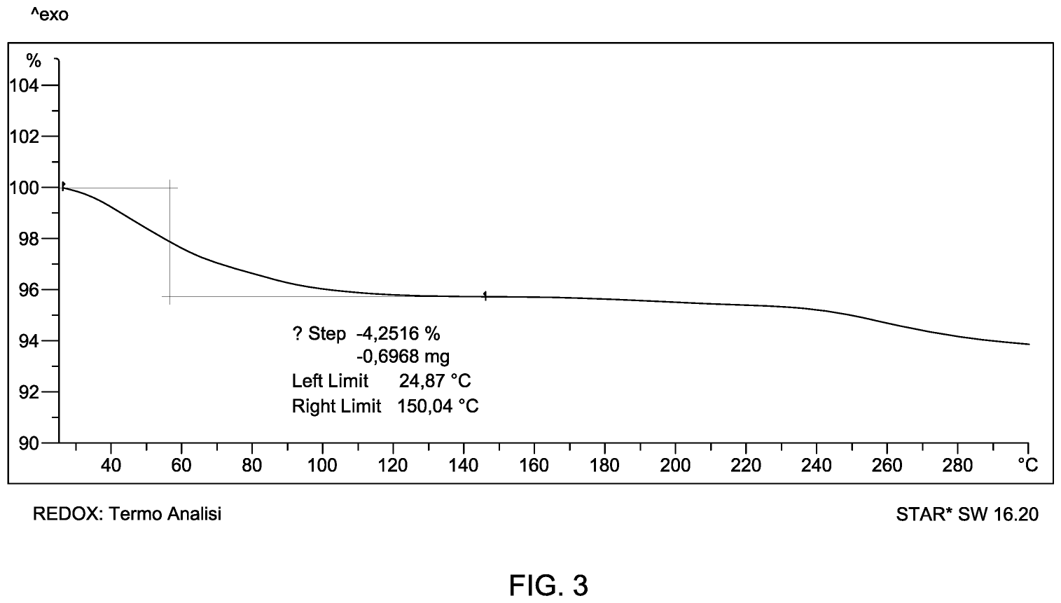

In some embodiments, Form A is characterized by one or more of the following:
(i) an XRPD pattern substantially similar to that depicted in FIG. 1;
(ii) a DSC pattern showing loss of water starting just above ambient temperature and continuing to about 150° C.;
(iii) a DSC pattern substantially similar to that depicted in FIG. 2;
(iv) a TGA pattern showing a weight loss of 4.3% up to 150° C.; and
(v) a TGA pattern substantially similar to that depicted in FIG. 3.

### Form B

In some embodiments, the present disclosure provides sodium chenodeoxycholate as Form B. In some embodiments, Form B is an anhydrate.

In some embodiments, Form B is characterized by one or more peaks in its XRPD pattern selected from those at about 6.75, about 8.14, about 9.79, about 14.02, about 16.10, and about 18.63 degrees 2-theta. In some embodiments, Form B is characterized by two or more peaks in its XRPD pattern selected from those at about 6.75, about 8.14, about 9.79, about 14.02, about 16.10, and about 18.63 degrees 2-theta. In some embodiments, Form B is characterized by three or more peaks in its XRPD pattern selected from those at about 6.75, about 8.14, about 9.79, about 14.02, about 16.10, and about 18.63 degrees 2-theta. In some embodiments, Form B is characterized by four or more peaks in its XRPD pattern selected from those at about 6.75, about 8.14, about 9.79, about 14.02, about 16.10, and about 18.63 degrees 2-theta. In some embodiments, Form B is characterized by five or more peaks in its XRPD pattern selected from those at about 6.75, about 8.14, about 9.79, about 14.02, about 16.10, and about 18.63 degrees 2-theta.

In some embodiments, Form B is characterized by peaks in its XRPD pattern selected from those at about 6.75, about 8.14, about 9.79, about 14.02, about 16.10, and about 18.63 degrees 2-theta. In some embodiments, Form B is characterized by peaks in its XRPD pattern at substantially all of:

2θ ± 0.2 [°]

6.75
8.14
9.79
11.33
13.27
13.56
14.02
14.79
15.05
16.10
16.37
16.78
17.74
18.63
19.71
20.00
20.35
21.89
22.90

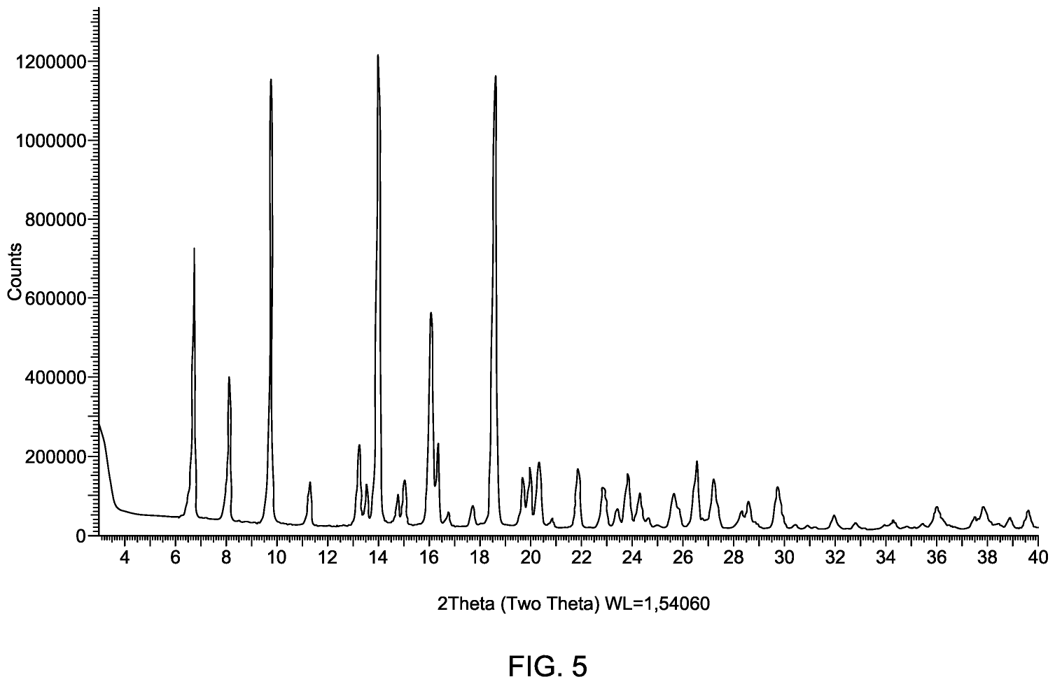

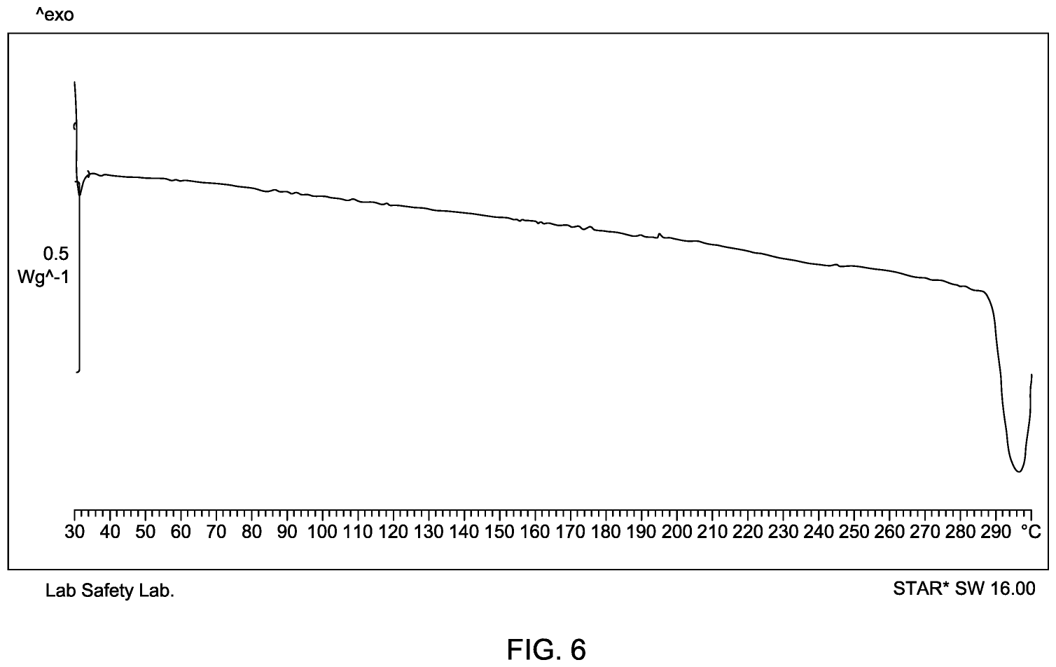

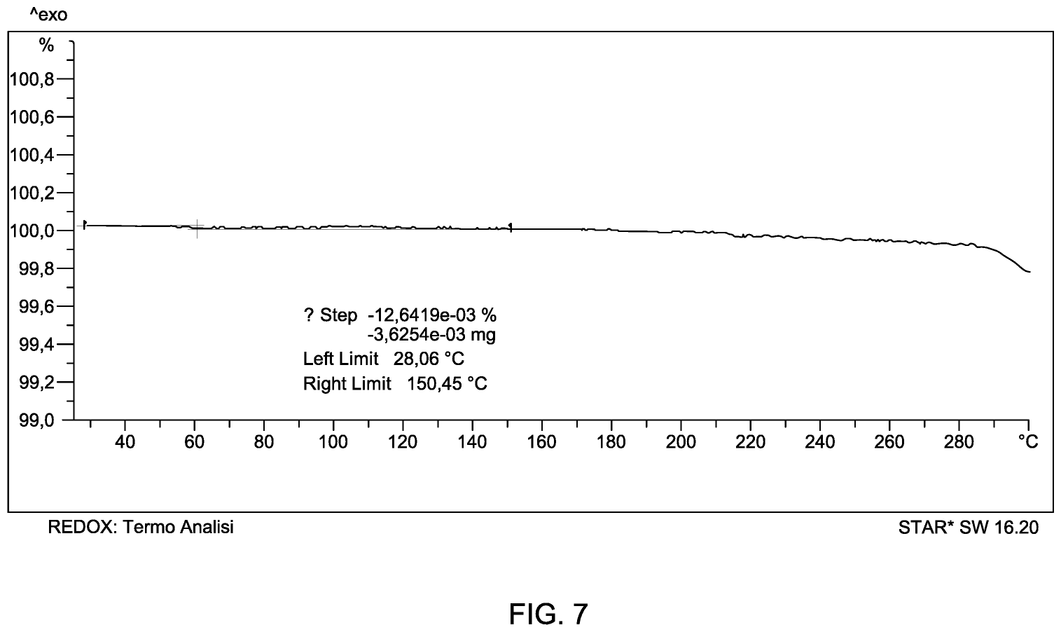

In some embodiments, Form B is characterized by one or more of the following:
(i) an XRPD pattern substantially similar to that depicted in FIG. 5;
(ii) a DSC pattern showing no thermal event from room temperature to around 288° C.;
(iii) a DSC pattern substantially similar to that depicted in FIG. 6;
(iv) a TGA pattern showing a weight loss of <0.1% up to 150° C.; and
(v) a TGA pattern substantially similar to that depicted in FIG. 7.

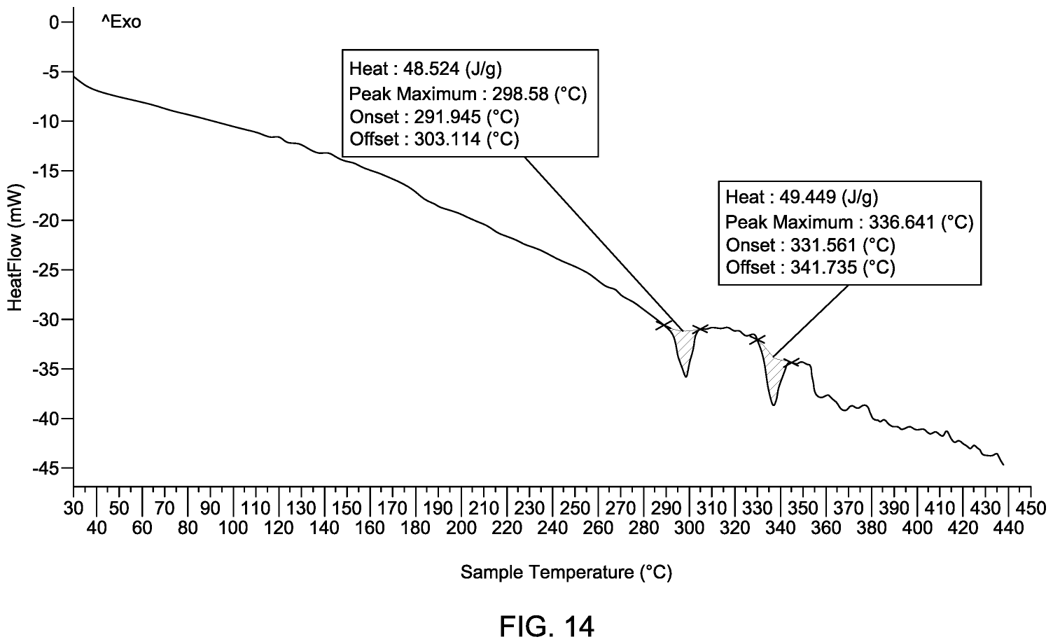

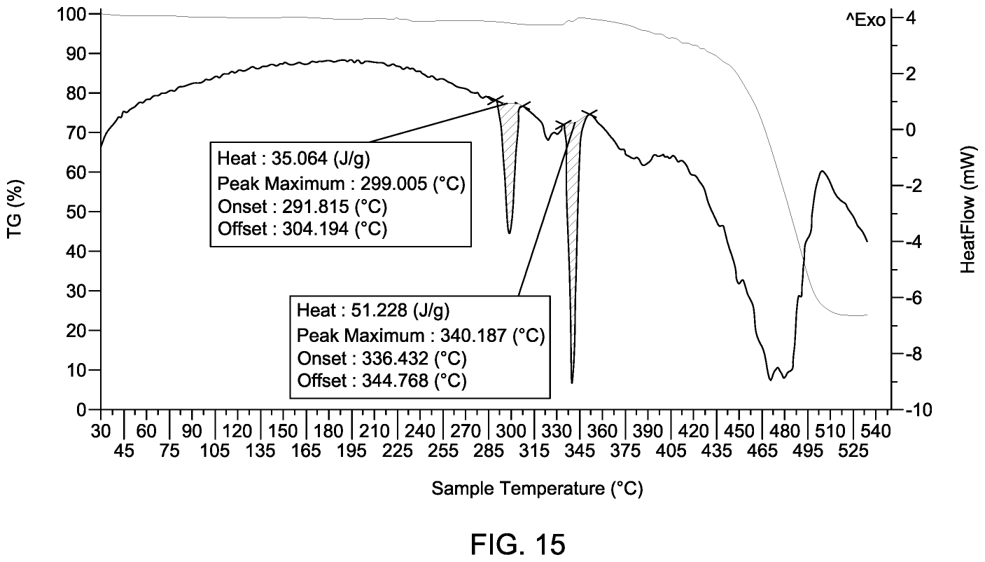

In some embodiments, Form B is characterized by one or more of the following:
(i) an XRPD pattern substantially similar to that depicted in FIG. 5;
(ii) a DSC pattern showing no thermal event from room temperature to around 288° C.;
(iii) a DSC pattern substantially similar to that depicted in FIG. 6;
(iv) a TGA pattern showing a weight loss of <0.1% up to 150° C.;
(v) a TGA pattern substantially similar to that depicted in FIG. 7;
(vi) a DSC pattern substantially similar to that depicted in FIG. 14;
(vii) a TGA pattern substantially similar to that depicted in FIG. 15; and
(viii) a DSC pattern substantially similar to that depicted in FIG. 15.

### Form S1

In some embodiments, the present disclosure provides sodium chenodeoxycholate as Form S1. In some embodiments, Form S1 is a solvate. In some embodiments, Form S1 is a methyl ethyl ketone solvate. In some embodiments, Form S1 is a methyl tert-butyl ether solvate. In some embodiments, Form S1 is a trifluoroethanol solvate. In some embodiments, Form S1 is an acetone solvate. In some embodiments, Form S1 is a hydrate.

In some embodiments, “Form S1” refers to one or more similar and/or isomorphic forms that are characterized by feature(s) described herein.

In some embodiments, Form S1 is characterized by one or more peaks in its XRPD pattern selected from those at about 5.45, about 5.80, about 7.46, about 9.76, about 12.40, about 14.88, and about 20.02 degrees 2-theta. In some embodiments, Form S1 is characterized by two or more peaks in its XRPD pattern selected from those at about 5.45, about 5.80, about 7.46, about 9.76, about 12.40, about 14.88, and about 20.02 degrees 2-theta. In some embodiments, Form S1 is characterized by three or more peaks in its XRPD pattern selected from those at about 5.45, about 5.80, about 7.46, about 9.76, about 12.40, about 14.88, and about 20.02 degrees 2-theta. In some embodiments, Form S1 is characterized by four or more peaks in its XRPD pattern selected from those at about 5.45, about 5.80, about 7.46, about 9.76, about 12.40, about 14.88, and about 20.02 degrees 2-theta. In some embodiments, Form S1 is characterized by five or more peaks in its XRPD pattern selected from those at about 5.45, about 5.80, about 7.46, about 9.76, about 12.40, about 14.88, and about 20.02 degrees 2-theta. In some embodiments, Form S1 is characterized by six or more peaks in its XRPD pattern selected from those at about 5.45, about 5.80, about 7.46, about 9.76, about 12.40, about 14.88, and about 20.02 degrees 2-theta.

In some embodiments, Form S1 is characterized by peaks in its XRPD pattern selected from those at about 5.45, about 5.80, about 7.46, about 9.76, about 12.40, about 14.88, and about 20.02 degrees 2-theta. In some embodiments, Form S1 is characterized by peaks in its XRPD pattern at substantially all of those listed in Table S1-A. In some embodiments, Form S1 is characterized by peaks in its XRPD pattern at substantially all of those listed in Table S1-B.

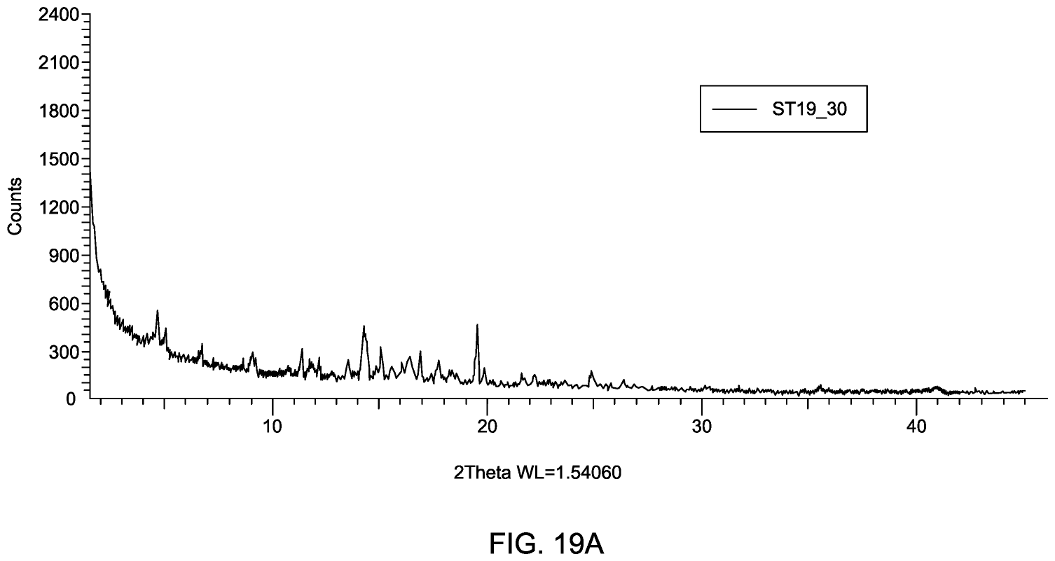

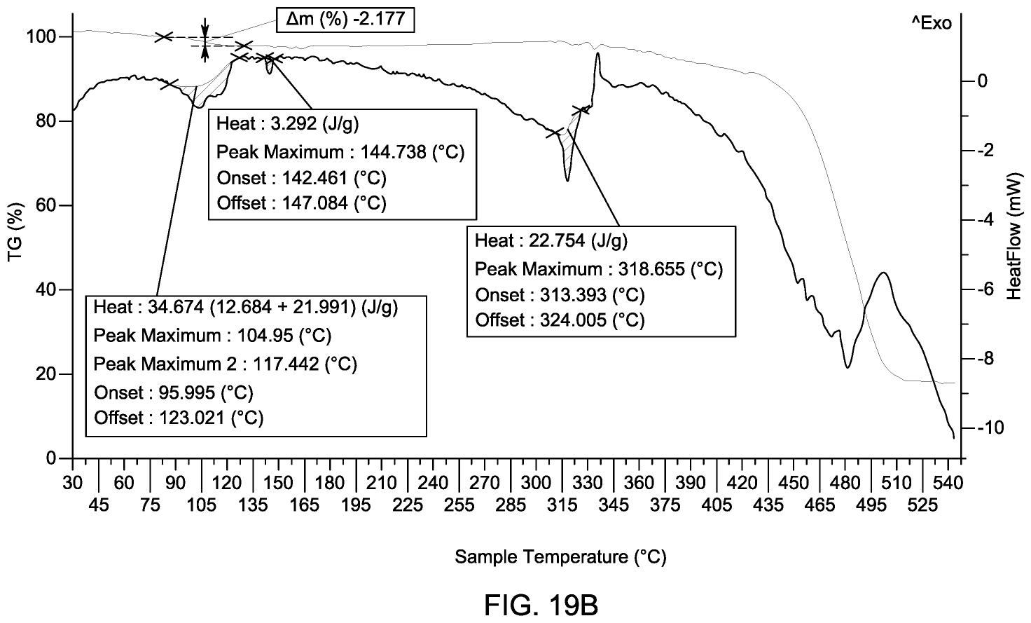

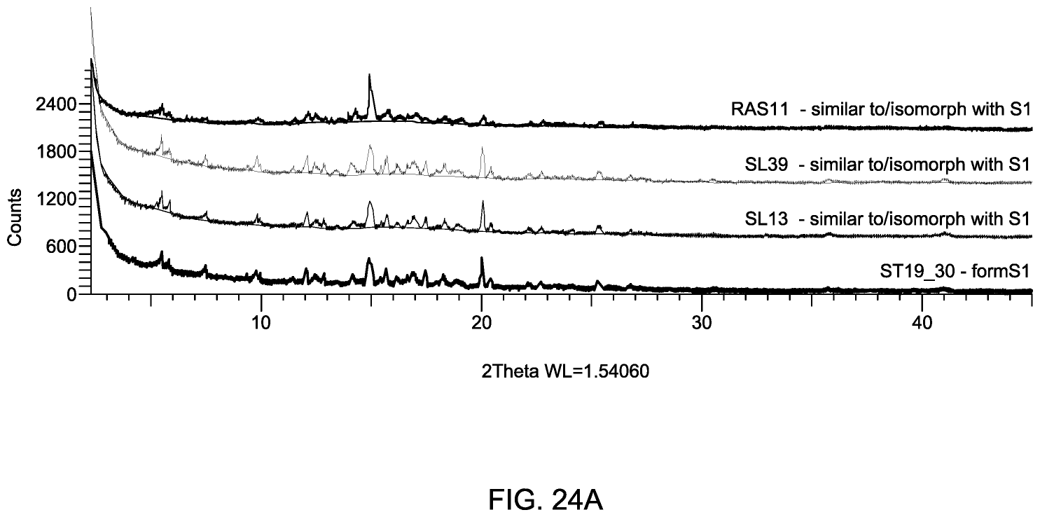

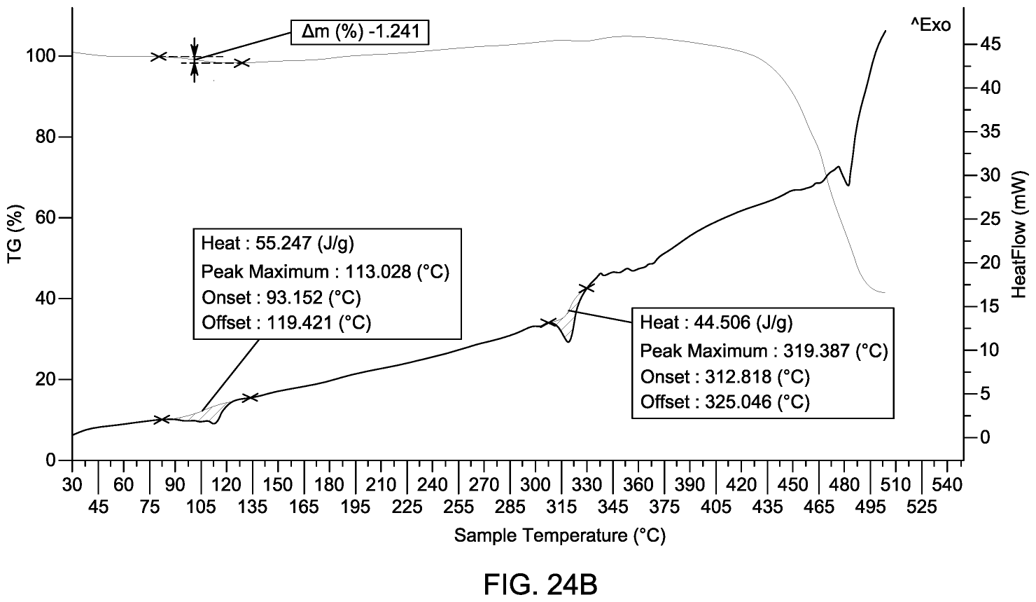

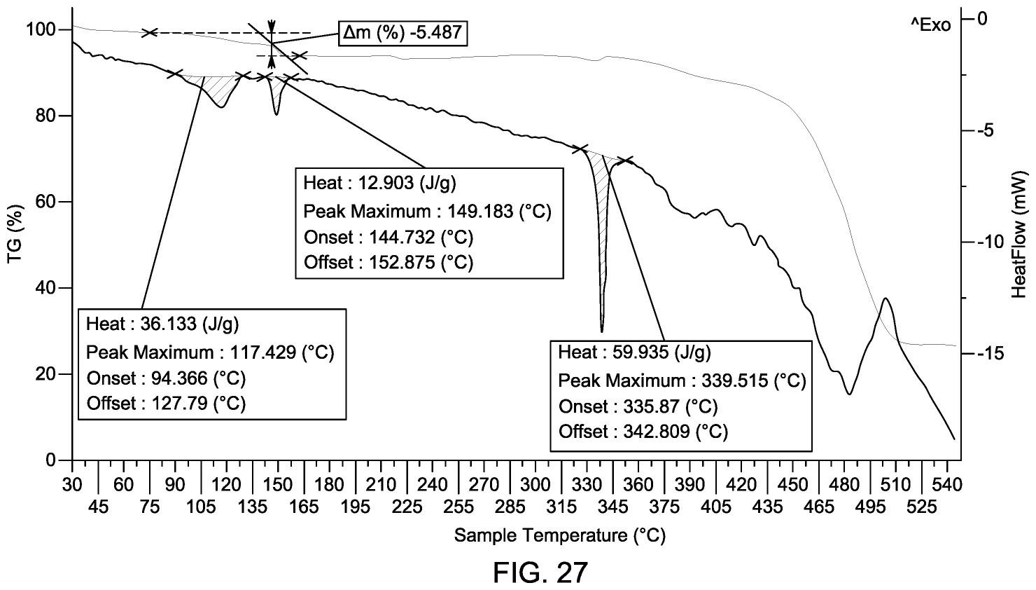

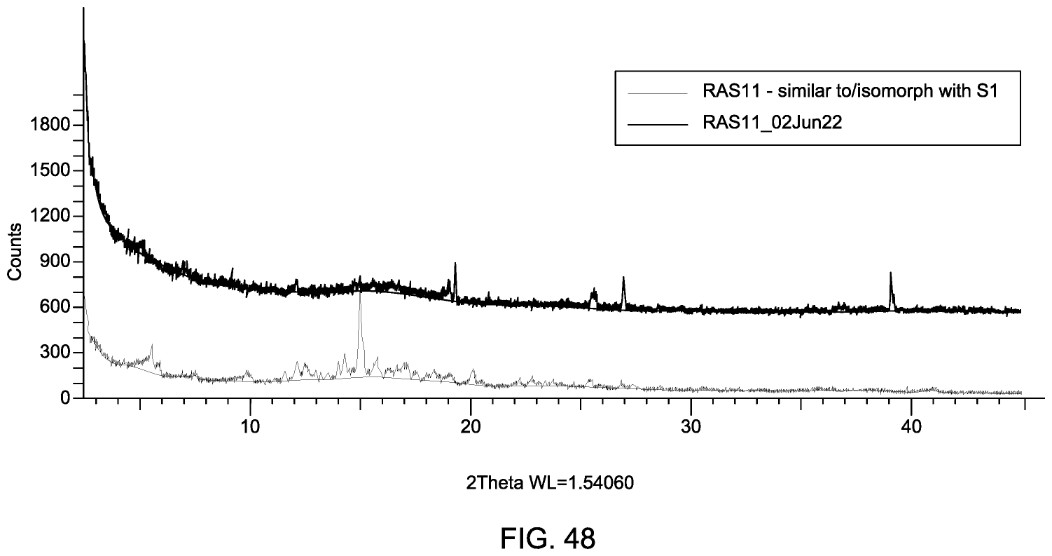

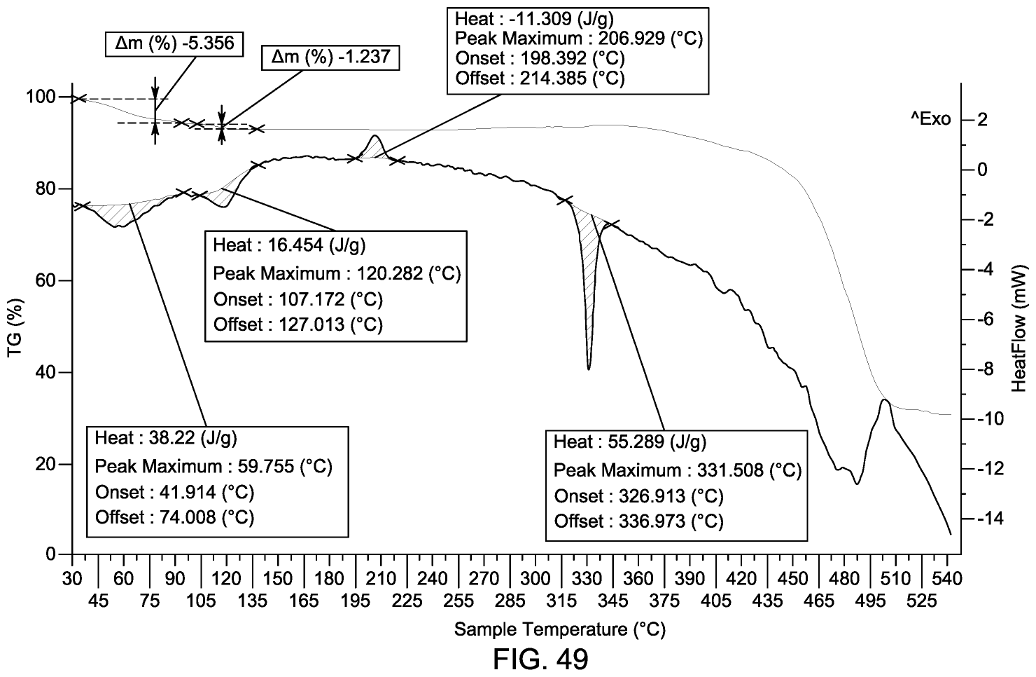

In some embodiments, Form S1 is characterized by one or more of the following:
(i) an XRPD pattern substantially similar to that depicted in FIG. 19A, FIG. 24A, and/or FIG. 48;
(ii) a DSC pattern showing a thermal event at about 96.0° C., about 142.5° C., and/or about 313.4° C.;
(iii) a DSC pattern substantially similar to that depicted in FIG. 19B, FIG. 24B, FIG. 27, and/or FIG. 49;
(iv) a TGA pattern showing a weight loss of about 2.177% up to about 150° C.; and
(v) a TGA pattern substantially similar to that depicted in FIG. 19B, FIG. 24B, FIG. 27, and/or FIG. 49.

### Form S2

In some embodiments, the present disclosure provides sodium chenodeoxycholate as Form S2. In some embodiments, Form S2 is a solvate. In some embodiments, Form S2 is an ethanol solvate.

In some embodiments, Form S2 is characterized by one or more peaks in its XRPD pattern selected from those at about 7.11, about 7.78, about 9.81, about 12.58, about 12.96, and about 13.54 degrees 2-theta. In some embodiments, Form S2 is characterized by two or more peaks in its XRPD pattern selected from those at about 7.11, about 7.78, about 9.81, about 12.58, about 12.96, and about 13.54 degrees 2-theta. In some embodiments, Form S2 is characterized by three or more peaks in its XRPD pattern selected from those at about 7.11, about 7.78, about 9.81, about 12.58, about 12.96, and about 13.54 degrees 2-theta. In some embodiments, Form S2 is characterized by four or more peaks in its XRPD pattern selected from those at about 7.11, about 7.78, about 9.81, about 12.58, about 12.96, and about 13.54 degrees 2-theta. In some embodiments, Form S2 is characterized by five or more peaks in its XRPD pattern selected from those at about 7.11, about 7.78, about 9.81, about 12.58, about 12.96, and about 13.54 degrees 2-theta.

In some embodiments, Form S2 is characterized by peaks in its XRPD pattern selected from those at about 7.11, about 7.78, about 9.81, about 12.58, about 12.96, and about 13.54 degrees 2-theta. In some embodiments, Form S2 is characterized by peaks in its XRPD pattern at substantially all of those listed in Table S2.

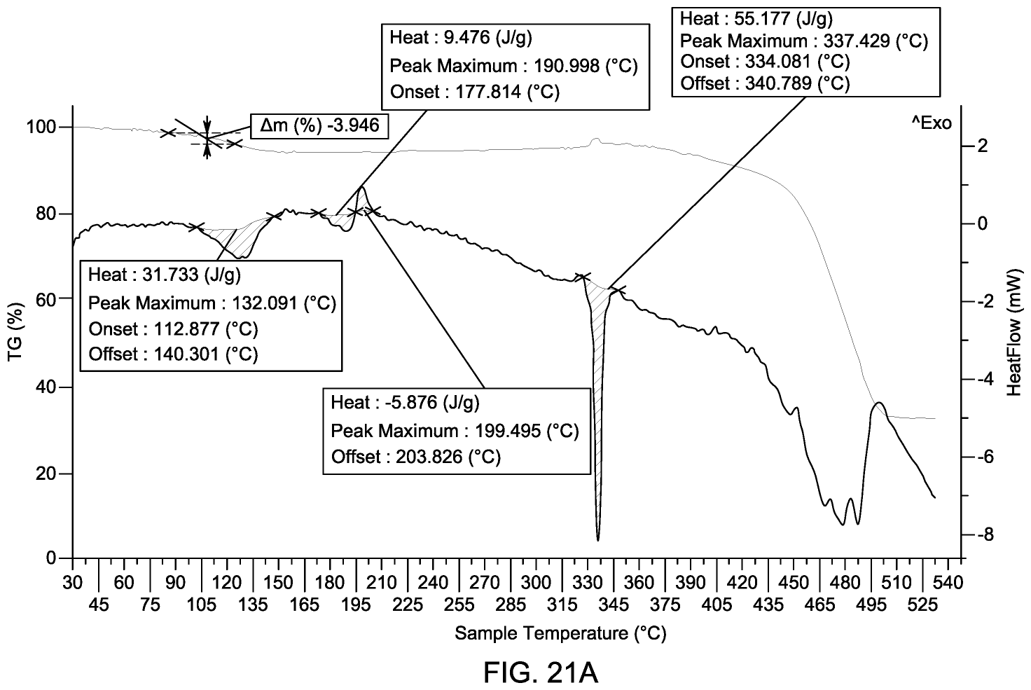

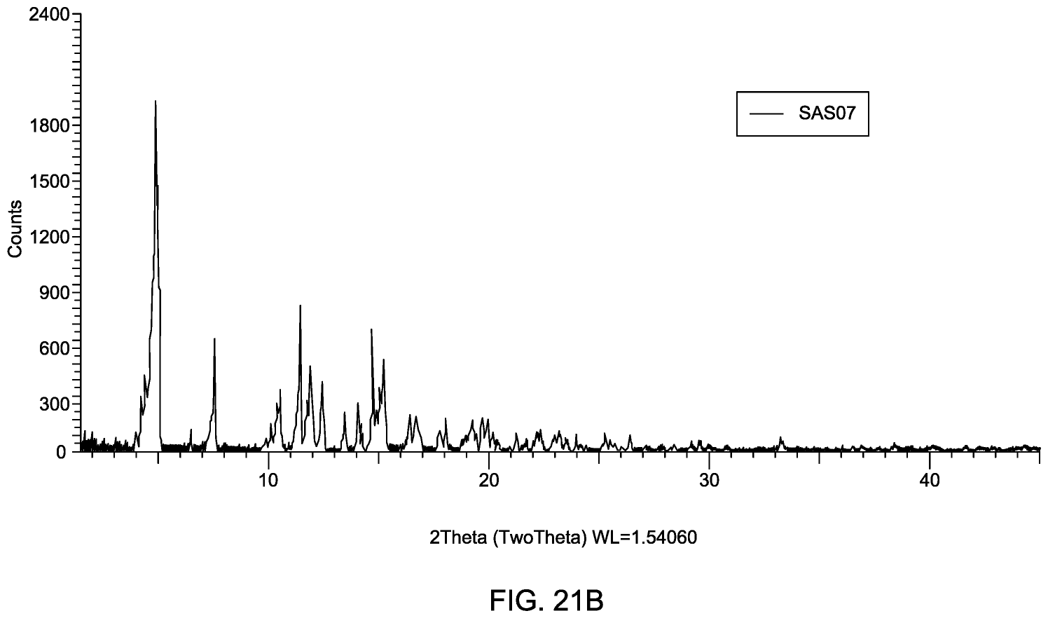

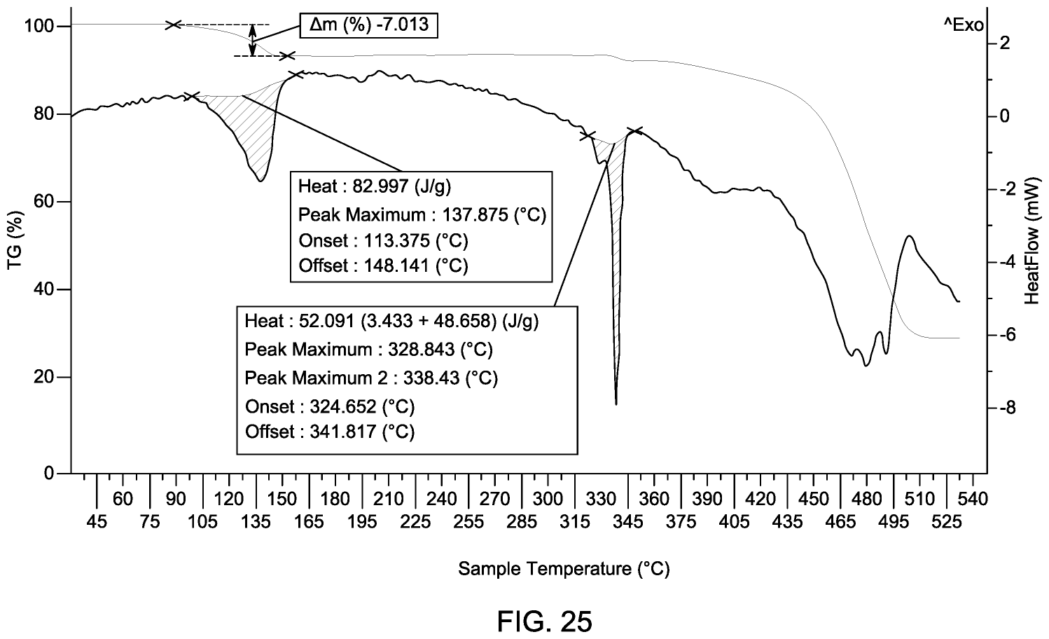

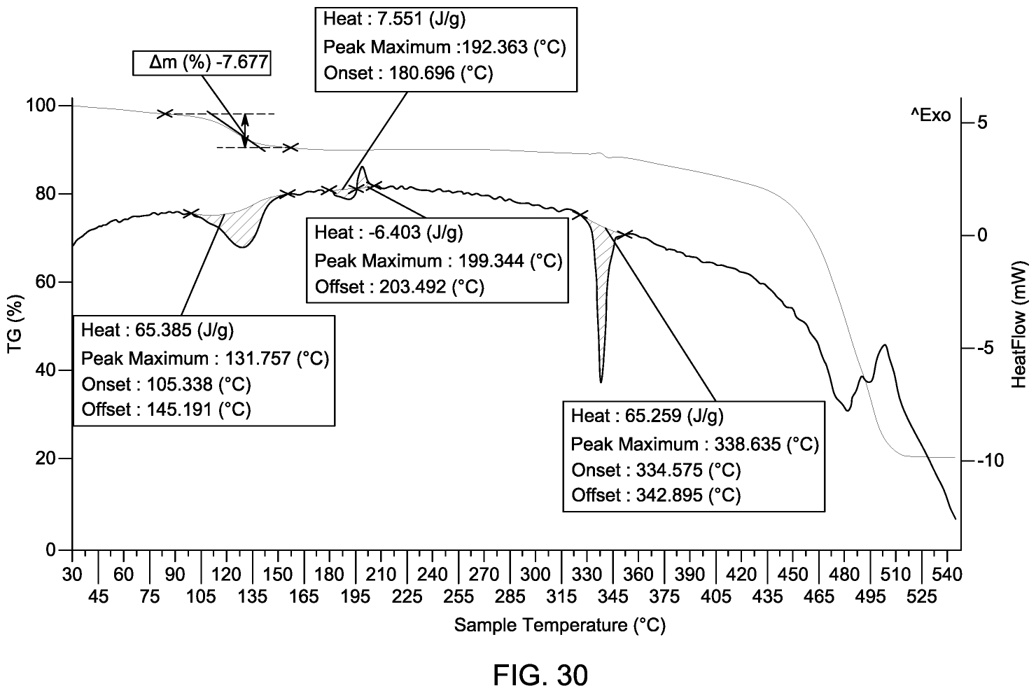

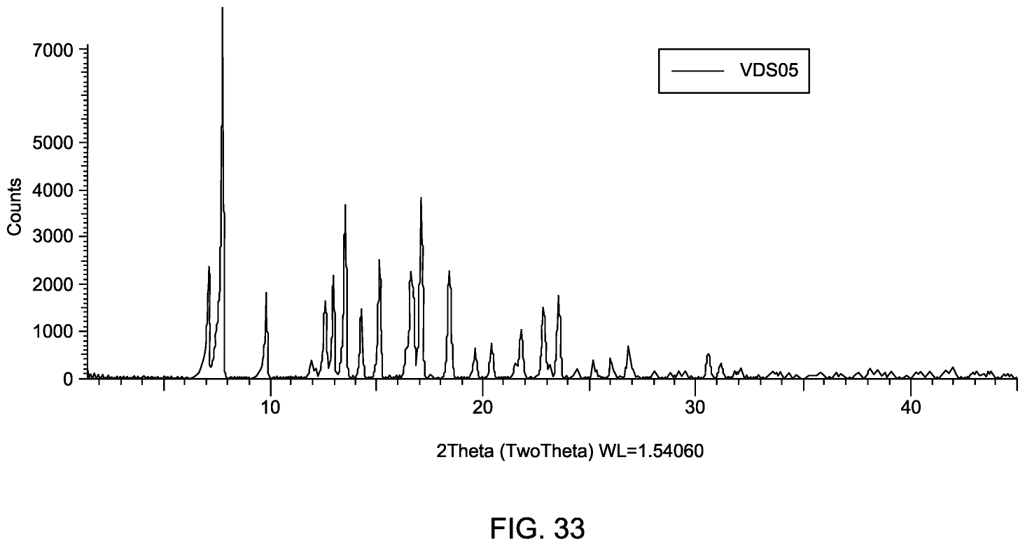

In some embodiments, Form S2 is characterized by one or more of the following:
(i) an XRPD pattern substantially similar to that depicted in FIG. 33;
(ii) a DSC pattern showing a thermal event at about 112.9° C., about 177.8° C., and/or about 334.1° C.;
(iii) a DSC pattern substantially similar to that depicted in FIG. 21A, FIG. 25, and/or FIG. 30;
(iv) a TGA pattern showing a weight loss of about 3.95% up to about 150° C.; and (v) a TGA pattern substantially similar to that depicted in FIG. 21A, FIG. 25, and/or FIG. 30.

### Form S3, Form S6, and Form S11

In some embodiments, the present disclosure provides sodium chenodeoxycholate as Form S3, Form S6, and/or Form S11. In some embodiments, Form S3 and/or Form S11 is an anhydrate. In some embodiments, Form S6 is a solvate. In some embodiments, Form S6 is an ethyl acetate solvate. In some embodiments, Form S3, Form S6, and Form S11 are similar to and/or isomorphic with each other. Therefore, in some embodiments, Form S3, Form S6, and/or Form S11 share one or more features described herein.

In some embodiments, Form S3, Form S6, and/or Form S11 are characterized by one or more peaks in its XRPD pattern selected from those at about 5.00, about 7.56, about 10.56, about 11.45, about 11.93, and about 12.46 degrees 2-theta. In some embodiments, Form S3, Form S6, and/or Form S11 are characterized by two or more peaks in its XRPD pattern selected from those at about 5.00, about 7.56, about 10.56, about 11.45, about 11.93, and about 12.46 degrees 2-theta. In some embodiments, Form S3, Form S6, and/or Form S11 are characterized by three or more peaks in its XRPD pattern selected from those at about 5.00, about 7.56, about 10.56, about 11.45, about 11.93, and about 12.46 degrees 2-theta. In some embodiments, Form S3, Form S6, and/or Form S11 are characterized by four or more peaks in its XRPD pattern selected from those at about 5.00, about 7.56, about 10.56, about 11.45, about 11.93, and about 12.46 degrees 2-theta. In some embodiments, Form S3, Form S6, and/or Form S11 are characterized by five or more peaks in its XRPD pattern selected from those at about 5.00, about 7.56, about 10.56, about 11.45, about 11.93, and about 12.46 degrees 2-theta.

In some embodiments, Form S3, Form S6, and/or Form S11 are characterized by peaks in its XRPD pattern selected from those at about 5.00, about 7.56, about 10.56, about 11.45, about 11.93, and about 12.46 degrees 2-theta. In some embodiments, Form S3, Form S6, and/or Form S11 are characterized by peaks in its XRPD pattern at substantially all of those listed in Table S3. In some embodiments, Form S3, Form S6, and/or Form S11 are characterized by peaks in its XRPD pattern at substantially all of those listed in Table S6. In some embodiments, Form S3, Form S6, and/or Form S11 are characterized by peaks in its XRPD pattern at substantially all of those listed in Table S11.

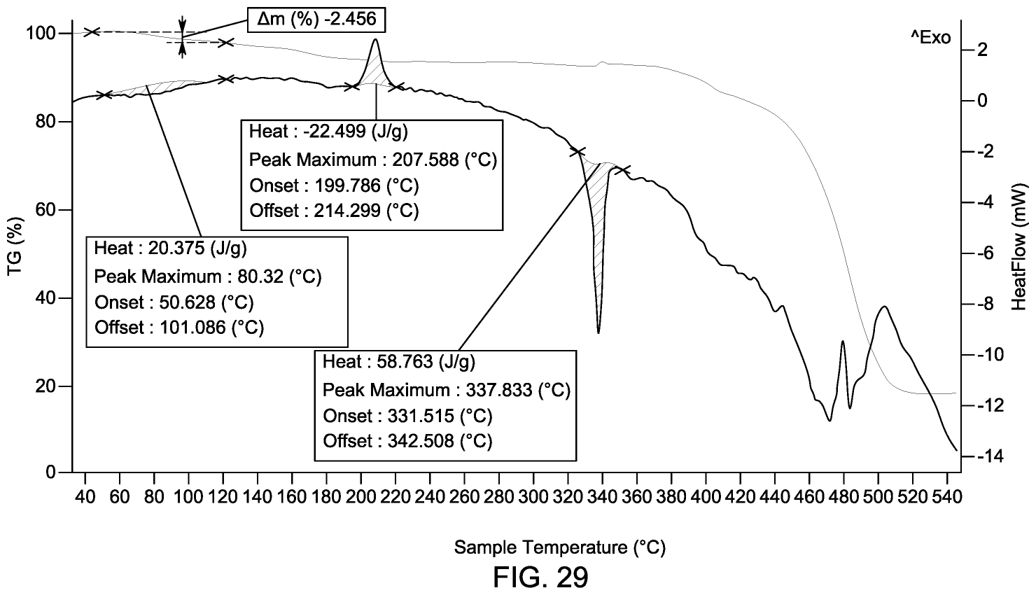

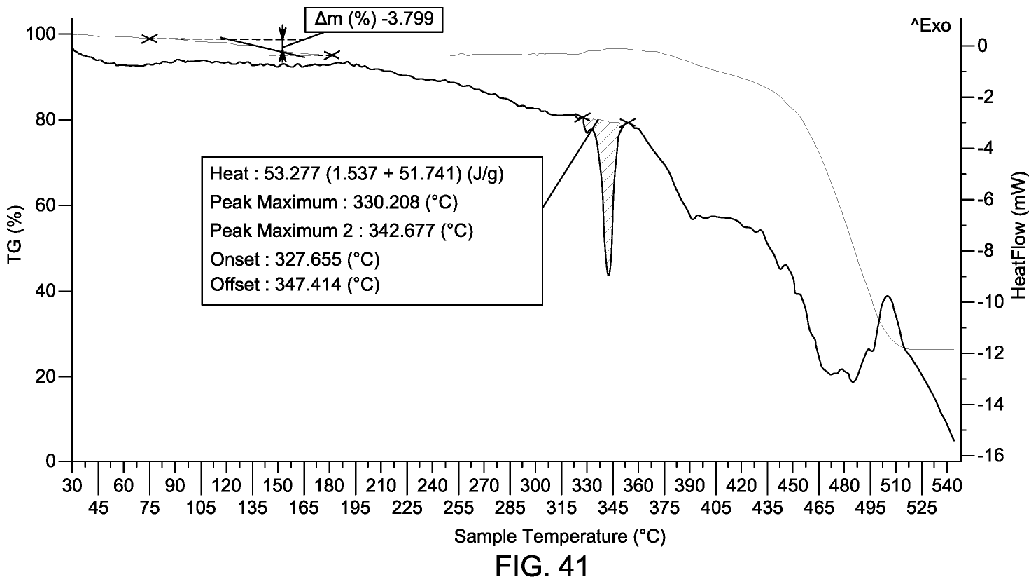

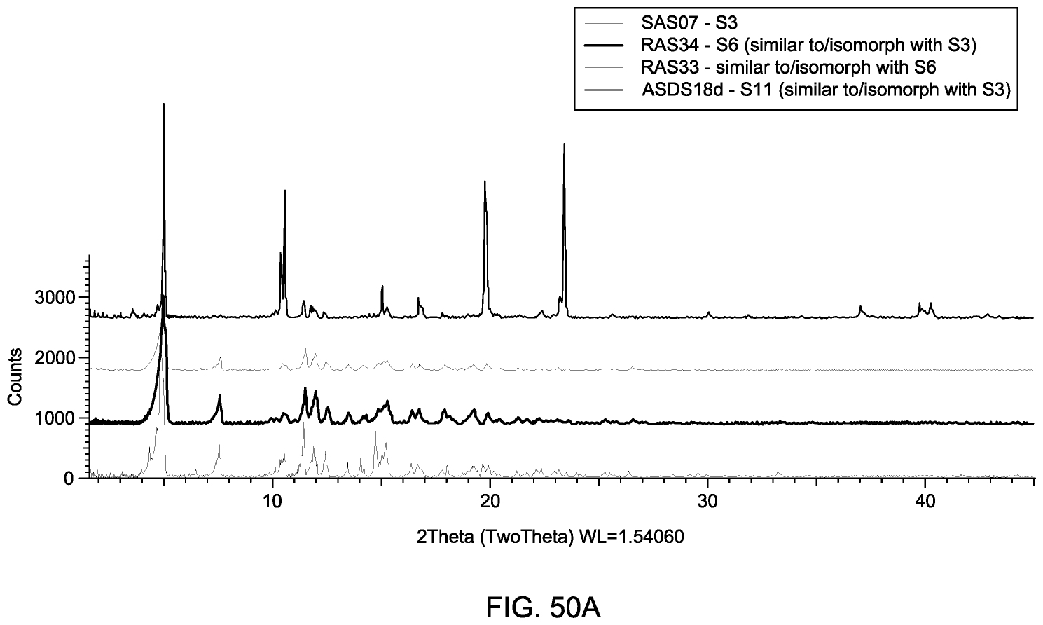

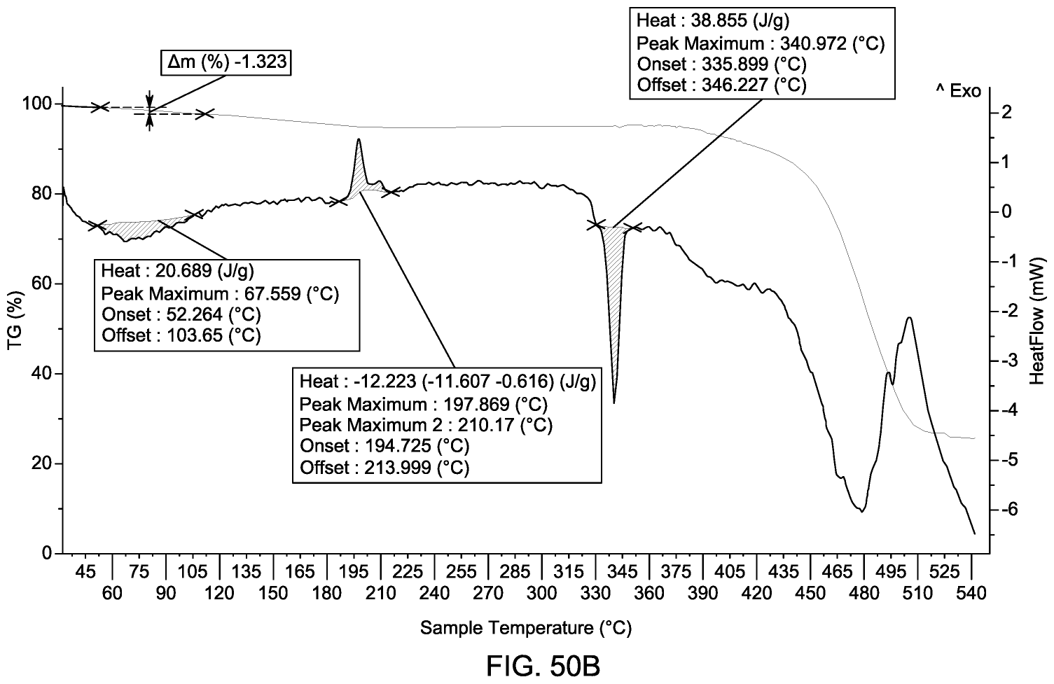

In some embodiments, Form S3, Form S6, and/or Form S11 are characterized by one or more of the following:
(i) an XRPD pattern substantially similar to that depicted in FIG. 21B and/or FIG. 50A;
(ii) a DSC pattern showing a thermal event at about 50.6° C., about 199.8° C., and/or about 331.5° C.;
(iii) a DSC pattern substantially similar to that depicted in FIG. 29 and/or FIG. 41;
(iv) a TGA pattern showing a weight loss of about 2.456% up to about 150° C.; and
(v) a TGA pattern substantially similar to that depicted in FIG. 29 and/or FIG. 41.

### Form S4

In some embodiments, the present disclosure provides sodium chenodeoxycholate as Form S4. In some embodiments, Form S4 is a solvate. In some embodiments, Form S4 is a 2,2,2-trifluoroethanol (TFE) solvate.

In some embodiments, Form S4 is characterized by one or more peaks in its XRPD pattern selected from those at about 7.07, about 7.65, about 9.70, about 13.43, about 15.02, about 16.52, and about 16.96 degrees 2-theta. In some embodiments, Form S4 is characterized by two or more peaks in its XRPD pattern selected from those at about 7.07, about 7.65, about 9.70, about 13.43, about 15.02, about 16.52, and about 16.96 degrees 2-theta. In some embodiments, Form S4 is characterized by three or more peaks in its XRPD pattern selected from those at about 7.07, about 7.65, about 9.70, about 13.43, about 15.02, about 16.52, and about 16.96 degrees 2-theta. In some embodiments, Form S4 is characterized by four or more peaks in its XRPD pattern selected from those at about 7.07, about 7.65, about 9.70, about 13.43, about 15.02, about 16.52, and about 16.96 degrees 2-theta. In some embodiments, Form S4 is characterized by five or more peaks in its XRPD pattern selected from those at about 7.07, about 7.65, about 9.70, about 13.43, about 15.02, about 16.52, and about 16.96 degrees 2-theta. In some embodiments, Form S4 is characterized by six or more peaks in its XRPD pattern selected from those at about 7.07, about 7.65, about 9.70, about 13.43, about 15.02, about 16.52, and about 16.96 degrees 2-theta.

In some embodiments, Form S4 is characterized by peaks in its XRPD pattern selected from those at about 7.07, about 7.65, about 9.70, about 13.43, about 15.02, about 16.52, and about 16.96 degrees 2-theta. In some embodiments, Form S4 is characterized by peaks in its XRPD pattern at substantially all of those listed in Table S4.

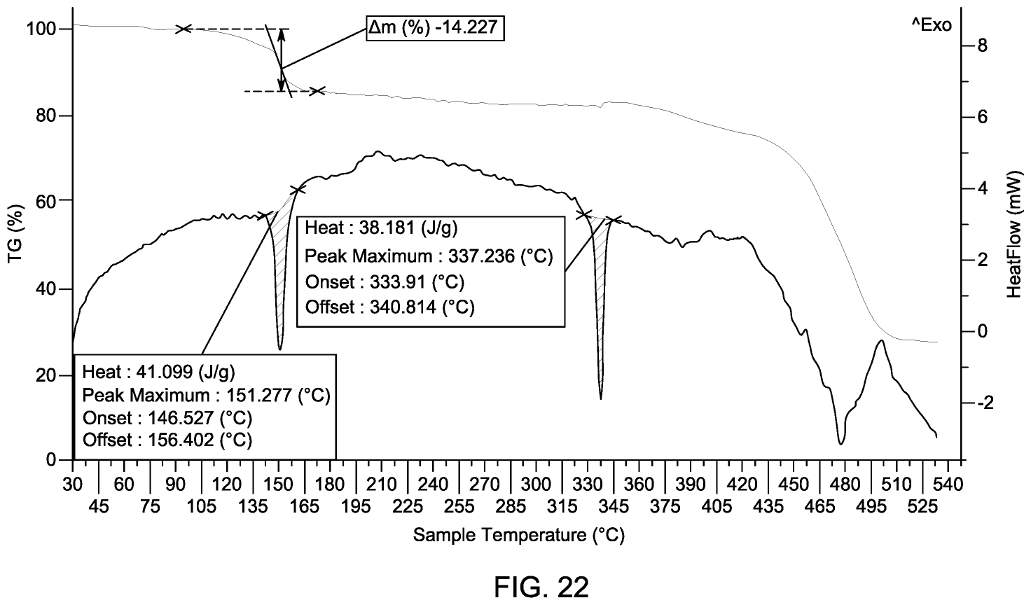

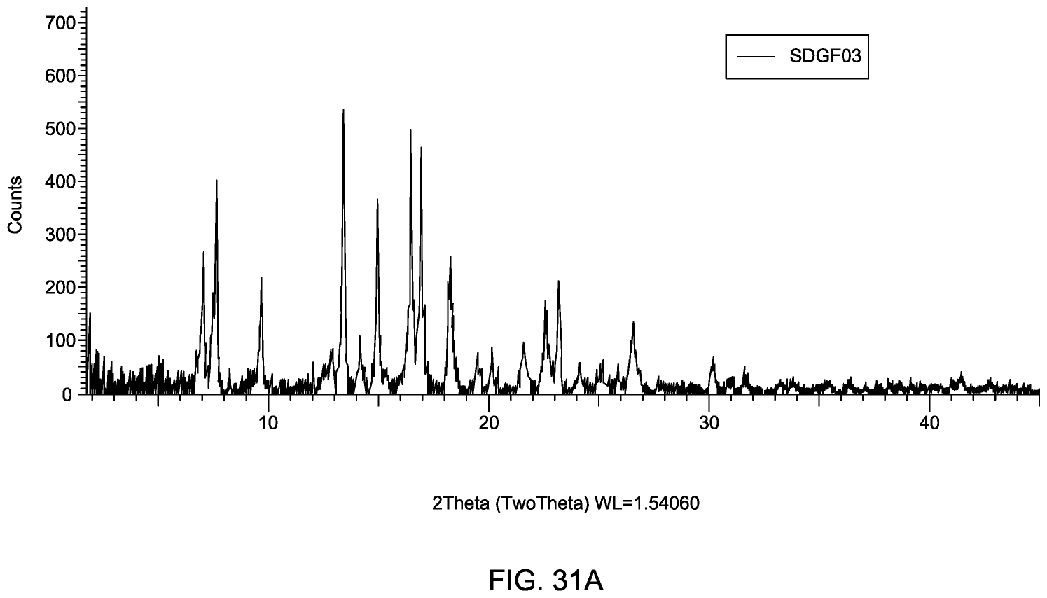

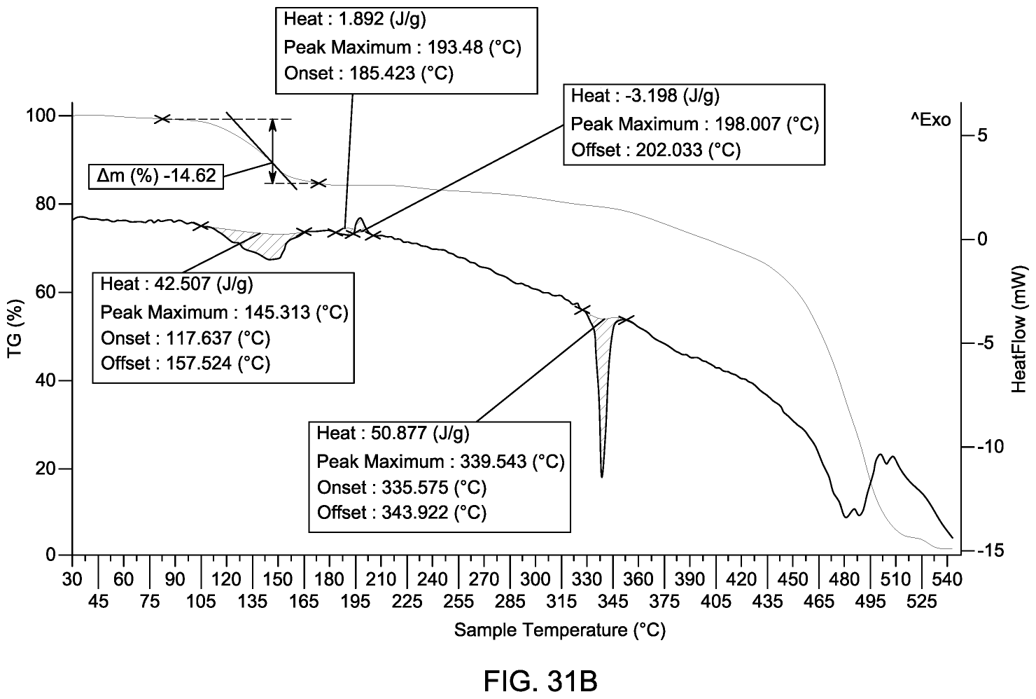

In some embodiments, Form S4 is characterized by one or more of the following:
(i) an XRPD pattern substantially similar to that depicted in FIG. 31A;
(ii) a DSC pattern showing a thermal event at about 146.5° C. and/or about 333.9° C.;
(iii) a DSC pattern substantially similar to that depicted in FIG. 22 and/or FIG. 31B;
(iv) a TGA pattern showing a weight loss of about 14.23% up to about 180° C.; and
(v) a TGA pattern substantially similar to that depicted in FIG. 22 and/or FIG. 31B.

### Form S5

In some embodiments, the present disclosure provides sodium chenodeoxycholate as Form S5. In some embodiments, Form S5 is a solvate. In some embodiments, Form S5 is a methanol solvate.

In some embodiments, Form S5 is characterized by one or more peaks in its XRPD pattern selected from those at about 7.11, about 8.63, about 12.08, about 12.75, about 13.46, about 14.25, and about 16.68 degrees 2-theta. In some embodiments, Form S5 is characterized by two or more peaks in its XRPD pattern selected from those at about 7.11, about 8.63, about 12.08, about 12.75, about 13.46, about 14.25, and about 16.68 degrees 2-theta. In some embodiments, Form S5 is characterized by three or more peaks in its XRPD pattern selected from those at about 7.11, about 8.63, about 12.08, about 12.75, about 13.46, about 14.25, and about 16.68 degrees 2-theta. In some embodiments, Form S5 is characterized by four or more peaks in its XRPD pattern selected from those at about 7.11, about 8.63, about 12.08, about 12.75, about 13.46, about 14.25, and about 16.68 degrees 2-theta. In some embodiments, Form S5 is characterized by five or more peaks in its XRPD pattern selected from those at about 7.11, about 8.63, about 12.08, about 12.75, about 13.46, about 14.25, and about 16.68 degrees 2-theta. In some embodiments, Form S5 is characterized by six or more peaks in its XRPD pattern selected from those at about 7.11, about 8.63, about 12.08, about 12.75, about 13.46, about 14.25, and about 16.68 degrees 2-theta.

In some embodiments, Form S5 is characterized by peaks in its XRPD pattern selected from those at about 7.11, about 8.63, about 12.08, about 12.75, about 13.46, about 14.25, and about 16.68 degrees 2-theta. In some embodiments, Form S5 is characterized by peaks in its XRPD pattern at substantially all of those listed in Table S5-A. In some embodiments, Form S5 is characterized by peaks in its XRPD pattern at substantially all of those listed in Table S5-B.

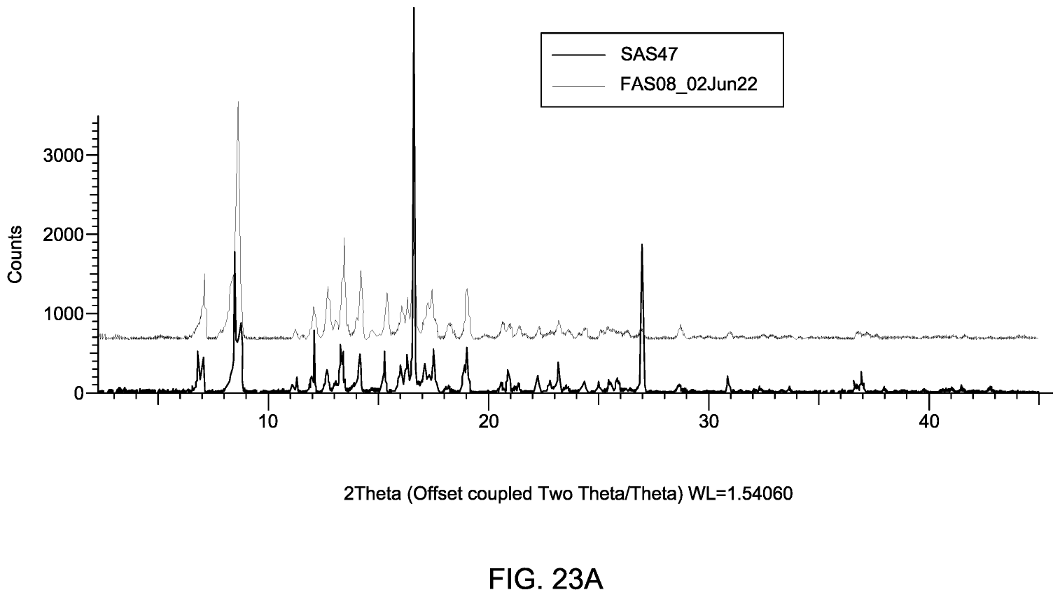

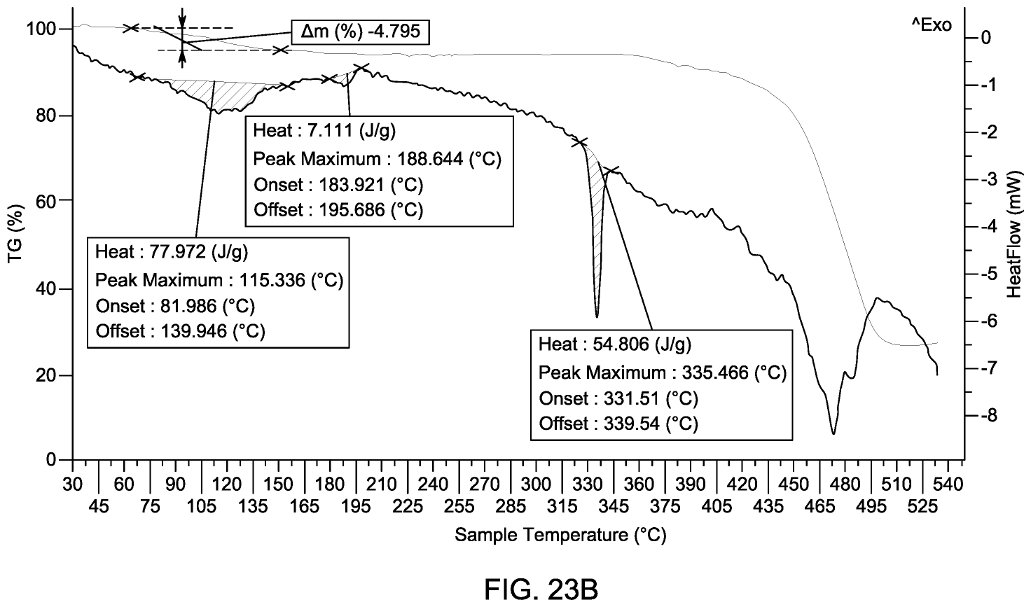

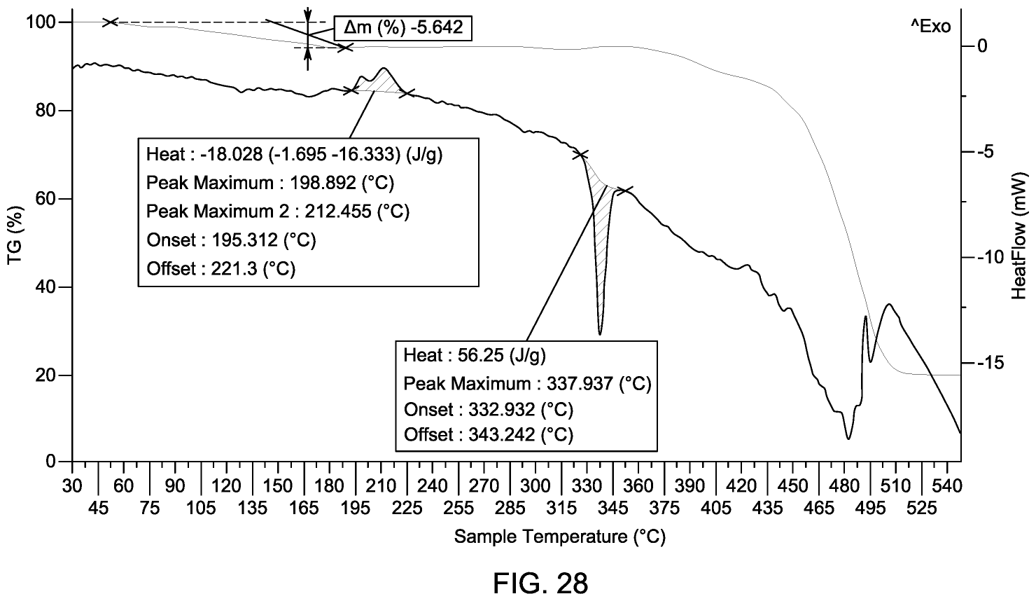

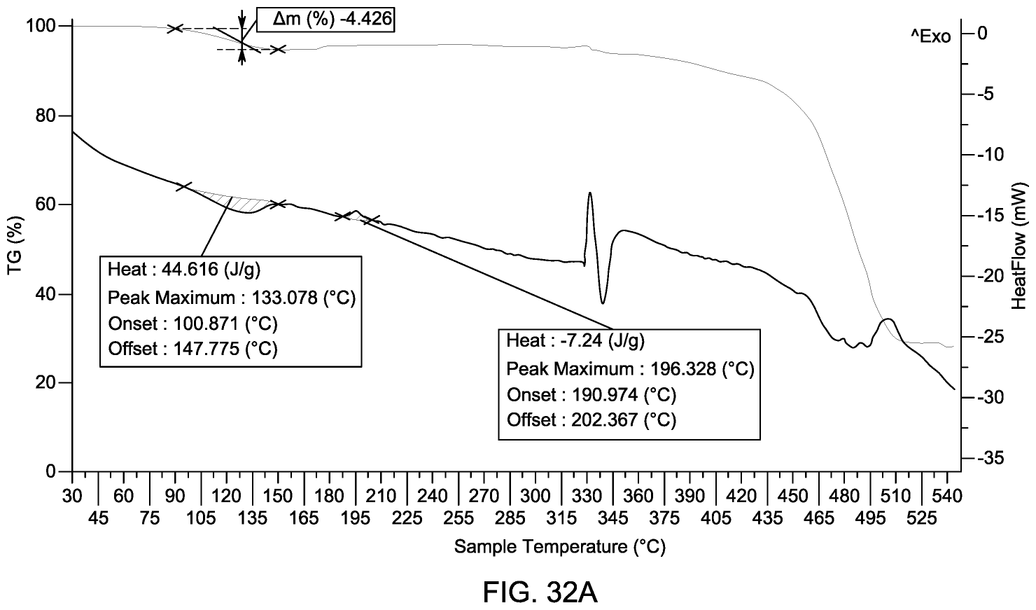

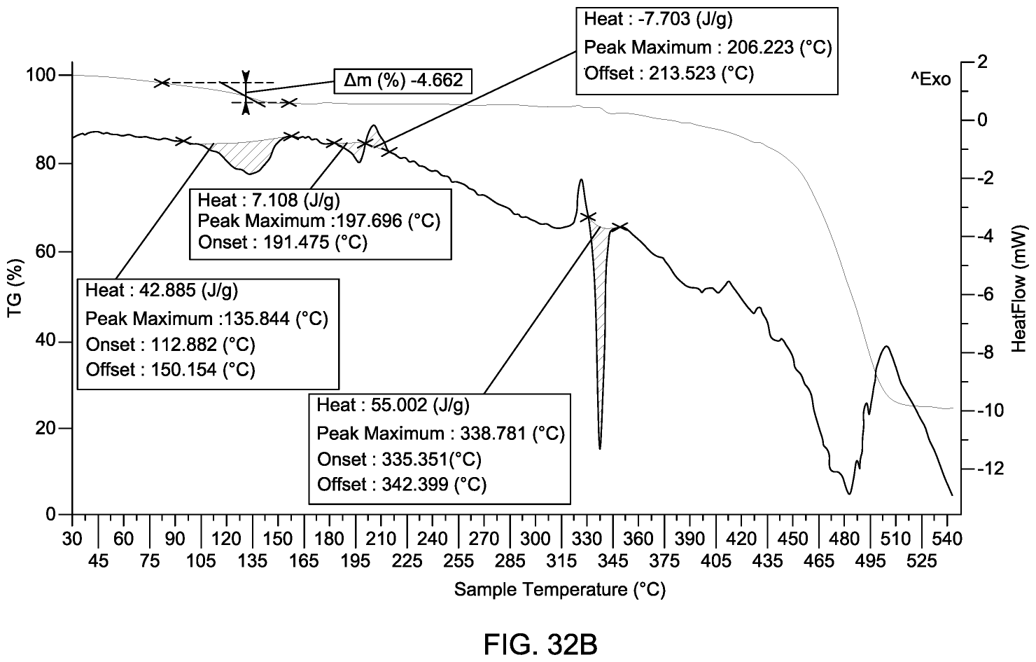

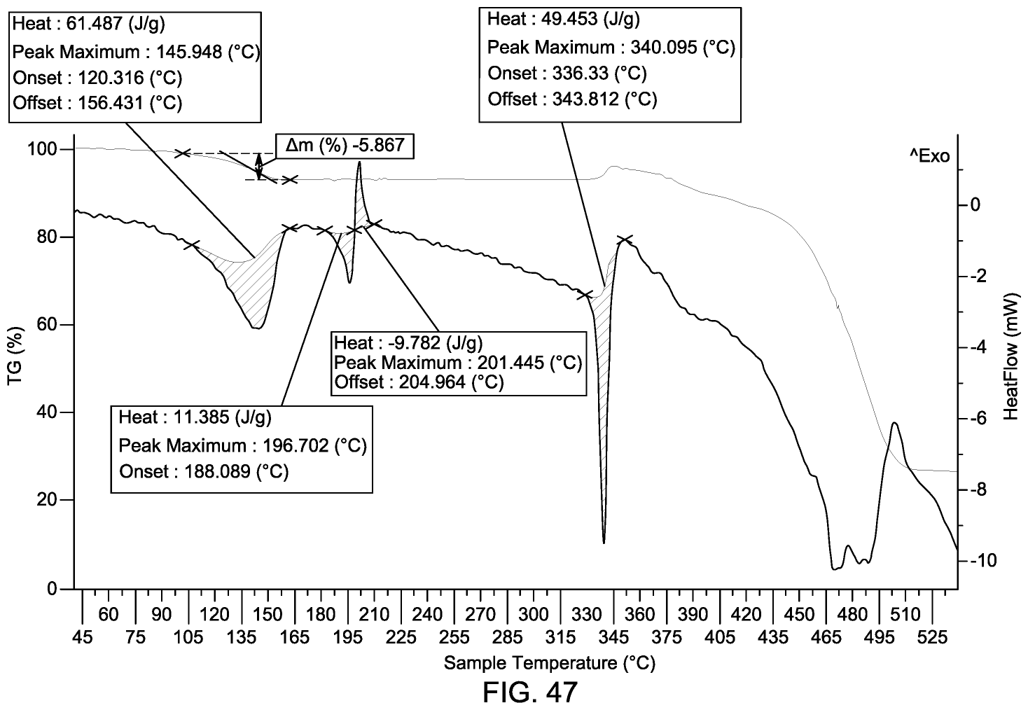

In some embodiments, Form S5 is characterized by one or more of the following:
(i) an XRPD pattern substantially similar to that depicted in FIG. 23A;
(ii) a DSC pattern showing a thermal event at about 82° C., about 183.9° C., and/or about 331.5° C.;
(iii) a DSC pattern substantially similar to that depicted in FIG. 23B, FIG. 28, FIG. 32A, FIG. 32B, and/or FIG. 47;
(iv) a TGA pattern showing a weight loss of about 4.79% up to about 180° C.; and
(v) a TGA pattern substantially similar to that depicted in FIG. 23B, FIG. 28, FIG. 32A, FIG. 32B, and/or FIG. 47.

### Form S7

In some embodiments, the present disclosure provides sodium chenodeoxycholate as Form S7. In some embodiments, Form S7 is a solvate. In some embodiments, Form S7 is an isopropanol solvate.

In some embodiments, Form S7 is characterized by one or more peaks in its XRPD pattern selected from those at about 8.47, about 9.90, about 14.36, about 15.26, about 17.00, and about 17.72 degrees 2-theta. In some embodiments, Form S7 is characterized by two or more peaks in its XRPD pattern selected from those at about 8.47, about 9.90, about 14.36, about 15.26, about 17.00, and about 17.72 degrees 2-theta. In some embodiments, Form S7 is characterized by three or more peaks in its XRPD pattern selected from those at about 8.47, about 9.90, about 14.36, about 15.26, about 17.00, and about 17.72 degrees 2-theta. In some embodiments, Form S7 is characterized by four or more peaks in its XRPD pattern selected from those at about 8.47, about 9.90, about 14.36, about 15.26, about 17.00, and about 17.72 degrees 2-theta. In some embodiments, Form S7 is characterized by five or more peaks in its XRPD pattern selected from those at about 8.47, about 9.90, about 14.36, about 15.26, about 17.00, and about 17.72 degrees 2-theta.

In some embodiments, Form S7 is characterized by peaks in its XRPD pattern selected from those at about 8.47, about 9.90, about 14.36, about 15.26, about 17.00, and about 17.72 degrees 2-theta. In some embodiments, Form S7 is characterized by peaks in its XRPD pattern at substantially all of those listed in Table S7.

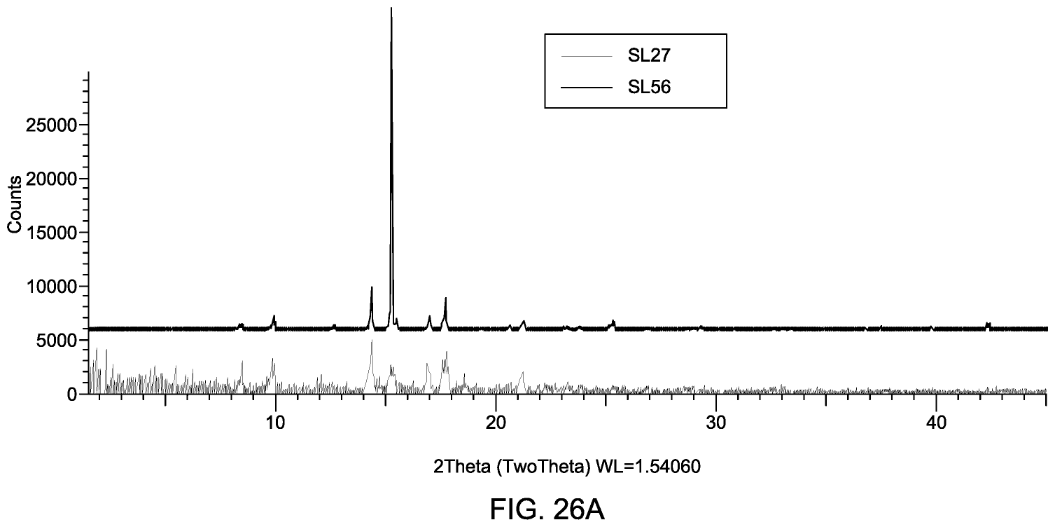

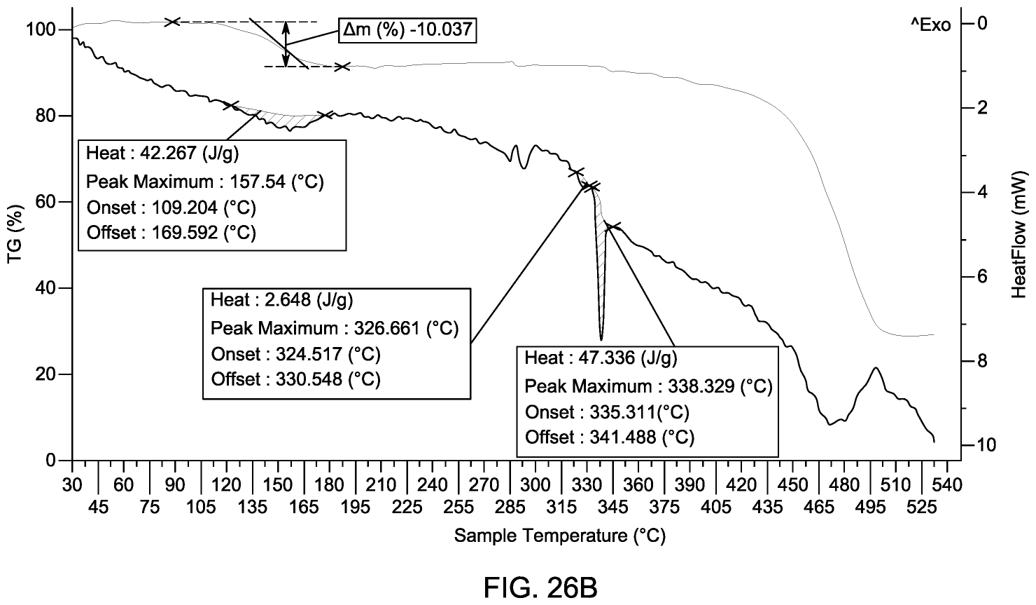

In some embodiments, Form S7 is characterized by one or more of the following:
(i) an XRPD pattern substantially similar to that depicted in FIG. 26A;
(ii) a DSC pattern showing a thermal event at about 109.2° C., about 324.5° C., and/or about 335.3° C.,
(iii) a DSC pattern substantially similar to that depicted in FIG. 26B;
(iv) a TGA pattern showing a weight loss of about 10.04% up to about 180° C.; and
(v) a TGA pattern substantially similar to that depicted in FIG. 26B.

### Form S9-a

In some embodiments, the present disclosure provides sodium chenodeoxycholate as Form S9-a. In some embodiments, Form S9-a is a solvate. In some embodiments, Form S9-a is a solvate of acetonitrile, water, or a combination thereof.

In some embodiments, Form S9-a is characterized by one or more peaks in its XRPD pattern selected from those at about 5.10, about 7.00, about 13.52, about 14.23, about 15.46, and about 18.78 degrees 2-theta. In some embodiments, Form S9-a is characterized by two or more peaks in its XRPD pattern selected from those at about 5.10, about 7.00, about 13.52, about 14.23, about 15.46, and about 18.78 degrees 2-theta. In some embodiments, Form S9-a is characterized by three or more peaks in its XRPD pattern selected from those at about 5.10, about 7.00, about 13.52, about 14.23, about 15.46, and about 18.78 degrees 2-theta. In some embodiments, Form S9-a is characterized by four or more peaks in its XRPD pattern selected from those at about 5.10, about 7.00, about 13.52, about 14.23, about 15.46, and about 18.78 degrees 2-theta. In some embodiments, Form S9-a is characterized by five or more peaks in its XRPD pattern selected from those at about 5.10, about 7.00, about 13.52, about 14.23, about 15.46, and about 18.78 degrees 2-theta.

In some embodiments, Form S9-a is characterized by peaks in its XRPD pattern selected from those at about 5.10, about 7.00, about 13.52, about 14.23, about 15.46, and about 18.78 degrees 2-theta. In some embodiments, Form S9-a is characterized by peaks in its XRPD pattern at substantially all of those listed in Table S9-a.

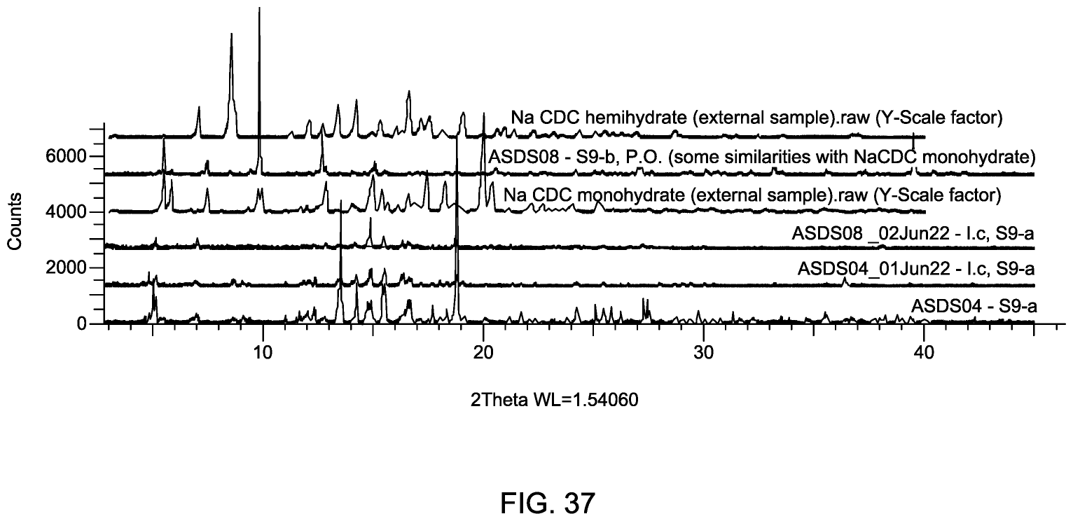

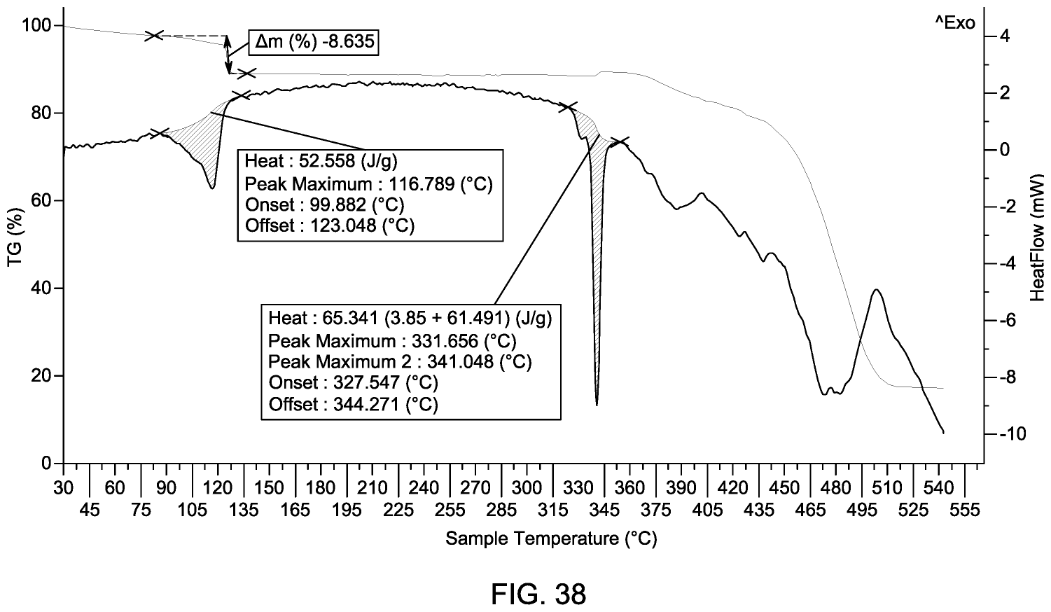

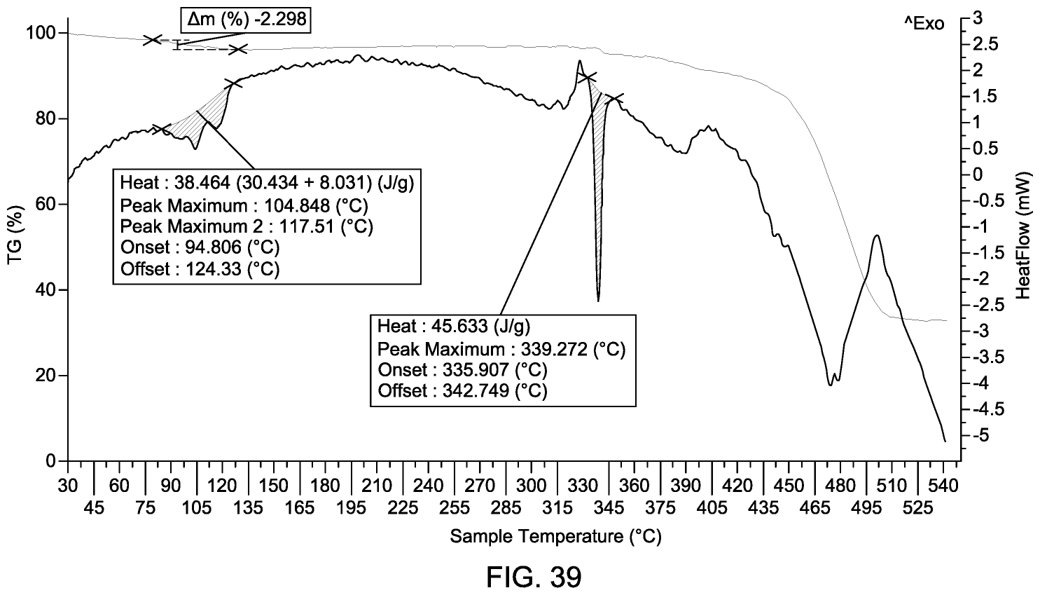

In some embodiments, Form S9-a is characterized by one or more of the following:
(i) an XRPD pattern substantially similar to that depicted in FIG. 37 (bottom spectrum);
(ii) a DSC pattern showing a thermal event at about 99.9° C. and/or about 328° C.;
(iii) a DSC pattern substantially similar to that depicted in FIG. 38 and/or FIG. 39;
(iv) a TGA pattern showing a weight loss of about 8.635% up to about 150° C.; and
(v) a TGA pattern substantially similar to that depicted in FIG. 38 and/or FIG. 39.

### Form S9-b

In some embodiments, the present disclosure provides sodium chenodeoxycholate as Form S9-b.

In some embodiments, Form S9-b is characterized by one or more peaks in its XRPD pattern selected from those at about 5.47, about 7.48, about 9.82, about 12.66, and about 15.07 degrees 2-theta. In some embodiments, Form S9-b is characterized by two or more peaks in its XRPD pattern selected from those at about 5.47, about 7.48, about 9.82, about 12.66, and about 15.07 degrees 2-theta. In some embodiments, Form S9-b is characterized by three or more peaks in its XRPD pattern selected from those at about 5.47, about 7.48, about 9.82, about 12.66, and about 15.07 degrees 2-theta. In some embodiments, Form S9-b is characterized by four or more peaks in its XRPD pattern selected from those at about 5.47, about 7.48, about 9.82, about 12.66, and about 15.07 degrees 2-theta. In some embodiments, Form S9-b is characterized by five or more peaks in its XRPD pattern selected from those at about 5.47, about 7.48, about 9.82, about 12.66, and about 15.07 degrees 2-theta.

In some embodiments, Form S9-b is characterized by peaks in its XRPD pattern selected from those at about 5.47, about 7.48, about 9.82, about 12.66, and about 15.07 degrees 2-theta. In some embodiments, Form S9-b is characterized by peaks in its XRPD pattern at substantially all of those listed in Table S9-b. In some embodiments, Form S9-b is characterized by an XRPD pattern substantially similar to that depicted in FIG. 37 (second spectrum from top).

### Form S10

In some embodiments, the present disclosure provides sodium chenodeoxycholate as Form S10. In some embodiments, Form S10 is a solvate. In some embodiments, Form S10 is a solvate of 2,2,2-trifluoroethanol (TFE), ethyl acetate, or a combination thereof.

In some embodiments, Form S10 is characterized by one or more peaks in its XRPD pattern selected from those at about 5.13, about 7.01, about 8.69, about 9.11, about 13.55, about 14.91, and about 15.53 degrees 2-theta. In some embodiments, Form S10 is characterized by two or more peaks in its XRPD pattern selected from those at about 5.13, about 7.01, about 8.69, about 9.11, about 13.55, about 14.91, and about 15.53 degrees 2-theta. In some embodiments, Form S10 is characterized by three or more peaks in its XRPD pattern selected from those at about 5.13, about 7.01, about 8.69, about 9.11, about 13.55, about 14.91, and about 15.53 degrees 2-theta. In some embodiments, Form S10 is characterized by four or more peaks in its XRPD pattern selected from those at about 5.13, about 7.01, about 8.69, about 9.11, about 13.55, about 14.91, and about 15.53 degrees 2-theta. In some embodiments, Form S10 is characterized by five or more peaks in its XRPD pattern selected from those at about 5.13, about 7.01, about 8.69, about 9.11, about 13.55, about 14.91, and about 15.53 degrees 2-theta. In some embodiments, Form S10 is characterized by six or more peaks in its XRPD pattern selected from those at about 5.13, about 7.01, about 8.69, about 9.11, about 13.55, about 14.91, and about 15.53 degrees 2-theta.

In some embodiments, Form S10 is characterized by peaks in its XRPD pattern selected from those at about 5.13, about 7.01, about 8.69, about 9.11, about 13.55, about 14.91, and about 15.53 degrees 2-theta. In some embodiments, Form S10 is characterized by peaks in its XRPD pattern at substantially all of those listed in Table S10.

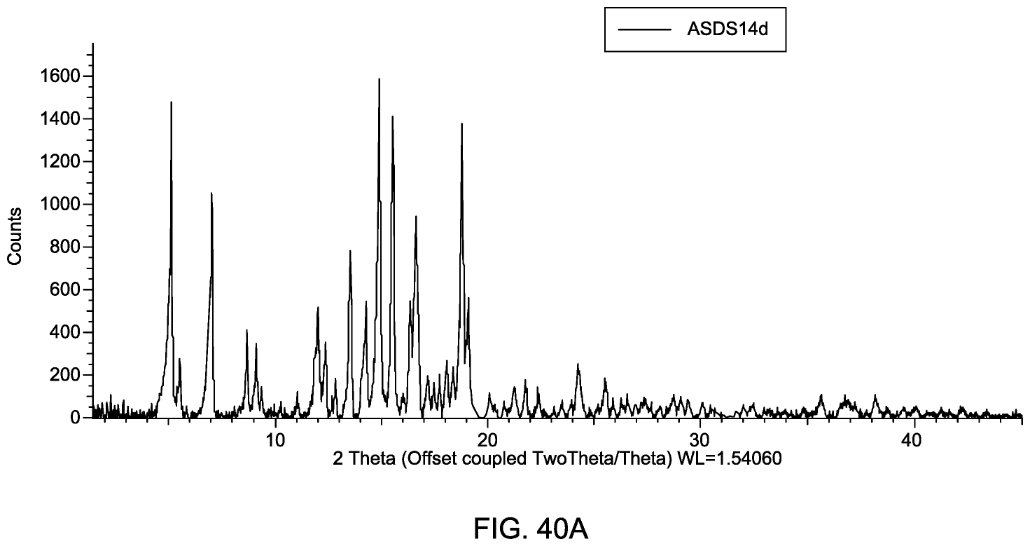

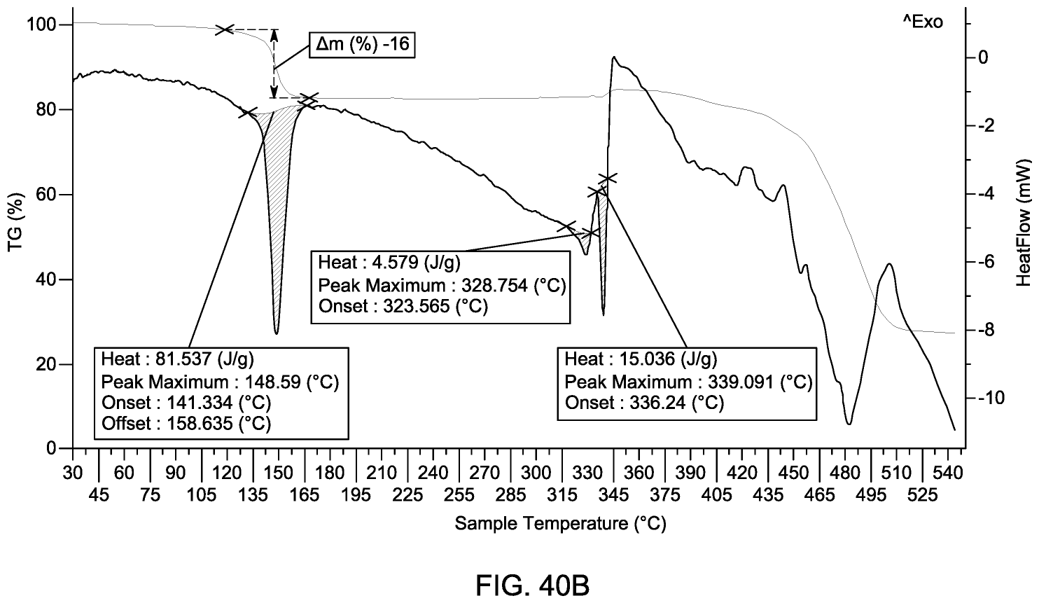

In some embodiments, Form S10 is characterized by one or more of the following:
(i) an XRPD pattern substantially similar to that depicted in FIG. 40A;
(ii) a DSC pattern showing a thermal event at about 141.3° C., about 324° C. and/or about 336° C.;
(iii) a DSC pattern substantially similar to that depicted in FIG. 40B;
(iv) a TGA pattern showing a weight loss of about 16% up to about 150° C.; and
(v) a TGA pattern substantially similar to that depicted in FIG. 40B.

### Form S12 and Form S15

In some embodiments, the present disclosure provides sodium chenodeoxycholate as Form S12 and/or Form 515. In some embodiments, Form S12 and/or Form S15 is a solvate. In some embodiments, Form S12 and/or Form S15 is a methyl tert-butyl ether (MTBE) solvate. In some embodiments, Form S12 and Form S15 are similar to and/or isomorphic with each other. Therefore, in some embodiments, Form S12 and/or Form S15 share one or more features described herein.

In some embodiments, Form S12 and/or Form S15 are characterized by one or more peaks in its XRPD pattern selected from those at about 4.82, about 5.22, about 5.89, about 10.81, about 13.00, about 15.00, and about 18.94 degrees 2-theta. In some embodiments, Form S12 and/or Form S15 are characterized by two or more peaks in its XRPD pattern selected from those at about 4.82, about 5.22, about 5.89, about 10.81, about 13.00, about 15.00, and about 18.94 degrees 2-theta. In some embodiments, Form S12 and/or Form S15 are characterized by three or more peaks in its XRPD pattern selected from those about 4.82, about 5.22, about 5.89, about 10.81, about 13.00, about 15.00, and about 18.94 degrees 2-theta. In some embodiments, Form S12 and/or Form S15 are characterized by four or more peaks in its XRPD pattern selected from those at about 4.82, about 5.22, about 5.89, about 10.81, about 13.00, about 15.00, and about 18.94 degrees 2-theta. In some embodiments, Form S12 and/or Form S15 are characterized by five or more peaks in its XRPD pattern selected from those at about 4.82, about 5.22, about 5.89, about 10.81, about 13.00, about 15.00, and about 18.94 degrees 2-theta. In some embodiments, Form S12 and/or Form S15 are characterized by six or more peaks in its XRPD pattern selected from those at about 4.82, about 5.22, about 5.89, about 10.81, about 13.00, about 15.00, and about 18.94 degrees 2-theta.

In some embodiments, Form S12 and/or Form S15 are characterized by peaks in its XRPD pattern selected from those at about 4.82, about 5.22, about 5.89, about 10.81, about 13.00, about 15.00, and about 18.94 degrees 2-theta. In some embodiments, Form S12 and/or Form S15 are characterized by peaks in its XRPD pattern at substantially all of those listed in Table S12. In some embodiments, Form S12 and/or Form S15 are characterized by peaks in its XRPD pattern at substantially all of those listed in Table 515.

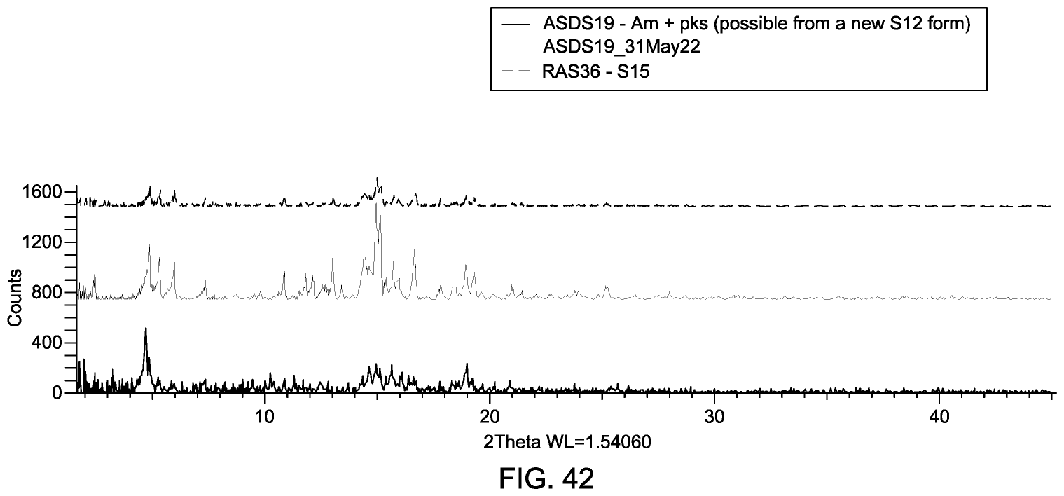

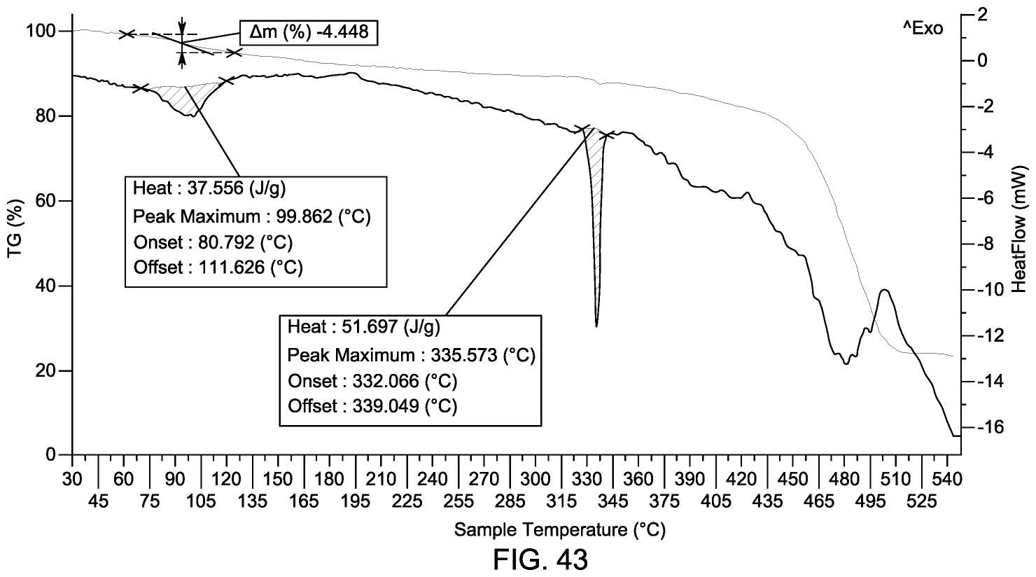

In some embodiments, Form S12 and/or Form S15 are characterized by one or more of the following:
(i) an XRPD pattern substantially similar to that depicted in FIG. 42;
(ii) a DSC pattern showing a thermal event at about 80.8° C., about 180° C., and/or about 332° C.;
(iii) a DSC pattern substantially similar to that depicted in FIG. 43;
(iv) a TGA pattern showing a weight loss of about 4.448% up to about 150° C.; and
(v) a TGA pattern substantially similar to that depicted in FIG. 43.

### Form S13

In some embodiments, the present disclosure provides sodium chenodeoxycholate as Form S13. In some embodiments, Form S13 is a solvate. In some embodiments, Form S13 is a solvate of 2,2,2-trifluoroethanol (TFE), diisopropyl ether, or a mixture thereof.

In some embodiments, Form S13 is characterized by one or more peaks in its XRPD pattern selected from those at about 4.95, about 7.53, about 9.85, about 11.55, about 12.12, and about 15.01 degrees 2-theta. In some embodiments, Form S14 is characterized by two or more peaks in its XRPD pattern selected from those at about 4.95, about 7.53, about 9.85, about 11.55, about 12.12, and about 15.01 degrees 2-theta. In some embodiments, Form S14 is characterized by three or more peaks in its XRPD pattern selected from those at about 4.95, about 7.53, about 9.85, about 11.55, about 12.12, and about 15.01 degrees 2-theta. In some embodiments, Form S14 is characterized by four or more peaks in its XRPD pattern selected from those at about 4.95, about 7.53, about 9.85, about 11.55, about 12.12, and about 15.01 degrees 2-theta. In some embodiments, Form S14 is characterized by five or more peaks in its XRPD pattern selected from those at about 4.95, about 7.53, about 9.85, about 11.55, about 12.12, and about 15.01 degrees 2-theta.

In some embodiments, Form S13 is characterized by peaks in its XRPD pattern selected from those at about 4.95, about 7.53, about 9.85, about 11.55, about 12.12, and about 15.01 degrees 2-theta. In some embodiments, Form S13 is characterized by peaks in its XRPD pattern at substantially all of those listed in Table S13.

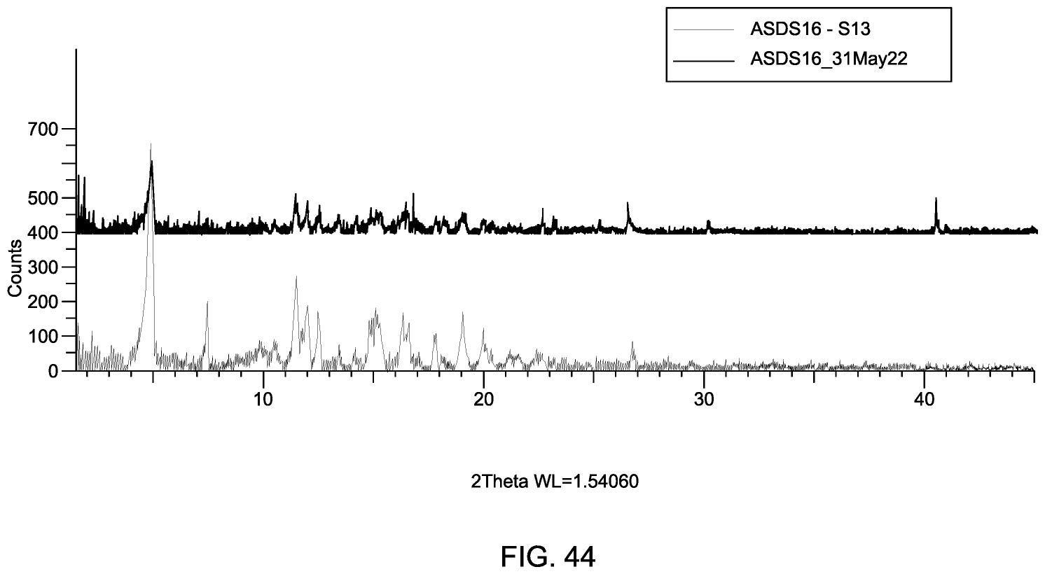

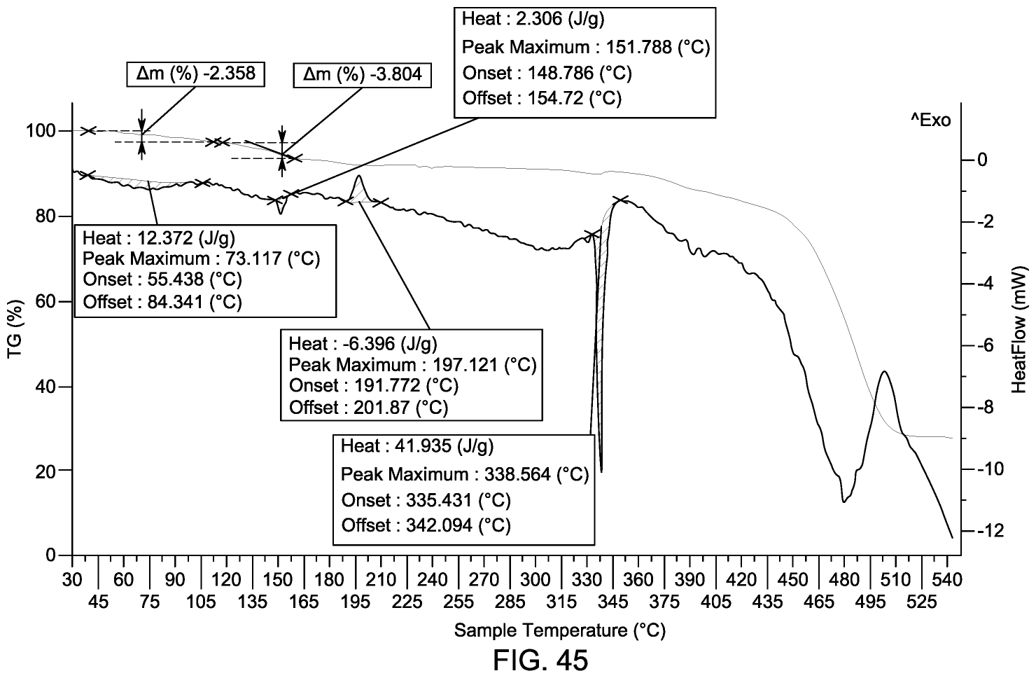

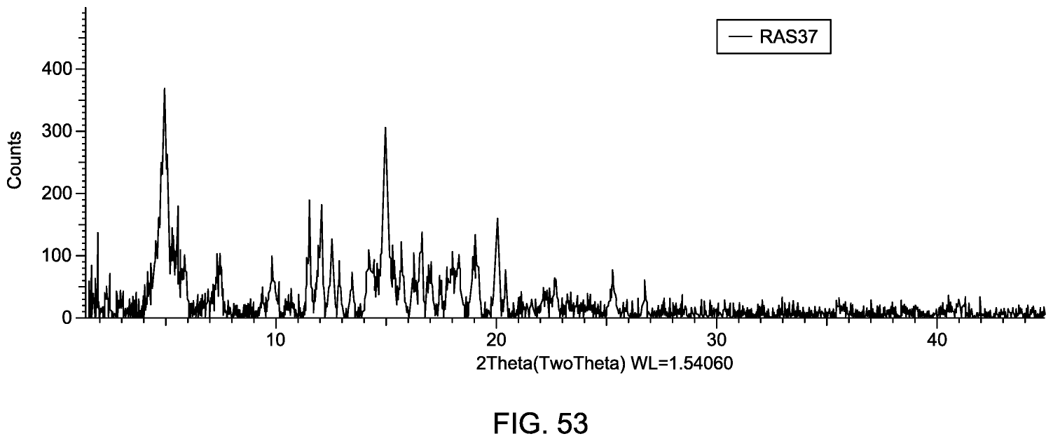

In some embodiments, Form S13 is characterized by one or more of the following:
(i) an XRPD pattern substantially similar to that depicted in FIG. 44 and/or FIG. 53;
(ii) a DSC pattern showing a thermal event at about 55.4° C., about 148.8° C., about 191.8° C. and/or about 335° C.;
(iii) a DSC pattern substantially similar to that depicted in FIG. 45;
(iv) a TGA pattern showing a weight loss of about 6.162% up to about 180° C.; and
(v) a TGA pattern substantially similar to that depicted in FIG. 45.

### Form S14

In some embodiments, the present disclosure provides sodium chenodeoxycholate as Form S14. In some embodiments, Form S14 is a solvate. In some embodiments, Form S14 is a 2,2,2-trifluoroethanol (TFE) solvate.

In some embodiments, Form S14 is characterized by one or more peaks in its XRPD pattern selected from those at about 5.15, about 5.50, about 7.05, about 12.04, about 14.90, and about 16.56 degrees 2-theta. In some embodiments, Form S14 is characterized by two or more peaks in its XRPD pattern selected from those at about 5.15, about 5.50, about 7.05, about 12.04, about 14.90, and about 16.56 degrees 2-theta. In some embodiments, Form S14 is characterized by three or more peaks in its XRPD pattern selected from those at about 5.15, about 5.50, about 7.05, about 12.04, about 14.90, and about 16.56 degrees 2-theta. In some embodiments, Form S14 is characterized by four or more peaks in its XRPD pattern selected from those at about 5.15, about 5.50, about 7.05, about 12.04, about 14.90, and about 16.56 degrees 2-theta. In some embodiments, Form S14 is characterized by five or more peaks in its XRPD pattern selected from those at about 5.15, about 5.50, about 7.05, about 12.04, about 14.90, and about 16.56 degrees 2-theta.

In some embodiments, Form S14 is characterized by peaks in its XRPD pattern selected from those at about 5.15, about 5.50, about 7.05, about 12.04, about 14.90, and about 16.56 degrees 2-theta. In some embodiments, Form S14 is characterized by peaks in its XRPD pattern at substantially all of those listed in Table 514.

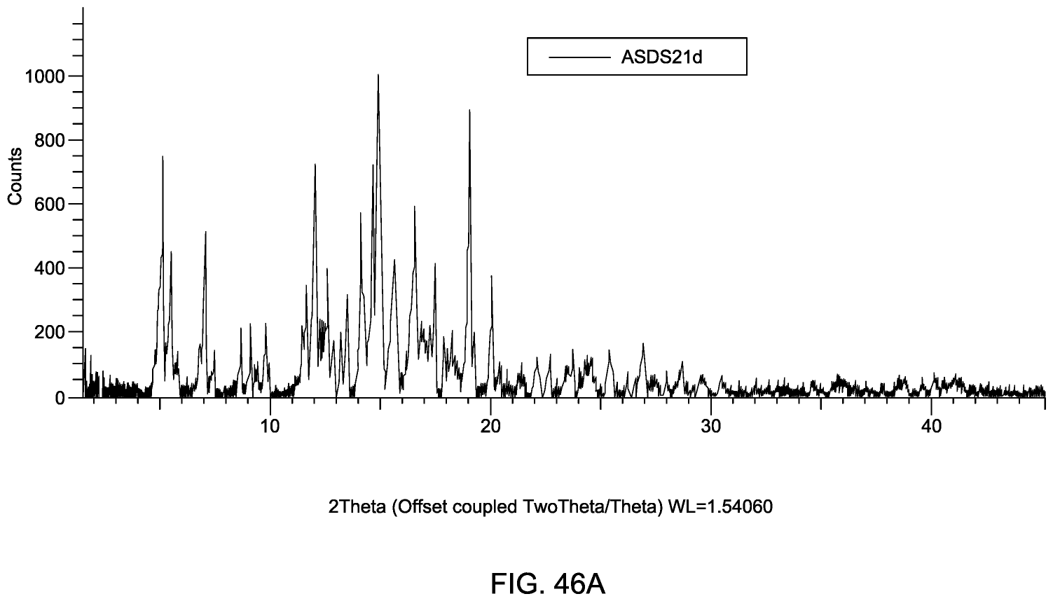

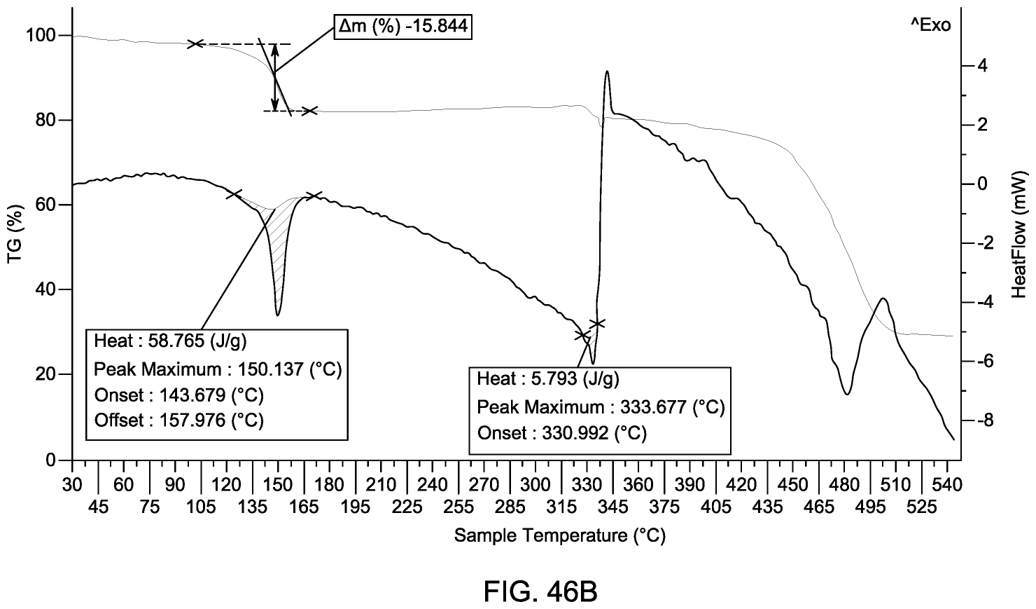

In some embodiments, Form S14 is characterized by one or more of the following:
(i) an XRPD pattern substantially similar to that depicted in FIG. 46A;
(ii) a DSC pattern showing a thermal event at about 143.7° C. and/or about 331° C.;
(iii) a DSC pattern substantially similar to that depicted in FIG. 46B;
(iv) a TGA pattern showing a weight loss of about 15.84% up to about 180° C.; and
(v) a TGA pattern substantially similar to that depicted in FIG. 46B.

### Preparing Provided Solid Forms

In some embodiments, the present disclosure provides methods of preparing crystalline solid forms of sodium chenodeoxycholate (NaCDC).

In some embodiments, a solid form of NaCDC is prepared by contacting CDCA (e.g., amorphous CDCA, crystalline CDCA, or a mixture thereof) with a suitable base, such as sodium hydroxide. In some embodiments, the present disclosure provides a method of preparing NaCDC comprising steps of: providing CDCA; and combining CDCA with a suitable base (e.g., sodium hydroxide), optionally in a suitable solvent, to provide NaCDC. In some embodiments, about 1.0, about 2.0, about 3.0, about 4.0, about 5.0, or more equivalents of suitable base (e.g., sodium hydroxide) are added.

In some embodiments, a solid form of NaCDC is prepared by dissolving NaCDC (e.g., amorphous NaCDC, crystalline NaCDC, or a mixture thereof) in a suitable solvent and then causing NaCDC to return to the solid phase. In some embodiments, a solid form of NaCDC is preparing by combining NaCDC (e.g., amorphous NaCDC, crystalline NaCDC, or a mixture thereof) in a suitable solvent under suitable conditions and isolating the solid form of NaCDC. In some embodiments, a solid form of NaCDC is prepared according to a method described herein (e.g., according to a slurry, slurry with sonication, slow evaporation, solvent drop grinding, vapor diffusion onto solids, anti-solvent vapor diffusion into solution, crash cooling, forward anti-solvent addition, or reverse anti-solvent addition method, as described in the Examples herein).

In some embodiments, a suitable solvent is methyl isobutyl ketone (MIBK), n-butanol, water, or a mixture thereof. In some embodiments, a suitable solvent is selected from acetone, acetonitrile, n-butyl acetate, diisopropyl ether, ethanol, 2-ethoxyethanol, ethyl acetate, ethyl ether, n-heptane, isopropyl acetate, methanol, methyl ethyl ketone, methyl tert-butyl ether, 2-propanol, 2,2,2-trifluoroethanol, toluene, water, or a mixture thereof.

In some embodiments, a method of preparing a solid form of NaCDC comprises a step of heating a mixture comprising NaCDC to a suitable temperature. In some embodiments, a method of preparing a solid form of NaCDC comprises a step of stirring a mixture comprising NaCDC at ambient temperature. In some embodiments, a method of preparing a solid form of NaCDC comprises step of cooling a mixture comprising NaCDC to a suitable temperature.

In some embodiments, a solid form of NaCDC precipitates from a mixture (e.g., a solution, suspension, or slurry). In some embodiments, a solid form of NaCDC crystallizes from a solution. In some embodiments, a solid form of NaCDC crystallizes from a solution following seeding of the solution (e.g., adding crystals of NaCDC to the solution). In some embodiments, a solid form of NaCDC precipitates or crystallizes from a mixture after cooling, addition of an anti-solvent, and/or removal of all or part of a solvent through methods such as evaporation, distillation, filtration, reverse osmosis, absorption, or reaction.

In some embodiments, a method of preparing a solid form of NaCDC comprises a step of isolating the solid form. It will be appreciated that a solid form of NaCDC may be isolated by any suitable means. In some embodiments, a solid form of NaCDC is separated from a supernatant by filtration. In some embodiments, a solid form of NaCDC is separated from a supernatant by decanting. In some embodiments, a solid form of NaCDC is separated from a supernatant by centrifugation.

In some embodiments, an isolated solid form of NaCDC is dried (e.g., in air or under reduced pressure, optionally at elevated temperature).

In some embodiments, a solid form of NaCDC is prepared by converting one solid form of NaCDC into another solid form of NaCDC. For example, in some embodiments, a solid form of NaCDC (e.g., any one of Forms S1, S2, S3, S4, S5, S6, S7, S9-a, S9-b, S10, S11, S12, S13, S14, and/or S15) is prepared by converting NaCDC Form B as described in the Examples herein.

In some embodiments, a solid form of NaCDC is prepared by a process comprising a step of combining CDCA in a suitable solvent (e.g., methyl isobutyl ketone). In some embodiments, the step of combining comprises stirring the mixture at a suitable temperature (e.g., ambient temperature). In some embodiments, the process further comprises adding a suitable base (e.g., sodium hydroxide, e.g., as an aqueous solution). In some embodiments, the process further comprises heating the mixture to reflux (e.g., azeotropic reflux). In some embodiments, the process further comprises distilling off a portion of the solvent, e.g., until a vapor temperature of about 117° C. is observed. In some embodiments, the process further comprises cooling the mixture to a suitable temperature (e.g., ambient temperature). In some embodiments, the process further comprises shaking or stirring the mixture (e.g., at ambient temperature). In some embodiments, the process further comprises isolating the solid form of NaCDC (e.g., Form A) by a suitable method, such as filtration. In some embodiments, a process comprises steps of: providing a mixture of chenodeoxycholic acid in methyl isobutyl ketone; adding to the mixture an aqueous solution of sodium hydroxide; heating the mixture; and removing the solvent to provide NaCDC Form A.

In some embodiments, the present disclosure provides a method of preparing a solid form of NaCDC comprising a step of combining CDCA in a suitable solvent (e.g., methyl isobutyl ketone). In some embodiments, the step of combining comprises stirring the mixture at a suitable temperature (e.g., ambient temperature). In some embodiments, the method further comprises adding a suitable base (e.g., sodium hydroxide, e.g., as an aqueous solution). In some embodiments, the method further comprises heating the mixture to reflux (e.g., azeotropic reflux). In some embodiments, the method further comprises distilling off a portion of the solvent, e.g., until a vapor temperature of about 117° C. is observed. In some embodiments, the method further comprises cooling the mixture to a suitable temperature (e.g., ambient temperature). In some embodiments, the method further comprises shaking or stirring the mixture (e.g., at ambient temperature). In some embodiments, the method further comprises isolating the solid form of NaCDC (e.g., Form A) by a suitable method, such as filtration. In some embodiments, a method comprises steps of: providing a mixture of chenodeoxycholic acid in methyl isobutyl ketone; adding to the mixture an aqueous solution of sodium hydroxide; heating the mixture; and removing the solvent to provide NaCDC Form A.

In some embodiments, a solid form of NaCDC is prepared by a process comprising a step of combining CDCA in a suitable solvent (e.g., n-butanol). In some embodiments, the step of combining comprises stirring the mixture at a suitable temperature (e.g., ambient temperature). In some embodiments, the process further comprises adding a suitable base (e.g., sodium hydroxide, e.g., as an aqueous solution). In some embodiments, the process further comprises heating the mixture to reflux (e.g., azeotropic reflux). In some embodiments, the process further comprises distilling off a portion of the solvent, e.g., until a vapor temperature of about 117° C. is observed. In some embodiments, the process further comprises cooling the mixture to a suitable temperature (e.g., ambient temperature). In some embodiments, the process further comprises shaking or stirring the mixture (e.g., at ambient temperature). In some embodiments, the process further comprises isolating the solid form of NaCDC (e.g., Form B) by a suitable method, such as filtration. In some embodiments, a process comprises steps of: providing a mixture of chenodeoxycholic acid in n-butanol; adding to the mixture an aqueous solution of sodium hydroxide; heating the mixture; and removing the solvent to provide NaCDC Form B.

In some embodiments, the present disclosure provides a method of preparing a solid form of NaCDC comprising a step of combining CDCA in a suitable solvent (e.g., n-butanol). In some embodiments, the step of combining comprises stirring the mixture at a suitable temperature (e.g., ambient temperature). In some embodiments, the method further comprises adding a suitable base (e.g., sodium hydroxide, e.g., as an aqueous solution). In some embodiments, the method further comprises heating the mixture to reflux (e.g., azeotropic reflux). In some embodiments, the method further comprises distilling off a portion of the solvent, e.g., until a vapor temperature of about 117° C. is observed. In some embodiments, the method further comprises cooling the mixture to a suitable temperature (e.g., ambient temperature). In some embodiments, the method further comprises shaking or stirring the mixture (e.g., at ambient temperature). In some embodiments, the method further comprises isolating the solid form of NaCDC (e.g., Form B) by a suitable method, such as filtration. In some embodiments, a method comprises steps of: providing a mixture of chenodeoxycholic acid in n-butanol; adding to the mixture an aqueous solution of sodium hydroxide; heating the mixture; and removing the solvent to provide NaCDC Form B.

### Compositions

In some embodiments, the present disclosure also provides compositions comprising one or more solid forms of CDCA and/or NaCDC. In some embodiments, provided compositions comprise amorphous CDCA, crystalline CDCA, amorphous NaCDC, crystalline NaCDC (e.g., Form A or Form B, or any other Form provided herein), or a mixture thereof. In some embodiments, provided compositions comprise NaCDC Form A. In some embodiments, provided compositions comprise NaCDC Form B.

In some embodiments, a provided composition comprising a crystalline solid form (e.g., NaCDC Form A or NaCDC Form B, or any other Form provided herein) is substantially free of impurities. As used herein, the term “substantially free of impurities” means that the composition contains no significant amount of extraneous matter. Such extraneous matter may include starting materials, residual solvents, or any other impurities that may result from the preparation of and/or isolation of a crystalline solid form. In some embodiments, the composition comprises at least about 90% by weight of a crystalline solid form (e.g., NaCDC Form A or NaCDC Form B, or any other Form provided herein). In some embodiments, the composition comprises at least about 95% by weight of a crystalline solid form (e.g., NaCDC Form A or NaCDC Form B, or any other Form provided herein). In some embodiments, the composition comprises at least about 99% by weight of a crystalline solid form (e.g., NaCDC Form A or NaCDC Form B, or any other Form provided herein).

In some embodiments, a provided composition comprising a crystalline solid form (e.g., NaCDC Form A or NaCDC Form B, or any other Form provided herein) is substantially pure (e.g., comprises at least about 95%, 97%, 97.5%, 98,% 98.5%, 99%, 99.5%, or 99.8% by weight of the crystalline solid form based on the total weight of the composition). In some embodiments, a composition comprising a crystalline solid form (e.g., NaCDC Form A or NaCDC Form B, or any other Form provided herein) comprises no more than about 5.0 percent of total organic impurities. In some embodiments, a composition comprising a crystalline solid form (e.g., NaCDC Form A or NaCDC Form B, or any other Form provided herein) comprises no more than about 3.0 percent of total organic impurities. In some embodiments, a composition comprising a crystalline solid form (e.g., NaCDC Form A or NaCDC Form B, or any other Form provided herein) comprises no more than about 1.5 percent of total organic impurities. In some embodiments, a composition comprising a crystalline solid form (e.g., NaCDC Form A or NaCDC Form B, or any other Form provided herein) comprises no more than about 1.0 percent of total organic impurities. In some embodiments, a composition comprising a crystalline solid form (e.g., NaCDC Form A or NaCDC Form B, or any other Form provided herein) comprises no more than about 0.5 percent of total organic impurities. In some embodiments, the percent of total organic impurities is measured by HPLC.

In some embodiments, a composition comprises a crystalline solid form (e.g., NaCDC Form A or NaCDC Form B, or any other Form provided herein) and an amorphous solid form (e.g., amorphous CDCA and/or amorphous NaCDC). In some embodiments, a composition comprising a crystalline solid form is substantially free of an amorphous solid form. As used herein, the term “substantially free of an amorphous solid form” means that the composition contains no significant amount of an amorphous solid form. In some embodiments, the composition comprises at least about 90% by weight of a crystalline solid form (e.g., NaCDC Form A or NaCDC Form B, or any other Form provided herein). In some embodiments, the composition comprises at least about 95% by weight of a crystalline solid form (e.g., NaCDC Form A or NaCDC Form B, or any other Form provided herein). In some embodiments, the composition comprises at least about 99% by weight of a crystalline solid form (e.g., NaCDC Form A or NaCDC Form B, or any other Form provided herein). In some embodiments, the composition comprises no more than about 10% by weight of an amorphous solid form (e.g., amorphous CDCA and/or amorphous NaCDC). In some embodiments, the composition comprises no more than about 5% by weight of an amorphous solid form (e.g., amorphous CDCA and/or amorphous NaCDC). In some embodiments, the composition comprises no more than about 1% by weight of an amorphous solid form (e.g., amorphous CDCA and/or amorphous NaCDC).

In some embodiments, a composition comprises a free acid form (e.g., CDCA) and a salt form (e.g., NaCDC). In some such embodiments, a free acid form is crystalline, amorphous, or a mixture thereof; in some such embodiments, a salt form is crystalline, amorphous, or a mixture thereof.

In some embodiments, a composition comprises a mixture of crystalline solid forms (e.g., a mixture of one or more crystalline forms of CDCA and/or NaCDC).

### Pharmaceutical Compositions

In some embodiments, the present disclosure provides a pharmaceutical composition comprising NaCDC (e.g., in a crystalline form, such as Form A or Form B, or any other Form provided herein), and a pharmaceutically acceptable carrier. In some embodiments, the present disclosure provides a pharmaceutical composition comprising a solid form of NaCDC (e.g., a solid form described herein) and a pharmaceutically acceptable carrier. In some embodiments, provided pharmaceutical compositions comprise NaCDC (i.e., in a suitable form, such as a crystalline form described herein) and one or more fillers, disintegrants, lubricants, glidants, anti-adherents, and/or anti-statics, etc.

Pharmaceutical compositions of the present disclosure may be administered orally, parenterally, by inhalation spray, topically, rectally, nasally, buccally, vaginally, intraperitoneally, intracisternally or via an implanted reservoir. In some embodiments, provided pharmaceutical compositions are administered orally, intraperitoneally or intravenously. In some embodiments, provided pharmaceutical compositions are administered orally.

In some embodiments, a provided pharmaceutical composition is an oral dosage form (e.g., a capsule or a tablet). In some embodiments, a provided pharmaceutical composition is a tablet. In some embodiments, a provided pharmaceutical composition is a bilayer tablet (e.g., as described herein). In some embodiments, a provided pharmaceutical composition is a capsule.

In some embodiments, a provided pharmaceutical composition is a solid pharmaceutical composition (e.g., a solid dosage form such as a capsule or tablet).

In some embodiments, provided solid forms of NaCDC are useful in the preparation of previously reported pharmaceutical compositions comprising CDCA or a salt thereof. For example, PCT Publication No. WO 2021/030474 (the entire contents of which are incorporated by reference herein) describes certain articles and compositions for delivery of therapeutic agents (including CDCA or a salt thereof) to the colon of a subject. Accordingly, in some embodiments, provided solid forms can be used to prepare the articles and compositions described therein.

In some embodiments, a provided pharmaceutical composition is an article (e.g., a tablet) comprising: a first portion comprising a secretion inducing agent (e.g., CDCA or a salt thereof, e.g., NaCDC, e.g., in a solid form described herein); a second portion, adjacent to the first portion, comprising a therapeutic agent (e.g., CDCA or a salt thereof, e.g., NaCDC, e.g., in a solid form described herein); and a degradable coating associated with the article. In some embodiments, a secretion inducing agent is configured to increase the water content in the colon of the subject. In some embodiments, a local concentration of a secretion inducing agent is at least 3 mM. In some embodiments, a secretion inducing agent is configured to increase an amount of intestinal fluid present in an intestine of a subject. In some embodiments, a secretion inducing agent is configured to fully dissolve within one-fifth of the distance between the ileocecal valve and the hepatic flexure. In some embodiments, a secretion inducing agent is configured to increase the motility of the gastrointestinal tract of a subject. In some embodiments, an article comprises a secretion inducing agent in an amount greater than or equal to 5 mg and less than or equal to 5 g. In some embodiments, an article comprises a secretion inducing agent in a wt % relative to the total weight of the article of greater than or equal to 10 wt % and less than or equal to 95 wt %. In some embodiments, an article is configured to release a therapeutic agent in a colon of a subject. In some embodiments, an article comprises a therapeutic agent in an amount greater than or equal to 10 mg and less than or equal to 10 g. In some embodiments, an article comprises a therapeutic agent in a wt % relative to the total weight of the article of greater than or equal to 10 wt % and less than or equal to 95 wt %. In some embodiments, a mass ratio of the secretion inducing agent to the therapeutic agent is from 10:90 to 90:10. In some embodiments, a mass ratio of the first portion to the second portion is from 1:1 to 1:99. In some embodiments, a degradable coating comprises Eudragit S100, Phloral, HPMC, or Duocoat. In some embodiments, an article further comprises hydroxypropylmethyl cellulose (HPMC). In some embodiments, an article further comprises magnesium stearate.

In some embodiments, a provided pharmaceutical composition is an article (e.g., a tablet) comprising: a first component configured to increase the amount of intestinal fluid present in the intestine of a subject; and a second component, associated with the first component, configured to release a therapeutic agent in the intestine of the subject. In some embodiments, a first component comprises CDCA or a salt thereof (e.g., NaCDC, e.g., in a solid form described herein). In some embodiments, a second component comprises CDCA or a salt thereof (e.g., NaCDC, e.g., in a solid form described herein). In some embodiments, an article is configured for release of a therapeutic agent in a portion of an intestine of a subject (e.g., the colon).

In some embodiments, a provided pharmaceutical composition is an article (e.g., a tablet) comprising: a first portion comprising a bile acid or salt thereof (e.g., CDCA or a salt thereof, e.g., NaCDC, e.g., in a solid form described herein), wherein the first portion is configured for immediate release in the colon of a subject; a second portion, adjacent to the first portion, the second portion comprising a bile acid or salt thereof (e.g., CDCA or a salt thereof, e.g., NaCDC, e.g., in a solid form described herein) and wherein the second portion is configured for extended release in the colon of a subject; and a degradable or erodible coating associated with the article. In some embodiments, an article is a pill, tablet, or capsule. In some embodiments, a first portion is configured to fully dissolve within one-fifth of the distance between the ileocecal valve and the hepatic flexure of the subject. In some embodiments, a first portion is configured to produce a local colonic concentration of a bile acid of at least 3 mM. In some embodiments, a second portion further comprises hydroxypropylmethyl cellulose (HPMC). In some embodiments, a coating is configured such that the bile acid or salt thereof is released from the first portion in the colon of a subject. In some embodiments, a coating is or comprises Eudragit S100.

In some embodiments, a ratio (e.g., a weight ratio or height ratio) of a first portion or component and a second portion or component is greater than or equal to 1:1, greater than or equal to 1:2, greater than or equal to 1:3, greater than or equal to 1:4, greater than or equal to 1:5, greater than or equal to 1:10, greater than or equal to 1:20, greater than or equal to 1:30, greater than or equal to 1:40, greater than or equal to 1:50, greater than or equal to 1:60, greater than or equal to 1:70, greater than or equal to 1:75, greater than or equal to 1:80, greater than or equal to 1:85, greater than or equal to 1:90, greater than or equal to 1:95, greater than or equal to 1:96, greater than or equal to 1:97, greater than or equal to 1:98, or greater than or equal to 1:99. In some embodiments, a ratio (e.g., a weight ratio or height ratio) of a first portion or component and a second portion or component is less than or equal to 1:1, less than or equal to 1:2, less than or equal to 1:3, less than or equal to 1:4, less than or equal to 1:5, less than or equal to 1:10, less than or equal to 1:20, less than or equal to 1:30, less than or equal to 1:40, less than or equal to 1:50, less than or equal to 1:60, less than or equal to 1:70, less than or equal to 1:75, less than or equal to 1:80, less than or equal to 1:85, less than or equal to 1:90, less than or equal to 1:95, less than or equal to 1:96, less than or equal to 1:97, less than or equal to 1:98, or less than or equal to 1:99. Combinations of the above-referenced ranges are also possible (e.g., less than or equal to 1:1 and greater than or equal to 1:99).

In some embodiments, a ratio (e.g., a weight ratio or height ratio) of a second portion or component and a first portion or component is greater than or equal to 1:1, greater than or equal to 1:2, greater than or equal to 1:3, greater than or equal to 1:4, greater than or equal to 1:5, greater than or equal to 1:10, greater than or equal to 1:20, greater than or equal to 1:30, greater than or equal to 1:40, greater than or equal to 1:50, greater than or equal to 1:60, greater than or equal to 1:70, greater than or equal to 1:75, greater than or equal to 1:80, greater than or equal to 1:85, greater than or equal to 1:90, greater than or equal to 1:95, greater than or equal to 1:96, greater than or equal to 1:97, greater than or equal to 1:98, or greater than or equal to 1:99. In some embodiments, a ratio (e.g., a weight ratio or height ratio) of a second portion or component and a first portion or component is less than or equal to 1:1, less than or equal to 1:2, less than or equal to 1:3, less than or equal to 1:4, less than or equal to 1:5, less than or equal to 1:10, less than or equal to 1:20, less than or equal to 1:30, less than or equal to 1:40, less than or equal to 1:50, less than or equal to 1:60, less than or equal to 1:70, less than or equal to 1:75, less than or equal to 1:80, less than or equal to 1:85, less than or equal to 1:90, less than or equal to 1:95, less than or equal to 1:96, less than or equal to 1:97, less than or equal to 1:98, or less than or equal to 1:99. Combinations of the above-referenced ranges are also possible (e.g., less than or equal to 1:1 and greater than or equal to 1:99).

In some embodiments, a provided pharmaceutical composition comprises a secretion inducing agent. In some embodiments, a secretion inducing agent is a chemical species that stimulates the release of increased intestinal fluid along the gastrointestinal tract (e.g., relative to the basal release of intestinal fluid and/or the basal release of intestinal fluid in response to a foreign body present in the gastrointestinal tract such as food). In this way, the increased amount of intestinal fluid enhances the solubility and/or absorption of a therapeutic agent. In some embodiments, a secretion inducing agent is a bile acid (e.g., CDCA) or salt thereof. In some embodiments, a secretion inducing agent is NaCDC. In some embodiments, a secretion inducing agent comprises bisacodyl, senna, sennoside, linaclotide, plecanatide, lubiprostone, 30 methylnaltrexone, naloxegol, polyethyleneglycol, lactulose, or prucalopride. In some embodiments, a secretion inducing agent may be a salt, such as magnesium citrate, magnesium hydroxide, or a bile salt, as non-limiting examples. Other secretion inducing agents are possible, as any chemical species that stimulates the release of intestinal fluid along the gastrointestinal tract may be function as a secretion inducing agent.

In some embodiments, a wt % of a secretion inducing agent relative to the total weight of the article or composition (e.g., a tablet, a capsule, etc.) is greater than or equal to 10 wt %, greater than or equal to 15 wt %, greater than or equal to 20 wt %, greater than or equal to 25 wt %, greater than or equal to 30 wt %, greater than or equal 40 wt %, greater than or equal to 50 wt %, greater than or equal to 60 wt %, greater than or equal to 70 wt %, greater than or equal to 75 wt %, greater than or equal to 80 wt %, greater than or equal 90 wt %, or greater than or equal to 95 wt %. In some embodiments, a wt % of a secretion-inducing agent relative to the total weight of an article or composition (e.g., a tablet, a capsule, etc.) is less than or equal to 95 wt %, less than or equal to 90 wt %, less than or equal to 80 wt %, less than or equal to 75 wt %, less than or equal to 70 wt %, less than or equal to 60 wt %, less than or equal to 50 wt %, less than or equal to 40 wt %, less than or equal to 30 wt %, less than or equal to 25 wt %, less than or equal to 20 wt %, less than or equal to 15 wt %, or less than or equal to 10 wt %. Combinations of the above-references ranges are also possible (e.g., greater than or equal to 10 wt % and less than or equal to 50 wt %). Other ranges are possible.

In some embodiments, an amount (e.g., mass) of a secretion-inducing agent present in an article or composition (e.g., a tablet, a capsule, etc.) is greater than or equal to 5 mg, greater than or equal to 10 mg, greater than or equal to 20 mg, greater than or equal to 20 mg, greater than or equal to 30 mg, greater than or equal to 50 mg, greater than or equal to 60 mg, greater than or equal to 70 mg, greater than or equal to 75 mg, greater than or equal to 80 mg, greater than or equal to 90 mg, greater than or equal to 95 mg, greater than or equal to 100 mg, greater than or equal to 250 mg, greater than or equal to 500 mg, greater than or equal to 750 mg, greater than or equal to 1 g, greater than or equal to 2 g, greater than or equal to 3 g, greater than or equal to 4 g, or greater than or equal to 5 g. In some embodiments, an amount of a secretion-inducing agent present in an article or composition (e.g., a tablet, a capsule, etc.) is less than or equal to 5 g, less than or equal to 4 g, less than or equal to 3 g, less than or equal to 2 g, less than or equal to 1 g, less than or equal to 750 mg, less than or equal to 500 mg, less than or equal to 250 mg, less than or equal to 100 mg, less than or equal to 95 mg, less than or 30 equal to 90 mg, less than or equal 80 mg, less than or equal to 75 mg, less than or equal to 70 mg, less than or equal to 60 mg, less than or equal to 50 mg, less than or equal to 40 mg, less than or equal to 30 mg, less than or equal to 25 mg, less than or equal to 20 mg, less than or equal to 10 mg, or less than or equal to 5 mg. Combinations of the above-referenced ranges are also possible (e.g., greater than or equal to 5 mg and less than or equal to 5 g). Other ranges are possible.

In some embodiments, a provided pharmaceutical composition comprises a therapeutic agent. In some embodiments, a therapeutic agent may be one or a combination of therapeutic, diagnostic, and/or enhancement agents, such as drugs, nutrients, microorganisms, in vivo sensors, and tracers. In some embodiments, a therapeutic agent is a nutraceutical, prophylactic, or diagnostic agent. Therapeutic agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action, such as increasing the amount of intestinal fluid present in the colon of a subject. For example, in some embodiments, therapeutic agents include chemicals traditionally regarded as drugs, vaccines, and biopharmaceuticals. Listings of examples of known therapeutic agents can be found, for example, in the United States Pharmacopeia (USP), Goodman and Gilman's The Pharmacological Basis of Therapeutics, 13th Ed., McGraw Hill, 2017; Katzung, B. and Vanderah, T. W. (eds.) Basic and Clinical Pharmacology, McGraw-Hill; 15th edition (Dec. 5, 2020); Prescriber's Digital Reference (pdr.net); The Merck Manual 20^{th} ed. (2018), Porter, R. E. (ed.), Wiley, or, in the case of animals, The Merck Veterinary Manual, 11th ed., Kahn, C. A. (ed.), Merck Manuals, 2016; and “Approved Drug Products with Therapeutic Equivalence and Evaluations,” published by the United States Food and Drug Administration (F.D.A.) (the “Orange Book”).

In some embodiments, a therapeutic agent is a bile acid (e.g., CDCA) or salt thereof. In some embodiments, a therapeutic agent is NaCDC. Non-limiting examples of bile acids include chenodeoxycholic acid, ursodeoxycholic acid, deoxycholic acid, taurocholic, glycocholic acid, cholic acid, taurochenodeoxycholic acid, glycochenodeoxycholic acid, deoxycholic acid, and lithocholic acid. Accordingly, in some embodiments, a therapeutic agent is selected from the group consisting of chenodeoxycholic acid, ursodeoxycholic acid, deoxycholic acid, taurocholic, glycocholic acid, cholic acid, taurochenodeoxycholic acid, glycochenodeoxycholic acid, deoxycholic acid, and lithocholic acid, or a salt thereof.

In some embodiments, a wt % of a therapeutic agent relative to the total weight of the article or composition (e.g., a tablet, a capsule, etc.) is greater than or equal to 10 wt %, greater than or equal to 15 wt %, greater than or equal to 20 wt %, greater than or equal to 25 wt %, greater than or equal to 30 wt %, greater than or equal 40 wt %, greater than or equal to 50 wt %, greater than or equal to 60 wt %, greater than or equal to 70 wt %, greater than or equal to 75 wt %, greater than or equal to 80 wt %, greater than or equal 90 wt %, or greater than or equal to 95 wt %. In some embodiments, a wt % of a therapeutic agent relative to the total weight of an article or composition (e.g., a tablet, a capsule, etc.) is less than or equal to 95 wt %, less than or equal to 90 wt %, less than or equal to 80 wt %, less than or equal to 75 wt %, less than or equal to 70 wt %, less than or equal to 60 wt %, less than or equal to 50 wt %, less than or equal to 40 wt %, less than or equal to 30 wt %, less than or equal to 25 wt %, less than or equal to 20 wt %, less than or equal to 15 wt %, or less than or equal to 10 wt %. Combinations of the above-references ranges are also possible (e.g., greater than or equal to 10 wt % and less than or equal to 50 wt %). Other ranges are possible.

In some embodiments, an amount (e.g., mass) of a therapeutic agent present in an article or composition (e.g., a tablet, a capsule, etc.) is greater than or equal to 10 mg, greater than or equal to 20 mg, greater than or equal to 25 mg, greater than or equal to 60 mg, greater than or equal to 70 mg, greater than or equal to 75 mg, greater than or equal to 80 mg, greater than or equal to 90 mg, greater than or equal to 95 mg, greater than or equal to 100 mg, greater than or equal to 250 mg, greater than or equal to 300 mg, greater than or equal to 400 mg, greater than or equal to 500 mg, greater than or equal to 750 mg, greater than or equal to 1 g, greater than or equal to 2 g, greater than or equal to 3 g, greater than or equal to 4 g, greater than or equal to 5 g, greater than or equal to 6 g, greater than or equal to 7 g, greater than or equal to 8 g, greater than or equal to 9 g, or greater than or equal to 10 g. In some embodiments, an amount (e.g., mass) of a therapeutic agent present in an article or composition (e.g., a tablet, a capsule, etc.) is less than or equal to 10 mg, less than or equal to 20 mg, less than or equal to 25 mg, less than or equal to 60 mg, less than or equal to 70 mg, less than or equal to 75 mg, less than or equal to 80 mg, less than or equal to 90 mg, less than or equal to 95 mg, less than or equal to 100 mg, less than or equal to 250 mg, less than or equal to 300 mg, less than or equal to 400 mg, less than or equal to 500 mg, less than or equal to 750 mg, less than or equal to 1 g, less than or equal to 2 g, less than or equal to 3 g, less than or equal to 4 g, less than or equal to 5 g, less than or equal to 6 g, less than or equal to 7 g, less than or equal to 8 g, less than or equal to 9 g, or less than or equal to 10 g. Combinations of the above-referenced ranges are also possible (e.g., greater than or equal to 10 mg and less than or equal to 10 g). Other ranges are possible.

In some embodiments, a mass ratio of a secretion inducing agent to a therapeutic agent is greater than or equal to 10:90, greater than or equal to 20:80, greater than or equal to 30:70, greater than or equal to 40:60, greater than or equal to 50:50, greater than or equal to 60:40, greater than or equal to 70:30, greater than or equal to 80:20, or greater than or equal to 90:10. In some embodiments, a mass ratio of a secretion-inducing agent to a therapeutic agent is less than or equal to 90:10, less than or equal to 80:20, less than 10 or equal to 70:30, less than or equal to 60:40, less than or equal to 50:50, less than or equal to 40:60, less than or equal to 30:70, less than or equal to 20:80, or less than or equal to 10:90. Combinations of the above-referenced ranges are also possible (e.g., greater than or equal to 10:90 and less than or equal to 30:70). Other ranges are possible.

In some embodiments, a provided pharmaceutical composition comprises a coating. In some embodiments, a coating is degradable and/or erodible (e.g., by gastrointestinal fluids under physiological conditions). In some embodiments, a coating is or comprises Eudragit S100. Any suitable coating that is configured to release a secretion inducing agent to the desired portion of the gastrointestinal tract (such as in the distal portion of ileum or the distal part of the colon, as a non-limiting example) may be used. Non-limiting examples of suitable degradable coatings include Eudragit S, Phloral, CODES, and Duocoat. In some embodiments, the coating is or comprises, for example, hydroxypropyl methylcellulose (HPMC). Other coatings are also possible.

In some embodiments, a provided pharmaceutical composition comprises one or more additional components (e.g., excipients). In some embodiments, additional components may contribute to stability of a composition, solubility of a composition, and/or the composition's ability to deliver a therapeutic agent to a desired portion of the gastrointestinal tract. In some embodiments, an additional component is magnesium stearate. In some embodiments, an additional component is hydroxypropylmethyl cellulose. In some embodiments, an additional component is Aerosil® 200 Pharma. In some embodiments, an additional component is selected from microcrystalline cellulose (e.g., Avicel PH102), croscarmellose sodium, copovidone, magnesium stearate, calcium hydrogen phosphate dihydrate, sodium starch glycolate, sodium stearyl fumarate, poly(ethylene oxide) (e.g., PolyOX WSR1105), and hydroxypropyl methylcellulose (e.g., HPMC K4M). In some embodiments, additional components may form a matrix around or within the composition. In some embodiments, additional components may control the release of one or more other components of the composition (e.g., secretion inducing agent or therapeutic agent). Non-limiting examples of suitable matrix forming and/or release controlling agents include methylcellulose, hydroxypropyl cellulose, hydroxypropyl methylcellulose, alginates, plant derived gums, chitosan, gelatin, pectin, carrageenans, polyacrylates, polyethylene oxides, and starch. Examples of hydrophobic matrix forming and/or release controlling agents include waxes, fatty acids, fatty alcohols and esters, glycerol esters, polyesteramide, ethyl cellulose, polyethylene, polypropylene, polythiourethane, polyvinylbutyral, polylactic acid, poly(lactide-coglycolide), cellulose acetate, and cellulose acetate butyrate. Other additional components that may assist with delivery of a secretion inducing agent or a therapeutic agent are also possible.

In some embodiments, a provided pharmaceutical composition may comprise a hydrophilizing agent. Non-limiting examples of suitable hydrophilizing agents include cyclodextrins, surfactants, solid buffers (e.g. sodium citrate/citric acid). Other examples of hydrophilizing agents are possible as the disclosure is not so limited.

In some embodiments, a provided pharmaceutical composition is configured such that, when administered to a subject, a secretion inducing agent is present along the gastrointestinal tract with a local concentration of at least 3 mM. In some embodiments, a local concentration is at least 5 mM. In some embodiments, a local concentration of at least at least 3 mM, at least 5 mM, at least 10 mM, at least 15 mM, at least 20 mM, at least 30 mM, or at least 50 mM is produced. In some embodiments, a local concentration of less than or equal to 100 mM, less than or equal to 50 mM, less than or equal to 30 mM, less than or equal to 20 mM, less than or equal to 15 mM, less than or equal to 10 mM, or less than or equal to 5 mM is produced. As described herein, “local concentration” refers to the amount of substance per unit volume at a position nearby the article. For example, the concentration of a secretion inducing agent along the entirety of the gastrointestinal tract may be substantially less than 3 mM, but the concentration of the secretion inducing agent in the vicinity of a provided composition or article may be equal to or greater than 3 mM.

In some embodiments, a provided pharmaceutical composition has a largest cross-sectional dimension (e.g., a diameter) of at least 10 mm, at least 12 mm, at least 14 mm, of at least 15 mm, of at least 18 mm, of at least 19 mm, of at least 21 mm, of at least 23 mm, or of at least 26. In some embodiments, an provided pharmaceutical composition has a largest cross-sectional dimension of at most 27 mm, of at most 24 mm, of at most 22 mm, of at most 20 mm, of at most 18 mm, of at most 16 mm, of at most 15 mm, or of at most 10 mm.

In some embodiments, a provided pharmaceutical composition is a bilayer tablet coated with Eudragit S100, the bilayer tablet comprising: a first layer comprising 81% NaCDC, 17% hydroxypropyl methylcellulose (HPMC) (MW: 120,000), and 2% magnesium stearate; and a second layer comprising 98% NaCDC and 2% magnesium stearate. In some such embodiments, a bilayer tablet comprises 400 mg NaCDC in a first layer. In some such embodiments, a bilayer tablet comprises 98 mg NaCDC in a second layer.

In some embodiments, a provided pharmaceutical composition is a bilayer tablet coated with Eudragit S100, the bilayer tablet comprising: a first layer comprising 87.5% NaCDC, 10% HPMC (MW: 120,000), 2% magnesium stearate, and 0.5% Aerosil 200; and a second layer comprising 97.5% NaCDC, 2% magnesium stearate, and 0.5% Aerosil 200. In some such embodiments, a bilayer tablet comprises 400 mg NaCDC in a first layer. In some such embodiments, a bilayer tablet comprises 100 mg NaCDC in a second layer.

In some embodiments, a provided pharmaceutical composition is prepared by (i) providing NaCDC in any suitable form such as a crystalline form described herein; and (ii) formulating the NaCDC with suitable excipients, to provide the pharmaceutical composition.

### Uses

The present disclosure provides uses for solid forms and compositions described herein. In some embodiments, provided solid forms and compositions thereof are useful in medicine (e.g., as therapy). In some embodiments, provided solid forms and compositions described herein are useful in research, e.g., as analytical tools and/or controls.

In some embodiments, the present disclosure provides methods of administering provided solid forms and compositions thereof to a subject in need thereof. In some embodiments, the present disclosure provides methods of administering provided solid forms and compositions thereof to a subject suffering from a gastrointestinal disorder (e.g., constipation, e.g., IBS-C). In some embodiments, the present disclosure provides methods of administering provided solid forms and compositions thereof to a subject suffering from a systemic disorder (e.g., one in which administration of therapy, e.g., a provided solid form or composition thereof, to the gastrointestinal tract may be effective to treat the systemic disorder). In some embodiments, the present disclosure provides methods of administering provided solid forms and compositions thereof to a subject in need of a colonoscopy (e.g., for diagnosis of colorectal lesions). In some embodiments, the present disclosure provides methods of administering provided solid forms and compositions thereof to a subject suffering from constipation, ulcerative colitis, Crohn's disease, diabetes, metabolic disorders, obesity, traveler's diarrhea, hepatic encephalopathy, or diseases associated with modulation of the biome. In some embodiments, such methods comprise administering a provided solid form or composition thereof orally to a subject.

It will be appreciated that, as used herein, “subject” refers an organism, typically a mammal (e.g., a human, a non-human mammal, a non-human primate, a primate, a mouse, a rat, a hamster, a gerbil, a cat, a dog, etc.). In some embodiments, a subject is a human.

In some embodiments, the present disclosure provides methods of delivering a therapeutic agent (e.g., a bile acid, e.g., CDCA or salt thereof) to the intestine (e.g., the colon) of a subject in need thereof. In some embodiments, such methods comprise administering a solid form or composition, e.g., a composition comprising or prepared from one or more solid forms provided herein, described herein. In some embodiments, such methods comprise administering a provided solid form or composition thereof orally to a subject.

In some embodiments, the present disclosure provides methods of treating a disease, disorder, or condition comprising administering a provided solid form or composition thereof to a subject in need thereof. As used herein, “treat,” “treatment,” or “treating” refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. Thus, in some embodiments, treatment may be prophylactic; in some embodiments, treatment may be therapeutic.

In some embodiments, the present disclosure provides methods of treating a gastrointestinal disorder (e.g., constipation, e.g., IBS-C) comprising administering a provided solid form or composition thereof to a subject in need thereof. In some embodiments, provided methods achieve certain desirable outcomes, such as, e.g., improved efficacy and/or reduced incidence of abdominal pain and/or cramping (e.g., as compared to another formulation of CDCA or salt thereof). In some embodiments, such methods comprise administering a provided solid form or composition thereof orally to a subject.

In some embodiments, the present disclosure provides methods of treating a systemic disorder (e.g., one in which administration of therapy, e.g., a provided solid form or composition thereof, to the gastrointestinal tract may be effective to treat the systemic disorder) comprising administering a provided solid form or composition thereof to a subject in need thereof. In some embodiments, the present disclosure provides methods of treating a disease, disorder, or condition selected from the group consisting of constipation, ulcerative colitis, Crohn's disease, diabetes, metabolic disorders, obesity, traveler's diarrhea, hepatic encephalopathy, and diseases associated with modulation of the biome, the method comprising administering a provided solid form or composition thereof to a subject in need thereof. In some embodiments, such methods comprise administering a provided solid form or composition thereof orally to a subject.

In some embodiments, the present disclosure provides methods of diagnosing colorectal lesions, comprising administering a provided solid form or composition thereof to a subject in need thereof. In some such embodiments, the subject has, is or will undergo a colonoscopy. In some embodiments, such methods comprise administering a provided solid form or composition thereof orally to a subject.

### Exemplary Embodiments

The following numbered embodiments, while non-limiting, are exemplary of certain aspects of the present disclosure:

1. A crystalline solid form of sodium chenodeoxycholate, wherein the solid form is selected from Form A and Form B.
2. The solid form of embodiment 1, wherein the solid form is Form A.
3. The solid form of embodiment 2, wherein the solid form is characterized by one or more peaks in its XRPD pattern selected from those at about 6.07, about 6.55, about 10.72, about 14.64, about 15.06, about 17.58, and about 18.34 degrees 2-theta.
4. The solid form of embodiment 2, wherein the solid form is characterized by peaks in its XRPD pattern selected from those at about 6.07, about 6.55, about 10.72, about 14.64, about 15.06, about 17.58, and about 18.34 degrees 2-theta.
5. The solid form of embodiment 2, wherein the solid form is characterized by peaks in its XRPD pattern at substantially all of:

2θ ± 0.2 [°]

6.07
6.55
8.36
9.92
10.72
11.55
11.93
12.99
13.36
14.64
15.06
15.54
15.93
16.68
16.99
17.31
17.58
18.07
18.34

6. The solid form of embodiment 2, wherein the solid form is characterized by one or more of.
(i) an XRPD pattern substantially similar to that depicted in FIG. 1;
(ii) a DSC pattern showing loss of water starting just above ambient temperature and continuing to about 150° C.;
(iii) a DSC pattern substantially similar to that depicted in FIG. 2;
(iv) a TGA pattern showing a weight loss of 4.3% up to 150° C.; and
(v) a TGA pattern substantially similar to that depicted in FIG. 3.
   7. The solid form of embodiment 1, wherein the solid form is Form B.
   8. The solid form of embodiment 7, wherein the solid form is characterized by one or more peaks in its XRPD pattern selected from those at about 6.75, about 8.14, about 9.79, about 14.02, about 16.10, and about 18.63 degrees 2-theta.
   9. The solid form of embodiment 7, wherein the solid form is characterized by peaks in its XRPD pattern selected from those at about 6.75, about 8.14, about 9.79, about 14.02, about 16.10, and about 18.63 degrees 2-theta.
   10. The solid form of embodiment 7, wherein the solid form is characterized by peaks in its XRPD pattern at substantially all of:

2θ ± 0.2 [°]

6.75
8.14
9.79
11.33
13.27
13.56
14.02
14.79
15.05
16.10
16.37
16.78
17.74
18.63
19.71
20.00
20.35
21.89
22.90

11. The solid form of embodiment 7, wherein the solid form is characterized by one or more of:
(i) an XRPD pattern substantially similar to that depicted in FIG. 5;
(ii) a DSC pattern showing no thermal event from room temperature to around 288° C.;
(iii) a DSC pattern substantially similar to that depicted in FIG. 6;
(iv) a TGA pattern showing a weight loss of <0.1% up to 150° C.; and
(v) a TGA pattern substantially similar to that depicted in FIG. 7.
   12. A crystalline solid form of sodium chenodeoxycholate obtainable from a process described herein.
   13. A crystalline solid form of sodium chenodeoxycholate prepared by a method comprising steps of: providing a mixture of chenodeoxycholic acid in methyl isobutyl ketone; adding to the mixture an aqueous solution of sodium hydroxide; heating the mixture (e.g., to azeotropic reflux); and removing the solvent to provide the crystalline solid form of sodium chenodeoxycholate.
   14. A crystalline solid form of sodium chenodeoxycholate prepared by a method comprising steps of: providing a mixture of chenodeoxycholic acid in n-butanol; adding to the mixture an aqueous solution of sodium hydroxide; heating the mixture (e.g., to azeotropic reflux); and removing the solvent to provide the crystalline solid form of sodium chenodeoxycholate.
   15. A crystalline solid form of sodium chenodeoxycholate, wherein the solid form is selected from Form S1, Form S2, Form S3, Form S4, Form S5, Form S6, Form S7, Form S9-a, Form S9-b, Form S10, Form S11, Form S12, Form S13, Form S14, and Form S15.
   16. The solid form of embodiment 15, wherein the solid form is Form S1.
   17. The solid form of embodiment 16, wherein the solid form is characterized by one or more peaks in its XRPD pattern selected from those at about 5.45, about 5.80, about 7.46, about 9.76, about 12.40, about 14.88, and about 20.02 degrees 2-theta.
   18. The solid form of embodiment 16, wherein the solid form is characterized by peaks in its XRPD pattern selected from those at about 5.45, about 5.80, about 7.46, about 9.76, about 12.40, about 14.88, and about 20.02 degrees 2-theta.
   19. The solid form of embodiment 16, wherein the solid form is characterized by peaks in its XRPD pattern at substantially all of those listed in Table S1-A or Table S1-B.
   20. The solid form of embodiment 16, wherein the solid form is characterized by one or more of.
(i) an XRPD pattern substantially similar to that depicted in FIG. 19A, FIG. 24A, and/or FIG. 48;
(ii) a DSC pattern showing a thermal event at about 96.0° C., about 142.5° C., and/or about 313.4° C.;
(iii) a DSC pattern substantially similar to that depicted in FIG. 19B, FIG. 24B, FIG. 27, and/or FIG. 49;
(iv) a TGA pattern showing a weight loss of about 2.177% up to about 150° C.; and
(v) a TGA pattern substantially similar to that depicted in FIG. 19B, FIG. 24B, FIG. 27, and/or FIG. 49.
   21. The solid form of embodiment 15, wherein the solid form is Form S2. 22. The solid form of embodiment 21, wherein the solid form is characterized by one or more peaks in its XRPD pattern selected from those at about 7.11, about 7.78, about 9.81, about 12.58, about 12.96, and about 13.54 degrees 2-theta.
   23. The solid form of embodiment 21, wherein the solid form is characterized by peaks in its XRPD pattern selected from those at about 7.11, about 7.78, about 9.81, about 12.58, about 12.96, and about 13.54 degrees 2-theta.
   24. The solid form of embodiment 21, wherein the solid form is characterized by peaks in its XRPD pattern at substantially all of those listed in Table S2.
   25. The solid form of embodiment 21, wherein the solid form is characterized by one or more of.
(i) an XRPD pattern substantially similar to that depicted in FIG. 33;
(ii) a DSC pattern showing a thermal event at about 112.9° C., about 177.8° C., and/or about 334.1° C.;
(iii) a DSC pattern substantially similar to that depicted in FIG. 21A, FIG. 25, and/or FIG. 30;
(iv) a TGA pattern showing a weight loss of about 3.95% up to about 150° C.; and
(v) a TGA pattern substantially similar to that depicted in FIG. 21A, FIG. 25, and/or FIG. 30.
   26. The solid form of embodiment 15, wherein the solid form is Form S3, Form S6, and/or Form S11.
   27. The solid form of embodiment 26, wherein the solid form is characterized by one or more peaks in its XRPD pattern selected from those at about 5.00, about 7.56, about 10.56, about 11.45, about 11.93, and about 12.46 degrees 2-theta.
   28. The solid form of embodiment 26, wherein the solid form is characterized by peaks in its XRPD pattern selected from those at about 5.00, about 7.56, about 10.56, about 11.45, about 11.93, and about 12.46 degrees 2-theta.
   29. The solid form of embodiment 26, wherein the solid form is characterized by peaks in its XRPD pattern at substantially all of those listed in Table S3, Table S6, or Table S11.
   30. The solid form of embodiment 26, wherein the solid form is characterized by one or more of:
(i) an XRPD pattern substantially similar to that depicted in FIG. 21B and/or FIG. 50A;
(ii) a DSC pattern showing a thermal event at about 50.6° C., about 199.8° C., and/or about 331.5° C.;
(iii) a DSC pattern substantially similar to that depicted in FIG. 29 and/or FIG. 41;
(iv) a TGA pattern showing a weight loss of about 2.456% up to about 150° C.; and
(v) a TGA pattern substantially similar to that depicted in FIG. 29 and/or FIG. 41.
   31. The solid form of embodiment 15, wherein the solid form is Form S4.
   32. The solid form of embodiment 31, wherein the solid form is characterized by one or more peaks in its XRPD pattern selected from those at about 7.07, about 7.65, about 9.70, about 13.43, about 15.02, about 16.52, and about 16.96 degrees 2-theta.
   33. The solid form of embodiment 31, wherein the solid form is characterized by peaks in its XRPD pattern selected from those at about 7.07, about 7.65, about 9.70, about 13.43, about 15.02, about 16.52, and about 16.96 degrees 2-theta.
   34. The solid form of embodiment 31, wherein the solid form is characterized by peaks in its XRPD pattern at substantially all of those listed in Table S4.
   35. The solid form of embodiment 31, wherein the solid form is characterized by one or more of.
(i) an XRPD pattern substantially similar to that depicted in FIG. 31A;
(ii) a DSC pattern showing a thermal event at about 146.5° C. and/or about 333.9° C.;
(iii) a DSC pattern substantially similar to that depicted in FIG. 22 and/or FIG. 31B;
(iv) a TGA pattern showing a weight loss of about 14.23% up to about 180° C.; and (v) a TGA pattern substantially similar to that depicted in FIG. 22 and/or FIG. 31B.
   36. The solid form of embodiment 15, wherein the solid form is Form S5.
   37. The solid form of embodiment 36, wherein the solid form is characterized by one or more peaks in its XRPD pattern selected from those at about 7.11, about 8.63, about 12.08, about 12.75, about 13.46, about 14.25, and about 16.68 degrees 2-theta.
   38. The solid form of embodiment 36, wherein the solid form is characterized by peaks in its XRPD pattern selected from those at about 7.11, about 8.63, about 12.08, about 12.75, about 13.46, about 14.25, and about 16.68 degrees 2-theta.
   39. The solid form of embodiment 36, wherein the solid form is characterized by peaks in its XRPD pattern at substantially all of those listed in Table S5.
   40. The solid form of embodiment 36, wherein the solid form is characterized by one or more of:
(i) an XRPD pattern substantially similar to that depicted in FIG. 23A;
(ii) a DSC pattern showing a thermal event at about 82° C., about 183.9° C., and/or about 331.5° C.;
(iii) a DSC pattern substantially similar to that depicted in FIG. 23B, FIG. 28, FIG. 32A, FIG. 32B, and/or FIG. 47;
(iv) a TGA pattern showing a weight loss of about 4.79% up to about 180° C.; and
(v) a TGA pattern substantially similar to that depicted in FIG. 23B, FIG. 28, FIG. 32A, FIG. 32B, and/or FIG. 47.
   41. The solid form of embodiment 15, wherein the solid form is Form S7.
   42. The solid form of embodiment 41, wherein the solid form is characterized by one or more peaks in its XRPD pattern selected from those at about 8.47, about 9.90, about 14.36, about 15.26, about 17.00, and about 17.72 degrees 2-theta.
   43. The solid form of embodiment 41, wherein the solid form is characterized by peaks in its XRPD pattern selected from those at about 8.47, about 9.90, about 14.36, about 15.26, about 17.00, and about 17.72 degrees 2-theta.
   44. The solid form of embodiment 41, wherein the solid form is characterized by peaks in its XRPD pattern at substantially all of those listed in Table S7.
   45. The solid form of embodiment 41, wherein the solid form is characterized by one or more of:
(i) an XRPD pattern substantially similar to that depicted in FIG. 26A;
(ii) a DSC pattern showing a thermal event at about 109.2° C., about 324.5° C., and/or about 335.3° C.;
(iii) a DSC pattern substantially similar to that depicted in FIG. 26B;
(iv) a TGA pattern showing a weight loss of about 10.04% up to about 180° C.; and
(v) a TGA pattern substantially similar to that depicted in FIG. 26B.
   46. The solid form of embodiment 15, wherein the solid form is Form S9-a.
   47. The solid form of embodiment 46, wherein the solid form is characterized by one or more peaks in its XRPD pattern selected from those at about 5.10, about 7.00, about 13.52, about 14.23, about 15.46, and about 18.78 degrees 2-theta.
   48. The solid form of embodiment 46, wherein the solid form is characterized by peaks in its XRPD pattern selected from those at about 5.10, about 7.00, about 13.52, about 14.23, about 15.46, and about 18.78 degrees 2-theta.
   49. The solid form of embodiment 46, wherein the solid form is characterized by peaks in its XRPD pattern at substantially all of those listed in Table S9-a.
   50. The solid form of embodiment 46, wherein the solid form is characterized by one or more of.
(i) an XRPD pattern substantially similar to that depicted in FIG. 37 (bottom spectrum);
(ii) a DSC pattern showing a thermal event at about 99.9° C. and/or about 328° C.;
(iii) a DSC pattern substantially similar to that depicted in FIG. 38 and/or FIG. 39;
(iv) a TGA pattern showing a weight loss of about 8.635% up to about 150° C.; and
(v) a TGA pattern substantially similar to that depicted in FIG. 38 and/or FIG. 39.
   51. The solid form of embodiment 15, wherein the solid form is Form S9-b.
   52. The solid form of embodiment 51, wherein the solid form is characterized by one or more peaks in its XRPD pattern selected from those at about 5.47, about 7.48, about 9.82, about 12.66, and about 15.07 degrees 2-theta.
   53. The solid form of embodiment 51, wherein the solid form is characterized by peaks in its XRPD pattern selected from those at about 5.47, about 7.48, about 9.82, about 12.66, and about 15.07 degrees 2-theta.
   54. The solid form of embodiment 51, wherein the solid form is characterized by peaks in its XRPD pattern at substantially all of those listed in Table S9-b.
   55. The solid form of embodiment 51, wherein the solid form is characterized by an XRPD pattern substantially similar to that depicted in FIG. 37 (bottom spectrum).
   56. The solid form of embodiment 15, wherein the solid form is Form S10.
   57. The solid form of embodiment 56, wherein the solid form is characterized by one or more peaks in its XRPD pattern selected from those at about 5.13, about 7.01, about 8.69, about 9.11, about 13.55, about 14.91, and about 15.53 degrees 2-theta.
   58. The solid form of embodiment 56, wherein the solid form is characterized by peaks in its XRPD pattern selected from those at about 5.13, about 7.01, about 8.69, about 9.11, about 13.55, about 14.91, and about 15.53 degrees 2-theta.
   59. The solid form of embodiment 56, wherein the solid form is characterized by peaks in its XRPD pattern at substantially all of those listed in Table S10.
   60. The solid form of embodiment 56, wherein the solid form is characterized by one or more of:
(i) an XRPD pattern substantially similar to that depicted in FIG. 40A;
(ii) a DSC pattern showing a thermal event at about 141.3° C., about 324° C. and/or about 336° C.;
(iii) a DSC pattern substantially similar to that depicted in FIG. 40B;
(iv) a TGA pattern showing a weight loss of about 16% up to about 150° C.; and
(v) a TGA pattern substantially similar to that depicted in FIG. 40B.
   61. The solid form of embodiment 15, wherein the solid form is Form S12 and/or Form S15.
   62. The solid form of embodiment 61, wherein the solid form is characterized by one or more peaks in its XRPD pattern selected from those at about 4.82, about 5.22, about 5.89, about 10.81, about 13.00, about 15.00, and about 18.94 degrees 2-theta.
   63. The solid form of embodiment 61, wherein the solid form is characterized by peaks in its XRPD pattern selected from those at about 4.82, about 5.22, about 5.89, about 10.81, about 13.00, about 15.00, and about 18.94 degrees 2-theta.
   64. The solid form of embodiment 61, wherein the solid form is characterized by peaks in its XRPD pattern at substantially all of those listed in Table S12 or Table S15.
   65. The solid form of embodiment 61, wherein the solid form is characterized by one or more of:
(i) an XRPD pattern substantially similar to that depicted in FIG. 42;
(ii) a DSC pattern showing a thermal event at about 80.8° C., about 180° C., and/or about 332° C.;
(iii) a DSC pattern substantially similar to that depicted in FIG. 43;
(iv) a TGA pattern showing a weight loss of about 4.448% up to about 150° C.; and
(v) a TGA pattern substantially similar to that depicted in FIG. 43.
   66. The solid form of embodiment 15, wherein the solid form is Form S13.
   67. The solid form of embodiment 66, wherein the solid form is characterized by one or more peaks in its XRPD pattern selected from those at about 4.95, about 7.53, about 9.85, about 11.55, about 12.12, and about 15.01 degrees 2-theta.
   68. The solid form of embodiment 66, wherein the solid form is characterized by peaks in its XRPD pattern selected from those at about 4.95, about 7.53, about 9.85, about 11.55, about 12.12, and about 15.01 degrees 2-theta.
   69. The solid form of embodiment 66, wherein the solid form is characterized by peaks in its XRPD pattern at substantially all of those listed in Table S13.
   70. The solid form of embodiment 66, wherein the solid form is characterized by one or more of:
(i) an XRPD pattern substantially similar to that depicted in FIG. 44 and/or FIG. 53;
(ii) a DSC pattern showing a thermal event at about 55.4° C., about 148.8° C., about 191.8° C. and/or about 335° C.;
(iii) a DSC pattern substantially similar to that depicted in FIG. 45;
(iv) a TGA pattern showing a weight loss of about 6.162% up to about 180° C.; and
(v) a TGA pattern substantially similar to that depicted in FIG. 45.
   71. The solid form of embodiment 15, wherein the solid form is Form S14.
   72. The solid form of embodiment 71, wherein the solid form is characterized by one or more peaks in its XRPD pattern selected from those at about 5.15, about 5.50, about 7.05, about 12.04, about 14.90, and about 16.56 degrees 2-theta.
   73. The solid form of embodiment 71, wherein the solid form is characterized by peaks in its XRPD pattern selected from those at about 5.15, about 5.50, about 7.05, about 12.04, about 14.90, and about 16.56 degrees 2-theta.
   74. The solid form of embodiment 71, wherein the solid form is characterized by peaks in its XRPD pattern at substantially all of those listed in Table S14.
   75. The solid form of embodiment 71, wherein the solid form is characterized by one or more of:
(i) an XRPD pattern substantially similar to that depicted in FIG. 46A;
(ii) a DSC pattern showing a thermal event at about 143.7° C. and/or about 331° C.;
(iii) a DSC pattern substantially similar to that depicted in FIG. 46B;
(iv) a TGA pattern showing a weight loss of about 15.84% up to about 180° C.; and
(v) a TGA pattern substantially similar to that depicted in FIG. 46B.
   76. A pharmaceutical composition comprising the solid form of any one of embodiments 1-75 and a pharmaceutically acceptable carrier.
   77. The pharmaceutical composition of embodiment 76, wherein the pharmaceutical composition is solid.
   78. The pharmaceutical composition of embodiment 76 or 77, wherein the pharmaceutical composition is formulated for oral administration.
   79. A pharmaceutical composition prepared by a method comprising steps of: providing the solid form of any one of embodiments 1-75; and formulating the solid form with suitable excipients to provide the pharmaceutical composition.
   80. A pharmaceutical composition comprising:
a first portion comprising a bile acid or salt thereof, wherein the first portion is configured for immediate release in the colon of a subject;
a second portion, adjacent to the first portion, the second portion comprising a bile acid or salt thereof and wherein the second portion is configured for extended release in the colon of a subject; and
a degradable or erodible coating associated with the pharmaceutical composition,
wherein at least one of the first portion and the second portion comprises the solid form of any one of embodiments 1-75.
   81. A pharmaceutical composition comprising:
a first portion comprising a bile acid or salt thereof, wherein the first portion is configured for immediate release in the colon of a subject;
a second portion, adjacent to the first portion, the second portion comprising a bile acid or salt thereof and wherein the second portion is configured for extended release in the colon of a subject; and
a degradable or erodible coating associated with the pharmaceutical composition, prepared by a method comprising steps of:
providing the solid form of any one of embodiments 1-75; and
formulating the solid form with suitable excipients to provide the pharmaceutical composition.
   82. The pharmaceutical composition of embodiment 80 or 81, wherein the pharmaceutical composition is a tablet.
   83. The pharmaceutical composition of any one of embodiments 80-82, wherein the bile acid or salt thereof in the first portion and the second portion is chenodeoxycholic acid or salt thereof.
   84. The pharmaceutical composition of embodiment 83, wherein the first portion and the second portion comprise the solid form of any one of embodiments 1-75.
   85. The pharmaceutical composition of any one of embodiments 80-84, wherein the coating is or comprises Eudragit S100.
   86. A method comprising administering the solid form of any one of embodiments 1-75 or the pharmaceutical composition of any one of embodiments 76-85 to a subject in need thereof.
   87. The method of embodiment 86, comprising orally administering the solid form of any one of embodiments 1-75 or the pharmaceutical composition of any one of embodiments 76-85.
   88. A method of treating a disease, disorder, or condition, comprising administering the solid form of any one of embodiments 1-75 or the pharmaceutical composition of any one of embodiments 76-85 to a subject in need thereof.
   89. The method of any one of embodiments 86-88, wherein the subject is suffering from a gastrointestinal disease, disorder, or condition.
   90. The method of any one of embodiments 86-88, wherein the subject is suffering from constipation.
   91. The method of any one of embodiments 86-88, wherein the subject is suffering from irritable bowel syndrome with constipation (IBS-C).
   92. A method of preparing the solid form of any one of embodiments 1-75 according to a method described herein.
   93. A method of preparing the solid form of any one of embodiments 1-14, wherein the method comprises steps of:
providing chenodeoxycholic acid; and
contacting chenodeoxycholic acid with a suitable base in a suitable solvent to provide the solid form.
   94. The method of embodiment 93, wherein the suitable base is sodium hydroxide.
   95. The method of embodiment 93, wherein the suitable solvent is selected from methyl isobutyl ketone, n-butanol, and water.
   96. A method of preparing the solid form of any one of embodiments 2-6, wherein the method comprises steps of:
providing a mixture of chenodeoxycholic acid in methyl isobutyl ketone;
adding to the mixture an aqueous solution of sodium hydroxide;
heating the mixture; and
removing the solvent to provide the solid form.
   97. A method of preparing the solid form of any one of embodiments 7-11, wherein the method comprises steps of:
providing a mixture of chenodeoxycholic acid in n-butanol;
adding to the mixture an aqueous solution of sodium hydroxide;
heating the mixture; and
removing the solvent to provide the solid form.
   98. The method of embodiment 96 or 97, wherein the mixture is heated to azeotropic reflux.
   99. A method of preparing the pharmaceutical composition of any one of embodiments 76-85, wherein the method comprises steps of:
providing the solid form of any one of embodiments 1-75; and
formulating the solid form with suitable excipients to provide the pharmaceutical composition.

### Examples

The Examples provided herein document and support certain aspects of the present disclosure but are not intended to limit the scope of any claim. The following non-limiting examples are provided to further illustrate certain teachings provided by the present disclosure. Those of skill in the art, in light of the present application, will appreciate that various changes can be made in the specific embodiments that are illustrated in the present Examples without departing from the spirit and scope of the present teachings.

### Materials & Methods—Examples 1-4

### X-ray Powder Diffraction (XRPD)

XRPD analyses were run on a Bruker D8 Advance diffractometer having a goniometer radius of 280 mm and working in Bragg-Brentano geometry. The radiation used was Ni-filtered CuKα (1.54 Å), the detector used was a silicon strip (LynxEye) detector. The range analyzed was 3-40° in 2θ with a step of 0.02°. The sample holder used was a zero-background silicon monocrystal. The data obtained were analyzed with Diffrac.Eva software version 4.3.0.1, Bruker AXS.

### Differential Scanning Calorimetry (DSC)

Differential Scanning Calorimetry (DSC) analyses were run on a Mettler-Toledo DSC-3 cell. Accurately weighed 3-7 mg samples were placed in aluminum pans with pierced lids and heated at 10° C./min from 30° C. to 300° C. under nitrogen flow. The data obtained were analyzed with Stare software version 16.00.

### Thermogravimetric Analysis (TGA)

Thermogravimetric analyses (TGA) were run on a Mettler-Toledo TGA2. Accurately weighed 15-30 mg samples were heated at 10° C./min from 25° C. to 300° C. under nitrogen flow. The data obtained were analyzed with Stare software version 16.20.

### Dynamic Vapor Sorption (DVS)

DVS measurements were performed with a SMS—DVS Intrinsic. The sample size was about 50 mg, accurately weighed. The measurement was performed at 25° C., the relative humidity (RH) range investigated was 40-90-0-90% RH with 10% RH steps. After each RH change, the mass of the sample was allowed to stabilize (waiting time 10 to 60 minutes, stabilization criterion ≤0.002 g for 10 minutes).

The hygroscopicity of the sample was calculated as follows:

$$\% Weight change = ( W 2 - W 1 ) W 1 \text{/·} 100$$

where:
W1: weight of sample at the start of the experiment (25° C. and 40% RH)
W2: weight of sample at 25° C. and 80% RH in the first sorption cycle High Pressure Liquid Chromatography (HPLC)

For the intrinsic dissolution studies, the following HPLC method was used:

| | |
|---|---|
| Column | Kromasil 100A C18 250 × 4.6 mm, 5 μm |
| Flow | 1 mL/min |
| Injection volume | 100 μL |
| Wavelength | 200 nm |
| Column temperature | 35° C. |
| Mobile phase | 480:520:1.7 Acetonitrile/water/phosphoric acid 85% (v/v) |
| Dissolution phase (DP) | 50:50 Acetonitrile/water |
| Standard solution | In a 100 mL volumetric flask, NaCDC (10 mg) reference standard |
| | was added, and then diluted to volume with DP (mother solution). |
| | 1 mL mother solution diluted to 100 mL with DP (solution A). 1 |
| | mL mother solution diluted to 50 mL with DP (solution B). 1 mL |
| | mother solution diluted to 20 mL with DP (solution C). A |
| | calibration curve was prepared by injecting solutions A, B, and C. |
| Test solution | At each specified time point, 2 mL of solution was withdrawn |
| | from vessel, filtered, and injected into HPLC. |
| Retention time of NaCDC | RT = 24.3 min |

### Example 1. Preparation & Characterization of Provided Solid Forms

### Sodium Chenodeoxycholate Form A

NaCDC Form A was prepared according to the following exemplary procedure: In a one-liter reactor equipped with a dean-stark apparatus CDCA (50 g) and methyl isobutyl ketone (2500 mL, 5 volumes) were loaded at 20° C.±5° C. The mixture was stirred, and then 18.7 g of NaOH solution 30% p/p in water was added. Once dissolution was completed, a slight exothermic reaction was observed. The solution was heated at azeotropic reflux for 2 to 3 hours to distill water. Once crystallization started to occur, the mixture was stirred at 115-117° C. for an additional 1 hour, then cooled at 20° C. ±5° C. and stirred for 2 hours at 20° C.±5° C. The mixture was filtered by washing the panel with 50 mL of methyl isobutyl ketone. The wet product (51.6 g) was dried at 50° C. under vacuum to obtain NaCDC Form A (47.8 g, 85.2% yield).

The XRPD pattern of Form A is shown in FIG. 1, and the corresponding data are summarized below:

| 2θ [°] | d-spacing [Å] |
|---|---|
| 6.07 | 14.6 |
| 6.55 | 13.5 |
| 8.36 | 10.6 |
| 9.92 | 8.9 |
| 10.72 | 8.2 |
| 11.55 | 7.66 |
| 11.93 | 7.41 |
| 12.99 | 6.81 |
| 13.36 | 6.62 |
| 14.64 | 6.04 |
| 15.06 | 5.88 |
| 15.54 | 5.70 |
| 15.93 | 5.56 |
| 16.68 | 5.31 |
| 16.99 | 5.21 |
| 17.31 | 5.12 |
| 17.58 | 5.04 |
| 18.07 | 4.91 |
| 18.34 | 4.83 |

As shown in FIG. 2, DSC analysis of Form A showed a loss of water starting just above ambient temperature and continuing to about 150° C. Karl-Fischer analysis showed 6.0 w/w % water content of Form A. TGA analysis of Form A (FIG. 3) showed a weight loss of 4.3% up to 150° C. Form A was determined to be a sesquihydrate.

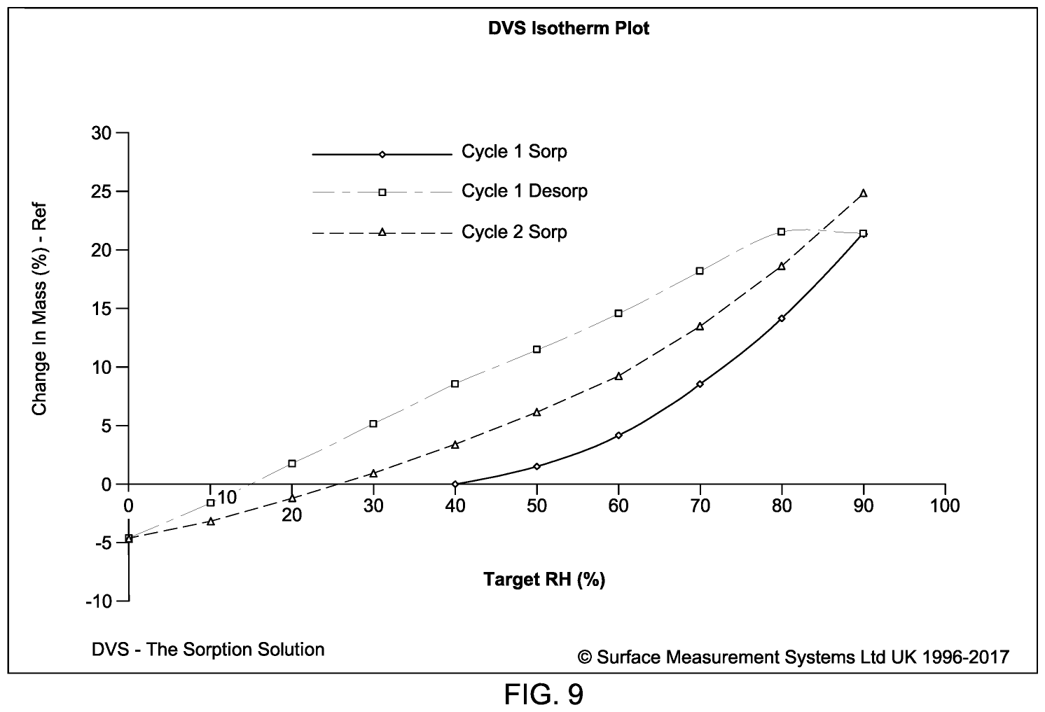

DVS of Form A showed that it was hygroscopic (14.1% weight change in the first sorption cycle, FIG. 9).

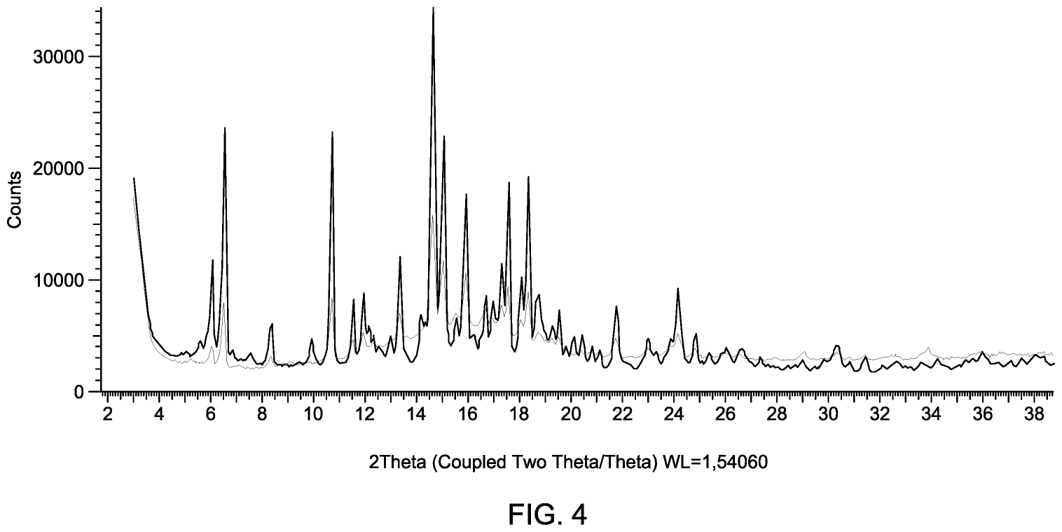

Exposure to moist air caused the water content of Form A to increase, eventually leading to partial amorphization and loss of crystallinity after 15-20 hours at 95% humidity (FIG. 4).

### Sodium Chenodeoxycholate Form B

NaCDC Form B was prepared according to the following exemplary procedure: In a one-liter reactor equipped with a Dean-Stark apparatus, CDCA (50 g) and n-butanol (300 mL) were loaded at 20° C.±5° C. The mixture was stirred, and then NaOH solution (18.7 g, 30% p/p in water) was added. The solution was heated at azeotropic reflux for 3-4 hours. Once crystallization started to occur, approx. 200 mL (4 volumes) of solvent was distilled in about 3-4 hours, until a vapor temperature of 117° C. was reached. The resulting suspension was cooled to 20° C.: 5° C. and shaken for 1 hour at 20° C.: 5° C. The solids were filtered and washed with n-butanol (50 mL). The wet solids were dried at 75° C. under vacuum to give NaCDC Form B (46.7 g, 88.5% yield).

The XRPD pattern of Form B is shown in FIG. 5, and the corresponding data are summarized below:

| 2θ [°] | d-spacing [Å] |
|---|---|
| 6.75 | 13.1 |
| 8.14 | 10.8 |
| 9.79 | 9.0 |
| 11.33 | 7.81 |
| 13.27 | 6.67 |
| 13.56 | 6.52 |
| 14.02 | 6.31 |
| 14.79 | 5.99 |
| 15.05 | 5.88 |
| 16.10 | 5.50 |
| 16.37 | 5.41 |
| 16.78 | 5.28 |
| 17.74 | 5.00 |
| 18.63 | 4.76 |
| 19.71 | 4.50 |
| 20.00 | 4.44 |
| 20.35 | 4.36 |
| 21.89 | 4.06 |
| 22.90 | 3.88 |

As shown in FIG. 6, DSC analysis of Form B showed no thermal event from room temperature to the wide melting event, starting around 288° C. Karl-Fischer analysis showed <0.1 w/w % water content for Form B. TGA analysis of Form B (FIG. 7) showed a weight loss of <0.1% up to 150° C. Form B was determined to be an anhydrate.

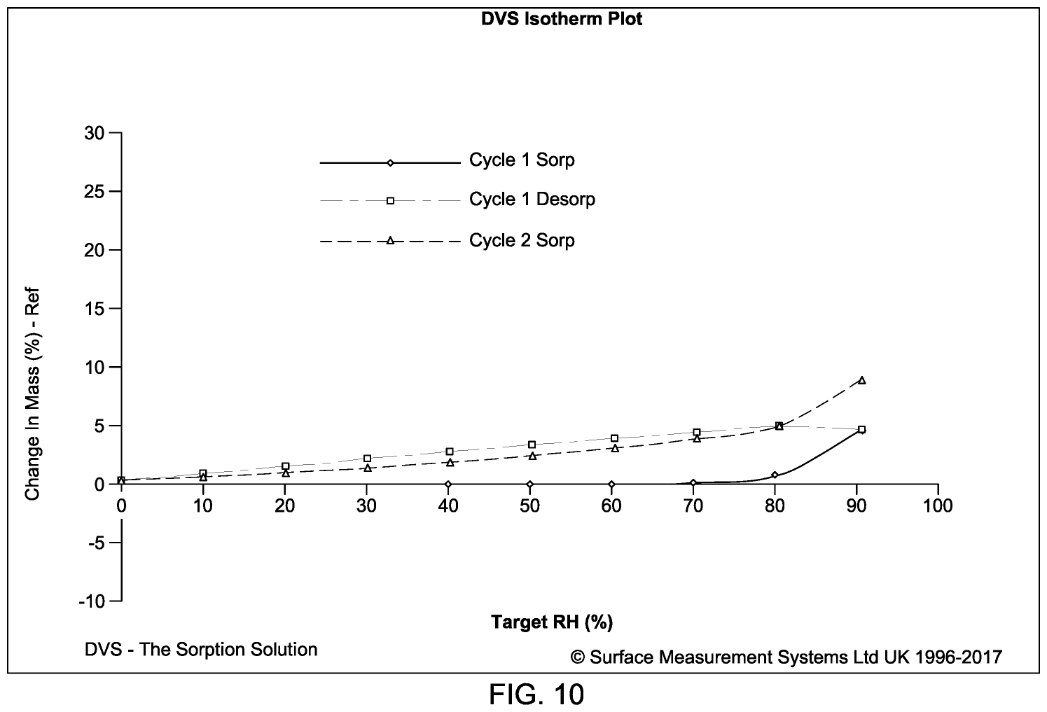

DVS of Form B showed that it was slightly hygroscopic (0.8% weight change in the first sorption cycle, FIG. 10).

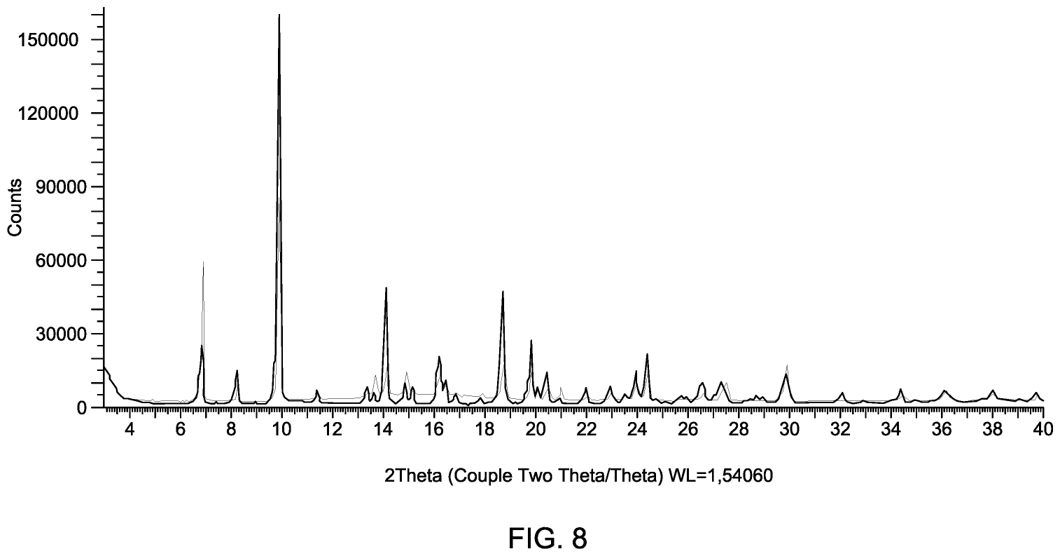

Exposure to moist air caused eventual amorphization after 15-20 hours at 95% humidity (FIG. 8), but loss of crystallinity was markedly slower than for Form A.

### Example 2. Stability Studies

In-process stability of NaCDC Form B was evaluated under two conditions at reflux, as described in Table 1 below. No change in the HPLC impurity profile was observed under either condition, indicating the product was stable under these conditions.

TABLE 1

| | | | | Karl Fischer of |
|---|---|---|---|---|
| Solvent | Conditions | HPLC Purity % | | distillate (%) |
| n-BuOH/water | Azeotropic | CDC (t₀) | 99.92 | — |
| (6/1) | reflux | Cholic acid (t₀) | 0.05 | |
| | for 24 h | CDC (t₂₄) | 99.92 | |
| | | Cholic acid (t₂₄) | 0.05 | |
| n-BuOH | Reflux | CDC (t₀) | 99.9 | <0.03 |
| | for 24 h | Cholic acid (t₀) | 0.05 | |
| | | CDC (t₂₄) | 99.9 | |
| | | Cholic acid (t₂₄) | 0.05 | |

### Example 3. Intrinsic Dissolution Studies

Intrinsic dissolution profiles of NaCDC Form A and Form B were determined from a drug disk of constant surface area using a USP rotating disk apparatus in phosphate buffer (pH 7.4) at 37° C. After compression (described below), no change in solid form based on XRPD was observed.

A typical apparatus consisted of a punch and a die, whose base is attached to a surface plate. The die had a cavity into which was placed a defined amount of material whose intrinsic dissolution rate is to be determined. The punch was then inserted in the die cavity and the test material was compressed with a hydraulic press. A non-disintegrating compact of the material was formed in the die cavity with a single face of defined area exposed on the bottom of the die. The die assembly was then attached to a shaft with a holder and the surface plate was removed. The shaft holding the die assembly was positioned into the dissolution medium at a distance not less than 1.0 cm from the bottom of the vessel. In this procedure, fluid flow was generated by the rotation of the die. The amount of material dissolved was measured as a function of time. In particular, the cumulative amount dissolved at each time point was corrected for losses due to sampling. If the amount versus time profiles showed curvature, only the initial linear portion of the profile was used to determine the dissolution rate. Method parameters are summarized below:

| | |
|---|---|
| Apparatus | Rotating Disk apparatus (USP) |
| Surface area | 0.5024 cm² (diameter = 0.8 cm) |
| Compression force | 2 tons for 2 minutes |
| Amount of material | 150 mg |
| Dissolution medium | USP Phosphate buffer pH 7.4, 500 mL |
| Dissolution medium temperature | 37° C. ± 0.5° C. |
| Rotation speed | 100 rpm ± 0.5 rpm |
| Sample volume | 2 mL |
| Filter | Whatman 0.45 μm TF Filter |

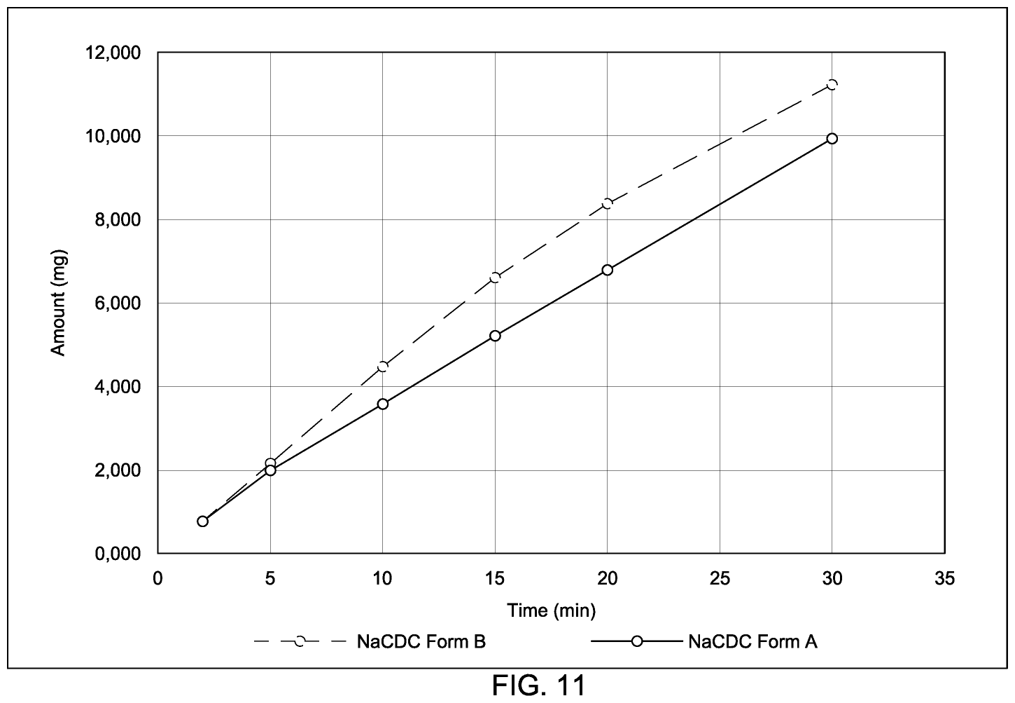

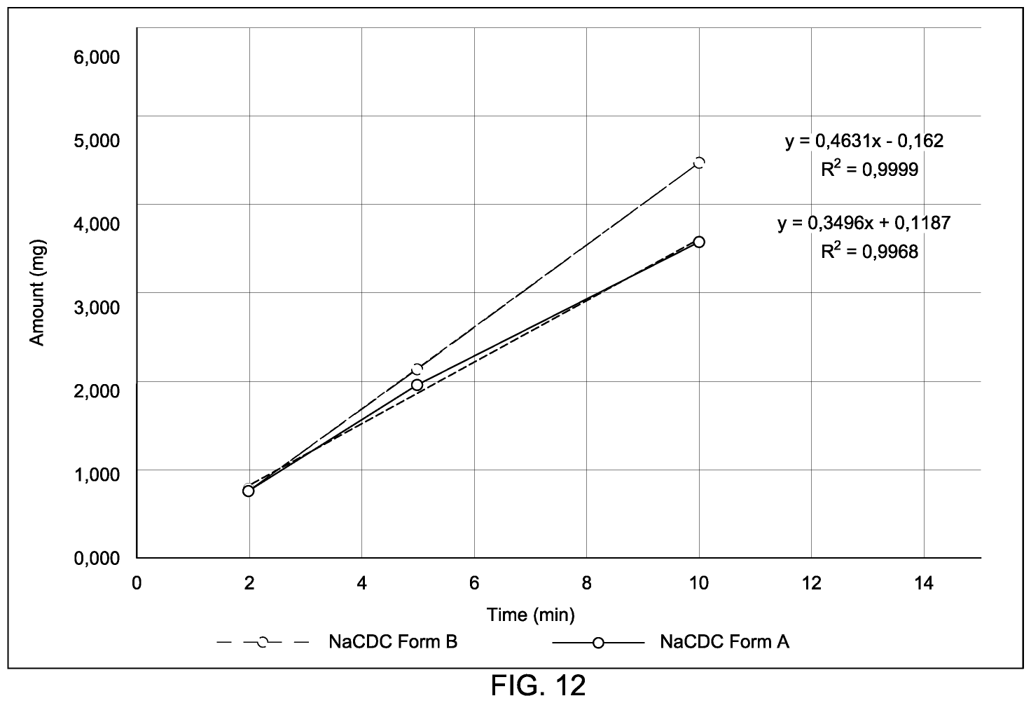

Intrinsic dissolution profiles of NaCDC Form A and Form B are shown in FIG. 11. Linear regression analysis of the profiles are shown in FIG. 12, and the intrinsic dissolution rates are summarized in Table 2. Notably, Form B had a higher intrinsic dissolution rate, while being less hygroscopic, than Form A.

TABLE 2

| Form | IDR (mg · min⁻¹ · cm⁻²) |
|---|---|
| A | 0.6959 |
| B | 0.9218 |

### Example 4. Solubility Curve Measurements

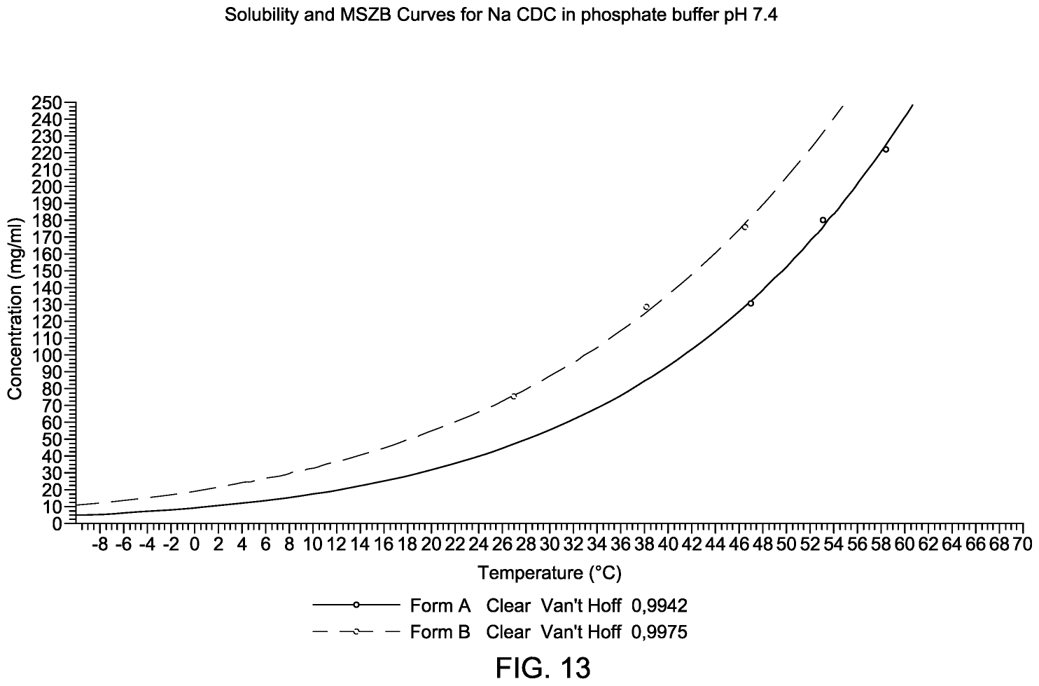

Solubility curve measurements of NaCDC Form A and Form B were performed using a Crystal16 automatic crystallizer (Technobis Crystallization Systems), having an array of 16 microreactors equipped with turbidimeters for the determination of clear and cloud points. Samples of four different concentrations for each crystalline form were prepared by weighing accurately different amounts of each sample and adding 1000 μL of phosphate buffer (pH 7.4) to each vial. The suspensions thus obtained were stirred using a magnetic stirrer and heated from 0° C. to 90° C. at 0.5° C./min. The clear point for each sample was determined and plotted to obtain a crystallization curve (FIG. 13). NaCDC Form B showed a slightly higher solubility compared to NaCDC Form A.

### Materials & Methods—Examples 5-10

### X-Ray Powder Diffraction (XRPD)

XRPD analysis was carried out using a Bruker D8 Discover diffractometer with DAVINCI configuration, in transmission mode (scan type: TwoTheta or Offset Coupled TwoTheta/Theta) scanning the samples between 1.5 and 45° 2θ angles, and using 7.58 minutes acquisition time (increment per step was 0.01°, time per step was 0.1 s, and generator voltage/generator amperage of 40 mA/40 kV to reach 1.6 kW power). Approximately 2-3 mg of each sample were used. The limit of detection for XRPD varied based on the sample crystallinity. In general, for crystalline compounds, the detection limit for XRPD was estimated at approximately 2% wt or even less.

### Differential Scanning Calorimetry (DSC)

Around 3.9 mg of sample was weighed into an aluminum DSC pan and sealed non-hermetically with an aluminum lid. The sample pan was then loaded into a Setaram DSC131 EVO (equipped with a cooler). Once a stable heat-flow response was obtained, the sample and reference were heated to 450° C. with rate of 10° C./min and the resulting heat flow response monitored. Nitrogen was used as the purge gas, at a flow rate of 40 cm³/min. Prior to analysis, the instrument was temperature and heat-flow calibrated using lead and indium reference standards. Sample analysis was carried out with the help of CALISTO software where the temperatures of thermal events were quoted as the onset temperature, measured according to the manufacturer's specifications.

Thermogravimetric Analysis Coupled with DSC (TG/DSC)

Between about 1.56 and 5.78 mg of sample was weighed into an open aluminum pan and loaded into a simultaneous Setaram LABSYS EVO thermo-gravimetric/differential scanning calorimeter (TG-DTA/DSC), and held at 30° C. for 15 minutes. The sample was then heated with a rate of 10° C./min from 30° C. to 550° C., during which time the change in sample weight was recorded along with any differential thermal events. Nitrogen was used as the purge gas, at a flow rate of ˜180 cm³/min. Prior to the analysis, the instrument was mass loss and temperature calibrated using copper sulfate pentahydrate and indium and lead reference standards. Sample analysis was carried out with the help of CALISTO software, where the corresponding mass loss and temperatures of thermal events were quoted as the onset temperature, measured according to the manufacturer's specifications. All analyses were carried out with a heating rate of 10° C./minute and background was subtracted.

### High Performance Liquid Chromatography (HPLC)

HPLC analysis was carried out on an Agilent 1260 Infinity chromatograph, using a Hichrom C18 column 100×4.6 mm, 3.5 m, at 40° C. Method details were as follows:

| | |
|---|---|
| Flow | 1.1 mL/min |
| Injection volume | 10 μL |
| Wavelength | 212 nm |
| Mobile phase | (A) Buffer pH = 2.5 (ortho-phosphoric acid |
| | adjusted with 1N KOH), sonicated before use |
| | (B) MeCN, HPLC grade, sonicated before use |
| Dissolution phase (DP) | 1:1 H₂O:MeOH (v/v) |
| Gradient | 0.0 min: 95% (A)/5% (B) |
| | 0.5 min: 95% (A)/5% (B) |
| | 4.0 min: 5% (A)/95% (B) |
| | 7.0 min: 5% (A)/95% (B) |
| | 9.0 min: 95% (A)/5% (B) |
| | 10.0 min: 95% (A)/5% (B) |

### Example 5. Additional Characterization of NaCDC Form B

An additional standalone DSC analysis of NaCDC Form B was performed, as shown in FIG. 14. This analysis revealed two endothermic events, one associated with a possible melting with phase transformation (Tₒₙₛₑₜ=291.9° C.) and another associated with a possible melting (Tₒₙₛₑₜ=331.6° C.).

TG/DSC analysis of NaCDC Form B was also performed, and the results are shown in FIG. 15. This analysis revealed two endothermic events, one associated with a possible melting with phase transformation (Tₒₙₛₑₜ=291.8° C. and Heat=35 J/g) and another associated with a possible melting (Tₒₙₛₑₜ=336.4° C. and Heat=51.2 J/g). The difference in Tₒₙₛₑₜ between the standalone DSC analysis and the TG/DSC analysis was attributed to difference in crucible type used (an open 100 μL aluminum crucible was used for TG/DSC, while a closed pierced 30 μL aluminum crucible was used for standalone DSC).

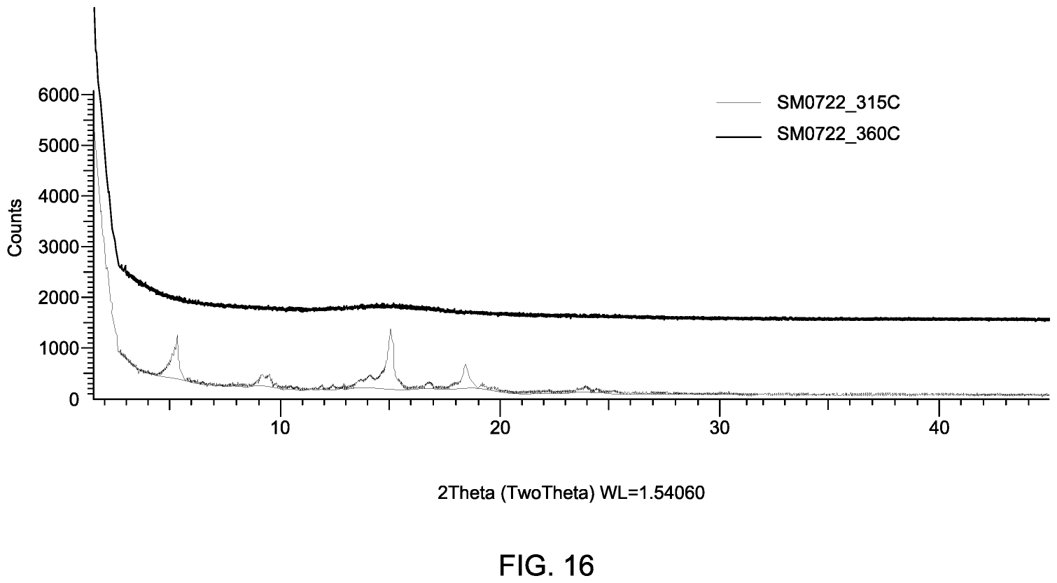

Based on the results obtained from the TG/DSC analysis, a sample of NaCDC Form B was heated at 315° C. and another sample at 360° C. After heating, the samples were cooled to room temperature and analyzed by XRPD (FIG. 16). The sample heated at 315° C. had a different diffractogram than the starting material. The material obtained after heating a sample at 360° C. was amorphous. HPLC analysis shows that the purity of the sample heated at 315° C. was 66.9%.

### Example 6. Photostability Studies

Photostability of NaCDC Form B was evaluated by exposing it in solid state and in MeOH solutions to UV 254 nm light for 48 hours in clear, amber, and aluminum foil wrapped vials. The solutions were prepared by weighing around 10 mg NaCDC Form B and mixing it with 500 μL MeOH. The solids and solutions were sampled after 48 hours and analyzed by HPLC, in order to assess degradation. In parallel, XRPD analysis was performed on the recovered solids. The experimental results are summarized in Table 3.

TABLE 3

| | | | Apparent | | | | | HPLC (%) |
|---|---|---|---|---|---|---|---|---|
| | | Mass | conc. | | Temp | | | at 212 nm, |
| Expt | Solvent | (mg) | (mg/mL) | Conditions | (° C.) | Observation | XRPD | after 48h |
| ST01 | N/A | 10.00 | N/A | UV 254 nm - solid - | RT | white solid | SM | 94.4 |
| | | | | clear vial | | | | |
| ST02 | N/A | 10.25 | N/A | UV 254 nm - solid - | | white solid | SM | 95.0 |
| | | | | amber vial | | | | |
| ST03 | N/A | 10.10 | N/A | UV 254 nm - solid - | | white solid | SM | 95.4 |
| | | | | control - dark | | | | |
| ST04 | MeOH | 10.05 | 20.10 | UV 254 nm - | | colorless | N/A | 91.4 |
| | | | | MeOH solution - | | solution | | |
| | | | | clear vial | | | | |
| ST05 | MeOH | 10.20 | 20.40 | UV 254 nm - | | colorless | N/A | 91.0 |
| | | | | MeOH solution - | | solution | | |
| | | | | amber vial | | | | |
| ST06 | MeOH | 10.06 | 20.12 | UV 254 nm - | | colorless | N/A | 94.2 |
| | | | | MeOH solution - | | solution | | |
| | | | | control - dark | | | | |

SM = starting material (i.e., NaCDC Form B)

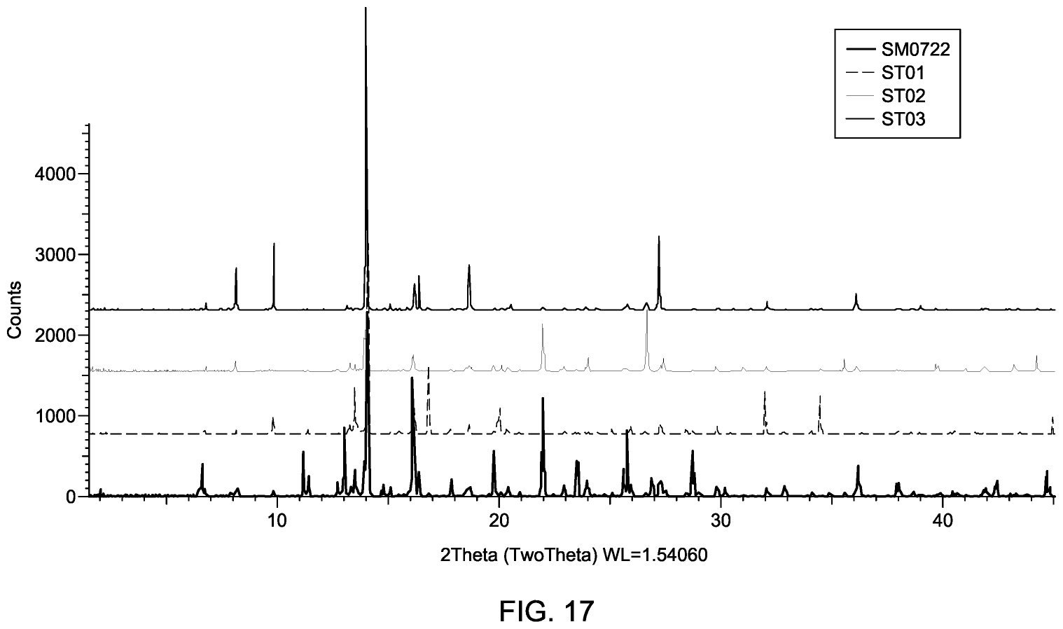

No form transformation was observed by XRPD in the experiments performed in the solid state, which indicated that NaCDC Form B is stable (FIG. 17). In solution, HPLC analysis indicated that the compound was stable, as evidenced by minimal decrease in purity (see Table 3).

### Example 7. Stability Studies in Slurry/Solution

Around 10 mg of NaCDC Form B was weighed into clear HPLC vials and the selected solvents (500 μL) were added to the vials. Experiments ST07, 09, 11, 13, 15, 17, 19 and 21 were kept stirring for 24 h at 30° C. and 60° C., while samples were taken after 4 h and 24 h for HPLC analysis. Subsequently, the same experiments were evaporated at 30° C. for 24 h under reduced pressure (21-22 mbar), and the solids were evaluated by HPLC and XRPD. Separately, the solutions and slurries from experiments ST10, 12, 14, 16, 18, 20 and 22 were stirred for 24 h at 30° C. and 60° C. (no samples were taken) and evaporated at 60° C. for 24 h under reduced pressure (20-21 mbar). The recovered materials were sampled for HPLC and XRPD analyses. The experimental results are summarized in Table 4.

TABLE 4

| | Slurry/solution experimental step | | | | | | | Evaporation step | | | | |
|---|---|---|---|---|---|---|---|---|---|---|---|---|
| | | | | | | HPLC (%) at | | | | | | HPLC (%) |
| | | | | | | 212 nm | | | | | | at |
| | | | Apparent | | | (before | | | | | | 212 nm |
| | Mass | | conc. | Type of | Temp | evap.) | | T_{evap.} | Evap. | | | (after |
| Expt | (mg) | Solvent | (mg/mL) | expt. | (° C.) | 4 h | 24 h | (° C.) | time | Observation | XRPD | evap.) |
| ST07 | 10.03 | Water | 20.06 | solution | RT | 100.0 | 96.2 | 30 | 24 h | glassy white | Am. | 97.8 |
| | | | | | | | | | | solid | | |
| ST08 | 10.15 | | 20.30 | | | N/A | N/A | 60 | 24 h | glassy white | Am. | 84.6 |
| | | | | | | | | | | solid | | |
| ST09 | 10.13 | | 20.26 | | 60 | 100.0 | 96.6 | 30 | 24 h | glassy white | Am. | 95.7 |
| | | | | | | | | | | solid | | |
| ST10 | 10.19 | | 20.38 | | | N/A | N/A | 60 | 24 h | glassy white | Am. | 90.0 |
| | | | | | | | | | | solid | | |
| ST11 | 10.12 | Methanol | 20.24 | | RT | 91.4 | 93.5 | 30 | 24 h | glassy white | Am. | 87.1 |
| | | | | | | | | | | solid | | |
| ST12 | 10.02 | | 20.04 | | | N/A | N/A | 60 | 24 h | glassy white | Am. | 90.5 |
| | | | | | | | | | | solid | | |
| ST13 | 10.1 | | 20.20 | | 60 | 94.8 | 90.3 | 30 | 24 h | glassy white | Am. | no peak~ |
| | | | | | | | | | | solid | | 5.36 min. |
| ST14 | 10.15 | | 20.30 | | | N/A | N/A | 60 | 24 h | glassy white | Am. | 92.5 |
| | | | | | | | | | | solid | | |
| ST15 | 10.16 | n- | 20.32 | slurry | RT | 89.9 | 100.0 | 30 | 24 h | white solid | SM | no peak~ |
| | | Heptane | | | | | | | | | | 5.36 min. |
| ST16 | 10.14 | | 20.28 | | | N/A | N/A | 60 | 24 h | white solid | SM | 84.5 |
| ST17 | 10.18 | | 20.36 | | 60 | 91.5 | 91.4 | 30 | 24 h | white solid | SM | 93.6 |
| ST18 | 10.14 | | 20.28 | | | N/A | N/A | 60 | 24 h | white solid | SM | 90.4 |
| ST19 | 10.13 | MEK | 20.26 | | RT | 100.0 | 97.7 | 30 | 24 h | white solid | S1 | 74.1 |
| ST20 | 10.15 | | 20.30 | | | N/A | N/A | 60 | 24 h | white solid | S1, P.O. | 86.1 |
| ST21 | 10.14 | | 20.28 | | 60 | 65.2 | 94.5 | 30 | 24 h | white solid | SM | 55.9 |
| ST22 | 10.05 | | 20.10 | | | N/A | N/A | 60 | 24 h | white solid | SM | 86.6 |

Am. = amorphous;
evap. = evaporation;
SM = starting material (i.e., NaCDC Form B);
P.O. = preferred orientation

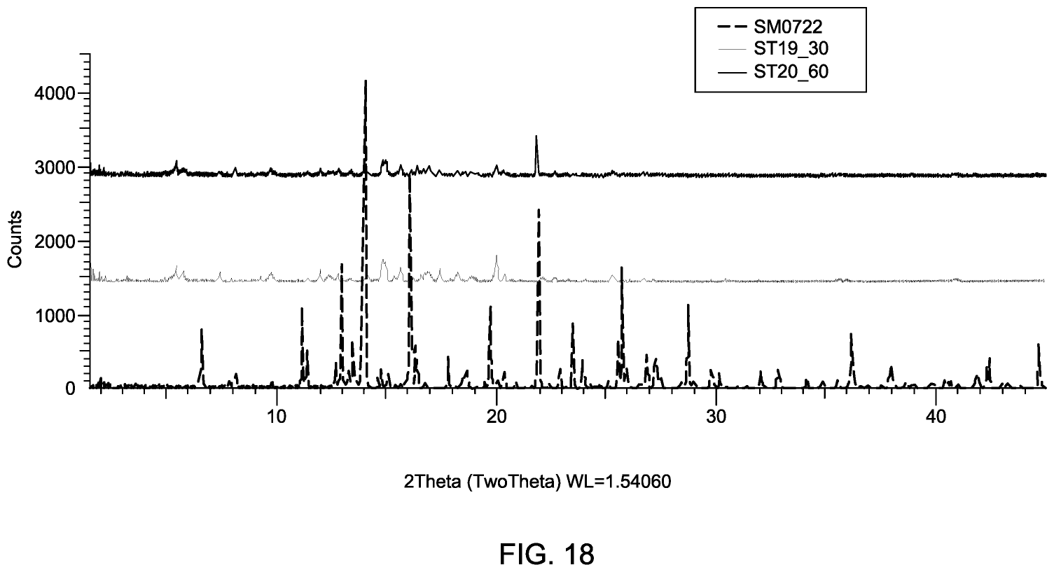

XRPD analysis indicated that NaCDC Form B is stable, as no form transformation was observed in most of the slurry experiments. Two exceptions, namely, ST19 and ST20 (in MEK evaporated at 30° C. and 60° C., respectively) were observed, and they yielded a new Form S1 (FIG. 18).

In general, the starting material remained stable in solution and/or slurry under the tested conditions. The HPLC analysis performed throughout the stability tests revealed purities ≥86.1% at 212 nm, except in the ST08 and ST16 experiments (which were evaporated at 60° C.) where the purity decreased to around 84.5%. Additionally, experiments ST19 and ST21 (which used MEK and were evaporated at 30° C.) indicated lower purity (74.1% and 55.9%), which might suggest the presence of an amorphous impurity not detectable via XRPD.

### Characterization of Form S1

Form S1 was obtained from experiments ST19 and ST20 (in MEK slurry at RT for 24 h and evaporated at 30° C. and 60° C., respectively).

The sample from ST19 was characterized by XRPD, as shown in FIG. 19A. The corresponding data are summarized in Table S1-A:

TABLE S1-A

| 2-theta | d-Spacing | Rel. |
|---|---|---|
| Angle (°) | (Å) | Intensity (%) |
| 5.45 | 16.19 | 40.10 |
| 5.80 | 15.23 | 28.37 |
| 7.46 | 11.84 | 31.83 |
| 9.30 | 9.50 | 13.13 |
| 9.76 | 9.05 | 26.09 |
| 9.94 | 8.89 | 29.50 |
| 11.02 | 8.02 | 11.33 |
| 11.45 | 7.72 | 19.71 |
| 12.02 | 7.36 | 39.58 |
| 12.40 | 7.13 | 26.84 |
| 12.83 | 6.89 | 31.81 |
| 14.19 | 6.24 | 30.62 |
| 14.88 | 5.95 | 79.59 |
| 15.00 | 5.90 | 63.43 |
| 15.40 | 5.75 | 13.86 |
| 15.65 | 5.66 | 47.94 |
| 16.13 | 5.49 | 19.00 |
| 16.60 | 5.33 | 29.11 |
| 16.79 | 5.28 | 31.66 |
| 16.91 | 5.24 | 29.10 |
| 17.45 | 5.08 | 47.12 |
| 17.93 | 4.94 | 12.64 |
| 18.27 | 4.85 | 35.33 |
| 18.93 | 4.68 | 16.61 |
| 19.11 | 4.64 | 12.67 |
| 20.02 | 4.43 | 100.00 |
| 20.38 | 4.35 | 33.20 |
| 22.17 | 4.01 | 10.78 |
| 22.68 | 3.92 | 14.34 |
| 25.24 | 3.53 | 24.64 |
| 25.90 | 3.44 | 8.72 |
| 26.75 | 3.33 | 12.68 |
| 27.19 | 3.28 | 5.82 |
| 35.64 | 2.52 | 6.80 |

The sample from S120 was characterized by TG/DSC, as shown in FIG. 19B. Three endothermic events were identified on DSC trace: The first event was associated with a desolvation process with Tₒₙₛₑₜ=96.0° C. and a mass loss of 2.177% (theoretical mass of 3.704% corresponds with 0.25 MEK molecules). As such, the mass loss may be attributed to a physical adsorption or residual one. The second event corresponded to either an artifact or a phase transformation considering the preferred orientation of the measured sample with Tₒₙₛₑₜ=142.5° C. and a heat of 3.29 J/g. The third event corresponded with the melting process of the form with Tₒₙₛₑₜ=313.4° C. The sample from ST19 displayed a similar TG/DSC trace, though did not display the second endotherm observed from ST20.

### Example 8. Solubility Studies

Around 10 mg of NaCDC Form B was weighed into 1.6 mL HPLC vials. 3-5 mm PTFE-covered cylindrical magnetic stirring bars were added to the vials. A qualitative solubility assessment study was performed by adding small successive aliquots of each selected solvent system to the corresponding vials, under stirring at RT, at ˜15 minutes intervals (50 μL volumes were added up to 200 μL, at 500 rpm; 100 μL volumes were added between 200 and 1000 μL total volume, at 700 rpm). The vials were visually inspected before each addition to check for the dissolution of the starting material, and the solvent addition was stopped when a complete dissolution was observed. The clear solutions were left to rest at RT. In the cases where a total volume of 1 mL was reached and dissolution did not occur (concentration of ˜10 mg/mL at RT), the vials were heated under stirring, first at 40° C. (except, experiment SAS23 with ethyl ether that was heated only at 30° C. for 1 h), and then at 50° C. The experiments were held at each temperature for 1 h, and then they were visually inspected to see if dissolution occurred. At the end of the experimental time, the magnetic stirring bars were removed, and all slurries or solutions were dried at different temperatures depending on the boiling points of each solvent used (i.e., at 30° C., 20-21 mbar or 50° C., 21 mbar, for 21 hours; with the exception of experiments SASO7, SAS33, SAS34, SAS35, SAS36 and SAS45 which were dried at 50° C. for 43 hours). All the resulting solids were analyzed by XRPD. For several selected experiments, HPLC analysis was performed. The solids that gave new XRPD patterns were investigated by TGA/DSC. The results are summarized in Table 6, and a description of the qualitative solubility terms is provided in Table 5.

TABLE 5

| | Volumes (10 μL) of solvent required |
|---|---|
| Descriptive term | for 1 part of compound (~10 mg) |
| Very soluble | Less than 5 |
| Freely soluble | From 5 to 10 |
| Soluble | From 10 to 30 |
| Sparingly soluble | From 30 to 100 |
| Slightly soluble | More than 100, dissolved at |
| | 30/40° C. or 50/60° C. |
| Insoluble | More than 100, undissolved at |
| | 30/40° C. or 50/60° C. |

TABLE 6

| | | | Apparent | | | | | | HPLC |
|---|---|---|---|---|---|---|---|---|---|
| | | Mass | Conc. | | | Evap. | | | (%) |
| Expt | Solvent | (mg) | (mg/mL) | Solubility | T dissol (° C.) | Cond. | Observation | XRPD | 212 nm |
| SAS01 | Water (H₂O) | 10.20 | 204.00 | Very | RT | A | white solid | Am. + few | 84.9 |
| | | | | soluble | | | | broad peaks: | |
| | | | | | | | | 3.75-6.38 & | |
| | | | | | | | | 12.45-18.13 | |
| SAS02 | Methanol (MeOH) | 10.06 | 100.60 | Freely | RT | A | white solid | Am. + pks. | |
| | | | | soluble | | | | | |
| SAS03 | Ethanol (EtOH) | 10.13 | 10.13 | Insoluble | Dissolved with | A | white solid | S2, P.O. | 71.5 |
| | | | | | few crystals at | | | | |
| | | | | | 50° C. | | | | |
| SAS04 | 2-Propanol | 10.12 | 10.12 | Insoluble | Fine slurry at | A | white solid | SM | 96.6 |
| | (2-PrOH) | | | | 50° C. | | | | |
| SAS05 | 2-Methyl-1- | 10.11 | 10.11 | Insoluble | Dissolved with | A | white solid | SM + pks. (and | 96.5 |
| | propanol | | | | few crystals at | | | broad peak: | |
| | (Isobutanol) | | | | 50° C. | | | 4.29-6.31) | |
| SAS06 | 2-Butanol | 10.05 | 10.05 | Insoluble | Fine slurry at | A | white solid | SM, P.O. or | 97.5 |
| | | | | | 50° C. | | | SM + pks. | |
| SAS07 | 2-Ethoxyethanol | 10.10 | 202.00 | Very | RT | C | white solid | S3 (likely a | 38.0 |
| | | | | soluble | | | | mixture with | |
| | | | | | | | | crystalline | |
| | | | | | | | | degradation | |
| | | | | | | | | product) | |
| SAS08 | 2,2,2- | 10.13 | 50.65 | Soluble | RT | A | white solid | S4 | 91.3 |
| | Trifluoroethanol | | | | | | | | |
| | (TFE) | | | | | | | | |
| SAS09 | 3-Methyl-1-butanol | 10.16 | 10.16 | Insoluble | Dissolved with | B | white solid | Am. + broad | 48.1 |
| | (Isoamyl alcohol) | | | | few flakes at | | | pks.: 3.75-6.38; | |
| | | | | | 50° C. | | | 13.82-14.40; | |
| | | | | | | | | 15.27-17.23 | |
| SAS10 | Cyclohexane | 10.15 | 10.15 | Insoluble | Fine slurry at | A | white solid | SM | |
| | | | | | 50° C. | | | | |
| SAS11 | Methylcyclohexane | 10.26 | 10.26 | Insoluble | Fine slurry at | A | white solid | SM | |
| | | | | | 50° C. | | | | |
| SAS12 | n-Heptane | 10.07 | 10.07 | Insoluble | Fine slurry at | A | white solid | SM | |
| | | | | | 50° C. | | | | |
| SAS13 | 2,2,4- | 10.06 | 10.06 | Insoluble | Fine slurry at | A | white solid | SM | |
| | Trimethylpentane | | | | 50° C. | | | | |
| | (Isooctane) | | | | | | | | |
| SAS14 | Toluene | 10.09 | 10.09 | Insoluble | Fine slurry at | B | white solid | SM | |
| | | | | | 50° C. | | | | |
| SAS15 | Xylenes | 10.07 | 10.07 | Insoluble | Fine slurry at | B | white solid | SM | |
| | | | | | 50° C. | | | | |
| SAS16 | Chlorobenzene | 10.01 | 10.01 | Insoluble | Fine slurry at | B | white solid | SM | |
| | | | | | 50° C. | | | | |
| SAS17 | Ethyl acetate | 10.01 | 10.01 | Insoluble | Slurry at 50° C. | A | white solid | SM | |
| | (EtOAc) | | | | | | | | |
| SAS18 | Propyl acetate | 10.17 | 10.17 | Insoluble | Slurry at 50° C. | A | white solid | SM | |
| SAS19 | Isopropyl acetate | 10.17 | 10.17 | Insoluble | Slurry at 50° C. | A | white solid | SM | |
| SAS20 | n-Butyl acetate | 10.12 | 10.12 | Insoluble | Slurry at 50° C. | B | white solid | SM | 96.3 |
| SAS21 | Isobutyl acetate | 10.17 | 10.17 | Insoluble | Slurry at 50° C. | B | white solid | SM | |
| SAS22 | tert-Butyl | 10.18 | 10.18 | Insoluble | Slurry at 50° C. | A | white solid | SM | |
| | methyl ether | | | | | | | | |
| SAS23 | Ethyl ether | 10.11 | 10.11 | Insoluble | Slurry at 30° C. | A | white solid | SM | |
| SAS24 | Diisopropyl ether | 10.19 | 10.19 | Insoluble | Fine slurry at | A | white solid | SM + pks. | 94.4 |
| | | | | | 50° C. | | | | |
| SAS25 | 1,4-Dioxane | 10.11 | 10.11 | Insoluble | Fine slurry at | A | white solid | SM | |
| | | | | | 50° C. | | | | |
| SAS26 | Anisole | 10.25 | 10.25 | Insoluble | Fine slurry at | B | white solid | SM | |
| | | | | | 50° C. | | | | |
| SAS27 | Tetrahydrofuran | 10.04 | 10.04 | Insoluble | Fine slurry at | A | white solid | SM | |
| | (THF) | | | | 50° C. | | | | |
| SAS18 | Propyl acetate | 10.17 | 10.17 | Insoluble | Slurry at 50° C. | A | white solid | SM | |
| SAS19 | Isopropyl acetate | 10.17 | 10.17 | Insoluble | Slurry at 50° C. | A | white solid | SM | |
| SAS20 | n-Butyl acetate | 10.12 | 10.12 | Insoluble | Slurry at 50° C. | B | white solid | SM | 96.3 |
| SAS21 | Isobutyl acetate | 10.17 | 10.17 | Insoluble | Slurry at 50° C. | B | white solid | SM | |
| SAS22 | tert-Butyl | 10.18 | 10.18 | Insoluble | Slurry at 50° C. | A | white solid | SM | |
| | methyl ether | | | | | | | | |
| SAS23 | Ethyl ether | 10.11 | 10.11 | Insoluble | Slurry at 30° C. | A | white solid | SM | |
| SAS24 | Diisopropyl | 10.19 | 10.19 | Insoluble | Fine slurry at | A | white solid | SM + pks. | 94.4 |
| | ether | | | | 50° C. | | | | |
| SAS25 | 1,4-Dioxane | 10.11 | 10.11 | Insoluble | Fine slurry at | A | white solid | SM | |
| | | | | | 50° C. | | | | |
| SAS26 | Anisole | 10.25 | 10.25 | Insoluble | Fine slurry at | B | white solid | SM | |
| | | | | | 50° C. | | | | |
| SAS27 | Tetrahydrofuran | 10.04 | 10.04 | Insoluble | Fine slurry at | A | white solid | SM | |
| | (THF) | | | | 50° C. | | | | |
| SAS28 | 2-Methyl- | 10.14 | 10.14 | Insoluble | Slurry at 50° C. | A | white solid | SM | |
| | tetrahydrofuran | | | | | | | | |
| | (2Me THF) | | | | | | | | |
| SAS29 | Acetone | 10.26 | 10.26 | Insoluble | Slurry at 50° C. | A | white solid | SM | |
| SAS30 | Methyl ethyl | 10.30 | 10.30 | Insoluble | Slurry at 50° C. | A | white solid | SM | |
| | ketone (MEK) | | | | | | | | |
| SAS31 | 4-Methyl-2- | 10.42 | 10.42 | Insoluble | Fine slurry at | B | white solid | SM | |
| | pentanone | | | | 50° C. | | | | |
| | (Isobutyl | | | | | | | | |
| | methyl ketone) | | | | | | | | |
| SAS32 | Acetonitrile | 10.37 | 10.37 | Insoluble | Slurry at 50° C. | A | white solid | SM | |
| | (MeCN) | | | | | | | | |
| SAS33 | Benzonitrile | 10.38 | 10.38 | Insoluble | Fine slurry at | C | white solid | SM + pks. | 68.0 |
| | | | | | 50° C. | | | | |
| SAS34 | N,N-Dimethyl- | 10.36 | 10.36 | Slightly | 40 | C | white solid | Am. | 1.2 |
| | acetamide | | | soluble | | | | | |
| SAS35 | N-Methyl- | 10.53 | 21.06 | Sparingly | RT | C | pale yellow | Am. | 1.5 |
| | pyrrolidone | | | soluble | | | gel | | |
| SAS36 | Dimethyl | 10.05 | 20.10 | Sparingly | RT | C | white solid | Am. | 32.4 |
| | sulfoxide | | | soluble | | | hygroscopic | | |
| | (DMSO) | | | | | | | | |
| SAS37 | MEK:Toluene | 10.38 | 10.38 | Insoluble | Fine slurry at | A | white solid | SM | |
| | (1:1 v/v) | | | | 50° C. | | | | |
| SAS38 | MEK:2-MeTHF | 10.30 | 10.30 | Insoluble | Fine slurry at | A | white solid | SM | |
| | (1:1 v/v) | | | | 50° C. | | | | |
| SAS39 | EtOH:MeCN | 10.16 | 10.16 | Insoluble | Slurry at 50° C. | A | white solid | S2 + pks. or S2 | 80.1 |
| | (1:1 v/v) | | | | | | | | |
| SAS40 | H₂O:THF | 10.10 | 202.00 | Very | RT | A | white solid | Am. + few | 77.5 |
| | (1:1 v/v) | | | soluble | | | | broad peaks: | |
| | | | | | | | | 3.75-6.38 & | |
| | | | | | | | | 12.45-18.13 | |
| SAS41 | H₂O:EtOH | 10.20 | 204.00 | Very | RT | A | white solid | Am. + few | 90.7 |
| | (1:1 v/v) | | | soluble | | | | broad pks: | |
| | | | | | | | | 3.75-6.38 & | |
| | | | | | | | | 12.45-18.13 | |
| SAS42 | H₂O:2-PrOH | 10.01 | 200.20 | Very | RT | A | white solid | Am. + few | 92.0 |
| | (1:1 v/v) | | | soluble | | | | broad pks: | |
| | | | | | | | | 3.75-6.38 & | |
| | | | | | | | | 12.45-18.13 | |
| SAS43 | H₂O:MeCN | 10.20 | 204.00 | Very | RT | A | white solid | Am. + few | 91.1 |
| | (1:1 v/v) | | | soluble | | | | broad pks: | |
| | | | | | | | | 3.75-6.38 & | |
| | | | | | | | | 12.45-18.13 | |
| SAS44 | H₂O:Acetone | 10.03 | 200.60 | Very | RT | A | white solid | Am. + few | 93.7 |
| | (1:1 v/v) | | | soluble | | | | broad pks: | |
| | | | | | | | | 3.75-6.38 & | |
| | | | | | | | | 12.45-18.13 | |
| SAS45 | H₂O:DMSO | 10.03 | 100.30 | Freely | RT | C | white solid | Am. | 30.2 |
| | (1:1 v/v) | | | soluble | | | | | |
| SAS46 | MeOH:EtOAc | 10.12 | 20.24 | Sparingly | RT | A | white solid | S5, P.O. or | 89.0 |
| | (1:1 v/v) | | | soluble | | | | S5 + SM | |
| | | | | | | | | traces, P.O. | |
| SAS47 | MeOH:Acetone | 10.16 | 20.32 | Sparingly | RT | A | white solid | S5 or S5 + | 91.0 |
| | (1:1 v/v) | | | soluble | | | | SM traces | |
| SAS48 | EtOH:n-Heptane | 10.06 | 10.06 | Insoluble | Slurry at 50° C. | A | white solid | S2 + pks. or | 90.1 |
| | (1:1 v/v) | | | | | | | S2 (similar | |
| | | | | | | | | with SAS39) | |
| SAS49 | MEK:MTBE | 10.08 | 10.08 | Insoluble | Slurry at 50° C. | A | white solid | SM | |
| | (1:1 v/v) | | | | | | | | |
| SAS50 | EtOAc:n-Heptane | 10.26 | 10.26 | Insoluble | Slurry at 50° C. | A | white solid | SM | |
| | (1:1 v/v) | | | | | | | | |

Am. = amorphous;
SM = starting material (i.e., NaCDC Form B);
P.O. = preferred orientation;
pks. = peaks;
Evaporation Conditions (Evap. Cond.):
A = 30° C., low pressure (final value = 20-21 mbar) for 21 hours;
B = 50° C., low pressure (final value = 21 mbar) for 21 hours;
C = 50° C., low pressure (final value = 21 mbar) for 43 hours

The majority of the tested solids displayed good purities by HPLC, except for the experiments with 2-ethoxyethanol, 3-methyl-1-butanol, benzonitrile, DMAc, NMP, DMSO, and H₂O:DMSO (1:1 v/v), where the purity dropped from 99.2% down to 1.2-68.0%.

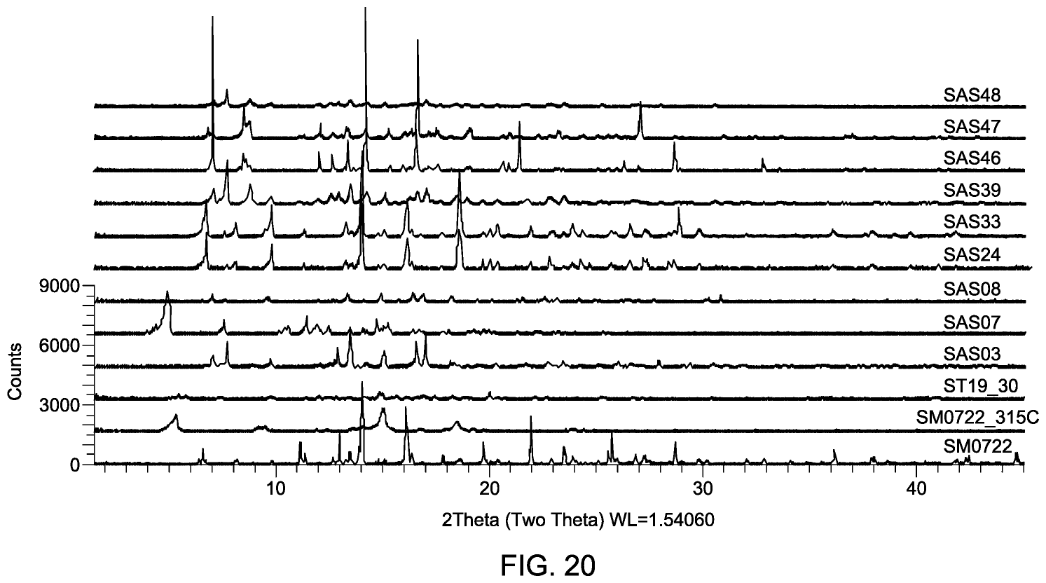

XRPD analysis revealed the formation of several new forms, as shown in FIG. 20.

### Characterization of Form S2

Form S2 was identified from the experiments with EtOH (SAS03) or equi-volumetric EtOH mixture with MeCN (SAS39) and n-heptane (SAS48). Based on the TG/DSC analysis of experiment SAS39 (FIG. 21A), Form S2 was assigned as a solvated form with a first endothermic event, suggesting desolvation, at 112.9° C. with a mass loss of 3.95% (attributed to 0.5 EtOH molecules, which corresponds with a theoretical mass of 5%). The second event represents a phase transformation with Tₒₙₛₑₜ at 177.8° C., followed by a recrystallization event at around 195° C., and finally melting at 334.1° C. (Tₒₙₛₑₜ).

### Characterization of Form S3

Form S3 was identified from a single experiment with 2-ethoxyethanol (SASO7). Form S3 was determined to likely be a mixture between a preponderant crystalline degradation product and NaCDC, based on the HPLC analysis (purity of 38% at 212 nm). XRPD analysis of the material from experiment SAS07 is shown in FIG. 21B, and the corresponding data are summarized in Table S3:

TABLE S3

| 2-theta | d-Spacing | Rel. |
|---|---|---|
| Angle (°) | (Å) | Intensity (%) |
| 3.94 | 22.40 | 7.89 |
| 4.20 | 21.00 | 16.64 |
| 4.37 | 20.22 | 23.50 |
| 4.67 | 18.93 | 39.81 |
| 4.88 | 18.08 | 100.00 |
| 5.00 | 17.66 | 78.92 |
| 6.48 | 13.62 | 7.54 |
| 7.56 | 11.68 | 34.74 |
| 10.12 | 8.74 | 9.23 |
| 10.42 | 8.48 | 15.19 |
| 10.56 | 8.37 | 18.34 |
| 11.45 | 7.72 | 47.54 |
| 11.68 | 7.57 | 5.62 |
| 11.81 | 7.49 | 11.64 |
| 11.93 | 7.41 | 24.57 |
| 12.46 | 7.10 | 22.22 |
| 13.49 | 6.56 | 11.75 |
| 14.08 | 6.29 | 15.82 |
| 14.23 | 6.22 | 10.40 |
| 14.73 | 6.01 | 41.20 |
| 15.08 | 5.87 | 16.29 |
| 15.25 | 5.81 | 27.14 |
| 16.44 | 5.39 | 11.54 |
| 16.72 | 5.30 | 9.34 |
| 17.82 | 4.97 | 7.27 |
| 18.08 | 4.90 | 11.70 |
| 18.82 | 4.71 | 2.85 |
| 18.98 | 4.67 | 4.20 |
| 19.29 | 4.60 | 9.70 |
| 19.48 | 4.55 | 6.19 |
| 19.75 | 4.49 | 8.69 |
| 19.97 | 4.44 | 10.04 |
| 20.22 | 4.39 | 6.29 |
| 20.41 | 4.35 | 3.85 |
| 21.30 | 4.17 | 5.64 |
| 21.74 | 4.08 | 1.85 |
| 22.21 | 4.00 | 5.74 |
| 22.44 | 3.96 | 5.98 |
| 23.03 | 3.86 | 5.34 |
| 23.25 | 3.82 | 5.34 |
| 23.52 | 3.78 | 2.60 |
| 24.02 | 3.70 | 5.74 |
| 25.34 | 3.51 | 4.87 |
| 25.58 | 3.48 | 3.50 |
| 26.45 | 3.37 | 6.11 |
| 29.24 | 3.05 | 2.37 |
| 29.61 | 3.01 | 3.32 |
| 30.04 | 2.97 | 2.18 |
| 33.25 | 2.69 | 4.64 |
| 36.05 | 2.49 | 2.24 |
| 39.16 | 2.30 | 1.70 |
| 41.66 | 2.17 | 1.89 |
| 44.31 | 2.04 | 1.81 |

### Characterization of Form S4

Form S4 was identified from a single experiment with 2,2,2-trifluoroethanol (SASO8). Based on TG/DSC analysis of experiment SASO8 (FIG. 22), Form S4 was assigned as a solvated form with a first endothermic event, suggesting desolvation, at 146.5° C. with a mass loss of 14.23% (attributed to 1 TFE molecule, which corresponds with a theoretical mass of 19.44%). The second event represents melting at 333.9° C. (Tₒₙₛₑₜ).

### Characterization of Form S5

Form S5 was identified from two experiments with equi-volumetric MeOH mixture with EtOAc (SAS46) and n-heptane (SAS47). An XRPD spectrum of the material obtained from experiment SAS47 is shown in FIG. 23A, and the corresponding data are summarized in Table S5-A:

TABLE S5-A

| 2-theta | d-Spacing | Rel. |
|---|---|---|
| Angle (°) | (Å) | Intensity (%) |
| 6.82 | 12.96 | 8.24 |
| 7.01 | 12.59 | 6.44 |
| 8.51 | 10.38 | 27.24 |
| 8.72 | 10.14 | 13.29 |
| 11.14 | 7.94 | 1.33 |
| 11.31 | 7.82 | 3.53 |
| 12.01 | 7.36 | 2.85 |
| 12.10 | 7.31 | 14.03 |
| 12.70 | 6.97 | 4.13 |
| 13.09 | 6.76 | 2.11 |
| 13.34 | 6.63 | 6.01 |
| 14.19 | 6.24 | 8.21 |
| 14.77 | 5.99 | 0.80 |
| 15.30 | 5.79 | 9.29 |
| 16.05 | 5.52 | 5.84 |
| 16.35 | 5.42 | 8.63 |
| 16.64 | 5.32 | 100.00 |
| 17.16 | 5.16 | 5.64 |
| 17.54 | 5.05 | 9.10 |
| 18.10 | 4.90 | 1.33 |
| 18.24 | 4.86 | 1.64 |
| 18.97 | 4.67 | 5.73 |
| 19.09 | 4.65 | 10.87 |
| 20.64 | 4.30 | 1.82 |
| 20.95 | 4.24 | 3.54 |
| 21.41 | 4.15 | 2.03 |
| 22.31 | 3.98 | 3.61 |
| 22.84 | 3.89 | 1.39 |
| 23.24 | 3.82 | 7.45 |
| 23.61 | 3.77 | 1.70 |
| 24.41 | 3.64 | 2.74 |
| 25.10 | 3.55 | 2.18 |
| 25.58 | 3.48 | 1.68 |
| 25.92 | 3.43 | 3.52 |
| 27.06 | 3.29 | 39.98 |
| 28.73 | 3.10 | 1.80 |
| 29.00 | 3.08 | 0.84 |
| 30.99 | 2.88 | 4.18 |
| 31.56 | 2.83 | 0.47 |
| 32.20 | 2.78 | 0.87 |
| 32.46 | 2.76 | 0.80 |
| 33.69 | 2.66 | 0.63 |
| 36.09 | 2.49 | 0.85 |
| 36.74 | 2.44 | 1.19 |
| 37.03 | 2.43 | 5.21 |
| 38.02 | 2.37 | 1.58 |
| 39.87 | 2.26 | 0.72 |
| 40.71 | 2.21 | 0.74 |
| 41.10 | 2.19 | 1.08 |
| 41.56 | 2.17 | 1.67 |
| 42.89 | 2.11 | 1.72 |

Based on the TG/DSC analysis of experiment SAS47 (FIG. 23B), Form S5 was assigned as a solvated form with a first endothermic event, suggesting desolvation, at around 82° C. with a mass loss of 4.79% (attributed to 0.5 MeOH molecules, which corresponds with a theoretical mass of 3.59%). The second event represents a phase transformation with Tₒₙₛₑₜ at 183.9° C., and lastly, melting at 331.5° C. (Tₒₙₛₑₜ).

### Example 9. Polymorph Screening

### Slurry

Around 15 mg of NaCDC Form B was weighted into HPLC vials, and the appropriate volume of solvent was added at RT. Slurries were aged for 7 days under stirring (500 rpm), at multiple temperatures: 5° C., 25-30° C., 40° C., and 50° C. All solids were air-dried on filter paper and then analyzed by XRPD. Depending on the results obtained by XRPD and material availability, some experiments were analyzed by HTPLC and TG/DSC. The results from the slurry experiments are summarized in Table 7.

TABLE 7

| | | | | | | | | HPLC |
|---|---|---|---|---|---|---|---|---|
| | | | Apparent | | | | | purity |
| | Mass | | conc. | T | | | | (%) at |
| Expt | (mg) | Solvent | (mg/mL) | (° C.) | Comments | Observation | XRPD | 212 nm |
| SL01 | 15.16 | Ethanol (EtOH) | 37.90 | 5 | | white solid | S2 | 89.70 |
| SL02 | 15.06 | 2-Propanol | 37.65 | 5 | | white solid | SM | |
| | | (2-PrOH) | | | | | | |
| SL03 | 15.21 | 2-Methyl-1- | 38.03 | 5 | | white solid | SM | |
| | | propanol | | | | | | |
| | | (Isobutanol) | | | | | | |
| SL04 | 15.01 | 2-Butanol | 37.53 | 5 | | white solid | SM | |
| SL05 | 15.14 | Methylcyclohexane | 37.85 | 5 | the initial | white crystals | SM, P.O. | |
| | | | | | homogenous slurry | | | |
| | | | | | became solution | | | |
| | | | | | with formed white | | | |
| | | | | | crystals into it | | | |
| SL06 | 15.01 | n-Heptane | 37.53 | 5 | | white solid | SM, l.c. | |
| SL07 | 15.07 | 2,2,4- | 37.68 | 5 | | white solid | SM | |
| | | Trimethylpentane | | | | | | |
| | | (Isooctane) | | | | | | |
| SL08 | 15.04 | Toluene | 37.60 | 5 | | white solid | SM | |
| SL09 | 15.14 | Xylenes | 37.85 | 5 | | white solid | SM | |
| SL10 | 15.04 | Ethyl acetate | 37.60 | 5 | | white solid | SM | |
| | | (EtOAc) | | | | | | |
| SL11 | 15.04 | Isopropyl acetate | 37.60 | 5 | | white solid | SM | |
| SL12 | 15.18 | n-Butyl acetate | 37.95 | 5 | | white solid | SM | |
| SL13 | 15.06 | tert-Butyl | 37.65 | 5 | | white solid | Similar to/ | 87.21 |
| | | methyl ether | | | | | isomorphic | |
| | | | | | | | with S1, l.c. | |
| SL14 | 15.13 | Diisopropyl ether | 37.83 | 5 | | white solid | SM | |
| SL15 | 15.01 | Tetrahydrofuran | 37.53 | 5 | | white solid | SM, l.c. | |
| | | (THF) | | | | | | |
| SL16 | 15.30 | 2-Methyl- | 38.25 | 5 | | white solid | SM | |
| | | tetrahydrofuran | | | | | | |
| SL17 | 15.23 | Acetone | 38.08 | 5 | | white solid | SM | |
| SL18 | 15.15 | Methyl ethyl | 37.88 | 5 | | white solid | SM | |
| | | ketone (MEK) | | | | | | |
| SL19 | 15.26 | Acetonitrile | 38.15 | 5 | | white solid | SM, l.c. | |
| | | (MeCN) | | | | | | |
| SL20 | 15.16 | MEK:Toluene | 37.90 | 5 | | white solid | SM | |
| | | (1:1 v/v) | | | | | | |
| SL21 | 15.25 | MEK:2-MeTHF | 38.13 | 5 | | white solid | SM | |
| | | (1:1 v/v) | | | | | | |
| SL22 | 15.02 | EtOH:MeCN | 37.55 | 5 | | white solid | S2, l.c. | 91.93 |
| | | (1:1 v/v) | | | | | | |
| SL23 | 15.00 | EtOH:n-Heptane | 37.50 | 5 | | white solid | S2 | 93.64 |
| | | (1:1 v/v) | | | | | | |
| SL24 | 15.04 | MEK:MTBE | 37.60 | 5 | | white solid | SM | |
| | | (1:1 v/v) | | | | | | |
| SL25 | 15.22 | EtOAc:n-Heptane | 38.05 | 5 | | white solid | SM | |
| | | (1:1 v/v) | | | | | | |
| SL26 | 15.16 | Ethanol (EtOH) | 37.90 | 25-30 | | white solid | S2 | 92.34 |
| SL27 | 15.02 | 2-Propanol | 37.55 | 25-30 | | white solid | l.c., S7 | 88.33 |
| | | (2-PrOH) | | | | | | |
| SL28 | 15.07 | 2-Methyl-1- | 37.68 | 25-30 | | white solid | l.c., SM | |
| | | propanol | | | | | | |
| | | (Isobutanol) | | | | | | |
| SL29 | 15.16 | 2-Butanol | 37.90 | 25-30 | | white solid | SM, l.c. | |
| SL30 | 15.26 | Cyclohexane | 38.15 | 25-30 | | white solid | SM | |
| SL31 | 15.29 | Methylcyclohexane | 38.23 | 25-30 | after 5 days of | white solid | SM, l.c. | |
| | | | | | aging, volume of | | | |
| | | | | | solvent decreased | | | |
| | | | | | at around half of | | | |
| | | | | | initial quantity; | | | |
| | | | | | on the 7th day - | | | |
| | | | | | solvent almost | | | |
| | | | | | evap./consumed | | | |
| | | | | | from the reaction | | | |
| | | | | | mixture | | | |
| SL32 | 15.02 | n-Heptane | 37.55 | 25-30 | on the 7th day - | white solid | SM | |
| | | | | | solvent almost | | | |
| | | | | | evap./consumed | | | |
| | | | | | from the reaction | | | |
| | | | | | mixture | | | |
| SL33 | 15.03 | 2,2,4- | 37.58 | 25-30 | | white solid | SM | |
| | | Trimethylpentane | | | | | | |
| | | (Isooctane) | | | | | | |
| SL34 | 15.23 | Toluene | 38.08 | 25-30 | | white solid | SM | |
| SL35 | 15.46 | Xylenes | 38.65 | 25-30 | | white solid | SM | |
| SL36 | 15.02 | Ethyl acetate | 37.55 | 25-30 | | white solid | SM | |
| | | (EtOAc) | | | | | | |
| SL37 | 15.08 | Isopropyl acetate | 37.70 | 25-30 | | white solid | SM | |
| SL38 | 15.30 | n-Butyl acetate | 38.25 | 25-30 | | white solid | SM | |
| SL39 | 15.00 | tert-Butyl | 37.50 | 25-30 | | white solid | Similar to/ | 84.05 |
| | | methyl ether | | | | | isomorphic | |
| | | | | | | | with S1, l.c. | |
| SL40 | 15.21 | Diisopropyl ether | 38.03 | 25-30 | | white solid | SM | |
| SL41 | 15.06 | Tetrahydrofuran | 37.65 | 25-30 | | white solid | SM, l.c. | |
| | | (THF) | | | | | | |
| SL42 | 15.27 | 2-Methyl- | 38.18 | 25-30 | | white solid | SM (P.O. | |
| | | tetrahydrofuran | | | | | with 1 pk. | |
| | | | | | | | at around | |
| | | | | | | | 32.1 deg.) | |
| SL43 | 15.08 | Acetone | 37.70 | 25-30 | | white solid | SM | |
| SL44 | 15.30 | Methyl ethyl | 38.25 | 25-30 | | white solid | SM | |
| | | ketone (MEK) | | | | | | |
| SL45 | 15.30 | Acetonitrile | 38.25 | 25-30 | | white solid | SM | |
| | | (MeCN) | | | | | | |
| SL46 | 15.22 | MEK:Toluene | 38.05 | 25-30 | | white solid | SM | |
| | | (1:1 v/v) | | | | | | |
| SL47 | 15.33 | MEK:2-MeTHF | 38.33 | 25-30 | | white solid | SM | |
| | | (1:1 v/v) | | | | | | |
| SL48 | 15.22 | EtOH:MeCN | 38.05 | 25-30 | | white solid | S2 | 83.87 |
| | | (1:1 v/v) | | | | | | |
| SL49 | 15.25 | EtOH:n-Heptane | 38.13 | 25-30 | | white solid | S2 | 93.06 |
| | | (1:1 v/v) | | | | | | |
| SL50 | 15.16 | MEK:MTBE | 37.90 | 25-30 | | white solid | SM | |
| | | (1:1 v/v) | | | | | | |
| SL51 | 15.39 | EtOAc:n-Heptane | 38.48 | 25-30 | | white solid | SM | |
| | | (1:1 v/v) | | | | | | |
| SL52 | 15.24 | Ethanol (EtOH) | 50.80 | 40 | after 4 days, the | white solid | S2, P.O. | 87.98 |
| | | | | | solid almost dried | | | |
| | | | | | on vial walls + | | | |
| | | | | | clear solution; | | | |
| | | | | | solid was put into | | | |
| | | | | | solution with a | | | |
| | | | | | spatula and | | | |
| | | | | | remained as slurry; | | | |
| | | | | | on 5th day of | | | |
| | | | | | aging, volume of | | | |
| | | | | | solvent decreased | | | |
| | | | | | at around half of | | | |
| | | | | | initial quantity; on | | | |
| | | | | | 7th day - solvent | | | |
| | | | | | almost evap./ | | | |
| | | | | | consumed from the | | | |
| | | | | | reaction mixture | | | |
| SL53 | 15.02 | 2-Methyl-1- | 50.07 | 40 | | white solid | SM | |
| | | propanol | | | | | | |
| | | (Isobutanol) | | | | | | |
| SL54 | 15.18 | tert-Butyl | 50.60 | 40 | | white solid | SM | |
| | | methyl ether | | | | | | |
| SL55 | 15.40 | Acetone | 51.33 | 40 | | white solid | SM | |
| SL56 | 15.06 | 2-Propanol | 50.20 | 50 | after 4 days, solid | small white | S7 | 84.92 |
| | | (2-PrOH) | | | almost dried on | crystals | | |
| | | | | | vial walls + clear | | | |
| | | | | | solution; solid was | | | |
| | | | | | put into solution | | | |
| | | | | | with a spatula and | | | |
| | | | | | remained as slurry | | | |
| SL57 | 15.33 | 2-Butanol | 51.10 | 50 | | small white | SM + pks., | |
| | | | | | | crystals | P.O. | |
| SL58 | 15.02 | Cyclohexane | 12.52 | 50 | after 1 day: extra | white solid | SM (better | |
| | | | | | 300 μL of solv.; | | crystal. | |
| | | | | | after another 2 | | comp. with | |
| | | | | | days: extra 300 μL | | SL30) | |
| | | | | | of solv. & next day | | | |
| | | | | | after the 2nd add.: | | | |
| | | | | | extra 300 μL of solv. | | | |
| SL59 | 15.48 | Methylcyclohexane | 25.80 | 50 | on 4th day: extra | white solid | SM | |
| | | | | | 300 μL of solv. | | | |
| SL60 | 15.48 | n-Heptane | 17.20 | 50 | after 1 day: extra | white solid | l.c., SM | |
| | | | | | 300 μL of solv.; | | | |
| | | | | | after another 2 | | | |
| | | | | | days: extra 300 μL | | | |
| | | | | | of solv.; on 7th | | | |
| | | | | | day - solvent | | | |
| | | | | | almost evap./ | | | |
| | | | | | consumed from the | | | |
| | | | | | reaction mixture | | | |
| SL61 | 15.06 | 2,2,4- | 50.20 | 50 | | white solid | SM, low | |
| | | Trimethylpentane | | | | | cryst. mat. | |
| | | (Isooctane) | | | | | with P.O. | |
| SL62 | 15.00 | Toluene | 50.00 | 50 | | white solid | SM | |
| SL63 | 15.28 | Xylenes | 50.93 | 50 | | white solid | SM (better | |
| | | | | | | | crystal. | |
| | | | | | | | comp. with | |
| | | | | | | | SL09 & 35) | |
| SL64 | 15.12 | Ethyl acetate | 50.40 | 50 | | white solid | SM | |
| | | (EtOAc) | | | | | | |
| SL65 | 15.20 | Isopropyl acetate | 50.67 | 50 | | white solid | SM (better | |
| | | | | | | | cryst. comp. | |
| | | | | | | | with SL37) | |
| SL66 | 15.11 | n-Butyl acetate | 50.37 | 50 | | white solid | SM | |
| SL67 | 15.07 | Diisopropyl ether | 50.23 | 50 | | white solid | SM | |
| SL68 | 15.03 | Tetrahydrofuran | 50.10 | 50 | | white solid | SM | 78.16 |
| | | (THF) | | | | | | |
| SL69 | 15.17 | 2-Methyl- | 50.57 | 50 | | white solid | SM | |
| | | tetrahydrofuran | | | | | | |
| SL70 | 15.47 | Methyl ethyl | 25.78 | 50 | on 6th day: extra | white solid | SM, h.c. | 90.88 |
| | | ketone (MEK) | | | 300 μL of solv. | | | |
| SL71 | 15.16 | Acetonitrile | 50.53 | 50 | | white solid | SM (better | |
| | | (MeCN) | | | | | crystal. | |
| | | | | | | | comp. | |
| | | | | | | | with SL45) | |
| SL72 | 15.31 | MEK:Toluene | 51.03 | 50 | | white solid | SM (better | |
| | | (1:1 v/v) | | | | | crystal. | |
| | | | | | | | comp. with | |
| | | | | | | | SL20 & 46) | |
| SL73 | 15.28 | MEK:2-MeTHF | 50.93 | 50 | | white solid | SM (better | |
| | | (1:1 v/v) | | | | | crystal. | |
| | | | | | | | comp. with | |
| | | | | | | | SL21 & 47) | |
| SL74 | 15.30 | EtOH:MeCN | 51.00 | 50 | after 4 days, solid | small white | S2, P.O. | 78.36 |
| | | (1:1 v/v) | | | almost dried on | crystals | | |
| | | | | | vial walls + clear | | | |
| | | | | | solution; solid was | | | |
| | | | | | put into solution | | | |
| | | | | | with a spatula and | | | |
| | | | | | remained as slurry | | | |
| SL75 | 15.30 | EtOH:n-Heptane | 51.00 | 50 | after 4 days, solid | small white | S2, P.O. or | 85.10 |
| | | (1:1 v/v) | | | almost dried on | crystals | S2 + pks., | |
| | | | | | vial walls + clear | | P.O. | |
| | | | | | solution; solid was | | | |
| | | | | | put into solution | | | |
| | | | | | with a spatula and | | | |
| | | | | | remained as slurry | | | |
| SL76 | 15.30 | MEK:MTBE | 51.00 | 50 | | white solid | SM (better | |
| | | (1:1 v/v) | | | | | crystal. | |
| | | | | | | | comp. with | |
| | | | | | | | SL24 & 50) | |
| SL77 | 15.25 | EtOAc:n-Heptane | 50.83 | 50 | | white solid | SM | |
| | | (1:1 v/v) | | | | | | |

Am. = amorphous;
SM = starting material (i.e., NaCDC Form B);
P.O. = preferred orientation;
pks. = peaks

Based on the XRPD data, the majority of the material recovered from the slurry experiments, returned the same crystalline phase as the starting material (NaCDC Form B). In some experiments, three different forms were identified—Form S1 (or a form isomorphic with Form S1), Form S2, and Form S7.

### Characterization of Form S1

Form S1 (or a form similar to and/or isomorphic with Form S1) was obtained from experiments SL13 and SL39 with tert-butyl methyl ether at 5° C. and 25-30° C., respectively. HPLC analysis of both materials displayed good purity—87.2% (SL13) and 84.0% (SL39) at 212 nm. XRPD analysis of these samples is shown in FIG. 24A, and the corresponding data for the sample from experiment SL39 are summarized in Table S1-B:

TABLE S1-B

| 2-theta | d-Spacing | Rel. |
|---|---|---|
| Angle (°) | (Å) | Intensity (%) |
| 5.49 | 16.10 | 54.17 |
| 5.82 | 15.17 | 37.62 |
| 7.49 | 11.79 | 38.55 |
| 9.39 | 9.41 | 13.11 |
| 9.82 | 9.00 | 48.66 |
| 9.95 | 8.88 | 16.00 |
| 11.48 | 7.70 | 22.76 |
| 12.07 | 7.33 | 47.50 |
| 12.43 | 7.12 | 31.30 |
| 12.55 | 7.05 | 21.25 |
| 12.88 | 6.87 | 34.25 |
| 13.38 | 6.61 | 13.81 |
| 14.10 | 6.28 | 34.28 |
| 14.93 | 5.93 | 88.12 |
| 15.46 | 5.73 | 24.46 |
| 15.71 | 5.64 | 53.20 |
| 16.15 | 5.48 | 29.22 |
| 16.62 | 5.33 | 33.26 |
| 16.93 | 5.23 | 47.84 |
| 17.48 | 5.07 | 44.48 |
| 17.96 | 4.93 | 22.26 |
| 18.31 | 4.84 | 39.72 |
| 18.71 | 4.74 | 20.13 |
| 18.96 | 4.68 | 22.45 |
| 20.05 | 4.43 | 100.00 |
| 20.44 | 4.34 | 35.98 |
| 22.25 | 3.99 | 8.83 |
| 22.72 | 3.91 | 22.86 |
| 23.36 | 3.81 | 6.81 |
| 23.94 | 3.71 | 10.57 |
| 24.13 | 3.69 | 12.36 |
| 25.31 | 3.52 | 25.44 |
| 26.76 | 3.33 | 9.74 |
| 29.91 | 2.98 | 6.57 |
| 30.45 | 2.93 | 9.70 |
| 30.51 | 2.93 | 5.94 |
| 35.71 | 2.51 | 11.00 |
| 40.89 | 2.21 | 9.06 |

Based on TG/DSC analysis of SL39 (air-dried on filter paper at least 2h before use it for thermal analysis) showed two endothermic events (FIG. 24B): the first event is associated with a desolvation process with Tₒₙₛₑₜ=93.2° C. and a mass loss of 1.241% (theoretical mass of 2.19% corresponds with 0.125 MTBE molecules). As such, the observed mass loss may be attributed to a physical adsorption or residual one. A second event corresponds with melting of the form with Tₒₙₛₑₜ 312.8° C.

### Characterization of Form S2

Form S2 was obtained from all slurry experiments that involved ethanol (i.e., experiments SL01, SL26, and SL52 at 5° C., 25-30° C., and 40° C., respectively), or from mixtures with ethanol at 5° C., 25-30° C., and 50° C. (experiments SL22, SL48, and SL74 with EtOH:MeCN 1:1 v/v, and experiments SL23, SL49, and SL75 with EtOH:n-heptane 1:1 v/v, respectively).

TG/DSC analysis of the material obtained from experiment SL26 (FIG. 25) displayed two endothermic events: The first event is associated with desolvation with Tₒₙₛₑₜ=113.4° C. and a mass loss of 7.013% (theoretical mass of 5% corresponds with 0.75 molecules of EtOH). The second event was attributed to melting, having a Tₒₙₛₑₜ=324.6° C. The presence of two maximum temperatures associated with melting may be attributed to non-homogeneity of the material (e.g., a possible mixture of small and large particles). Based on the data obtained, Form S2 was assigned as a solvate of NaCDC.

### Characterization of Form S7

Form S7 was obtained from experiments SL27 and SL56 with 2-PrOH at 25-30° C. and 50° C., respectively. XRPD spectra of the materials obtained from experiments SL27 and SL56 are shown in FIG. 26A, and the corresponding data from experiment SL56 are summarized in Table S7:

TABLE S7

| 2-theta | d-Spacing | Rel. |
|---|---|---|
| Angle (°) | (Å) | Intensity (%) |
| 5.05 | 17.50 | 0.18 |
| 8.19 | 10.79 | 0.12 |
| 8.35 | 10.58 | 0.73 |
| 8.47 | 10.43 | 0.76 |
| 9.71 | 9.10 | 0.28 |
| 9.90 | 8.93 | 1.68 |
| 10.25 | 8.62 | 0.25 |
| 12.64 | 7.00 | 0.61 |
| 12.95 | 6.83 | 0.07 |
| 13.30 | 6.65 | 0.14 |
| 13.76 | 6.43 | 0.17 |
| 14.36 | 6.16 | 6.07 |
| 14.65 | 6.04 | 0.07 |
| 15.26 | 5.80 | 100.00 |
| 15.50 | 5.71 | 1.81 |
| 17.00 | 5.21 | 1.90 |
| 17.57 | 5.04 | 1.53 |
| 17.72 | 5.00 | 4.20 |
| 19.31 | 4.59 | 0.23 |
| 20.62 | 4.30 | 0.70 |
| 21.22 | 4.18 | 0.79 |
| 21.30 | 4.17 | 1.31 |
| 22.31 | 3.98 | 0.06 |
| 23.09 | 3.85 | 0.28 |
| 23.26 | 3.82 | 0.25 |
| 23.81 | 3.73 | 0.33 |
| 24.82 | 3.58 | 0.04 |
| 25.22 | 3.53 | 0.38 |
| 25.36 | 3.51 | 1.43 |
| 25.85 | 3.44 | 0.14 |
| 26.90 | 3.31 | 0.12 |
| 27.27 | 3.27 | 0.09 |
| 28.23 | 3.16 | 0.05 |
| 28.70 | 3.11 | 0.19 |
| 29.00 | 3.08 | 0.12 |
| 29.35 | 3.04 | 0.20 |
| 30.05 | 2.97 | 0.08 |
| 30.86 | 2.90 | 0.09 |
| 32.32 | 2.77 | 0.19 |
| 32.57 | 2.75 | 0.21 |
| 32.99 | 2.71 | 0.08 |
| 33.03 | 2.71 | 0.06 |
| 33.32 | 2.69 | 0.08 |
| 34.90 | 2.57 | 0.09 |
| 35.59 | 2.52 | 0.05 |
| 36.22 | 2.48 | 0.05 |
| 36.70 | 2.45 | 0.35 |
| 37.48 | 2.40 | 0.58 |
| 38.59 | 2.33 | 0.05 |
| 39.42 | 2.28 | 0.16 |
| 39.76 | 2.27 | 0.31 |
| 41.20 | 2.19 | 0.08 |
| 42.28 | 2.14 | 1.16 |
| 42.40 | 2.13 | 0.75 |
| 44.01 | 2.06 | 0.05 |

Based on the TG/DSC analysis of the material obtained from experiment SL56 (air-dried on filter paper at least 2 h before thermal analysis), three endothermic events were observed (FIG. 26B). The first event is attributed to desolvation with Tₒₙₛₑₜ=109.2° C. and a mass loss of 10.037% (theoretical mass of 12.66% corresponds with one molecule of 2-PrOH). The second event likely corresponds to a phase transformation (Tₒₙₛₑₜ=324.5° C. and a heat of 2.65 J/g). The third event represents melting with Tₒₙₛₑₜ=335.3° C. Based on the similar melting temperature of Form S7 with that of NaCDC Form B (Tₒₙₛₑₜ=336.4° C. and Max T=340.2° C.), Form S7 was assigned as a solvated form of the anhydrous NaCDC Form B.

Slurry with Sonication

Around 15 mg of NaCDC Form B was weighed into HPLC vials and 0.4 mL of corresponding solvents were added at RT. The slurries were sonicated for 15 minutes and then left to cool at 5° C. for 30 minutes—this cycle was repeated 6 times. At the end of the sixth cycle, all experiments were air-dried on filter paper and the recovered solids were analyzed by XRPD. Based on the XRPD data, some experiments were analyzed by HPLC. The results from slurry at 5° C. with sonication and thermal cycling experiments (SLS) are presented in Table 8.

TABLE 8

| | | | Initial | | | HPLC |
|---|---|---|---|---|---|---|
| | | | apparent | | | purity |
| | Mass | | conc. | | | (%) at |
| Expt | (mg) | Solvent | (mg/mL) | Observation | XRPD | 212 nm |
| SLS01 | 15.19 | 2-Propanol | 37.98 | white solid | SM | |
| | | (2-PrOH) | | | | |
| SLS02 | 15.05 | 2-Butanol | 37.63 | white solid | SM | |
| SLS03 | 15.13 | Ethyl acetate | 37.83 | white solid | SM + pks., | 90.42 |
| | | (EtOAc) | | | P.O. | |
| SLS04 | 15.02 | Isopropyl acetate | 37.55 | white solid | SM | |
| SLS05 | 15.43 | Diisopropyl ether | 38.58 | white solid | SM | |
| SLS06 | 15.45 | Acetone | 38.63 | white solid | SM | |
| SLS07 | 15.21 | Methyl ethyl | 38.03 | white solid | SM | |
| | | ketone (MEK) | | | | |
| SLS08 | 15.08 | EtOH:MeCN | 37.70 | white solid | S2 | 79.23 |
| | | (1:1 v/v) | | | | |
| SLS09 | 15.06 | EtOH:n-Heptane | 37.65 | white solid | S2 | 89.68 |
| | | (1:1 v/v) | | | | |

SM = starting material (i.e., NaCDC Form B);
P.O. = preferred orientation;
pks. = peaks

Based on the XRPD data, the majority of the material recovered from the slurry experiments, returned the same crystalline phase as the starting material (NaCDC Form B). Form S2 was obtained from experiments SLS08 and SLS09 with EtOH:MeCN 1:1 v/v and EtOH:n-heptane 1:1 v/v, respectively. HPLC analysis of both materials displayed good purity at 79.2% (SLS08) and 89.7% (SLS09) at 212 nm.

### Slow Evaporation

Five stock solutions (with MeOH, water, 2,2,2-trifluoroethanol, MeOH:EtOAc (1:1 v/v) and MeOH:acetone (1:1 v/v)) were prepared with NaCDC Form B at RT. The final concentration of each solution was near that obtained from the Solubility Studies described in Example 8. Around 19-23 mg of NaCDC Form B was weighed into 1.6 mL HPLC vials (or 8 mL flask in the case of stock solutions with MeOH:EtOAc and MeOH:acetone) and dissolved in appropriate solvents at RT. In the case of the stock solution with water, around 40 mg NaCDC Form B was used. The experiments were kept at RT for 1 h with stirring (500 rpm) and then the solutions were filtered with 0.45 μm PTFE filters. During filtration of the stock solution with water, a foaming phenomenon was observed, and therefore, filtration was performed slowly and in several steps. The filtered solutions were then evaporated slowly under atmospheric pressure at 25° C. (experiments EV01-05) or at 50° C. (experiments EV06-09). After complete evaporation of the solvent, the solids were recovered and analyzed by XRPD. Depending on the results obtained from the XRPD analysis and also material availability, some experiments were analyzed by HPLC and TG/DSC. The results from slow evaporative crystallizations (EV) are summarized in Table 9.

TABLE 9

| | | | | | | | | | HPLC |
|---|---|---|---|---|---|---|---|---|---|
| | | | Solution | | | | | | purity |
| | | Conc. | volume | T_{evap.} | | Solids | | | (%) at |
| Expt | Solvent | (mg/mL) | (μL) | (° C.) | Comments | obtained after | Observation | XRPD | 212 nm |
| EV01 | Methanol (MeOH) | 100.80 | ~220 | 25-30 | almost evap. | 4 days at 25-30° | glassy white | Am. + few broad | 91.70 |
| | | | | | overnight | C. and atm. pres. | solid | pks: 3.6-6.3 & | |
| | | | | | | | | 12.1-19.5 deg. | |
| EV02 | Water (H₂O) | 204.39 | 200 | | almost evap. | 5 days at 25-30° | colorless | Am. + few broad | 93.71 |
| | | | | | after 4 days | C. and atm. pres. | flakes | pks: 3.6-6.3 & | |
| | | | | | | | | 12.1-19.5 deg. | |
| EV03 | 2,2,2- | 50.88 | 460 | | almost evap. | 7 days at 25-30° | sticky white | Similar to/ | 88.27 |
| | Trifluoroethanol | | | | after 4 days; | C. and atm. pres. | solid | isomorphic with | |
| | (TFE) | | | | still as solid + | | | S1, l.c. | |
| | | | | | gel after other | | | | |
| | | | | | 2 days | | | | |
| EV04 | MeOH:EtOAc | 20.49 | 920 | 50 | | 4 days at 25-30° | colorless | Am. + few broad | 86.46 |
| | (1:1 v/v) | | | | | C. and atm. pres. | crystals | pks: 3.6-6.3 & | |
| | | | | | | | | 12.1-19.5 deg. | |
| EV05 | MeOH:Acetone | 20.19 | 930 | | | 4 days at 25-30° | colorless | Am. + few broad | 90.22 |
| | (1:1 v/v) | | | | | C. and atm. pres. | flakes | pks: 3.6-6.3 & | |
| | | | | | | | | 12.1-19.5 deg. | |
| EV06 | Water (H₂O) | 204.39 | 200 | | | several hours | colorless | Am. + broad pk: | 93.24 |
| | | | | | | (overnight) at | flakes + | 3.6-6.3 deg. | |
| | | | | | | 50° C. and atm. | white | | |
| | | | | | | pres. | crystals | | |
| EV07 | 2,2,2- | 50.88 | 460 | | almost evap. | 4 days at 50° C. | white | S4 + SM, P.O. | 91.69 |
| | Trifluoroethanol | | | | overnight | and atm. pres. | crystals | | |
| | (TFE) | | | | | | | | |
| EV08 | MeOH:EtOAc | 20.49 | 910 | | | several hours | white | S6 (similar to or | 71.43 |
| | (1:1 v/v) | | | | | (overnight) at | crystals | isomorph with S3 | |
| | | | | | | 50° C. and atm. | | from SAS07) P.O. | |
| | | | | | | pres. | | | |
| EV09 | MeOH:Acetone | 20.19 | 930 | | almost evap. | 4 days at 50° C. | white solid | S5 | 87.00 |
| | (1:1 v/v) | | | | overnight | and atm. pres. | | | |

Am. = amorphous;
l.c. = low crystallinity;
SM = starting material (i.e., NaCDC Form B);
P.O. = preferred orientation;
pks. = peaks

Based on the XRPD data, the majority of the materials recovered from these experiments were in an amorphous phase. In some experiments, four different forms were identified—Form S1 (or a form isomorphic with Form S1), Form S4, Form S5, and Form S6.

### Characterization of Form S1

Form S1 (or a form similar to and/or isomorphic with Form S1) was obtained from experiment EV03 with 2,2,2-trifluoroethanol at 25-30° C. HPLC analysis of the material displayed good purity of 88.3% at 212 nm. TG/DSC analysis of the material obtained from experiment EV03 displayed three endothermic events (FIG. 27). The first two events were attributed to desolvation with Tₒₙₛₑₜ=94.4° C. and Tₒₙₛₑₜ=144.7° C., respectively, and a cumulative mass loss of 5.487% (theoretical mass of 4.86% corresponds with 0.25 TFE molecules). The third event corresponds to melting of the form with Tₒₙₛₑₜ=335.9° C.

### Characterization of Form S4

Form S4 (in mixture with SM and preferred orientation) was obtained from experiment EVO7 with TFE after slow evaporation at 50° C. The material had a purity of 91.7%, as judged by HPLC at 212 nm. Being obtained from an experiment with TFE, the result suggests that Form S4 is a solvated form.

### Characterization of Form S5

Form S5 was obtained from experiment EV09 with MeOH:acetone 1:1 v/v at 50° C. Based on TG/DSC analysis of the material obtained from experiment EV09, two events were observed (FIG. 28). Before the first event, a mass loss of 5.642% was observed on the TG curve without being attributed to a well-defined desolvation process. This may be due to a physically adsorbed solvent (for 1 molecule of MeOH, the theoretical mass is calculated at 7.17%). A possible recrystallization event was observed with Tₒₙₛₑₜ=195.3° C. (and 2 maximum temperatures at 198.9° C. and 212.5° C.). A melting event was observed with Tₒₙₛₑₜ=332.9° C.

### Characterization of Form S6

Form S6 was obtained from experiment EV08 with MeOH:EtOAc 1:1 v/v at 50° C. Form S6 was similar to Form S3 from SASO7 experiment. HPLC purity of the material was determined to be 71.4% (with a main impurity of 18% at a retention time of 3.81 min) at 212 nm. Based on TG/DSC analysis of the material obtained from experiment EV08, three events were observed (FIG. 29). The first, broad endothermic event is associated with desolvation, having a Tₒₙₛₑₜ=50.6° C. and a mass loss of 2.456% (theoretical mass of 2.191% corresponds with 0.125 EtOAc molecules). The second, exothermic event is associated with a recrystallization (Tₒₙₛₑₜ=199.8° C.), while the third, endothermic event is associated with melting, having a Tₒₙₛₑₜ=331.5° C.

### Solvent Drop Grinding

Around 20 mg of NaCDC Form B was ground together with 10 μL aliquots of the corresponding solvent for 10 minutes at RT using an agate mortar with pestle. When total evaporation of the solvent was observed, another aliquot of 10 μL solvent was added and this procedure was repeated until experiment completion time. All solids were analyzed by XRPD. For the experiments where phase transformation was observed via XRPD, additional analyses of TG/DSC and/or HPLC were performed. The results from solvent drop grinding experiments (SDGR) are presented in Table 10.

TABLE 10

| | | | | | | | HPLC |
|---|---|---|---|---|---|---|---|
| | | | Volume of | Grinding | | | purity |
| | Mass | | solvent | time | | | (%) at |
| Expt | (mg) | Solvent | (μL) | (min.) | Observation | XRPD | 212 nm |
| SDGR01 | 20.00 | Ethanol (EtOH) | 140 | 10 | white solid | S2, l.c. | 83.36 |
| SDGR02 | 20.03 | 2-Propanol | 80 | 10 | white solid | SM + similar | 81.26 |
| | | | | | | S1 or | |
| | | | | | | isomorphic | |
| | | | | | | form of S1 | |
| SDGR03 | 20.04 | 2,2,2- | 90 | 10 | white solid | S4 | 90.87 |
| | | Trifluoroethanol | | | | | |
| | | (TFE) | | | | | |
| SDGR04 | 20.00 | Diisopropyl ether | 160 | 10 | white solid | SM, l.c. | |
| SDGR05 | 20.11 | MeOH:EtOAc | 260 | 10 | white solid | S5 | 82.04 |
| | | (1:1 v/v) | | | | | |
| SDGR06 | 20.00 | MeOH:Acetone | 240 | 10 | white solid | S5 | 85.56 |
| | | (1:1 v/v) | | | | | |
| SDGR07 | 20.00 | EtOH:MeCN | 190 | 10 | white solid | S2, l.c. | 86.54 |
| | | (1:1 v/v) | | | | | |
| SDGR08 | 20.07 | EtOH:n-Heptane | 220 | 10 | white solid | S2, l.c. | 90.36 |
| | | (1:1 v/v) | | | | | |

l.c. = low crystallinity;
SM = starting material (i.e., NaCDC Form B)

Based on the XRPD data, four new forms were identified: Form S1 (or isomorphic with Form S1) (in mixture with NaCDC Form B), Form S2, Form S4, and Form S5. Characterization of Form S1

Form S1 (or a form similar to and/or isomorphic with Form S1) was obtained, as a mixture with NaCDC Form B, from experiment SDGR02 with 2-PrOH. This material had a purity of 81.3%, as judged by HPLC at 212 nm.

### Characterization of Form S2

Form S2 was obtained from three experiments that involved ethanol (SDGR01) or a mixture with ethanol (SDGR07—EtOH:MeCN 1:1 v/v and SDGR08—EtOH:n-heptane 1:1 v/v, respectively). The purity of the tested materials, as determined by HPLC, was >86.5% (except the material from experiment SDGR01, which had a purity of 83.4%) at 212 nm.

Based on the TG/DSC analysis of the material obtained from experiment SDGR01 (FIG. 30), Form S2 was assigned as an EtOH-solvated form with a first endothermic event, suggesting desolvation at Tₒₙₛₑₜ=105.3° C. and a mass loss of 7.677% (that can be attributed with 0.75 EtOH molecules that correspond with a theoretical mass of 5%). The second event represents a phase transformation with Tₒₙₛₑₜ at 180.7° C., followed by a recrystallization event at around 200° C., and finally a melting event at 334.6° C. (Tₒₙₛₑₜ). These results are similar to those obtained for the material from experiments SAS48 and SDGR07.

### Characterization of Form S4

Form S4 was obtained from experiment SDGR03 with TFE. The material displayed a good HPLC purity of 90.9% at 212 nm. The results suggested that Form S4 is a TFE-solvated form. An XRPD spectrum of the material obtained from experiment SDGR03 is shown in FIG. 31A, and the corresponding data are summarized in Table S4:

TABLE S4

| 2-theta | d-Spacing | Rel. |
|---|---|---|
| Angle (°) | (Å) | Intensity (%) |
| 7.07 | 12.50 | 48.86 |
| 7.65 | 11.55 | 68.47 |
| 9.70 | 9.11 | 43.30 |
| 12.02 | 7.36 | 15.22 |
| 13.43 | 6.59 | 100.00 |
| 14.19 | 6.24 | 14.94 |
| 15.02 | 5.89 | 65.17 |
| 15.36 | 5.76 | 9.36 |
| 16.52 | 5.36 | 94.59 |
| 16.96 | 5.22 | 87.06 |
| 18.32 | 4.84 | 49.28 |
| 19.52 | 4.54 | 13.77 |
| 20.20 | 4.39 | 17.98 |
| 21.64 | 4.10 | 16.49 |
| 22.65 | 3.92 | 32.05 |
| 23.21 | 3.83 | 43.27 |
| 24.25 | 3.67 | 6.76 |
| 25.94 | 3.43 | 6.12 |
| 26.62 | 3.35 | 24.31 |
| 30.17 | 2.96 | 7.81 |
| 30.26 | 2.95 | 15.35 |
| 31.69 | 2.82 | 9.92 |
| 39.17 | 2.30 | 4.67 |
| 41.45 | 2.18 | 6.01 |

Based on the TG/DSC analysis of the material (FIG. 31B), Form S4 is likely solvated with TFE, with a first endothermic event suggesting desolvation at Tₒₙₛₑₜ=117.6° C. and a mass loss of 14.620% (that can be attributed to 1 molecule of TFE that corresponds with a theoretical mass of 19.44%). The second event represents a phase transformation with Tₒₙₛₑₜ at 185.4° C., followed by a recrystallization event at around 200° C., and finally, a melting event at 335.6° C. (Tₒₙₛₑₜ). These results are similar to those obtained from experiment SAS08.

### Characterization of Form S5

Form S5 was obtained from two experiments, SDGR05 with MeOH:EtOAc 1:1 v/v and SDGR06 with MeOH:acetone 1:1 v/v. The material had a HPLC purity of 87% at 212 nm. Based on the TG/DSC analysis of the material obtained from experiment SDGR05 (FIG. 32A), Form S5 is a solvated form with a first endothermic event, suggesting a desolvation, at around 101° C. and a mass loss of 4.426% (that can be attributed to 0.5 MeOH molecules that corresponds to a theoretical mass of 3.59%). The second event represents a recrystallization with Tₒₙₛₑₜ at 190.9° C., and lastly, a melting event at around 333° C. (Tₒₙₛₑₜ).

Vapor Diffusion onto Solids

Around 20 mg of NaCDC Form B were weighed into 1.6 mL HPLC vials. Each of the aforementioned vials was inserted individually into 40 mL vessels containing initially 0.5 mL of the corresponding solvent. The vessels were isolated from light and atmosphere and allowed to stand for 12 days, in a closed cabinet at 25° C. or in an oven at 50° C. After the experimental time, the majority of the samples remained solids/wet solids (experiments VDS02-12), which were harvested and analyzed by XRPD. Experiment VDS01 resulted in a fine suspension after 12 days and was dried at 30° C., 21 mbar overnight. All solids were analyzed by XRPD, and depending on the results obtained, some experiments were also analyzed by HPLC and TG/DSC. The results from vapor diffusion onto solids (VDS) are summarized in Table 11.

TABLE 11

| | | | Initial | | | | | | HPLC |
|---|---|---|---|---|---|---|---|---|---|
| | | | solvent | | | Solid obtained | | | purity |
| | Mass | | volume | T | | by/Remained | | | (%) at |
| Expt | (mg) | Solvent | (μL) | (° C.) | Comments | as solid by | Observation | XRPD | 212 nm |
| VDS01 | 20.07 | Methanol (MeOH) | 500 | 25 | became wet | evap. at 30° C., | white solid | S5 | 89.99 |
| | | | | | solid overnight; | 21 mbar, overnight | | | |
| | | | | | after 12 days | | | | |
| | | | | | resulted as a | | | | |
| | | | | | fine suspension | | | | |
| VDS02 | 20.52 | Diisopropyl | 500 | | | diffusion at 25° C. | white solid | SM + pks., | |
| | | ether | | | | after 12 days | | P.O. | |
| VDS03 | 20.25 | Ethyl acetate | 500 | | | diffusion at 25° C. | white solid | SM, P.O. | |
| | | (EtOAc) | | | | after 12 days | | | |
| VDS04 | 20.42 | Acetone | 500 | | | diffusion at 25° C. | white solid | SM + pks., | |
| | | | | | | after 12 days | | P.O. | |
| VDS05 | 20.05 | Ethanol | 500 | 50 | | diffusion at 50° C. | wet white | S2 | 92.11 |
| | | (EtOH) | | | | after 12 days | solid | | |
| VDS06 | 20.43 | 2-Propanol | 500 | | | diffusion at 50° C. | wet white | SM, P.O. | |
| | | (2-PrOH) | | | | after 12 days | solid | | |
| VDS07 | 20.2 | 2-Butanol | 500 | | | diffusion at 50° C. | wet white | SM + pks., | |
| | | | | | | after 12 days | solid | P.O. | |
| VDS08 | 20.16 | Diisopropyl | 500 | | | diffusion at 50° C. | white solid | SM, P.O. | |
| | | ether | | | | after 12 days | | | |
| VDS09 | 20.43 | 2,2,2- | 500 | | after 12 days | diffusion at 50° C. | white solid | S4 | 90.89 |
| | | Trifluoroethanol | | | of diffusion | after 12 days; | | | |
| | | (TFE) | | | resulted as | redissolved under | | | |
| | | | | | slurry; | storage at RT | | | |
| | | | | | suspension was | | | | |
| | | | | | air-dried on | | | | |
| | | | | | filter paper | | | | |
| VDS10 | 20.17 | Ethyl acetate | 500 | | | diffusion at 50° C. | white solid | SM + pks., | |
| | | (EtOAc) | | | | after 12 days | | P.O. | |
| VDS11 | 20.43 | Acetonitrile | 500 | | | diffusion at 50° C. | white solid | SM, P.O. | |
| | | (MeCN) | | | | after 12 days | | | |
| VDS12 | 20.4 | Methyl ethyl | 500 | | | diffusion at 50° C. | white solid | SM + pks. | 93.65 |
| | | ketone (MEK) | | | | after 12 days | | (possible | |
| | | | | | | | | from S8), | |
| | | | | | | | | P.O. | |

SM = starting material (i.e., NaCDC Form B);
P.O. = preferred orientation;
pks. = peaks

Based on the XRPD data, the majority of the materials recovered from these experiments were in the same crystalline phase as the starting material (NaCDC Form B). Additionally, four different forms were identified—Form S2, Form S4, Form S5, and Form S8 (in mixture with SM).

### Characterization of Form S2

Form S2 was obtained from experiment VDS05 with EtOH after 12 days of diffusion at 50° C. (and remained stable under storage conditions at RT). The HPLC purity of the tested material was 92.1% at 212 nm. TG/DSC analysis of this material was consistent with that obtained from experiments SAS48, SDGR01, and SDGR07. XRPD analysis of this material is shown in FIG. 33, and the corresponding data summarized in Table S2:

TABLE S2

| 2-theta | d-Spacing | Rel. |
|---|---|---|
| Angle (°) | (Å) | Intensity (%) |
| 7.11 | 12.42 | 30.70 |
| 7.78 | 11.36 | 100.00 |
| 9.81 | 9.01 | 23.13 |
| 11.95 | 7.40 | 4.64 |
| 12.11 | 7.30 | 2.13 |
| 12.58 | 7.03 | 20.93 |
| 12.96 | 6.82 | 28.37 |
| 13.54 | 6.53 | 48.82 |
| 14.28 | 6.20 | 19.86 |
| 15.14 | 5.85 | 34.18 |
| 15.93 | 5.56 | 0.80 |
| 16.45 | 5.38 | 8.73 |
| 16.64 | 5.32 | 28.53 |
| 17.09 | 5.18 | 49.03 |
| 17.50 | 5.06 | 1.14 |
| 18.41 | 4.81 | 29.26 |
| 19.63 | 4.52 | 8.40 |
| 20.39 | 4.35 | 9.49 |
| 21.53 | 4.12 | 3.68 |
| 21.77 | 4.08 | 14.08 |
| 22.82 | 3.89 | 21.10 |
| 23.10 | 3.85 | 4.61 |
| 23.53 | 3.78 | 23.00 |
| 23.92 | 3.72 | 0.51 |
| 24.26 | 3.67 | 1.74 |
| 24.41 | 3.64 | 2.25 |
| 25.23 | 3.53 | 4.00 |
| 25.30 | 3.52 | 4.10 |
| 26.06 | 3.42 | 4.78 |
| 26.42 | 3.37 | 2.59 |
| 26.78 | 3.33 | 7.94 |
| 26.91 | 3.31 | 5.96 |
| 27.28 | 3.27 | 0.94 |
| 28.03 | 3.18 | 1.31 |
| 28.79 | 3.10 | 1.25 |
| 29.18 | 3.06 | 1.94 |
| 29.48 | 3.03 | 1.97 |
| 29.75 | 3.00 | 0.94 |
| 30.57 | 2.92 | 6.80 |
| 31.14 | 2.87 | 4.01 |
| 31.87 | 2.81 | 1.31 |
| 32.10 | 2.79 | 2.94 |
| 32.84 | 2.72 | 1.23 |
| 33.55 | 2.67 | 1.55 |
| 33.86 | 2.64 | 1.37 |
| 34.25 | 2.62 | 1.47 |
| 34.61 | 2.59 | 0.93 |
| 35.81 | 2.51 | 1.10 |
| 36.49 | 2.46 | 1.31 |
| 37.39 | 2.40 | 0.66 |
| 37.55 | 2.39 | 1.97 |
| 38.10 | 2.36 | 2.29 |
| 38.47 | 2.34 | 2.34 |
| 38.77 | 2.32 | 1.27 |
| 39.09 | 2.30 | 1.83 |
| 39.79 | 2.26 | 0.79 |
| 40.31 | 2.24 | 1.07 |
| 40.45 | 2.23 | 1.51 |
| 40.93 | 2.20 | 1.79 |
| 41.66 | 2.17 | 2.14 |
| 41.96 | 2.15 | 2.84 |
| 42.90 | 2.11 | 1.61 |
| 43.08 | 2.10 | 2.14 |
| 43.77 | 2.07 | 1.07 |
| 44.43 | 2.04 | 1.22 |
| 44.69 | 2.03 | 0.78 |

### Characterization of Form S4

Form S4 was obtained from experiment VDS09 with TFE after 12 days of diffusion at 50° C. (the formed suspension redissolved under storage conditions at RT). The material had a HPLC purity of 90.9% at 212 nm.

### Characterization of Form S5

Form S5 was obtained from experiment VDS01 with MeOH after vacuum drying of the fine suspension that resulted after 12 days of diffusion at RT. HPLC purity of the material was around 90% at 212 nm. Based on the TG/DSC analysis of VDS01 (FIG. 32B), Form S5 was assigned as a solvated form with a first endothermic event, suggesting desolvation, at around 101° C. and a mass loss of 4.426% (that can be attributed with 0.5 MeOH molecules that corresponds with a theoretical mass of 3.59%). The second event represents a recrystallization event with Tₒₙₛₑₜ at 190.9° C., and lastly, a melting event was observed at around 333° C. (Tₒₙₛₑₜ).

### Characterization of Form S8

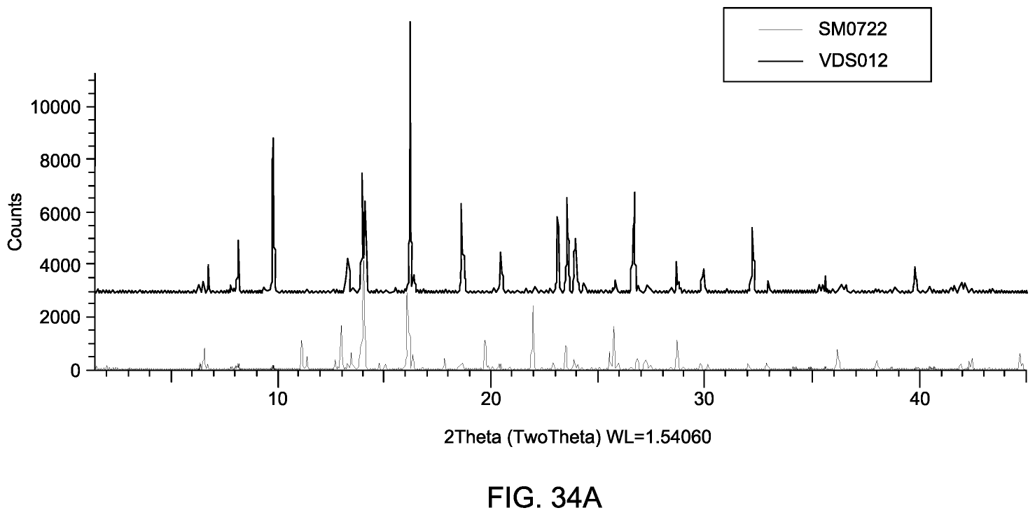

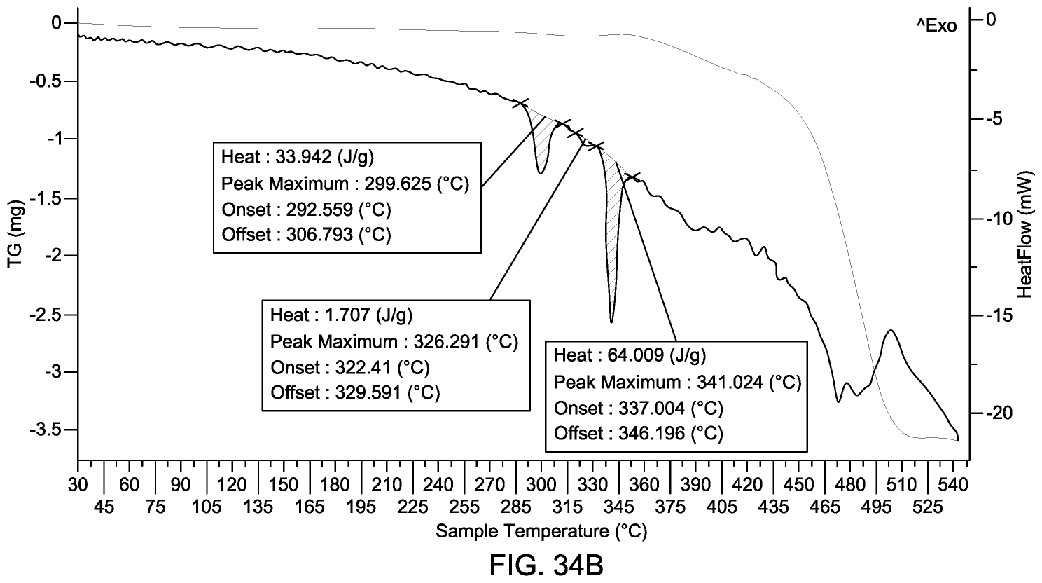

Form S8 was obtained in mixture with starting material (NaCDC Form B) from experiment VDS12 with MEK after 12 days of diffusion at 50° C. (and remained stable under storage condition at RT). The HPLC purity of the tested material was 93.7% at 212 nm. An XRPD spectrum of the material from experiment VDS12 is shown in FIG. 34A, as compared to NaCDC Form B, and the corresponding data are summarized in Table S8:

TABLE S8

| 2-theta | d-Spacing | Rel. |
|---|---|---|
| Angle (°) | (Å) | Intensity (%) |
| 6.34 | 13.93 | 2.04 |
| 6.53 | 13.53 | 2.84 |
| 6.77 | 13.05 | 6.85 |
| 7.84 | 11.26 | 2.45 |
| 8.18 | 10.80 | 15.35 |
| 9.39 | 9.41 | 1.40 |
| 9.78 | 9.03 | 51.42 |
| 11.37 | 7.78 | 0.43 |
| 12.65 | 6.99 | 0.45 |
| 12.76 | 6.93 | 0.82 |
| 13.19 | 6.71 | 7.04 |
| 13.29 | 6.66 | 11.26 |
| 13.54 | 6.54 | 1.19 |
| 13.97 | 6.34 | 40.71 |
| 14.14 | 6.26 | 15.90 |
| 14.63 | 6.05 | 0.83 |
| 14.83 | 5.97 | 0.40 |
| 15.16 | 5.84 | 0.36 |
| 15.53 | 5.70 | 1.31 |
| 16.05 | 5.52 | 3.00 |
| 16.20 | 5.47 | 100.00 |
| 16.40 | 5.40 | 5.79 |
| 16.86 | 5.26 | 0.61 |
| 17.87 | 4.96 | 0.49 |
| 18.65 | 4.75 | 8.68 |
| 19.84 | 4.47 | 0.32 |
| 20.12 | 4.41 | 1.40 |
| 20.43 | 4.34 | 14.74 |
| 21.62 | 4.11 | 1.43 |
| 22.02 | 4.03 | 1.70 |
| 22.77 | 3.90 | 1.30 |
| 23.17 | 3.84 | 14.22 |
| 23.56 | 3.77 | 36.40 |
| 23.98 | 3.71 | 15.31 |
| 24.37 | 3.65 | 3.51 |
| 25.16 | 3.54 | 0.48 |
| 25.80 | 3.45 | 4.24 |
| 26.54 | 3.36 | 9.31 |
| 26.62 | 3.35 | 23.98 |
| 26.70 | 3.34 | 33.23 |
| 26.91 | 3.31 | 1.53 |
| 27.34 | 3.26 | 1.71 |
| 28.29 | 3.15 | 0.28 |
| 28.46 | 3.13 | 1.19 |
| 28.69 | 3.11 | 11.97 |
| 28.73 | 3.10 | 2.87 |
| 29.98 | 2.98 | 6.44 |
| 31.04 | 2.88 | 1.07 |
| 32.24 | 2.77 | 23.64 |
| 32.92 | 2.72 | 3.78 |
| 33.85 | 2.65 | 0.35 |
| 35.31 | 2.54 | 2.61 |
| 35.45 | 2.53 | 2.70 |
| 35.56 | 2.52 | 5.12 |
| 35.92 | 2.50 | 0.96 |
| 36.29 | 2.47 | 2.13 |
| 36.37 | 2.47 | 2.03 |
| 36.54 | 2.46 | 2.29 |
| 36.91 | 2.43 | 0.49 |
| 37.96 | 2.37 | 1.12 |
| 38.21 | 2.35 | 0.33 |
| 38.63 | 2.33 | 0.70 |
| 38.85 | 2.32 | 1.74 |
| 38.98 | 2.31 | 1.01 |
| 39.77 | 2.26 | 10.24 |
| 40.41 | 2.23 | 1.19 |
| 40.51 | 2.23 | 1.40 |
| 40.66 | 2.22 | 0.66 |
| 41.07 | 2.20 | 0.87 |
| 41.51 | 2.17 | 0.75 |
| 41.60 | 2.17 | 1.91 |
| 41.89 | 2.15 | 2.30 |
| 42.00 | 2.15 | 2.64 |
| 42.13 | 2.14 | 2.37 |
| 42.44 | 2.13 | 1.04 |
| 43.00 | 2.10 | 0.31 |
| 43.39 | 2.08 | 0.90 |
| 43.86 | 2.06 | 0.53 |
| 44.79 | 2.02 | 0.28 |

Based on TG/DSC analysis of experiment VDS12, three endothermic events were observed (FIG. 34B). A first event is associated with a phase transformation or a melting process of Form S8 at Tₒₙₛₑₜ=292.6° C. A small second event was observed that could suggest a phase transformation or the presence of an impurity (or can be attributed with the heterogeneity of the material) with Tₒₙₛₑₜ=322.4° C. Lastly, a melting event at Tₒₙₛₑₜ=337.0° C. was observed. Considering that no mass loss was observed on the TG curve, Form S8 was assigned as an anhydrous form of NaCDC.

Anti-solvent Vapor Diffusion into Solutions

Three solutions were prepared with NaCDC Form B based on the solubility determined in the Solubility Studies of Example 8 at RT (Water: 203.41 mg/mL, MeOH: 100.37 mg/mL, and TFE: 50.66 mg/mL). The experiments were kept at RT for 1 h under stirring (500 rpm) until complete dissolution of the starting material, and then the solutions were filtered with 0.45 μm PTFE filters (with the exception of water stock solution, which was filtered with 0.2 μm Nylon filter in order to avoid the foaming phenomenon encountered during the EV experiments).

A set volume of the concentrated solutions was pipetted inside clear HPLC vials that were then inserted individually into 40 mL vessels containing initially 2 mL of anti-solvent. Each of the corresponding solvents was added as detailed in Table 12 below. The diffusion systems were let to stand in a closed cabinet at 25° C. or in an oven at 50° C. for 12 days. After the experimental time, many of the samples had yielded solids that were harvested and analyzed by XRPD. The rest of the solutions (ASDS0, 02, 05, 14, 17, 18 and 21) or suspension (ASDS6) were dried at 30° C., 21 mbar overnight. All recovered solids were analyzed by XRPD. Based on the obtained results, the experiments were analyzed by HPLC. Depending on the material availability, some were selected for TG/DSC. The results from anti-solvent vapor diffusion into solutions (ASDS) are summarized in Table 12.

TABLE 12

| | | | | Anti-solvent | | | | | | | HPLC |
|---|---|---|---|---|---|---|---|---|---|---|---|
| | | | Solution | | Initial | | | Solids | | | purity |
| | | Conc. | volume | Anti- | volume | T | | obtained | | | (%) at |
| Expt | Solvent | (mg/mL) | (μL) | solvent | (mL) | (° C.) | Comments | by | Observ. | XRPD | 212 nm |
| ASDS01 | Water | 203.41 | 180 | Ethanol | 2 | 25 | | evap. at | white | Am. + few broad | 97.56 |
| | (H₂O) | | | | | | | 30° C., | solid | pks: 3.9-6.4 & | |
| | | | | | | | | 21 mbar, | | 11.3-18.2 deg. | |
| | | | | | | | | overnight | | | |
| ASDS02 | | | 180 | THF | 2 | | | evap. at | white | Am. + few broad | 95.36 |
| | | | | | | | | 30° C., | solid | pks: 3.9-6.4 & | |
| | | | | | | | | 21 mbar, | | 11.3-18.2 deg. + | |
| | | | | | | | | overnight | | 1 pk. at 20 deg. | |
| ASDS03 | | | 100 | Acetone | 2 | | small pp. formed | diffusion at | white | similar S1 or | 89.05 |
| | | | | | | | after 5 days of | 25° C. after | solid | isomorphic | |
| | | | | | | | diffusion | 12 days | | form of S1 | |
| ASDS04 | | | 100 | Acetonitrile | 2 | | small pp. started | diffusion at | white | S9-a, P.O. | 92.43 |
| | | | | | | | to form over | 25° C. after | solid | | |
| | | | | | | | weekend | 12 days | | | |
| ASDS05 | | | 100 | Ethanol | 2 | 50 | | evap. at | white | Am. + few broad | 90.08 |
| | | | | | | | | 30° C., | solid | pks: 3.9-6.4 & | |
| | | | | | | | | 21 mbar, | | 11.3-18.2 deg. | |
| | | | | | | | | overnight | | | |
| ASDS06 | | | 100 | THF | 2 | | after 12 days | evap. at | white | Am. + few broad | 91.35 |
| | | | | | | | of diffusion | 30° C., | solid | pks: 3.9-6.4 & | |
| | | | | | | | resulted as | 21 mbar, | | 11.3-18.2 deg. | |
| | | | | | | | fine suspension | overnight | | | |
| ASDS07 | | | 100 | Acetone | 2 | | pp. started | diffusion at | white | similar S1 or | 91.16 |
| | | | | | | | to form over | 50° C. after | crystals | isomorphic form | |
| | | | | | | | weekend | 12 days | | of S1 + SM, P.O. | |
| ASDS08 | | | ~80 | Acetonitrile | 2 | | pp. started | diffusion at | white | S9-b, P.O. | 86.93 |
| | | | | | | | to form over | 50° C. after | solid | (similar to | |
| | | | | | | | weekend | 12 days | | NaCDC | |
| | | | | | | | | | | monohydrate) | |
| ASDS09 | MeOH | 100.37 | 450 | Acetone | 2 | 25 | pp. started | diffusion at | white | S5, P.O. | 93.34 |
| | | | | | | | to form over | 25° C. after | crystals | | |
| | | | | | | | weekend | 12 days | | | |
| ASDS10 | | | 450 | Ethyl | 2 | | pp. started | diffusion at | white | S5, P.O. | 94.32 |
| | | | | ether | | | to form over | 25° C. after | crystals | | |
| | | | | | | | weekend | 12 days | | | |
| ASDS11 | | | 450 | tert-Butyl | 2 | | pp. started | diffusion at | white | S5 + pks., P.O. | 96.19 |
| | | | | methyl ether | | | to form over | 25° C. after | crystals | | |
| | | | | | | | weekend | 12 days | | | |
| ASDS12 | | | 450 | Acetone | 2 | 50 | pp. started | diffusion at | white | l.c., few pks. | 94.31 |
| | | | | | | | to form over | 50° C. after | crystals | (possible from S5) | |
| | | | | | | | weekend | 12 days | | | |
| ASDS13 | | | 450 | tert-Butyl | 2 | | pp. started | diffusion at | white | S5 + pks., P.O. | 95.28 |
| | | | | methyl ether | | | to form over | 50° C. after | crystals | | |
| | | | | | | | weekend | 12 days | | | |
| ASDS14 | TFE | 50.66 | 370 | Ethyl | 2 | 25 | | evap. at | wet white | S10 | 91.44 |
| | | | | acetate | | | | 30° C., | solid | | |
| | | | | | | | | 21 mbar, | | | |
| | | | | | | | | overnight | | | |
| ASDS15 | | | 370 | tert-Butyl | 2 | | pp. started | diffusion at | white | Am. + few broad | 94.60 |
| | | | | methyl ether | | | to form over | 25° C. after | solid | pks: 3.9-5.8, | |
| | | | | | | | weekend | 12 days | | 10.7-12.2 & | |
| | | | | | | | | | | 13.8-17.5 deg. | |
| ASDS16 | | | 370 | Diisopropyl | 2 | | pp. started | diffusion at | white | S13, broad pks. | 92.44 |
| | | | | ether | | | to form over | 25° C. after | solid | (amorphization | |
| | | | | | | | weekend | 12 days | | tendency) | |
| ASDS17 | | | 370 | Acetone | 2 | | | evap. at | wet white | S14, l.c. | 89.30 |
| | | | | | | | | 30° C., | solid | | |
| | | | | | | | | 21 mbar, | | | |
| | | | | | | | | overnight | | | |
| ASDS18 | | | 370 | Ethyl | 2 | 50 | pp. formed | evap. at | white | S11 (similar to | 75.65 |
| | | | | acetate | | | after 6 days | 30° C., | crystals | or isomorph with | |
| | | | | | | | of diffusion | 21 mbar, | | S3), P.O. | |
| | | | | | | | | overnight | | | |
| ASDS19 | | | 370 | tert-Butyl | 2 | | pp. started | diffusion at | white | Am. + pks. | 97.07 |
| | | | | methyl ether | | | to form over | 50° C. after | solid | (possible S12) | |
| | | | | | | | weekend | 12 days | | | |
| ASDS20 | | | 370 | Diisopropyl | 2 | | pp. started | diffusion at | white | S4, P.O. | 95.25 |
| | | | | ether | | | to form over | 50° C. after | solid | | |
| | | | | | | | weekend | 12days | | | |
| ASDS21 | | | 370 | Acetone | 2 | | | evap. at | sticky | S14 | 69.68 |
| | | | | | | | | 30° C., | solid | | |
| | | | | | | | | 21 mbar, | | | |
| | | | | | | | | overnight | | | |

Am. = amorphous;
l.c. = low crystallinity;
SM = starting material (i.e., NaCDC Form B);
P.O. = preferred orientation;
pks. = peaks;
pp. = precipitate

Based on XRPD results, eight different forms were identified from these experiments—Form S1 (or isomorphic with Form S1), Form S5, Form S9-a, Form S9-b, Form S10, Form S11 (or isomorphic with Form S3), Form S12 (as mixture with amorphous phase), Form S13, and Form S14.

### Characterization of Forms S1 and S14

Form S1 (or a form similar to and/or isomorphic with Form S1) was obtained from experiments ASDS03 and ASDS07 with water (and acetone as anti-solvent) after 12 days of diffusion at 25° C. and 50° C., respectively. HPLC purity of these materials was determined to be ≥89.1% at 212 nm.

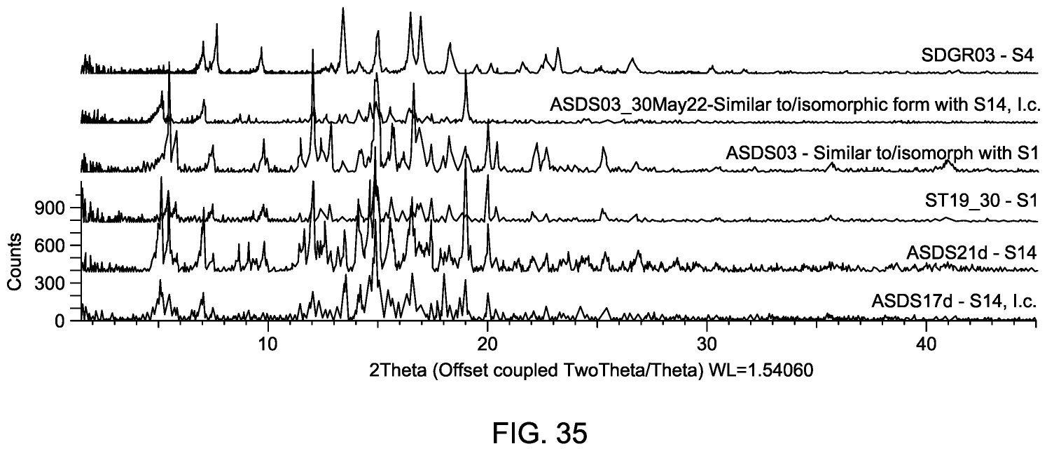

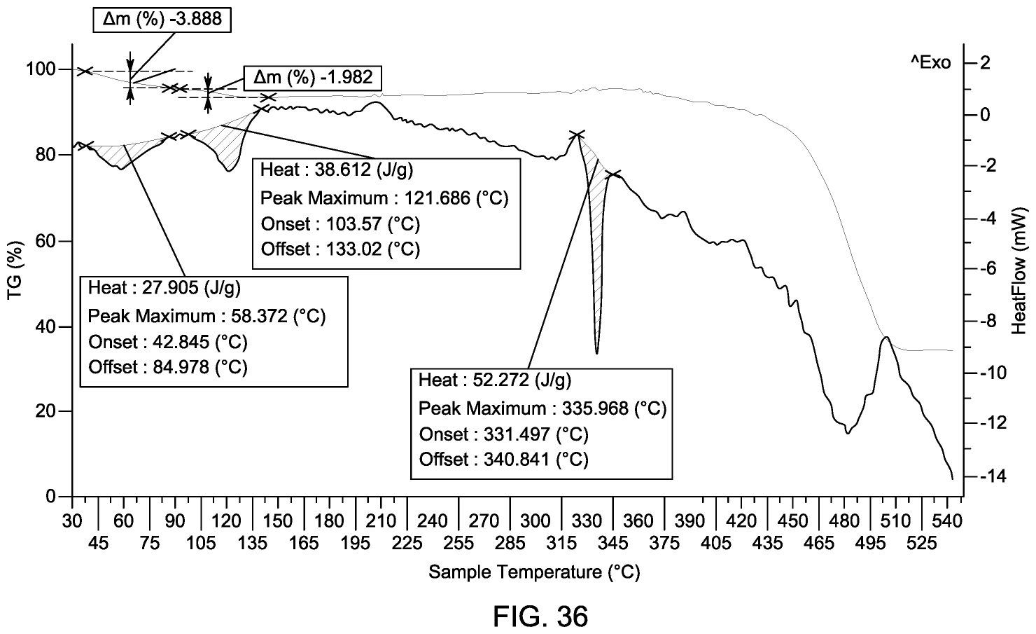

The precipitate that formed in the water solution in the presence of acetone (as anti-solvent) was stored at RT for up to 3 weeks before it was subjected to thermal analysis. In this case, the majority of ASDS03 solid was dried at RT on filter paper up to 2 h and after that the recovered material was analyzed by XRPD. The results indicated that the material had transformed into Form S14 (or a form a similar to and/or isomorphic with Form S14) (FIG. 35). TG/DSC analysis (FIG. 36) of this material resulted in an assignment of it as a mixed solvate with acetone and water. As such, the first endothermic event suggested a desolvation event with Tₒₙₛₑₜ=42.8° C. and a mass loss of 3.888% (that can be attributed with 0.25 acetone molecules that corresponds with a theoretical mass of 3.07%). The second endothermic event suggested another desolvation event with Tₒₙₛₑₜ=103.6° C. and a mass loss of 1.982% (that can be attributed with 0.5 molecules of water that corresponds with a theoretical mass of 2.08%). Also, a possible recrystallization event was observed on the DSC trace at around 190° C., and lastly, a melting event at around 332° C. (Tₒₙₛₑₜ) was observed. The melting Tₒₙₛₑₜ of this sample was different from the starting material (NaCDC Form B) meaning that Form S14 (or a form similar to and/or isomorphic with Form S14) is likely a solvate of an another anhydrous form.

### Characterization of Form S5

Form S5 was obtained from experiments ASDS09 and ASDS12 with MeOH (and acetone as anti-solvent) after 12 days of diffusion at 25° C. or 50° C., respectively, and from experiment ASDS10 with MeOH (and ethyl ether as anti-solvent) after 12 days of diffusion at 25° C. Also, Form S5 was obtained with extra peaks and preferred orientation from experiments ASDS11 and ASDS13 with MeOH (and MTBE as anti-solvent) at 25° C. or 50° C., respectively. HPLC purity of tested materials indicated high purity, >93.3% at 212 nm.

### Characterization of Forms S9-a and S9-b

Form S9-a (with preferred orientation) was obtained from experiment ASDS04 with water (and acetonitrile as anti-solvent) after 12 days of diffusion at 25° C. Form S9-b (with preferred orientation but similar to a NaCDC monohydrate) was obtained from experiment ASDS08 with the same system solvent/anti-solvent after 12 days of diffusion at 50° C. HPLC purity of both materials was good (>86.9%) at 212 nm.

The precipitates that formed in the water solution in the presence of acetonitrile (as anti-solvent) from experiments ASDS04 and ASDS08 were stored at RT up to 24 days before being subjected to thermal analysis. In this case, the majority of solids from experiments ASDS04 and ASDS08 were dried at RT on filter paper for 1-2 h, and after that the recovered materials were analyzed by XRPD. The results indicated that the material from experiment ASDS04 remained unchanged (but with low crystallinity) after storage, while the material from experiment ASDS08 had transformed into a form similar to Form S9-a (and with low crystallinity) (FIG. 37). The corresponding data for the initial material obtained from experiment ASDS04 (Table S9-a) and ASDS08 (Table S9-b) are summarized below:

TABLE S9-a

| 2-theta | d-Spacing | Rel. |
|---|---|---|
| Angle (°) | (Å) | Intensity (%) |
| 4.64 | 19.05 | 2.53 |
| 4.85 | 18.22 | 3.83 |
| 5.03 | 17.56 | 4.32 |
| 5.10 | 17.33 | 8.30 |
| 5.34 | 16.55 | 1.89 |
| 5.46 | 16.17 | 1.45 |
| 6.78 | 13.03 | 1.41 |
| 7.00 | 12.62 | 3.34 |
| 7.47 | 11.82 | 0.57 |
| 8.42 | 10.49 | 0.58 |
| 8.66 | 10.20 | 0.99 |
| 9.12 | 9.69 | 2.99 |
| 9.39 | 9.42 | 2.49 |
| 9.51 | 9.30 | 0.76 |
| 10.99 | 8.04 | 3.44 |
| 11.50 | 7.69 | 1.52 |
| 11.65 | 7.59 | 5.06 |
| 12.01 | 7.36 | 4.27 |
| 12.34 | 7.17 | 6.77 |
| 12.66 | 6.99 | 1.33 |
| 12.80 | 6.91 | 2.02 |
| 13.38 | 6.61 | 7.06 |
| 13.52 | 6.54 | 60.20 |
| 14.23 | 6.22 | 19.11 |
| 14.74 | 6.01 | 8.69 |
| 14.87 | 5.95 | 8.85 |
| 15.46 | 5.73 | 10.96 |
| 15.69 | 5.64 | 1.01 |
| 16.07 | 5.51 | 1.50 |
| 16.34 | 5.42 | 3.64 |
| 16.44 | 5.39 | 6.05 |
| 16.58 | 5.34 | 10.33 |
| 16.69 | 5.31 | 10.05 |
| 17.14 | 5.17 | 1.70 |
| 17.48 | 5.07 | 1.36 |
| 17.70 | 5.01 | 8.45 |
| 18.06 | 4.91 | 2.78 |
| 18.32 | 4.84 | 6.50 |
| 18.78 | 4.72 | 100.00 |
| 19.13 | 4.64 | 1.98 |
| 19.66 | 4.51 | 0.47 |
| 20.07 | 4.42 | 1.74 |
| 21.18 | 4.19 | 1.84 |
| 21.69 | 4.09 | 5.34 |
| 22.08 | 4.02 | 1.02 |
| 22.35 | 3.97 | 1.55 |
| 23.09 | 3.85 | 1.51 |
| 23.22 | 3.83 | 1.94 |
| 23.46 | 3.79 | 0.67 |
| 23.79 | 3.74 | 2.51 |
| 24.25 | 3.67 | 5.54 |
| 24.89 | 3.57 | 1.66 |
| 25.08 | 3.55 | 9.74 |
| 25.43 | 3.50 | 7.44 |
| 25.79 | 3.45 | 7.62 |
| 26.23 | 3.40 | 5.05 |
| 26.52 | 3.36 | 1.29 |
| 26.71 | 3.34 | 1.76 |
| 27.22 | 3.27 | 3.00 |
| 27.31 | 3.26 | 4.99 |
| 27.46 | 3.25 | 12.07 |
| 27.66 | 3.22 | 1.56 |
| 28.03 | 3.18 | 0.95 |
| 28.74 | 3.10 | 2.60 |
| 29.05 | 3.07 | 1.51 |
| 29.35 | 3.04 | 2.25 |
| 29.78 | 3.00 | 4.07 |
| 30.04 | 2.97 | 1.86 |
| 30.42 | 2.94 | 1.55 |
| 30.76 | 2.90 | 3.11 |
| 31.31 | 2.85 | 5.51 |
| 31.66 | 2.82 | 1.61 |
| 32.12 | 2.78 | 2.36 |
| 32.22 | 2.78 | 2.21 |
| 32.43 | 2.76 | 1.17 |
| 32.63 | 2.74 | 0.51 |
| 33.15 | 2.70 | 0.74 |
| 33.50 | 2.67 | 3.19 |
| 33.66 | 2.66 | 1.38 |
| 33.85 | 2.65 | 2.82 |
| 34.04 | 2.63 | 1.40 |
| 34.68 | 2.58 | 0.88 |
| 35.13 | 2.55 | 0.55 |
| 35.50 | 2.53 | 4.83 |
| 36.46 | 2.46 | 1.43 |
| 36.71 | 2.45 | 2.39 |
| 37.17 | 2.42 | 0.87 |
| 37.78 | 2.38 | 2.17 |
| 38.07 | 2.36 | 0.67 |
| 38.23 | 2.35 | 4.20 |
| 38.76 | 2.32 | 3.84 |
| 38.95 | 2.31 | 2.06 |
| 39.38 | 2.29 | 3.45 |
| 40.03 | 2.25 | 0.85 |
| 41.19 | 2.19 | 0.89 |
| 41.49 | 2.17 | 0.45 |
| 42.28 | 2.14 | 3.27 |
| 43.16 | 2.09 | 0.46 |
| 43.44 | 2.08 | 1.35 |
| 43.54 | 2.08 | 1.17 |
| 43.88 | 2.06 | 1.91 |
| 44.67 | 2.03 | 1.87 |

TABLE S9-b

| 2-theta | d-Spacing | Rel. |
|---|---|---|
| Angle (°) | (Å) | Intensity (%) |
| 5.28 | 16.72 | 3.03 |
| 5.47 | 16.13 | 18.88 |
| 5.87 | 15.04 | 2.93 |
| 7.23 | 12.21 | 2.28 |
| 7.48 | 11.81 | 8.17 |
| 8.47 | 10.43 | 2.48 |
| 9.42 | 9.38 | 3.22 |
| 9.82 | 9.00 | 100.00 |
| 10.21 | 8.65 | 0.87 |
| 11.52 | 7.68 | 0.96 |
| 12.06 | 7.33 | 1.64 |
| 12.41 | 7.13 | 1.93 |
| 12.66 | 6.98 | 28.10 |
| 12.83 | 6.89 | 5.02 |
| 14.59 | 6.06 | 2.04 |
| 14.95 | 5.92 | 4.47 |
| 15.07 | 5.87 | 9.25 |
| 15.52 | 5.70 | 2.21 |
| 16.65 | 5.32 | 1.16 |
| 16.99 | 5.21 | 2.18 |
| 17.24 | 5.14 | 0.95 |
| 18.33 | 4.84 | 1.46 |
| 18.71 | 4.74 | 2.39 |
| 18.81 | 4.71 | 1.87 |
| 19.70 | 4.50 | 0.86 |
| 20.06 | 4.42 | 0.88 |
| 20.55 | 4.32 | 4.07 |
| 21.11 | 4.21 | 0.75 |
| 22.12 | 4.02 | 1.18 |
| 22.64 | 3.92 | 1.06 |
| 22.77 | 3.90 | 1.57 |
| 24.16 | 3.68 | 3.77 |
| 25.04 | 3.55 | 1.89 |
| 25.21 | 3.53 | 1.53 |
| 25.39 | 3.50 | 2.47 |
| 25.95 | 3.43 | 1.11 |
| 26.25 | 3.39 | 0.64 |
| 26.43 | 3.37 | 1.24 |
| 26.77 | 3.33 | 0.71 |
| 27.02 | 3.30 | 4.69 |
| 27.11 | 3.29 | 4.16 |
| 27.41 | 3.25 | 1.80 |
| 27.63 | 3.23 | 0.62 |
| 27.64 | 3.23 | 0.61 |
| 29.04 | 3.07 | 0.77 |
| 29.39 | 3.04 | 2.32 |
| 30.10 | 2.97 | 4.33 |
| 30.47 | 2.93 | 1.69 |
| 30.63 | 2.92 | 2.29 |
| 30.94 | 2.89 | 0.88 |
| 31.61 | 2.83 | 1.06 |
| 32.14 | 2.78 | 3.83 |
| 33.15 | 2.70 | 13.19 |
| 33.55 | 2.67 | 1.98 |
| 34.38 | 2.61 | 0.39 |
| 35.55 | 2.52 | 3.53 |
| 37.05 | 2.42 | 1.79 |
| 37.30 | 2.41 | 3.37 |
| 37.92 | 2.37 | 0.60 |
| 38.40 | 2.34 | 0.82 |
| 39.22 | 2.30 | 1.52 |
| 39.51 | 2.28 | 32.04 |
| 40.13 | 2.25 | 1.30 |
| 40.43 | 2.23 | 3.05 |
| 41.04 | 2.20 | 1.05 |
| 41.23 | 2.19 | 1.32 |
| 41.89 | 2.15 | 1.06 |
| 43.57 | 2.08 | 0.89 |
| 44.08 | 2.05 | 0.68 |

Based on the TG/DSC analysis, the sample from experiment ASDS04 that had been stored for 22 days (FIG. 38) was assigned as a solvate with water or MeCN or a mixed solvate. As such, the first endothermic event suggested a desolvation event with Tₒₙₛₑₜ=99.9° C. and a mass loss of 8.635% a (that could be attributed with 2 molecules of water, which corresponds with a theoretical mass of 8.32%, or with 1 molecule of MeCN, which corresponds with a theoretical mass of 9.01%). Lastly, a melting event was observed with Tₒₙₛₑₜ at around 328° C.

Based on the TG/DSC analysis, the sample from experiment ASDS08 that had been stored for 23 days (FIG. 39) was assigned as a solvate with water or MeCN or a mixed solvate. As such, the first endothermic event suggested a desolvation event with Tₒₙₛₑₜ=94.8° C. and a mass loss of 2.298% (that can be attributed to a 0.5 molecules of water, which corresponds with a theoretical mass of 2.08%, or with 0.25 molecules of MeCN, which corresponds with a theoretical mass of 2.25%). Lastly, a melting event was observed with Tₒₙₛₑₜ at around 336° C.

### Characterization of Forms S10 and S11

Form S10 was obtained from experiment ASDS14 with TFE (and EtOAc as anti-solvent) after 12 days of diffusion at 25° C. Form S11 (or a form similar to and/or isomorphic with Form S3) was obtained from experiment ASDS18 with the same system solvent/anti-solvent after 12 days of diffusion at 50° C. Both samples were evaporated at 30° C., 21 mbar overnight. HPLC purity of the material from experiment ASDS14 was 91.4%, while for the material from experiment ASDS18, the purity was decreased to 75.6% (at 212 nm).

An XRPD spectrum of the material from experiment ASDS14 is shown in FIG. 40A, and the corresponding data are summarized in Table S10:

TABLE S10

| 2-theta | d-Spacing | Rel. |
|---|---|---|
| Angle (°) | (Å) | Intensity (%) |
| 5.13 | 17.22 | 91.64 |
| 5.50 | 16.05 | 15.05 |
| 5.84 | 15.11 | 3.08 |
| 7.01 | 12.60 | 65.02 |
| 8.69 | 10.16 | 24.72 |
| 9.11 | 9.70 | 22.44 |
| 9.34 | 9.46 | 9.48 |
| 10.28 | 8.59 | 3.93 |
| 11.04 | 8.01 | 8.07 |
| 11.70 | 7.56 | 6.86 |
| 12.03 | 7.35 | 32.13 |
| 12.36 | 7.15 | 23.55 |
| 12.66 | 6.98 | 7.59 |
| 12.84 | 6.89 | 13.29 |
| 13.55 | 6.53 | 49.72 |
| 14.20 | 6.23 | 25.42 |
| 14.25 | 6.21 | 30.03 |
| 14.91 | 5.94 | 100.00 |
| 15.53 | 5.70 | 88.28 |
| 16.03 | 5.53 | 5.14 |
| 16.37 | 5.41 | 34.10 |
| 16.62 | 5.33 | 62.70 |
| 17.16 | 5.16 | 13.17 |
| 17.47 | 5.07 | 11.51 |
| 17.72 | 5.00 | 14.18 |
| 18.07 | 4.91 | 17.76 |
| 18.38 | 4.82 | 12.56 |
| 18.78 | 4.72 | 84.13 |
| 19.07 | 4.65 | 38.06 |
| 19.22 | 4.61 | 3.09 |
| 20.09 | 4.42 | 7.92 |
| 20.78 | 4.27 | 4.34 |
| 21.27 | 4.17 | 9.75 |
| 21.75 | 4.08 | 10.82 |
| 22.37 | 3.97 | 8.54 |
| 23.47 | 3.79 | 5.28 |
| 23.93 | 3.72 | 4.38 |
| 24.26 | 3.67 | 12.80 |
| 25.16 | 3.54 | 2.58 |
| 25.54 | 3.49 | 10.08 |
| 25.88 | 3.44 | 6.33 |
| 26.28 | 3.39 | 7.09 |
| 26.55 | 3.35 | 8.18 |
| 26.94 | 3.31 | 4.21 |
| 27.38 | 3.26 | 5.70 |
| 28.08 | 3.18 | 2.48 |
| 28.75 | 3.10 | 6.24 |
| 29.06 | 3.07 | 7.89 |
| 29.40 | 3.04 | 5.59 |
| 30.09 | 2.97 | 3.59 |
| 30.45 | 2.93 | 2.89 |
| 32.02 | 2.79 | 3.54 |
| 32.41 | 2.76 | 2.44 |
| 34.75 | 2.58 | 2.93 |
| 35.57 | 2.52 | 5.45 |
| 36.50 | 2.46 | 4.41 |
| 38.12 | 2.36 | 6.60 |
| 38.69 | 2.33 | 3.02 |
| 39.46 | 2.28 | 2.37 |
| 41.25 | 2.19 | 3.39 |

An XRPD spectrum of the material from experiment ASDS18 is shown in FIG. 50A, and the corresponding data are summarized in Table S11:

TABLE S11

| 2-theta | d-Spacing | Rel. |
|---|---|---|
| Angle (°) | (Å) | Intensity (%) |
| 3.61 | 24.42 | 2.40 |
| 4.73 | 18.65 | 5.46 |
| 4.99 | 17.68 | 95.41 |
| 6.44 | 13.71 | 1.30 |
| 10.18 | 8.68 | 2.92 |
| 10.41 | 8.49 | 27.01 |
| 10.60 | 8.34 | 56.86 |
| 11.46 | 7.72 | 7.17 |
| 11.77 | 7.51 | 5.72 |
| 12.02 | 7.36 | 4.47 |
| 12.42 | 7.12 | 1.90 |
| 14.25 | 6.21 | 1.26 |
| 14.71 | 6.02 | 1.26 |
| 15.09 | 5.87 | 16.45 |
| 15.28 | 5.79 | 4.83 |
| 16.81 | 5.27 | 2.99 |
| 16.87 | 5.25 | 5.50 |
| 17.84 | 4.97 | 2.66 |
| 19.00 | 4.67 | 1.83 |
| 19.27 | 4.60 | 1.29 |
| 19.84 | 4.47 | 42.02 |
| 20.41 | 4.35 | 2.04 |
| 22.36 | 3.97 | 3.22 |
| 22.46 | 3.95 | 3.91 |
| 23.30 | 3.81 | 6.15 |
| 23.44 | 3.79 | 100.00 |
| 25.70 | 3.46 | 2.08 |
| 26.59 | 3.35 | 0.73 |
| 30.13 | 2.96 | 3.03 |
| 31.97 | 2.80 | 1.11 |
| 37.05 | 2.42 | 6.38 |
| 37.19 | 2.42 | 1.63 |
| 37.59 | 2.39 | 1.17 |
| 39.78 | 2.26 | 8.51 |
| 40.01 | 2.25 | 4.25 |
| 40.28 | 2.24 | 7.85 |
| 42.91 | 2.11 | 1.26 |

Based on the TG/DSC analysis (FIG. 40B), the material obtained from experiment ASDS14 was assigned as a solvate with TFE or EtOAc or mixed solvate. As such, the first endothermic event suggested a desolvation event with Tₒₙₛₑₜ=141.3° C. and a mass loss of 16% (that can be attributed to 1 molecule of TFE, which corresponds with a theoretical mass of 19.44%, or with 1 molecule of EtOAc, which corresponds with a theoretical mass of 17.53%). A first melting step was observed with Tₒₙₛₑₜ at around 324° C., and a possible recrystallization and the final melting step were observed with Tₒₙₛₑₜ at around 336° C.

Based on the TG/DSC analysis (FIG. 41), the material obtained from experiment ASDS18 was assigned as an anhydrous form with some residual solvent or physical adsorption of solvent. As such, a mass loss of 3.799% was observed on the TG curve, but not attributed with a well-defined event. A melting event with Tₒₙₛₑₜ around 328° C. was observed.

### Characterization of Form S12

Form S12 (as mixture with amorphous phase) was obtained from experiment ASDS19 with TFE (and MTBE as anti-solvent) after 12 days of diffusion at 50° C. HPLC purity of the sample was determined to be 97.1% at 212 nm.

The precipitate that formed into the TFE solution in the presence of MTBE (as anti-solvent) was stored at RT for 22 days before being subjected to thermal analysis. In this case, the majority of the material from experiment ASDS19 was dried at RT on filter paper up to 2 h, and after that the recovered material was analyzed by XRPD. The results indicated that the material that had been stored for 22 days transformed into Form S15 (also identified in experiment RAS36) (FIG. 42). Considering that Form S12 was obtained as mixture with amorphous phase and with low crystallinity, there is a possibility that Form S12 is the same as Form S15. The XRPD data from experiment ASDS19 before storage for 22 days is summarized in Table S12, and the XRPD data from experiment ASDS19 after storage for 22 days is summarized in Table S15.

TABLE S12

| 2-theta | d-Spacing | Rel. |
|---|---|---|
| Angle (°) | (Å) | Intensity (%) |
| 4.68 | 18.89 | 100.00 |
| 4.84 | 18.25 | 78.35 |
| 9.40 | 9.40 | 19.71 |
| 11.27 | 7.85 | 29.69 |
| 14.64 | 6.05 | 42.28 |
| 14.95 | 5.92 | 54.47 |
| 15.62 | 5.67 | 31.96 |
| 16.06 | 5.52 | 31.07 |
| 18.36 | 4.83 | 18.79 |
| 18.93 | 4.68 | 37.10 |
| 19.60 | 4.52 | 14.72 |

TABLE S15

| 2-theta | d-Spacing | Rel. |
|---|---|---|
| Angle (°) | (Å) | Intensity (%) |
| 4.82 | 18.32 | 58.95 |
| 5.22 | 16.92 | 37.13 |
| 5.89 | 14.99 | 40.21 |
| 7.27 | 12.15 | 16.38 |
| 8.67 | 10.19 | 7.77 |
| 9.76 | 9.05 | 16.78 |
| 10.81 | 8.17 | 30.51 |
| 11.80 | 7.50 | 46.80 |
| 12.07 | 7.33 | 30.72 |
| 12.61 | 7.01 | 11.50 |
| 13.00 | 6.81 | 72.36 |
| 13.41 | 6.60 | 27.64 |
| 14.43 | 6.13 | 57.79 |
| 15.00 | 5.90 | 100.00 |
| 15.37 | 5.76 | 25.39 |
| 15.74 | 5.63 | 54.42 |
| 15.90 | 5.57 | 29.41 |
| 16.65 | 5.32 | 75.88 |
| 17.79 | 4.98 | 28.01 |
| 18.36 | 4.83 | 21.60 |
| 18.94 | 4.68 | 56.32 |
| 19.26 | 4.60 | 49.26 |
| 19.60 | 4.52 | 9.96 |
| 20.14 | 4.41 | 13.64 |
| 21.00 | 4.23 | 29.94 |
| 21.37 | 4.15 | 7.42 |
| 22.71 | 3.91 | 6.92 |
| 23.90 | 3.72 | 13.87 |
| 24.83 | 3.58 | 13.43 |
| 25.16 | 3.54 | 22.79 |
| 26.46 | 3.37 | 11.14 |
| 27.49 | 3.24 | 9.02 |
| 27.95 | 3.19 | 12.48 |
| 28.67 | 3.11 | 5.58 |
| 30.99 | 2.88 | 5.86 |
| 38.51 | 2.34 | 8.12 |
| 40.55 | 2.22 | 9.30 |

Based on the TG/DSC analysis (FIG. 43), the material from experiment ASDS19 that was stored for 22 days was assigned as a solvate with MTBE. As such, the first endothermic event suggests a desolvation with Tₒₙₛₑₜ=80.8° C. and a mass loss of 4.448% (that can be attributed with 0.25 molecules of MTBE, which corresponds with a theoretical mass of 4.38%). An inflexion point at around 180° C. was observed and might suggest a recrystallization. Lastly, a melting event with Tₒₙₛₑₜ at around 332° C. was observed.

### Characterization of Form S13

Form S13 (which displayed with broad peaks and a tendency for amorphization) was obtained from experiment ASDS16 with TFE (and diisopropyl ether as anti-solvent) after 12 days of diffusion at 25° C. HPLC purity of the sample was 92.4% at 212 nm.

The precipitate that formed into the TFE solution in the presence of DIPE (as anti-solvent) was stored at RT for 22 days before being subjected to thermal analysis. In this case, the majority of material obtained from experiment ASDS16 was dried at RT on filter paper up to 2 h, and after that, the recovered material was analyzed by XRPD. The results indicated that the material that had been stored for 22 days remained unchanged, resulting in a low crystalline phase with some extra peaks or preferred orientation (FIG. 44).

Based on the TG/DSC analysis (FIG. 45), Form S13 was assigned as a mixed solvate with DIPE and TFE. As such, the first, endothermic event suggested a desolvation with Tₒₙₛₑₜ=55.4° C. and a mass loss of 2.358% (that can be attributed to 0.125 molecules of DIPE, which corresponds with a theoretical mass of 2.47%). The second, endothermic event suggested another desolvation with Tₒₙₛₑₜ=148.8° C. and a mass loss of 3.804% (that can be attributed to 0.25 molecules of TFE, which corresponds with a theoretical mass of 4.86%). The third, exothermic event suggests a recrystallization event with Tₒₙₛₑₜ=191.8° C. Lastly, a melting event was observed with Tₒₙₛₑₜ at around 335° C.

### Characterization of Form S14

Form S14 was obtained from two experiments—ASDS17 with TFE (and acetone as anti-solvent) after 12 days of diffusion at 25° C. and from ASDS21 with the same system solvent/anti-solvent after 12 days of diffusion at 50° C. Both samples were evaporated at 30° C., 21 mbar overnight. HPLC purity of the material from experiment ASDS17 was 89.3%, while HPLC purity of the material from experiment ASDS21 was around 70% (212 nm). XRPD analysis of the material obtained from experiment ASDS21 is shown in FIG. 46A, and the corresponding data are summarized in Table S14:

TABLE S14

| 2-theta | d-Spacing | Rel. |
|---|---|---|
| Angle (°) | (Å) | Intensity (%) |
| 4.84 | 18.25 | 10.94 |
| 5.02 | 17.59 | 40.93 |
| 5.15 | 17.16 | 68.95 |
| 5.50 | 16.05 | 41.94 |
| 5.81 | 15.21 | 10.95 |
| 6.86 | 12.88 | 14.35 |
| 7.05 | 12.53 | 37.59 |
| 7.48 | 11.82 | 12.71 |
| 8.69 | 10.17 | 20.46 |
| 9.13 | 9.68 | 21.71 |
| 9.35 | 9.45 | 6.41 |
| 9.81 | 9.01 | 17.50 |
| 9.95 | 8.88 | 11.27 |
| 11.48 | 7.70 | 16.96 |
| 11.64 | 7.60 | 32.12 |
| 12.04 | 7.34 | 63.07 |
| 12.28 | 7.20 | 25.38 |
| 12.62 | 7.01 | 40.90 |
| 12.85 | 6.88 | 18.12 |
| 13.20 | 6.70 | 19.15 |
| 13.49 | 6.56 | 32.10 |
| 14.13 | 6.26 | 54.20 |
| 14.17 | 6.25 | 21.38 |
| 14.64 | 6.05 | 74.69 |
| 14.90 | 5.94 | 100.00 |
| 15.03 | 5.89 | 56.51 |
| 15.44 | 5.74 | 16.17 |
| 15.60 | 5.68 | 39.55 |
| 16.20 | 5.47 | 9.53 |
| 16.42 | 5.39 | 28.45 |
| 16.56 | 5.35 | 53.05 |
| 16.88 | 5.25 | 21.88 |
| 17.24 | 5.14 | 20.44 |
| 17.46 | 5.08 | 38.82 |
| 17.90 | 4.95 | 17.48 |
| 18.27 | 4.85 | 21.11 |
| 19.02 | 4.66 | 90.26 |
| 19.21 | 4.62 | 22.02 |
| 20.04 | 4.43 | 34.29 |
| 20.40 | 4.35 | 11.13 |
| 20.71 | 4.28 | 8.94 |
| 21.43 | 4.14 | 10.61 |
| 22.09 | 4.02 | 11.74 |
| 22.69 | 3.92 | 12.67 |
| 23.69 | 3.75 | 12.35 |
| 24.29 | 3.66 | 9.80 |
| 24.55 | 3.62 | 9.48 |
| 25.37 | 3.51 | 14.83 |
| 26.19 | 3.40 | 5.99 |
| 26.51 | 3.36 | 7.79 |
| 26.89 | 3.31 | 14.08 |
| 27.95 | 3.19 | 5.65 |
| 28.66 | 3.11 | 12.57 |
| 29.14 | 3.06 | 5.98 |
| 30.44 | 2.93 | 5.46 |
| 32.03 | 2.79 | 6.10 |
| 32.52 | 2.75 | 6.30 |
| 34.46 | 2.60 | 3.14 |
| 39.46 | 2.28 | 4.07 |
| 40.00 | 2.25 | 7.85 |

Based on TG/DSC analysis of the material from experiment ASDS17 (FIG. 46B), Form S14 was assigned as a solvated form with a first endothermic event suggesting a desolvation at 143.7° C. and a mass loss of 15.84% (that can be attributed with 1 TFE molecule, which corresponds with a theoretical mass of 19.44%). The second event represents a melting event at 331° C. (Tₒₙₛₑₜ). The mass loss and Tₒₙₛₑₜ of the desolvation event are similar with those of Form S4, but the melting Tₒₙₛₑₜ is different, meaning that Form S14 may be a mono-solvate with TFE but of an anhydrous form other than NaCDC Form B.

### Crash Cooling

Ten stock solutions (with water; H₂O:THF 1:1 v/v; H₂O:EtOH 1:1 v/v; H₂O:2-PrOH 1:1 v/v; H₂O:MeCN 1:1 v/v; H₂O:acetone 1:1 v/v; methanol; 2,2,2-trifluoroethanol; MeOH:EtOAc 1:1 v/v; and MeOH:acetone 1:1 v/v) were prepared at 1.5 times solubility (determined from the Solubility Studies of Example 8) at RT under stirring at 700 rpm and heated at 40° C. for 1 hour. Mixtures that did not dissolve at 40° C. were heated at 50° C. with extra stirring time or extra solvent volume. The exact concentration for each stock solution is indicated in Table 13. After the aforementioned time, the solutions were completely dissolved and the hot solutions were filtered with 0.45 m PTFE filters (or 0.2 m Nylon filter for the case of water stock solution) and kept at the working temperature.

After filtration, determined volumes of each solution (to have, in the end, 196.91-65.72 mg of NaCDC per experiment) were added in appropriate vials that were further placed at 25° C. (experiments CL01-10), at 5° C. (experiments CL11-19) or at −20° C. (experiments CL20-23), aged for 7 days, and checked periodically. After the time elapsed, no precipitation occurred, and the solutions were vacuum evaporated at 30° C., low pressure (final pressure=20-21 mbar) for ˜18 h (experiments CL01, 02, 05, 06, 10, 11, 13, 14, 15, 16, 19, 20, 22 and 23) or for ˜41 h (experiments CL03, 04, 08, 09, 12, 17 and 18) or for 4.5 days (experiments CL07 and 21) and analyzed by XRPD. Based on the results obtained, some experiments were also analyzed by HPLC or TG/DSC. The results from crash cooling crystallizations (CL) are summarized in Table 14.

TABLE 13

| | | | | Dissolution at 40° C. | | | Dissolution at 50° C. after | | | |
|---|---|---|---|---|---|---|---|---|---|---|
| | | | | after 1 h with 700 rpm | | | 1 h*/2 h with 700 rpm | | | |
| | | | | | | Final | | | | Final |
| | | Vol. | Initial | | | conc. at | | | Extra | conc. at |
| | | stock | conc. | | | 40° C. | | | vol. | 50° C. |
| No. | Solvent | (mL) | (mg/mL) | Y/N | Comments | (mg/mL) | Y/N | Comments | (mL) | (mg/mL) |
| sol. 1 | Water (H₂O) | 2.86 | 305.70 | No | not | — | Yes | * after 1 h with | — | 305.70 |
| | | | | | completely | | | 700 rpm, mixture | | |
| | | | | | dissolved | | | was completely | | |
| | | | | | | | | dissolved | | |
| sol. 2 | H₂O:THF | 0.38 | 302.74 | Yes | | 302.74 | — | — | — | — |
| | (1:1 v/v) | | | | | | | | | |
| sol. 3 | H₂O:EtOH | 0.38 | 303.34 | Yes | | 303.34 | — | — | — | — |
| | (1:1 v/v) | | | | | | | | | |
| sol. 4 | H₂O:2-PrOH | 0.39 | 298.73 | Yes | | 298.73 | — | — | — | — |
| | (1:1 v/v) | | | | | | | | | |
| sol. 5 | H₂O:MeCN | 0.38 | 306.96 | Yes | | 306.96 | — | — | — | — |
| | (1:1 v/v) | | | | | | | | | |
| sol. 6 | H₂O:Acetone | 0.38 | 302.68 | Yes | | 302.68 | — | — | — | — |
| | (1:1 v/v) | | | | | | | | | |
| sol. 7 | Methanol | 4.00 | 150.95 | No | almost | — | Yes | after 1 h with | 1 | 120.76 |
| | (MeOH) | | | | dissolved | | | 700 rpm, the | | |
| | | | | | | | | mixture was almost | | |
| | | | | | | | | dissolved; extra | | |
| | | | | | | | | 1 mL of MeOH was | | |
| | | | | | | | | added and left | | |
| | | | | | | | | under stirring | | |
| | | | | | | | | for another 1 h | | |
| sol. 8 | 2,2,2- | 9.10 | 75.85 | No | almost | — | Yes | after 1 h with | 2 | 62.18 |
| | Trifluoroethanol | | | | dissolved | | | 700 rpm, the | | |
| | (TFE) | | | | | | | mixture was almost | | |
| | | | | | | | | dissolved; extra | | |
| | | | | | | | | 2 mL of TFE was | | |
| | | | | | | | | added and left | | |
| | | | | | | | | under stirring | | |
| | | | | | | | | for another 1 h | | |
| sol. 9 | MeOH:EtOAc | 2.28 | 30.33 | No | not | — | Yes | after 1 h with | 1 | 21.07 |
| | (1:1 v/v) | | | | dissolved | | | 700 rpm, the | | |
| | | | | | | | | mixture was almost | | |
| | | | | | | | | dissolved; extra 2 | | |
| | | | | | | | | mL of MeOH:EtOAc | | |
| | | | | | | | | was added and left | | |
| | | | | | | | | under stirring for | | |
| | | | | | | | | another 1 h | | |
| sol. 10 | MeOH:Acetone | 2.28 | 30.43 | No | not | — | Yes | after 1 h with | 1 | 21.14 |
| | (1:1 v/v) | | | | dissolved | | | 700 rpm, the | | |
| | | | | | | | | mixture was almost | | |
| | | | | | | | | dissolved; extra 2 | | |
| | | | | | | | | mL of MeOH:Acetone | | |
| | | | | | | | | was added and left | | |
| | | | | | | | | under stirring for | | |
| | | | | | | | | another 1 h | | |

TABLE 14

| | | | | | | | | HPLC |
|---|---|---|---|---|---|---|---|---|
| | | | Solvent | End | | | | purity |
| | | Conc. | volume | temp. | Solids | | | (%) at |
| Expt | Solvent | (mg/mL) | (μL) | (° C.) | obtained by | Observation | XRPD | 212 nm |
| CL01 | Water (H₂O) | 305.70 | 130 | 25 | evaporation at | glassy | Am. + few broad | |
| | | | | | 30 C., 20-21 mbar, | colorless | pks.: 3.7-6.5 & | |
| | | | | | around 18 h | material | 13.3-18.6 deg. | |
| CL02 | H₂O:THF | 302.74 | 180 | | evaporation at | white solid | Am. with a broad | |
| | (1:1 v/v) | | | | 30 C., 20-21 mbar, | | pk.: 3.7-6.5 | |
| | | | | | around 18 h | | | |
| CL03 | H₂O:EtOH | 303.34 | 185 | | evaporation at | white solid | Am. + pks. (some | 95.96 |
| | (1:1 v/v) | | | | 30 C., 20-21 mbar, | | of them from S2) | |
| | | | | | around 41 h | | | |
| CL04 | H₂O:2-PrOH | 298.73 | 220 | | evaporation at | white solid | Am. + pks. | 96.23 |
| | (1:1 v/v) | | | | 30 C., 20-21 mbar, | | | |
| | | | | | around 41 h | | | |
| CL05 | H₂O:MeCN | 306.96 | 200 | | evaporation at | white solid | Am. + few broad | |
| | (1:1 v/v) | | | | 30 C., 20-21 mbar, | | pks.: 3.7-6.5 & | |
| | | | | | around 18 h | | 13.3-18.6 deg. | |
| CL06 | H₂O:Acetone | 302.68 | 190 | | evaporation at | glassy white | Am. + few broad | |
| | (1:1 v/v) | | | | 30 C., 20-21 mbar, | solid | pks.: 3.7-6.5 & | |
| | | | | | around 18 h | | 13.3-18.6 deg. | |
| CL07 | Methanol | 120.76 | 250 | | evaporation at | white solid | Am. + few broad | |
| | (MeOH) | | | | 30 C., 20-21 mbar, | | pks: 3.7-6.5 & | |
| | | | | | 4.5 days | | 13.3-18.6 deg. | |
| CL08 | 2,2,2- | 62.18 | 400 | | evaporation at | white solid | Am. + few broad | |
| | Trifluoroethanol | | | | 30 C., 20-21 mbar, | | pks.: 3.7-6.5 & | |
| | (TFE) | | | | around 41 h | | 13.3-18.6 deg. | |
| CL09 | MeOH:EtOAc | 21.07 | 850 | | evaporation at | white solid | Am. + few broad | |
| | (1:1 v/v) | | | | 30 C., 20-21 mbar, | | pks.: 3.7-6.5 & | |
| | | | | | around 41 h | | 13.3-18.6 deg. | |
| CL10 | MeOH:Acetone | 21.14 | 800 | | evaporation at | white solid | S5, P.O. | 88.54 |
| | (1:1 v/v) | | | | 30 C., 20-21 mbar, | | | |
| | | | | | around 18 h | | | |
| CL11 | H₂O:THF | 302.74 | 180 | 5 | evaporation at | white solid | Am. with a broad | |
| | (1:1 v/v) | | | | 30 C., 20-21 mbar, | | pk.: 3.7-6.5 | |
| | | | | | around 18 h | | | |
| CL12 | H₂O:EtOH | 303.34 | 180 | | evaporation at | white solid | Am. + 1 pk. at | |
| | (1:1 v/v) | | | | 30 C., 20-21 mbar, | | 18.4 deg. | |
| | | | | | around 41 h | | | |
| CL13 | H₂O:2-PrOH | 298.73 | 220 | | evaporation at | white solid | Am. + few broad | |
| | (1:1 v/v) | | | | 30 C., 20-21 mbar, | | pks.: 3.7-6.5 & | |
| | | | | | around 18 h | | 13.3-18.6 deg. | |
| CL14 | H₂O:MeCN | 306.96 | 190 | | evaporation at | white solid | Am. + few broad | |
| | (1:1 v/v) | | | | 30 C., 20-21 mbar, | | pks: 3.7-6.5 & | |
| | | | | | around 18 h | | 13.3-18.6 deg. | |
| CL15 | H₂O:Acetone | 302.68 | 190 | | evaporation at | glassy white | Am. + few broad | |
| | (1:1 v/v) | | | | 30 C., 20-21 mbar, | solid | pks.: 3.7-6.5 & | |
| | | | | | around 18 h | | 13.3-18.6 deg. | |
| CL16 | Methanol | 120.76 | 250 | | evaporation at | white solid | S5, P.O. | 91.22 |
| | (MeOH) | | | | 30 C., 20-21 mbar, | | | |
| | | | | | around 18 h | | | |
| CL17 | 2,2,2- | 62.18 | 400 | | evaporation at | white solid | Am. + few broad | |
| | Trifluoroethanol | | | | 30 C., 20-21 mbar, | | pks.: 3.7-6.5 & | |
| | (TFE) | | | | around 41 h | | 13.3-18.6 deg. | |
| CL18 | MeOH:EtOAc | 21.07 | 850 | | evaporation at | white solid | Am. | |
| | (1:1 v/v) | | | | 30 C., 20-21 mbar, | | | |
| | | | | | around 41 h | | | |
| CL19 | MeOH:Acetone | 21.14 | 800 | | evaporation at | white solid | Am. + few pks. | 92.89 |
| | (1:1 v/v) | | | | 30 C., 20-21 mbar, | | (possible from S5) | |
| | | | | | around 18 h | | | |
| CL20 | Methanol | 120.76 | 250 | −20 | evaporation at | white solid | Am. + 3 pks. at | 93.97 |
| | (MeOH) | | | | 30 C., 20-21 mbar, | | 8.6, 17.2 & | |
| | | | | | around 18 h | | 44.4 deg. | |
| CL21 | 2,2,2- | 62.18 | 400 | | evaporation at | white solid | Am. + few broad | |
| | Trifluoroethanol | | | | 30 C., 20-21 mbar, | | pks.: 3.7-6.5 & | |
| | (TFE) | | | | 4.5 days | | 13.3-18.6 deg. | |
| CL22 | MeOH:EtOAc | 21.07 | ~850 | | evaporation at | white solid | Am. + pks. (some | 84.84 |
| | (1:1 v/v) | | | | 30 C., 20-21 mbar, | | of them from S5) | |
| | | | | | around 18 h | | | |
| CL23 | MeOH:Acetone | 21.14 | ~800 | | evaporation at | white solid | S5, P.O. | 90.24 |
| | (1:1 v/v) | | | | 30 C., 20-21 mbar, | | | |
| | | | | | around 18 h | | | |

Am. = amorphous;
P.O. = preferred orientation;
pks. = peaks

Based on the XRPD data, most experiments resulted in amorphous phase material. One different form (Form S5) was identified from the majority of experiments that involved MeOH as a solvent.

### Characterization of Form S5

Form S5 was obtained with preferred orientation from experiment CL16 with MeOH after vacuum drying of the remained solution after 7 days of cooling at 5° C.; or from experiments CL10 and CL23 with MeOH:acetone 1:1 v/v after vacuum drying of the remained solutions after 7 days of cooling at 25 and −20° C. Also, Form S5 was obtained with low crystallinity in mixture with amorphous phase from experiments CL19 (with MeOH:acetone 1:1 v/v after vacuum drying of the remained solution after 7 days of cooling at 5° C.) and CL22 (with MeOH:EtOAc 1:1 v/v after vacuum drying of the remained solution after 7 days of cooling at −20° C.). HPLC purity of these samples was generally good (>88.5%) at 212 nm (except for the CL22 experiment with 84.8%).

Based on the TG/DSC analysis of the material from experiment CL16 (FIG. 47), Form S5 was assigned as a solvated form with a first endothermic event suggesting a desolvation with Tₒₙₛₑₜ=120.3° C. and a mass loss of 5.867% (that can be attributed to 0.5 MeOH molecules, which corresponds with a theoretical mass of 3.59%). The second event was observed (likely a phase transformation) with Tₒₙₛₑₜ=188.1° C. A third, exothermic event that represents a recrystallization with T_{offset} at 205° C. was observed, and lastly, a melting event was observed at around 336° C. (Tₒₙₛₑₜ).

### Forward Anti-Solvent Addition

Three stock solutions (with water; methanol; and 2,2,2-trifluoroethanol) were prepared at 1.5 times solubility (determined from the Solubility Studies of Example 8) at RT under stirring at 700 rpm and heated at 40° C. for 1 hour. Not all mixtures dissolved at 40° C., and some of them were heated at 50° C. with extra stirring time or extra volume. The exact concentration for each stock solution is provided in Table 13. After the aforementioned time, the solutions were completely dissolved and the hot solutions were filtered with 0.45 m PTFE filters (or 0.2 μm Nylon filter for the case of water stock solution) and kept at the working temperature. The appropriate anti-solvents were added at 25° C. (500 rpm) in 3 steps with around 10 minutes delay in between, at a total volumetric ratio solvent/anti-solvent of 0.5, 1, and 5, respectively. After complete addition of the anti-solvents, the experiments were left to stir at 25° C. for 1 day, and the ones that yielded solids were harvested and analyzed by XRPD. Finally, the experiments that did not produce solids (experiments FASO1, 02, 03, 04) were left at 5° C. under stirring (500 rpm) for 1 week and checked periodically. After the time elapsed, no precipitation occurred, and the solutions were vacuum evaporated at 30° C., low pressure (final pressure=20-21 mbar) for ˜18 h (experiments FASO1 and 04) or for 4.5 days (experiments FASO2 and 03) and analyzed by XRPD. Based on the results obtained, some experiments were also analyzed by HPLC or TG/DSC. The results from forward anti-solvent addition at 25° C. (FAS) are presented in Table 15.

TABLE 15

| | | | Solvent | Anti- | AS | |
|---|---|---|---|---|---|---|
| | | Conc. | volume | Solvent | Volume | |
| Expt | Solvent | (mg/mL) | (μL) | (AS) | (μL) | Comments |
| FAS01 | Water (H₂O) | 305.70 | 300 | Ethanol | 1500 | |
| | | | | (EtOH) | | |
| FAS02 | | | 300 | 2-Propanol | 1500 | |
| | | | | (2-PrOH) | | |
| FAS03 | | | 300 | 2-Methyl-1- | 1500 | |
| | | | | propanol | | |
| FAS04 | | | 300 | THF | 1500 | |
| FAS05 | | | 300 | Acetone | 1500 | |
| FAS06 | | | 300 | MeCN | 1500 | after 2nd AS |
| | | | | | | add.: |
| | | | | | | opalescent |
| | | | | | | suspension |
| FAS07 | MeOH | 120.76 | 150 | Toluene | 750 | after 3rd AS |
| | | | | | | add.: |
| | | | | | | opalescent |
| | | | | | | solution |
| FAS08 | | | 150 | Ethyl acetate | 750 | |
| | | | | (EtOAc) | | |
| FAS09 | | | 150 | tert-Butyl | 750 | |
| | | | | methyl ether | | |
| FAS10 | | | 150 | Acetone | 750 | after 2nd AS |
| | | | | | | add.: |
| | | | | | | opalescent |
| | | | | | | solution; after |
| | | | | | | 10 minutes of |
| | | | | | | ageing became pp. |
| FAS11 | | | 150 | Methyl ethyl | 750 | |
| | | | | ketone (MEK) | | |
| FAS12 | | | 150 | MeCN | 750 | |
| FAS13 | 2,2,2- | 62.18 | 380 | Toluene | 1900 | aging at RT |
| | Trifluoroethanol | | | | | overnight: |
| | (TFE) | | | | | opalescent |
| | | | | | | solution |
| FAS14 | | | 380 | Ethyl | 1900 | |
| | | | | acetate | | |
| | | | | (EtOAc) | | |
| FAS15 | | | 380 | Isopropyl | 1900 | after around |
| | | | | acetate | | 10 min. of 3rd |
| | | | | | | AS add.: |
| | | | | | | opalescent |
| | | | | | | solution |
| FAS16 | | | 380 | n-Butyl | 1900 | |
| | | | | acetate | | |
| FAS17 | | | 380 | tert-Butyl | 1900 | |
| | | | | methyl ether | | |
| FAS18 | | | 380 | Diisopropyl | 1900 | |
| | | | | ether | | |
| FAS19 | | | 380 | Acetone | 1900 | |

| | | | | HPLC |
|---|---|---|---|---|
| | | | | purity |
| | Solids | | | (%) at |
| Expt | obtained by | Observation | XRPD | 212 nm |
| FAS01 | evaporation at | white solid | Am. | |
| | 30 C., 20-21 | | | |
| | mbar, around 18 h | | | |
| FAS02 | evaporation at | white solid | Am. | |
| | 30 C., 20-21 | | | |
| | mbar, 4.5 d ays | | | |
| FAS03 | evaporation at | white solid | similar to | |
| | 30 C., 20-21 | | CL01d (Am. + | |
| | mbar, 4.5 days | | few broad pks. | |
| | | | 3.7-6.5 & | |
| | | | 13.3-18.6 deg.) | |
| FAS04 | evaporation at | white solid | similar to | |
| | 30 C., 20-21 | | CL01d (Am. + | |
| | mbar, around 18 h | | few broad pks: | |
| | | | 3.7-6.5 & | |
| | | | 13.3-18.6 deg.) | |
| FAS05 | 3rd AS addition | white solid | similar to | |
| | at RT | | CL01d (Am. + | |
| | | | few broad pks.: | |
| | | | 3.7-6.5 & | |
| | | | 13.3-18.6 deg.) | |
| FAS06 | 3rd AS addition | white solid | Am. | |
| | at RT | | | |
| FAS07 | ageing at RT | white solid | S5, broad pks. | 96.81 |
| | overnight | | & l.c. | |
| FAS08 | 2nd AS addition | white solid | S5, l.c. | 92.45 |
| | at RT | | | |
| FAS09 | 2nd AS addition | white solid | l.c., S5 | 80.32 |
| | at RT | | | |
| FAS10 | before 3rd AS | white solid | l.c., S5 + 1 pk. | 84.49 |
| | addition at RT | | at 26.7 deg. | |
| FAS11 | 2nd AS addition | white solid | l.c., S5 | 91.33 |
| | at RT | | | |
| FAS12 | 2nd AS addition | white solid | l.c., S5 | 94.84 |
| | at RT | | | |
| FAS13 | aging at RT | white solid | S4 | 69.31 |
| | around 24 h | | | |
| FAS14 | aging at RT | white solid | SM, l.c. | |
| | after around 30 | | | |
| | min. of 3^{rd} AS | | | |
| | addition | | | |
| FAS15 | aging at RT | white solid | S4 | 93.75 |
| | after around 30 | | | |
| | min. of 3rd AS | | | |
| | addition | | | |
| FAS16 | aging at RT | white solid | SM | |
| | overnight | | | |
| FAS17 | 1st AS addition | white solid | S4 | 88.85 |
| | at RT | | | |
| FAS18 | 1st AS addition | white solid | S4, l.c. | 88.89 |
| | at RT | | | |
| FAS19 | aging at RT | white solid | SM (similar | |
| | after around 30 | | to SAS20) | |
| | min. of 3^{rd} AS | | | |
| | addition | | | |

Am. = amorphous;
l.c. = low crystallinity;
SM = starting material (i.e., NaCDC Form B);
P.O. = preferred orientation;
pks. = peaks

Based on the XRPD data, all of the experiments with water (and different anti-solvents) resulted in amorphous phase material. Two different forms were identified (Form S4 and Form S5) from the majority of experiments that involved MeOH or TFE as solvents.

### Characterization of Form S4

Form S4 was obtained from the majority of experiments with TFE: experiment FAS13 with toluene as anti-solvent; experiment FAS15 with iPrOAc; experiment FAS17 with MTBE; and experiment FAS18 with DIPE, respectively. All tested materials displayed a good HPLC purity (≥88.8%, except for experiment FAS13 with 69.3%) at 212 nm. Being obtained from the experiments with TFE, Form S4 was assigned as a solvated form of it.

### Characterization of Form S5

Form S5 was obtained (generally, with low crystallinity) from all FAS experiments with MeOH (experiment FAS07 with toluene as anti-solvent; experiment FASO8 with EtOAc; experiment FASO9 with MTBE; experiment FAS10 with acetone; experiment FAS11 with MEK; and experiment FAS12 with MeCN). HPLC purity of the materials good (>91.3%, except for experiment FASO9 with 80.3%) at 212 nm. An XPRD spectrum of the material obtained from experiment FASO8 is shown in FIG. 23A, and the corresponding data are summarized in Table S5-B.

TABLE S5-B

| 2-theta | d-Spacing | Rel. |
|---|---|---|
| Angle (°) | (Å) | Intensity (%) |
| 7.11 | 12.43 | 28.39 |
| 7.87 | 11.22 | 3.84 |
| 7.87 | 11.22 | 3.68 |
| 8.63 | 10.24 | 100.00 |
| 11.26 | 7.85 | 4.50 |
| 12.08 | 7.32 | 14.34 |
| 12.75 | 6.94 | 22.59 |
| 13.11 | 6.75 | 8.06 |
| 13.46 | 6.57 | 42.61 |
| 14.03 | 6.31 | 9.35 |
| 14.25 | 6.21 | 29.40 |
| 14.77 | 5.99 | 3.18 |
| 15.41 | 5.75 | 19.63 |
| 16.12 | 5.50 | 13.33 |
| 16.37 | 5.41 | 17.35 |
| 16.68 | 5.31 | 45.13 |
| 17.29 | 5.13 | 15.57 |
| 17.48 | 5.07 | 19.89 |
| 17.65 | 5.02 | 9.01 |
| 18.29 | 4.85 | 7.39 |
| 19.07 | 4.65 | 21.56 |
| 20.74 | 4.28 | 7.04 |
| 21.01 | 4.22 | 5.63 |
| 21.48 | 4.13 | 7.22 |
| 22.36 | 3.97 | 5.84 |
| 22.90 | 3.88 | 2.44 |
| 23.27 | 3.82 | 8.79 |
| 23.74 | 3.75 | 3.55 |
| 24.18 | 3.68 | 2.10 |
| 24.45 | 3.64 | 3.90 |
| 25.22 | 3.53 | 3.44 |
| 25.49 | 3.49 | 6.99 |
| 26.04 | 3.42 | 2.79 |
| 26.38 | 3.38 | 4.21 |
| 27.03 | 3.30 | 3.35 |
| 28.80 | 3.10 | 6.09 |
| 29.41 | 3.03 | 0.84 |
| 29.70 | 3.01 | 1.55 |
| 31.08 | 2.88 | 2.33 |
| 32.61 | 2.74 | 1.68 |
| 34.17 | 2.62 | 1.58 |
| 36.85 | 2.44 | 2.34 |
| 37.67 | 2.39 | 1.22 |
| 41.19 | 2.19 | 1.40 |

### Reverse Anti-Solvent Addition

Three stock solutions (with water; methanol; and 2,2,2-trifluoroethanol) were prepared at 1.5 times solubility (as determined in the Solubility Studies of Example 8) at RT under stirring at 700 rpm and heated at 40° C. for 1 hour. Not all mixtures dissolved at 40° C., and some of them were heated at 50° C. with extra stirring time or extra volume. The exact concentration for each stock solution is presented in Table 13. After the aforementioned time, the solutions were completely dissolved and the hot solutions were filtered with 0.45 m PTFE filters (or 0.2 m Nylon filter for the case of water stock solution) and kept at the working temperature.

Appropriate volumes of anti-solvents were added to HPLC vials or 8 mL flasks and equilibrated at 5° C. or 25° C. and hot filtered solutions (determined volumes of each solution to had, in the end, around 23 mg of NaCDC per experiment or around 39 mg per experiment in case of water solutions) were added dropwise in the appropriate vials, under stirring (500 rpm), the volumetric ratio between stock solution and anti-solvent being 1:10 (or at around 1:12 in case of experiments with water). All experiments were left stirring overnight at 5° C. or 25° C., and the ones that yielded solids were harvested, air-dried on filter paper, and analyzed by XRPD. The rest of the experiments were left to stir at 5° C. or 25° C. for 6 more days and checked periodically. If no precipitation occurred, the solutions were vacuum dried at 30° C., low pressure (final pressure=20-21 mbar) for ˜18 h (experiments RAS01, 04, 07, 10, 13 and 25) or for 4.5 days (experiments RAS2, 03, 08 and 09) and analyzed by XRPD. Based on the results obtained, some experiments were also analyzed by HPLC or TG/DSC. The results from reverse anti-solvent addition at 5° C. or 25° C. (RAS) are summarized in Table 16.

TABLE 16

| | | | Solvent | Anti- | | AS | |
|---|---|---|---|---|---|---|---|
| | | Conc. | volume | solvent | AS temp. | volume | |
| Expt | Solvent | (mg/mL) | (μL) | (AS) | (° C.) | (μL) | Comments |
| RAS01 | Water (H₂O) | 305.70 | 130 | Ethanol | 5 | 1500 | |
| | | | | (EtOH) | | | |
| RAS02 | | | 130 | 2-Propanol | | 1500 | |
| | | | | (2- PrOH) | | | |
| RAS03 | | | 130 | 2-Methyl-1- | | 1500 | |
| | | | | propanol | | | |
| RAS04 | | | 130 | THF | | 1500 | |
| RAS05 | | | 130 | Acetone | | 1500 | |
| RAS06 | | | 130 | MeCN | | 1500 | |
| RAS07 | | | 130 | Ethanol | 25 | 1500 | |
| | | | | (EtOH) | | | |
| RAS08 | | | 130 | 2-Propanol | | 1500 | |
| | | | | (2- PrOH) | | | |
| RAS09 | | | 130 | 2-Methyl-1- | | 1500 | |
| | | | | propanol | | | |
| RAS10 | | | 130 | THF | | 1500 | pp. after AS |
| | | | | | | | add. at 25 C. |
| | | | | | | | but rediss. |
| | | | | | | | instantly |
| RAS11 | | | 130 | Acetone | | 1500 | |
| RAS12 | | | 130 | MeCN | | 1500 | |
| RAS13 | Methanol | 120.76 | 190 | Toluene | 5 | 1900 | |
| RAS14 | | | 190 | Ethyl | | 1900 | |
| | | | | acetate | | | |
| | | | | (EtOAc) | | | |
| RAS15 | | | 190 | tert-Butyl | | 1900 | |
| | | | | methyl ether | | | |
| RAS16 | | | 190 | Acetone | | 1900 | |
| RAS17 | | | 190 | Methyl ethyl | | 1900 | |
| | | | | ketone (MEK) | | | |
| RAS18 | | | 190 | MeCN | | 1900 | |
| RAS19 | | | 190 | Toluene | 25 | 1900 | |
| RAS20 | | | 190 | Ethyl | | 1900 | |
| | | | | acetate | | | |
| | | | | (EtOAc) | | | |
| RAS21 | | | 190 | tert-Butyl | | 1900 | |
| | | | | methyl ether | | | |
| RAS22 | | | 190 | Acetone | | 1900 | |
| RAS23 | | | 190 | Methyl ethyl | | 1900 | |
| | | | | ketone (MEK) | | | |
| RAS24 | | | 190 | MeCN | | 1900 | |
| RAS25 | 2,2,2- | 62.18 | 380 | Toluene | 5 | 3800 | |
| | Trifluoroethanol | | | | | | |
| | (TFE) | | | | | | |
| RAS26 | | | 380 | Ethyl | | 3800 | |
| | | | | acetate | | | |
| | | | | (EtOAc) | | | |
| RAS27 | | | 380 | Isopropyl | | 3800 | |
| | | | | acetate | | | |
| RAS28 | | | 380 | n-Butyl | | 3800 | |
| | | | | acetate | | | |
| RAS29 | | | 380 | tert-Butyl | | 3800 | |
| | | | | methyl ether | | | |
| RAS30 | | | 380 | Diisopropyl | | 3800 | |
| | | | | ether | | | |
| RAS31 | | | 380 | Acetone | | 3800 | |
| RAS32 | | | 380 | Toluene | 25 | 3800 | opalescent |
| | | | | | | | solution after |
| | | | | | | | around 15 min. |
| | | | | | | | of AS add. |
| RAS33 | | | 380 | Ethyl | | 3800 | |
| | | | | acetate | | | |
| | | | | (EtOAc) | | | |
| RAS34 | | | 380 | Isopropyl | | 3800 | |
| | | | | acetate | | | |
| RAS35 | | | 380 | n-Butyl | | 3800 | |
| | | | | acetate | | | |
| RAS36 | | | 380 | tert-Butyl | | 3800 | |
| | | | | methyl ether | | | |
| RAS37 | | | 380 | Diisopropyl | | 3800 | |
| | | | | ether | | | |
| RAS38 | | | 380 | Acetone | | 3800 | |

| | | | | HPLC |
|---|---|---|---|---|
| | | | | purity |
| | Solids | | | (%) at |
| Expt | obtained by | Observation | XRPD | 212 nm |
| RAS01 | evaporation at | white solid | Am. | |
| | 30° C., 20-21 | | | |
| | mbar, around 18 h | | | |
| RAS02 | evaporation at | white solid | Am. | |
| | 30° C., 20-21 | | | |
| | mbar, 4.5 days | | | |
| RAS03 | evaporation at | white solid | similar to | |
| | 30° C., 20-21 | | CL02d (Am. | |
| | mbar, 4.5 days | | with a broad | |
| | | | pk: 3.7-6.5) | |
| RAS04 | evaporation at | white solid | Am. | |
| | 30° C., 20-21 | | | |
| | mbar, around 18 h | | | |
| RAS05 | AS addition at | white solid | l.c., similar S1 | 94.03 |
| | 5° C. | | or isomorphic | |
| | | | form of S1 | |
| | | | (from ASDS03) | |
| RAS06 | AS addition at | white solid | Am. | |
| | 5° C. | | | |
| RAS07 | evaporation at | white solid | similar to | |
| | 30° C., 20-21 | | CL01d (Am. + | |
| | mbar, around 18 h | | few broad pks: | |
| | | | 3.7-6.5 & | |
| | | | 13.3-18.6 deg.) | |
| RAS08 | evaporation at | white solid | Am. | |
| | 30° C., 20-21 | | | |
| | mbar, 4.5 days | | | |
| RAS09 | evaporation at | white solid | similar to | |
| | 30° C., 20-21 | | CL01d (Am. + | |
| | mbar, 4.5 days | | few broad pks: | |
| | | | 3.7-6.5 & | |
| | | | 13.3-18.6 deg.) | |
| RAS10 | evaporation at | white solid | Am. | |
| | 30° C., 20-21 | | | |
| | mbar, around 18 h | | | |
| RAS11 | AS addition at | white solid | similar S1 or | 97.38 |
| | 25° C. | | isomorphic | |
| | | | form of S1, l.c. | |
| | | | (from ASDS03) | |
| RAS12 | AS addition at | white solid | Am. | |
| | 25° C. | | | |
| RAS13 | evaporation at | white solid | Am. | |
| | 30° C., 20-21 | | | |
| | mbar, around 18 h | | | |
| RAS14 | AS addition at | white solid | S5, l.c. | 95.61 |
| | 5° C. | | | |
| RAS15 | AS addition at | white solid | S5 | 91.87 |
| | 5° C. | | | |
| RAS16 | AS addition at | white solid | l.c., S5 | 93.50 |
| | 5° C. | | | |
| RAS17 | AS addition at | white solid | S5 | 87.28 |
| | 5° C. | | | |
| RAS18 | AS addition at | white solid | S5, l.c. | 97.56 |
| | 5° C. | | | |
| RAS19 | AS addition at | white solid | S5 | 96.48 |
| | 25° C. after | | | |
| | around 2 min. | | | |
| RAS20 | AS addition at | white solid | S5 | 95.27 |
| | 25 C. | | | |
| RAS21 | AS addition at | white solid | S5 | |
| | 25° C. | | | |
| RAS22 | AS addition at | white solid | S5 | 94.97 |
| | 25° C. | | | |
| RAS23 | AS addition at | white solid | S5 | 91.91 |
| | 25° C. | | | |
| RAS24 | AS addition at | white solid | S5, l.c. | 95.46 |
| | 25° C. | | | |
| RAS25 | evaporation at | white solid | Am. | |
| | 30° C., 20-21 | | | |
| | mbar, around 18 h | | | |
| RAS26 | AS addition at | white solid | SM | |
| | 5° C. | | | |
| RAS27 | AS addition at | white solid | SM | |
| | 5° C. | | | |
| RAS28 | AS addition at | white solid | l.c., S4 | 93.64 |
| | 5° C. | | | |
| RAS29 | AS addition at | white solid | l.c., S4 + pks. | 89.43 |
| | 5° C. | | | |
| RAS30 | AS addition at | white solid | S4 + pks. | 91.39 |
| | 5° C. | | | |
| RAS31 | AS addition at | white solid | SM | |
| | 5° C. | | | |
| RAS32 | aging at 25° C. | white solid | S4 | 95.73 |
| | overnight (fine | | | |
| | pp.) | | | |
| RAS33 | aging at 25° C. | white solid | similar to or | 96.13 |
| | after around 10 | | isomorph with S6 | |
| | min. of AS add. | | (from EV08) P.O. | |
| | (fine pp.) | | | |
| RAS34 | aging at 25° C. | white solid | similar to or | 82.27 |
| | after around 10 | | isomorph with | |
| | min. of AS add. | | S3 (from SAS07) | |
| | (fine pp.) | | | |
| RAS35 | aging at 25° C. | white solid | SM | |
| | after around 10 | | | |
| | min. of AS add. | | | |
| | (fine pp.) | | | |
| RAS36 | AS addition at | white solid | S15 | 85.95 |
| | 25° C. | | | |
| RAS37 | AS addition at | white solid | S13 + pks. | 81.69 |
| | 25° C. | | | |
| RAS38 | aging at 25° C. | white solid | SM | |
| | after around 10 | | | |
| | min. of AS add. | | | |
| | (fine pp.) | | | |

Am. = amorphous;
l.c. = low crystallinity;
SM = starting material (i.e., NaCDC Form B);
P.O. = preferred orientation;
pks. = peaks;
pp. = precipitate

Based on the XRPD data, many of the materials recovered were in an amorphous phase or, in several cases, were obtained as starting material (NaCDC Form B). Also, seven different forms were identified—Form S1 (or similar to and/or isomorphic with Form S1), Form S3 (or similar to and/or isomorphic with Form S3), Form S4, Form S5, Form S6 (or similar to and/or isomorphic with Form S6), Form S13, and Form S15.

### Characterization of Form S1

Form S1 (or a form similar to and/or isomorphic with Form S1) was obtained from experiments RAS05 and RAS11 (water stock solution and acetone as anti-solvent) at 5° C. and 25° C., respectively. HPLC purity of the materials was good (≥94.0%) at 212 nm. An XRPD pattern of the material from experiment RAS11 is shown in FIG. 24A.

The precipitate that formed into the water solution in the presence of acetone (as anti-solvent) was stored at RT for 2 weeks before being subjected to thermal analysis. In this case, the majority of material obtained from experiment RAS11 was dried at RT on filter paper for 1-2 h, and after that the recovered material was analyzed by XRPD. The results indicated that the stored material had a high tendency of amorphization with several peaks (some of them similar to and/or isomorphic with Form S1) (FIG. 48).

Based on the TG/DSC analysis (FIG. 49), the material from experiment RAS11 that had been stored for 2 weeks was assigned as a mixed solvate with acetone and water. As such, the first endothermic event suggests a desolvation with Tₒₙₛₑₜ=41.9° C. and a mass loss of 5.356% (that can be attributed to 0.5 acetone molecules, which corresponds to a theoretical mass of 6.14%). The second endothermic event suggests another desolvation with Tₒₙₛₑₜ=107.2° C. and a mass loss of 1.237% (that can be attributed with 0.25 molecules of water, which corresponds to a theoretical mass of 1.04%). A recrystallization event was observed at around 198.4° C., and lastly, a melting event was observed at around 327° C. (Tₒₙₛₑₜ). The melting Tₒₙₛₑₜ of this sample was different from the starting material (NaCDC Form B), meaning that the form may be a solvate of another potential anhydrous form.

### Characterization of Form S3

Form S3 (or a form similar to and/or isomorphic with Form S3) was obtained from experiment RAS34 (TFE stock solution and iPrOAc as anti-solvent) at 25° C. HPLC purity of the material was 82.3% at 212 nm. An XRPD spectrum of the material obtained from experiment RAS34 is shown in FIG. 50A.

The precipitate that formed into the TFE solution in the presence of iPrOAC (as anti-solvent) was stored at RT for 2 weeks before being subjected to thermal analysis. In this case, the majority of material from experiment RAS34 dried at RT on filter paper for 1-2 h, and after that the recovered material was analyzed by XRPD. The results indicated that the material from experiment RAS34 that had been stored remained unchanged, and was Form S3 (or a form similar to and/or isomorphic with Form S3).

Based on TG/DSC analysis (FIG. 50B), the material obtained from experiment RAS34 that had been stored for 2 weeks was assigned as a solvate with TFE or iPrOAc. As such, the first, endothermic event suggested a desolvation with Tₒₙₛₑₜ=52.3° C. and a mass loss of 1.323% (that can be attributed to 0.0625 molecules of TFE or iPrOAc, which corresponds to a theoretical mass of 1.21% or 1.23%, respectively), likely residual solvent. The second, exothermic event suggested a recrystallization event and is observed at around 195° C. Lastly, a melting event at around 336° C. (Tₒₙₛₑₜ) was observed.

### Characterization of Form S4

Form S4 was obtained from four experiments, all using TFE stock solution: experiment RAS28 (with low crystallinity; nBuOAc as anti-solvent at 5° C.), experiment RAS29 (with low crystallinity & extra peaks; MTBE as anti-solvent at 5° C.), experiment RAS30 (with extra peaks; DIPE as anti-solvent at 5° C.), and experiment RAS32 (toluene as anti-solvent at 25° C.). The materials had good HPLC purities (>89.4%) at 212 nm.

### Characterization of Form S5

Form S5 was obtained from the majority of experiments with MeOH stock solution and different anti-solvents: experiments RAS14 and RAS20 with EtOAc at 5 and 25° C.; experiments RAS15 and RAS21 at 5 and 25° C.; experiments RAS16 and RAS22 with acetone at 5 and 25° C.; experiments RAS17 and RAS23 with MEK at 5 and 25° C.; experiments RAS18 and RAS24 with MeCN at 5 and 25° C.; and experiment RAS19 with toluene at 25° C. In some cases, the Form S5 was obtained with low crystallinity (i.e., in experiments RAS14, 16, 18, 24). HPLC purities of the materials were good (>87.3%) at 212 nm.

### Characterization of Form S6

Form S6 (or a form similar to and/or isomorphic with Form S6) was obtained, with preferred orientation, from experiment RAS33 (TFE stock solution and EtOAc as anti-solvent) at 25° C. HPLC purity of the material was 96.1% at 212 nm. An XRPD spectrum of the material from experiment RAS33 is shown in FIG. 50A, and the corresponding data are summarized in Table S6:

TABLE S6

| 2-theta | d-Spacing | Rel. |
|---|---|---|
| Angle (°) | (Å) | Intensity (%) |
| 4.97 | 17.75 | 100.00 |
| 7.61 | 11.60 | 22.58 |
| 9.95 | 8.88 | 3.88 |
| 10.18 | 8.68 | 3.93 |
| 10.56 | 8.37 | 7.07 |
| 10.68 | 8.28 | 8.44 |
| 11.51 | 7.68 | 27.48 |
| 12.00 | 7.37 | 26.11 |
| 12.55 | 7.05 | 12.77 |
| 13.55 | 6.53 | 8.38 |
| 14.21 | 6.23 | 5.10 |
| 14.34 | 6.17 | 6.85 |
| 14.91 | 5.94 | 11.00 |
| 15.32 | 5.78 | 18.36 |
| 16.46 | 5.38 | 11.07 |
| 16.76 | 5.28 | 11.47 |
| 17.94 | 4.94 | 10.74 |
| 19.11 | 4.64 | 7.70 |
| 19.27 | 4.60 | 11.18 |
| 19.93 | 4.45 | 9.76 |
| 20.45 | 4.34 | 3.85 |
| 21.33 | 4.16 | 5.33 |
| 21.75 | 4.08 | 3.87 |
| 22.22 | 4.00 | 2.69 |
| 22.27 | 3.99 | 4.04 |
| 23.67 | 3.76 | 2.51 |
| 25.36 | 3.51 | 3.38 |
| 26.63 | 3.34 | 2.80 |

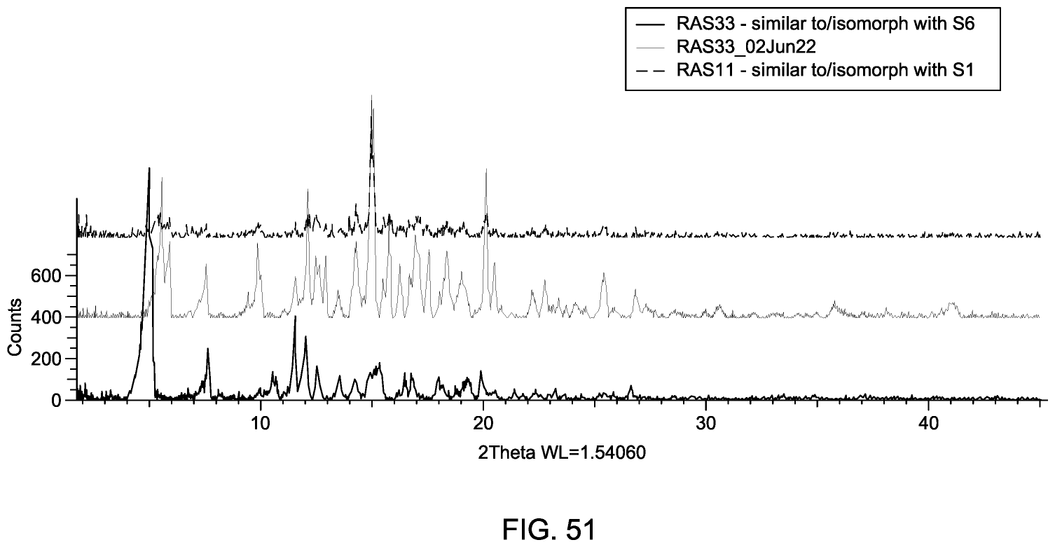

The precipitate that formed into the TFE solution in the presence of EtOAc (as anti-solvent) was stored at RT for 2 weeks before being subjected to thermal analysis. In this case, the majority of material obtained from experiment RAS33 was dried at RT on filter paper for 1-2 h, and after that the recovered material was analyzed by XRPD. The results indicated that the material from experiment RAS33 that had been stored for 2 weeks transformed into Form S1 (or a form similar to and/or isomorphic with Form S1) (FIG. 51).

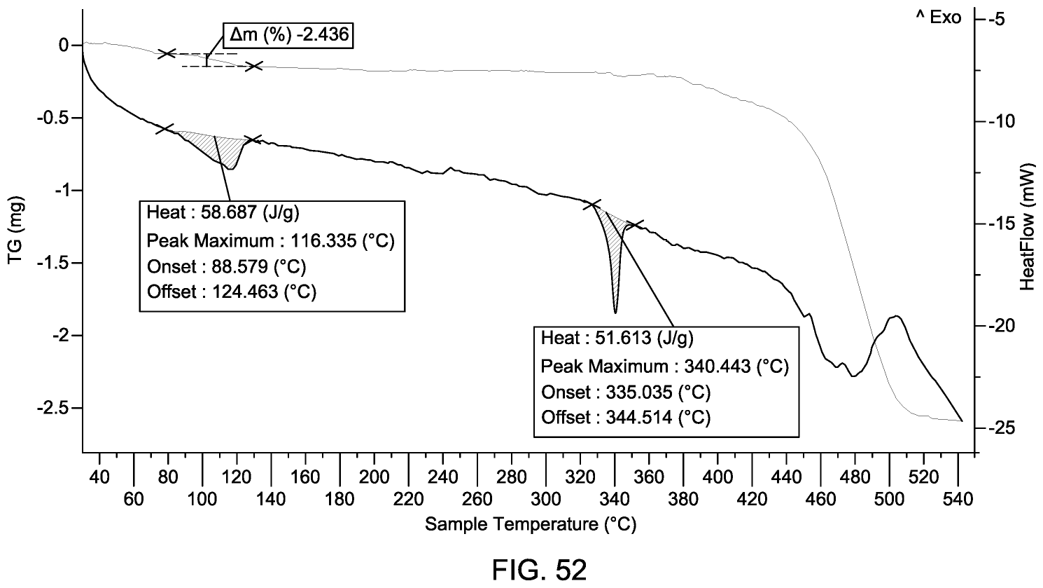

Based on TG/DSC analysis (FIG. 52), the material from experiment RAS33 that had been stored for 2 weeks was assigned as a solvate with EtOAc. As such, the first, endothermic event suggested a desolvation with Tₒₙₛₑₜ=88.6° C. and a mass loss of 2.436% (that can be attributed to 0.125 molecules of EtOAc, which corresponds to a theoretical mass of 2.191%). A melting event at around 335° C. (Tₒₙₛₑₜ) was observed.

### Characterization of Form S13

Form S13 (with extra peaks) was obtained from experiment RAS37 with TFE (and DIPE as anti-solvent) at 25° C. HPLC purity of the sample was 81.7% at 212 nm. XRPD analysis of the material obtained from experiment RAS37 is shown in FIG. 53, and the corresponding data are summarized in Table S13:

TABLE S13

| 2-theta | d-Spacing | Rel. |
|---|---|---|
| Angle (°) | (Å) | Intensity (%) |
| 4.95 | 17.83 | 100.00 |
| 5.54 | 15.95 | 34.11 |
| 5.85 | 15.11 | 17.49 |
| 7.53 | 11.74 | 29.34 |
| 9.40 | 9.40 | 15.53 |
| 9.85 | 8.97 | 28.76 |
| 10.59 | 8.35 | 9.97 |
| 11.47 | 7.71 | 25.72 |
| 11.55 | 7.65 | 44.98 |
| 12.02 | 7.36 | 24.86 |
| 12.12 | 7.30 | 49.01 |
| 12.56 | 7.04 | 25.90 |
| 12.89 | 6.86 | 23.08 |
| 13.48 | 6.56 | 14.54 |
| 14.25 | 6.21 | 32.09 |
| 14.29 | 6.19 | 26.43 |
| 15.01 | 5.90 | 83.87 |
| 15.07 | 5.88 | 66.94 |
| 15.74 | 5.63 | 34.71 |
| 16.34 | 5.42 | 24.76 |
| 16.65 | 5.32 | 23.17 |
| 17.54 | 5.05 | 20.23 |
| 18.32 | 4.84 | 22.05 |
| 20.08 | 4.42 | 42.55 |
| 20.44 | 4.34 | 15.79 |
| 21.64 | 4.10 | 9.17 |
| 22.23 | 4.00 | 11.34 |
| 22.76 | 3.90 | 14.07 |
| 25.36 | 3.51 | 15.89 |
| 26.83 | 3.32 | 19.81 |
| 30.85 | 2.90 | 8.18 |
| 35.54 | 2.52 | 6.74 |

### Characterization of Form S15

Form S15 was obtained from experiment RAS36 with TFE (and MTBE as anti-solvent) at 25° C. HPLC purity of the sample was 86% at 212 nm.

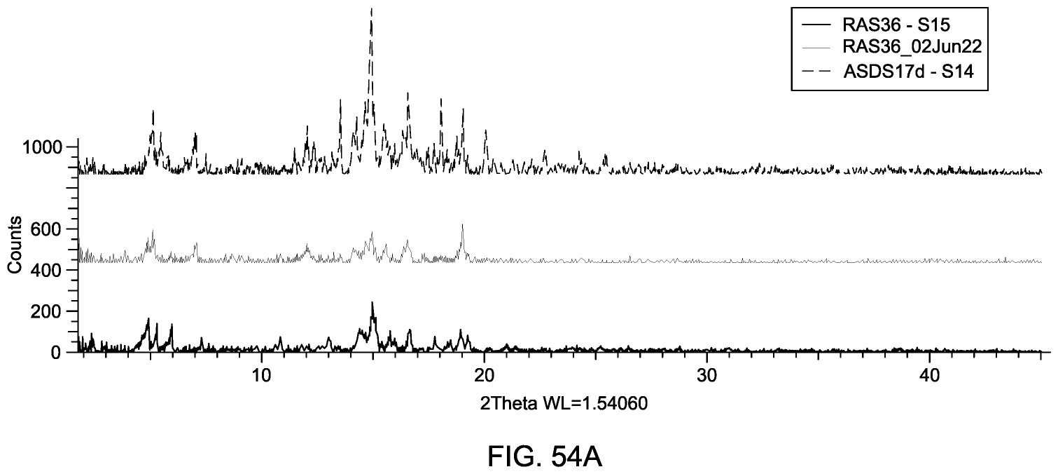

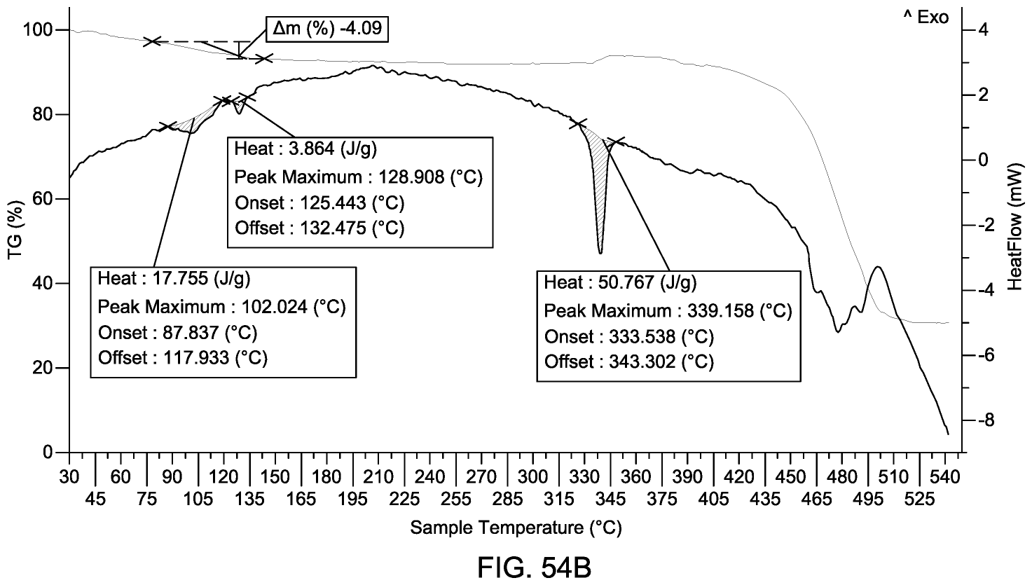

The precipitate that formed into the TFE solution in the presence of MTBE (as anti-solvent) was stored at RT for 2 weeks before being subjected to thermal analysis. In this case, the majority of the material obtained from experiment RAS36 that had been stored for 2 weeks was dried at RT on filter paper for 1-2 h, and after that the recovered material was analyzed by XRPD. The results indicated that the material obtained from experiment RAS36 that had been stored for 2 weeks transformed into Form S14 (or a form similar to and/or isomorphic with form with Form S14) or into a mixture of Form S15 and another form (FIG. 54A).

Based on TG/DSC analysis (FIG. 54B), the material obtained from experiment RAS36 that had been stored for 2 weeks was assigned as a mixed solvate with MTBE and TFE. As such, the first, broad, endothermic event suggests a desolvation with Tₒₙₛₑₜ=87.8° C. The second event suggested a desolvation with Tₒₙₛₑₜ=125.4° C. Both processes combined are assigned a mass loss of 4.09%. An inflexion point at around 185° C. might suggest a recrystallization. Lastly, a melting event was observed with Tₒₙₛₑₜ at around 334° C.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A crystalline solid form of sodium chenodeoxycholate, wherein the solid form is anhydrous and is characterized by one or more peaks in its XRPD pattern selected from those at about 6.75, about 8.14, about 9.79, about 14.02, about 16.10, and about 18.63 degrees 2-theta.

2. The solid form of claim 1, wherein the solid form is characterized by peaks in its XRPD pattern selected from those at about 6.75, about 8.14, about 9.79, about 14.02, about 16.10, and about 18.63 degrees 2-theta.

3. The solid form of claim 2, wherein the solid form is characterized by peaks in its XRPD pattern at substantially all of:
2θ ± 0.2 [°]
6.75
8.14
9.79
11.33
13.27
13.56
14.02
14.79
15.05
16.10
16.37
16.78
17.74
18.63
19.71
20.00
20.35
21.89
22.90.

4. The solid form of any one of claims 1-3, wherein the solid form is characterized by one or more of:
(i) an XRPD pattern substantially similar to that depicted in FIG. 5;
(ii) a DSC pattern showing no thermal event from room temperature to around 288° C.;
(iii) a DSC pattern substantially similar to that depicted in FIG. 6;
(iv) a TGA pattern showing a weight loss of <0.1% up to 150° C.; or
(v) a TGA pattern substantially similar to that depicted in FIG. 7.

5. A crystalline solid form of sodium chenodeoxycholate prepared by a method comprising steps of: providing a mixture of chenodeoxycholic acid in n-butanol; adding to the mixture an aqueous solution of sodium hydroxide; heating the mixture (e.g., to azeotropic reflux); and removing the solvent to provide the crystalline solid form of sodium chenodeoxycholate.

6. A crystalline solid form of sodium chenodeoxycholate, wherein the solid form is a hydrate and is characterized by one or more peaks in its XRPD pattern selected from those at about 6.07, about 6.55, about 10.72, about 14.64, about 15.06, about 17.58, and about 18.34 degrees 2-theta.

7. The solid form of claim 6, wherein the solid form is characterized by peaks in its XRPD pattern selected from those at about 6.07, about 6.55, about 10.72, about 14.64, about 15.06, about 17.58, and about 18.34 degrees 2-theta.

8. The solid form of claim 7, wherein the solid form is characterized by peaks in its XRPD pattern at substantially all of:
2θ ± 0.2 [°]
6.07
6.55
8.36
9.92
10.72
11.55
11.93
12.99
13.36
14.64
15.06
15.54
15.93
16.68
16.99
17.31
17.58
18.07
18.34.

9. The solid form of any one of claims 6-8, wherein the solid form is characterized by one or more of:
(i) an XRPD pattern substantially similar to that depicted in FIG. 1;
(ii) a DSC pattern showing loss of water starting just above ambient temperature and continuing to about 150° C.;
(iii) a DSC pattern substantially similar to that depicted in FIG. 2;
(iv) a TGA pattern showing a weight loss of 4.3% up to 150° C.; or
(v) a TGA pattern substantially similar to that depicted in FIG. 3.

10. A crystalline solid form of sodium chenodeoxycholate prepared by a method comprising steps of: providing a mixture of chenodeoxycholic acid in methyl isobutyl ketone; adding to the mixture an aqueous solution of sodium hydroxide; heating the mixture (e.g., to azeotropic reflux); and removing the solvent to provide the crystalline solid form of sodium chenodeoxycholate.

11. A crystalline solid form of sodium chenodeoxycholate, wherein the solid form is:
(i) characterized by one or more peaks in its XRPD pattern selected from those at about 5.45, about 5.80, about 7.46, about 9.76, about 12.40, about 14.88, and about 20.02 degrees 2-theta;
(ii) characterized by one or more peaks in its XRPD pattern selected from those at about 7.11, about 7.78, about 9.81, about 12.58, about 12.96, and about 13.54 degrees 2-theta;
(iii) characterized by one or more peaks in its XRPD pattern selected from those at about 5.00, about 7.56, about 10.56, about 11.45, about 11.93, and about 12.46 degrees 2-theta;
(iv) characterized by one or more peaks in its XRPD pattern selected from those at about 7.07, about 7.65, about 9.70, about 13.43, about 15.02, about 16.52, and about 16.96 degrees 2-theta;
(v) characterized by one or more peaks in its XRPD pattern selected from those at about 7.11, about 8.63, about 12.08, about 12.75, about 13.46, about 14.25, and about 16.68 degrees 2-theta;
(vi) characterized by one or more peaks in its XRPD pattern selected from those at about 8.47, about 9.90, about 14.36, about 15.26, about 17.00, and about 17.72 degrees 2-theta;
(vii) characterized by one or more peaks in its XRPD pattern selected from those at about 5.10, about 7.00, about 13.52, about 14.23, about 15.46, and about 18.78 degrees 2-theta;
(viii) characterized by one or more peaks in its XRPD pattern selected from those at about 5.47, about 7.48, about 9.82, about 12.66, and about 15.07 degrees 2-theta;
(ix) characterized by one or more peaks in its XRPD pattern selected from those at about 5.13, about 7.01, about 8.69, about 9.11, about 13.55, about 14.91, and about 15.53 degrees 2-theta;
(x) characterized by one or more peaks in its XRPD pattern selected from those at about 4.82, about 5.22, about 5.89, about 10.81, about 13.00, about 15.00, and about 18.94 degrees 2-theta;
(xi) characterized by one or more peaks in its XRPD pattern selected from those at about 4.95, about 7.53, about 9.85, about 11.55, about 12.12, and about 15.01 degrees 2-theta; or
(xii) characterized by one or more peaks in its XRPD pattern selected from those at about 5.15, about 5.50, about 7.05, about 12.04, about 14.90, and about 16.56 degrees 2-theta.

12. The solid form of claim 11, wherein the solid form is characterized by one or more peaks in its XRPD pattern selected from those at about 5.45, about 5.80, about 7.46, about 9.76, about 12.40, about 14.88, and about 20.02 degrees 2-theta.

13. The solid form of claim 12, wherein the solid form is characterized by peaks in its XRPD pattern at substantially all of those listed in Table S1-A or Table S1-B.

14. The solid form of claim 11, wherein the solid form is characterized by one or more peaks in its XRPD pattern selected from those at about 7.11, about 7.78, about 9.81, about 12.58, about 12.96, and about 13.54 degrees 2-theta.

15. The solid form of claim 14, wherein the solid form is characterized by peaks in its XRPD pattern at substantially all of those listed in Table S2.

16. The solid form of claim 11, wherein the solid form is characterized by one or more peaks in its XRPD pattern selected from those at about 5.00, about 7.56, about 10.56, about 11.45, about 11.93, and about 12.46 degrees 2-theta.

17. The solid form of claim 16, wherein the solid form is characterized by peaks in its XRPD pattern at substantially all of those listed in Table S3, Table S6, or Table S11.

18. The solid form of claim 11, wherein the solid form is characterized by one or more peaks in its XRPD pattern selected from those at about 7.07, about 7.65, about 9.70, about 13.43, about 15.02, about 16.52, and about 16.96 degrees 2-theta.

19. The solid form of claim 18, wherein the solid form is characterized by peaks in its XRPD pattern at substantially all of those listed in Table S4.

20. The solid form of claim 11, wherein the solid form is characterized by one or more peaks in its XRPD pattern selected from those at about 7.11, about 8.63, about 12.08, about 12.75, about 13.46, about 14.25, and about 16.68 degrees 2-theta.

21. The solid form of claim 20, wherein the solid form is characterized by peaks in its XRPD pattern at substantially all of those listed in Table S5.

22. The solid form of claim 11, wherein the solid form is characterized by one or more peaks in its XRPD pattern selected from those at about 8.47, about 9.90, about 14.36, about 15.26, about 17.00, and about 17.72 degrees 2-theta.

23. The solid form of claim 22, wherein the solid form is characterized by peaks in its XRPD pattern at substantially all of those listed in Table S7.

24. The solid form of claim 11, wherein the solid form is characterized by one or more peaks in its XRPD pattern selected from those at about 5.10, about 7.00, about 13.52, about 14.23, about 15.46, and about 18.78 degrees 2-theta.

25. The solid form of claim 24, wherein the solid form is characterized by peaks in its XRPD pattern at substantially all of those listed in Table S9-a.

26. The solid form of claim 11, wherein the solid form is characterized by one or more peaks in its XRPD pattern selected from those at about 5.47, about 7.48, about 9.82, about 12.66, and about 15.07 degrees 2-theta.

27. The solid form of claim 26, wherein the solid form is characterized by peaks in its XRPD pattern at substantially all of those listed in Table S9-b.

28. The solid form of claim 11, wherein the solid form is characterized by one or more peaks in its XRPD pattern selected from those at about 5.13, about 7.01, about 8.69, about 9.11, about 13.55, about 14.91, and about 15.53 degrees 2-theta.

29. The solid form of claim 28, wherein the solid form is characterized by peaks in its XRPD pattern at substantially all of those listed in Table S10.

30. The solid form of claim 11, wherein the solid form is characterized by one or more peaks in its XRPD pattern selected from those at about 4.82, about 5.22, about 5.89, about 10.81, about 13.00, about 15.00, and about 18.94 degrees 2-theta.

31. The solid form of claim 30, wherein the solid form is characterized by peaks in its XRPD pattern at substantially all of those listed in Table S12 or Table S15.

32. The solid form of claim 11, wherein the solid form is characterized by one or more peaks in its XRPD pattern selected from those at about 4.95, about 7.53, about 9.85, about 11.55, about 12.12, and about 15.01 degrees 2-theta.

33. The solid form of claim 32, wherein the solid form is characterized by peaks in its XRPD pattern at substantially all of those listed in Table S13.

34. The solid form of claim 11, wherein the solid form is characterized by one or more peaks in its XRPD pattern selected from those at about 5.15, about 5.50, about 7.05, about 12.04, about 14.90, and about 16.56 degrees 2-theta.

35. The solid form of claim 34, wherein the solid form is characterized by peaks in its XRPD pattern at substantially all of those listed in Table S14.

36. A crystalline solid form of sodium chenodeoxycholate obtainable from a process described herein.

37. A pharmaceutical composition comprising the solid form of any one of claims 1-36 and a pharmaceutically acceptable carrier.

38. The pharmaceutical composition of claim 37, wherein the pharmaceutical composition is solid.

39. The pharmaceutical composition of claim 37 or 38, wherein the pharmaceutical composition is formulated for oral administration.

40. A pharmaceutical composition prepared by a method comprising steps of: providing the solid form of any one of claims 1-36; and formulating the solid form with suitable excipients to provide the pharmaceutical composition.

41. A pharmaceutical composition comprising:
a first portion comprising a bile acid or salt thereof, wherein the first portion is configured for immediate release in the colon of a subject;
a second portion, adjacent to the first portion, the second portion comprising a bile acid or salt thereof and wherein the second portion is configured for extended release in the colon of a subject; and
a degradable or erodible coating associated with the pharmaceutical composition,
wherein at least one of the first portion or the second portion comprises the solid form of any one of claims 1-36.

42. A pharmaceutical composition comprising:
a first portion comprising a bile acid or salt thereof, wherein the first portion is configured for immediate release in the colon of a subject;
a second portion, adjacent to the first portion, the second portion comprising a bile acid or salt thereof and wherein the second portion is configured for extended release in the colon of a subject; and
a degradable or erodible coating associated with the pharmaceutical composition,
wherein at least one of the first portion or the second portion comprises the solid form of any one of claims 1-36,
prepared by a method comprising steps of:
providing the solid form of any one of claims 1-36; and
formulating the solid form with suitable excipients to provide the pharmaceutical composition.

43. The pharmaceutical composition of claim 41 or 42, wherein the pharmaceutical composition is a tablet.

44. The pharmaceutical composition of any one of claims 41-43, wherein the bile acid or salt thereof in the first portion and the second portion is chenodeoxycholic acid or salt thereof.

45. The pharmaceutical composition of claim 44, wherein the first portion and the second portion comprise the solid form of any one of embodiments 1-36.

46. The pharmaceutical composition of any one of claims 41-45, wherein the coating is or comprises Eudragit S100.

47. A method comprising administering the solid form of any one of claims 1-36 or the pharmaceutical composition of any one of claims 37-46 to a subject in need thereof.

48. The method of claim 47, comprising orally administering the solid form of any one of claims 1-36 or the pharmaceutical composition of any one of claims 37-46.

49. A method of treating a disease, disorder, or condition, comprising administering the solid form of any one of claims 1-36 or the pharmaceutical composition of any one of claims 37-46 to a subject in need thereof.

50. The method of any one of claims 47-49, wherein the subject is suffering from a gastrointestinal disease, disorder, or condition.

51. The method of any one of claims 47-49, wherein the subject is suffering from constipation.

52. The method of any one of claims 47-49, wherein the subject is suffering from irritable bowel syndrome with constipation (IBS-C).

53. A method of preparing the solid form of any one of claims 1-10, wherein the method comprises steps of:
providing chenodeoxycholic acid; and
contacting chenodeoxycholic acid with a suitable base in a suitable solvent to provide the solid form.

54. The method of claim 53, wherein the suitable base is sodium hydroxide.

55. The method of claim 53 or 54, wherein the suitable solvent is selected from methyl isobutyl ketone, n-butanol, and water.

56. A method of preparing the pharmaceutical composition of any one of claims 37-46, wherein the method comprises steps of:
providing the solid form of any one of claims 1-36; and
formulating the solid form with suitable excipients to provide the pharmaceutical composition.
